(19) United States
(12) Patent Application Publication
(10) Pub. No.: US 2026/0308502 A1
(43) Pub. Date: Oct. 8, 2026

(54) STACKED BIOREACTOR FOR CELLULAR THERAPEUTICS

(71) Applicant: BRUKER CELLULAR ANALYSIS, INC., Emeryville, CA (US)

(72) Inventors: Jonathan Cloud Dragon Hubbard, Oakland, CA (US); Vivek Jadhav, Union City, CA (US); Darcy K. Kelly-Greene, Emeryville, CA (US); Kelek Olais, Emeryville, CA (US); Angel Navas Angeles, Emeryville, CA (US); Chase McRobie, Emeryville, CA (US)

(73) Assignee: BRUKER CELLULAR ANALYSIS, INC., Emeryville, CA (US)

(21) Appl. No.: 19/482,207

(22) PCT Filed: Jun. 7, 2024
(86) PCT No.: PCT/US2024/033152

§ 371 Date: Nov. 6, 2025

Related U.S. Application Data
(60) Provisional application No. 63/507,060, filed on Jun. 8, 2023.
(60) Provisional application No. 63/601,684, filed on Nov. 21, 2023.

(51) Int. Cl. C12M 1/00; C12M 1/04

(52) U.S. Cl. CPC C12M 23/58; C12M 23/24; C12M 29/04

(57) ABSTRACT

A bioreactor assembly, comprises a plurality of bioreactor units arranged in a flow sequence, and at least one flow path extending through the bioreactor units. The plurality of bioreactor units may be stacked on top of each other. Each of the plurality of bioreactor units may be configured for expanding a population of biological cells. The flow path(s) selectively extends through the plurality of bioreactor units, such that selected ones of the plurality of bioreactor units may be added to the flow path(s), and selected ones of the plurality of bioreactor units may be removed from the flow path(s).

### FIELD

The present disclosure relates generally to bioreactors, and more particularly, to bioreactors for expanding cell populations.

### BACKGROUND

Cellular therapeutics offer a potentially powerful approach to successfully treating many different diseases. To date, though, few cellular therapies have been approved for use in patients, in part due to the difficulties of manufacturing the therapies in a consistent and predictable manner. Moreover, the available approaches for cell therapy manufacturing have been cost-prohibitive and lack scalability. Product quality and release testing are a significant portion of both the cost and lead time for the manufacture and delivery of a cell therapy to a patient.

Adoptive immunotherapy with ex vivo-derived T lymphocytes (T-cells) is a rapidly progressing field that has application for the treatment of disease in a range of clinical settings, including viral diseases associated with immune-compromise and cancer. T lymphocytes (T-cells) play a specialized, critical role in the antigen-specific immune response. A single administration of adoptive T-cell therapy can require billions of human T-cells, and thus, optimized protocols for the scalable manufacture of human T lymphocytes (T-cells) are essential to maximize the therapeutic potential of patient-derived T-cells. Obtaining billions of T-cells for cell therapy requires a large-scale production of T-cells that involves ex vivo culturing and expansion of a population of donor T-cells many fold in a highly controlled environment to produce a sufficient number of T-cells. Typically, T-cells may be expanded in cell culture chamber (e.g., a bioreactor) in which a culture media may be introduced and removed to provide nutrient, reagents, pH modulation, gas diffusion, temperature control, removal of wastes, etc., thereby providing an optimal environment that promote growth and/or expansion of T-cell populations. It is important that such culture media be introduced into and removed from the bioreactor without disturbing the T-cell population.

Various types of bioreactors for culturing and expanding T-cells exist on the market. For example, static bioreactors, such as a cell culture flask, need to be mixed and require a lot of surface area exposed to air to get appropriate gas diffusion to the T-cells. Some static bioreactors are gas permeable, but have limited scalability and process control due to difficulty in rapidly replacing the perfusion media. Furthermore, such static bioreactors often require high media volumes, such that the seeding cell density is higher. Hollow-fiber bioreactors are largely designed for extracting soluble proteins and perfusion media from a suspension of T-cells, which applies a lot of shear stress to the T-cells, thereby hindering the growth and/or expansion of the T-cells.

There, thus, is a need for an improve bioreactor for expanding a population of T-cells that is scalable and allows for the introduction and removal of culture media without disturbing the T-cell population.

### SUMMARY

In accordance with one aspect of the present inventions, a bioreactor assembly comprises a plurality of bioreactor units (e.g., stacked on top of each other) arranged in a flow sequence. In one embodiment, each of the plurality of bioreactor units is configured for expanding a population of biological cells. The bioreactor assembly further comprises at least one flow path (e.g., at least two different flow paths (e.g., three different flow paths) extending through the bioreactor units. In embodiment, the flow path(s) selectively extend through the plurality of bioreactor units, such that selected ones of the plurality of bioreactor units may be added to the flow path(s), and selected ones of the plurality of bioreactor units may be removed from the flow path(s).

In one embodiment, each of the plurality of bioreactor units comprises at least one flow channel into which media can be flowed, at least one inlet port respectively fluidly coupled to the at least one flow channel, such that media can be respectively flowed into the at least one flow channel, and at least one outlet port respectively fluidly coupled to the at least one flow channel, such that media can be respectively flowed out of the at least one flow channel. Each of the outlet port(s) of each of the plurality of bioreactor units preceding the last bioreactor unit in the flow sequence is respectively fluidly coupled to an immediately succeeding bioreactor unit in the flow sequence, such that the flow path(s) respectively extend entirely through the flow channel(s) of each of the plurality of bioreactor units. The bioreactor assembly may further comprise a single housing that respectively contains the plurality of bioreactor units. The inlet port(s) and the outlet port(s) of each of the plurality of bioreactor units may be internal or external to the single housing. Alternatively, each of the plurality of bioreactor units may comprise a separate housing.

In one embodiment, the flow channel(s) comprise two flow channels (e.g., an upper flow channel and a lower flow channel), in which case, the inlet port(s) of each of the plurality of bioreactor units may comprise two inlet ports respectively fluidly coupled to the two flow channels, and the outlet port(s) of each of the plurality of bioreactor units may comprise two outlet ports respectively fluidly coupled to the two flow channels. The flow path(s) may comprise a first flow path extending in through the inlet port and out through the outlet port of a first one of the two flow channels of each of the plurality of bioreactor units, and a second flow path different from the first flow path extending in through the inlet port and out through the outlet port of a second one of the two flow channels of each of the plurality of bioreactor units. The flow path(s) may comprise a third flow path different from the first flow path and the second flow path extending in through the inlet port of the first flow channel, between the first flow channel and the second flow channel, and out through the outlet port of the second flow channel, of each of the plurality of bioreactor units.

In another embodiment, each of the plurality of bioreactor units may comprise a cell culture surface contained within the first flow channel of the respective bioreactor unit, first media containing biological cells can be flowed into the first flow channel, thereby disposing a population of biological cells on the cell culture surface of each of the plurality of bioreactor units, and second media containing one or more of nutrient, reagent, and gas can be flowed along the second flow path through the second flow channel of each of the plurality of bioreactor units, thereby facilitating the expansion of the populations of biological cells respectively within the plurality of bioreactor units. The third media can be flowed along the first flow path through the first flow channel of each of the plurality of bioreactor units, thereby harvesting the expanded populations of biological cells from the respective plurality of bioreactor units. In this embodiment, each of the plurality of bioreactor units may comprise a first membrane between the first flow channel and the second flow channel of the respective bioreactor unit. The first membrane is substantially permeable to the nutrient, reagent, and/or gas contained within the second media, and waste produced by the populations of biological cells, thereby facilitating expansion of the populations of biological cells within the first flow channels of the respective plurality of bioreactor units, while being substantially impermeable to the populations of biological cells. In this embodiment, the flow path(s) may comprise a third flow path extending in through the inlet port of the first flow channel, between the first flow channel and the second flow channel, and out through the outlet port of the second flow channel, of each of the plurality of bioreactor units. The first membrane of each of the plurality of bioreactor units may be a porous membrane configured for allowing perfusion of the first media along the third flow path from the first flow channels into the second flow channels of the respective plurality of bioreactor units, while filtering the populations of biological cells from the first media, such that the populations of biological cells are retained within the first flow channels of the respective plurality of bioreactor units. Each of the plurality of bioreactor units may optionally comprise a second membrane, in which case, the first flow channels are disposed between the first membranes and the second membranes of the respective plurality of bioreactor units, and wherein the second membrane of each of the plurality of bioreactor units is substantially permeable to gas from a space exterior to the first flow channels of the respective plurality of bioreactor units, thereby facilitating expansion of the populations of biological cells within the first flow channels of the respective plurality of bioreactor units, while being substantially impermeable to the populations of biological cells and the first media. The cell culture surface may be formed on the second membrane.

In accordance with a second aspect of the present inventions, a bioreactor system may comprise the aforementioned bioreactor assembly and a pump assembly configured for conveying media along the at least one flow path. The pump assembly comprises e.g., one or more peristaltic pumps. The bioreactor system may further comprise a valve assembly configured for selectively fluidly coupling ones of the plurality of bioreactor units to the at least one flow path, and selectively fluidly decoupling ones of the plurality of bioreactor units from the at least one flow path. In the case where each of the plurality of bioreactor units comprises flow channel(s), inlet port(s), and outlet port(s), the valve assembly may be configured for selectively fluidly coupling each of the at least one outlet port of each of the plurality of bioreactor units preceding the last bioreactor unit of the flow sequence respectively to the immediately succeeding bioreactor unit in the flow sequence.

In accordance with a third aspect of the present inventions, a method of operating the aforementioned bioreactor assembly is provided. The method comprises seeding the plurality of bioreactor units respectively with populations of biological cells (e.g., T lymphocytes (T-cells)), flowing first media (e.g., via pumping) containing one or more of nutrient, reagent, and gas along a first one of the at least one flow path, thereby facilitating the expansion of the populations of biological cells respectively within the plurality of bioreactor units, and harvesting the expanded populations of biological cells respectively from the plurality of bioreactor units. Seeding the plurality of bioreactor units respectively with the populations of biological cells may comprise flowing second media containing biological cells along a second one of the flow path(s) different from the first flow path. The method may optionally comprise harvesting the expanded populations of biological cells respectively from the plurality of bioreactor units comprises flowing third media along a third one of the flow path(s) different from the first flow path and the second flow path.

In the case where each of the plurality of bioreactor units comprises two flow channels, two inlet ports, and two outlet ports, the method may comprise flowing first media (e.g., via a pump) containing the biological cells (e.g., T lymphocytes (T-cells)) into the first flow channels of the respective plurality of bioreactor units, thereby respectively depositing the populations of biological cells on the cell culture surfaces of the respective plurality of bioreactor units, and flowing second media (e.g., via a pump) containing the one or more of nutrient, reagent, and gas along the second flow path through the second flow channel of each of the plurality of bioreactor units, thereby facilitating the expansion of the populations of biological cells within the second flow channels of the respective plurality of bioreactor units. The method may optionally comprise flowing third media along the first flow path into the first flow channels of the respective plurality of bioreactor units, such that the expanded populations of biological cells are suspended in the third media, and flowing the third media with the suspended expanded populations of biological cells along the first flow path out of the second flow channels of the respective plurality of bioreactor units.

In the case where each of the plurality of bioreactor units comprises a cell culture surface, a first membrane, and an additional third flow path, the method may further comprise flowing (e.g., via pumping) the first media containing the biological cells (e.g., T lymphocytes (T-cells)) along the third flow path into the first flow channels of the respective plurality of bioreactor units, and perfusing the first media along the third flow path from the first flow channels, through the first porous membranes, and into the second flow channels of the respective plurality of bioreactor units, while substantially retaining the biological cells within the first flow channels of the respective plurality of bioreactor units, such that the populations of biological cells are disposed on the cell culture surfaces of the respective plurality of bioreactor units, and flowing the perfused first media along the third flow path out of the second flow channels of the respective plurality of bioreactor units. Such method may further comprise flowing (e.g., via pumping) the second media containing the one or more of nutrient, reagent, and gas along the second flow path through the second flow channels of the respective plurality of bioreactor units; and diffusing the nutrient, reagent, and/or gas from the second flow channels, through the first membranes, and into the second flow channels, and diffusing the waste from the first flow channels, through the first membranes, and into the second flow channels, of the respective plurality of bioreactor units, thereby facilitating the expansion of the populations of biological cells within the first flow channels of the respective plurality of biological cells. An optional method may further comprise flowing (e.g., via pumping) third media along the first flow path into the first flow channels of the respective plurality of bioreactor units, such that the expanded populations of biological cells are suspended in the third media, and flowing the third media with the suspended expanded populations of biological cells along the first flow path out of the second flow channels of the respective plurality of bioreactor units.

In accordance with a fourth aspect of the present inventions, a method of expanding a population of biological cells using a plurality of bioreactor units is provided. The method comprises (a) adding a flow sequence of a plurality of bioreactor units to at least one flow path, (b) seeding the plurality of bioreactor units respectively with populations of biological cells, (c) flowing (e.g., via pumping) first media containing one or more of nutrient, reagent, and gas along a first one of the at least one flow path, thereby facilitating the expansion of the populations of biological cells respectively within the plurality of bioreactor units, and (d) harvesting the expanded populations of biological cells respectively from the plurality of bioreactor units. Step (b) may be performed for each preceding bioreactor unit subsequent to adding the flow sequence of the plurality of bioreactor units to the at least one flow path. In optional method further comprises (e) sequentially stacking the plurality of bioreactor units atop each other. In this case, steps (a)-(c) may be performed for each immediately preceding bioreactor unit in the flow sequence prior to stacking each of at least one succeeding bioreactor unit in the flow sequence atop the immediately preceding bioreactor unit. The plurality of bioreactor units may be stacked together prior to performing step (a) on a first one of the flow sequence of the plurality of bioreactor units.

In one method, seeding the plurality of bioreactor units respectively with the populations of biological cells comprises flowing second media containing biological cells along a second one of the at least one flow path different from the first flow path. In another method, harvesting the expanded populations of biological cells respectively from the plurality of bioreactor units comprises flowing third media along a third one of the at least one flow path different from the first flow path and the second flow path. In still another method, the flow sequence of the plurality of bioreactor units is increased over time by incrementally adding at least one succeeding bioreactor unit to the at least one flow path, and the steps (b) and (c) are performed for at least one preceding bioreactor unit in the flow sequence prior to each addition of the at least one succeeding bioreactor unit in the flow sequence to the at least one flow path.

Seeding each of the succeeding bioreactor unit(s) with a respective population of biological cells may comprise isolating at least one aliquot of biological cells from at least one expanded population of biological cells respectively within the preceding bioreactor unit(s), and seeding each respective one of the succeeding bioreactor unit(s) with the aliquot(s) of biological cells respectively isolated from the preceding bioreactor unit(s). Isolating the aliquot(s) of the biological cells from the expanded population(s) of biological cells within the preceding bioreactor unit(s) may comprise flowing third media through the preceding bioreactor unit(s), such that the aliquot(s) of biological cells is suspended in the third media, and seeding the respective succeeding bioreactor unit(s) with the aliquot(s) of biological cells respectively isolated in the preceding bioreactor unit(s) comprises flowing the third media containing the suspended aliquot(s) of biological cells from the preceding bioreactor unit(s) into each of the succeeding bioreactor unit(s). At least one multilayer expanded population of biological cells may be respectively within the preceding bioreactor unit(s), in which case, isolating the aliquot(s) of biological cells from the expanded population of biological cell(s) respectively within the preceding bioreactor unit(s) may comprise isolating at least one upper layer of the multilayer expanded population(s) of biological cells, such that a monolayer or one or more lower layers of the expanded population(s) of biological cells remain respectively within the preceding bioreactor unit(s).

In another method, each of the plurality of bioreactor units comprises two flow channels, a first membrane disposed between the two flow channels, and a cell culture surface contained within the first flow channel. In this case, the step (b) of seeding the plurality of bioreactor units respectively with the populations of biological cells may comprise flowing second media containing biological cells into the first flow channels of the respective plurality of bioreactor units, thereby respectively disposing the populations of biological cells on the cell culture surfaces of the respective plurality of bioreactor units. The step (c) of flowing the first media containing one or more of nutrient, reagent, and gas along the first flow path may comprise flowing the first media through the second flow channels of the respective plurality of bioreactor units, such that the one or more of nutrient, reagent, and gas diffuses from the second flow channels, through the first membranes, and into the second flow channels, and waste produced by the populations of biological cells diffuses from the first flow channels, through the first membranes, and into the second flow channels, of the respective plurality of bioreactor units, thereby facilitating the expansion of the populations of biological cells within the first flow channels of the respective plurality of biological cells. The step (d) of harvesting the expanded populations of biological cells respectively from the plurality of bioreactor units may comprise (d1) flowing third media along the first flow path into the first flow channels of the respective plurality of bioreactor units, such that the expanded populations of biological cells are suspended in the third media, and (d2) flowing the third media with the suspended expanded populations of biological cells along the first flow path out of the second flow channels of the respective plurality of bioreactor units.

The first membrane may be a porous membrane, in which case, the step (b) of seeding the plurality of bioreactor units respectively with the populations of biological cells may comprise (b1) flowing the second media containing the biological cells along forward portion of the second flow path into the first flow channels of the respective plurality of bioreactor units; (b2) perfusing the second media along bypass portions of the second flow path from the first flow channels, through the porous membranes, and into the second flow channels, of the respective plurality of bioreactor units, while substantially retaining the biological cells within the first flow channels of the respective plurality of bioreactor units, such that the populations of biological cells are disposed on the cell culture surfaces of the respective plurality of bioreactor units; (b3) flowing the perfused second media along return portion of the second flow path out of the second flow channels of the respective plurality of bioreactor units. Steps (b1)-(b3) may be incrementally performed in each of the plurality of bioreactor units in the order of the flow sequence.

The cell culture surfaces may be disposed on the respective porous membranes of the respective plurality of bioreactor units, in which case, the incremental perfusion of the second media with the biological cells along the bypass portion of the second flow path from the first flow channel, through the porous membrane, and into the second flow channel may urge the biological cells towards the cell culture surface on the porous membrane via the action of convection of a current bioreactor unit preceding the last bioreactor unit in the flow sequence until at least one layer of the biological cells are disposed on the cell culture surface, thereby substantially blocking perfusion of the second media through the porous membrane of the current bioreactor unit, such that the second media with substantially no biological cells initially flows along the forward portion of the second flow path out of the current bioreactor unit into a next unseeded bioreactor unit in the flow sequence, and such that the second media with substantially all of the biological cells subsequently flows further along the forward portion of the second flow path out of the current bioreactor unit into the next unseeded bioreactor unit. Each of the plurality of bioreactor units may optionally comprise a second membrane, with the first flow channels disposed between the first membranes and the second membranes of the respective plurality of bioreactor units. In this case, the method may further comprise (e) diffusing gas from a space exterior to the first flow channels, through the second membranes, and into the first flow channels, of the respective plurality of bioreactor units, thereby facilitating the expansion of the populations of biological cells within the first flow channels of the respective plurality of biological cells.

In accordance with a fifth aspect of the present inventions, a bioreactor system comprises at least one bioreactor, each configured for containing a population of biological cells and comprising a first flow channel, a second flow channel, a porous membrane disposed between the first and second flow channels. In one embodiment, the porous membrane of each of the bioreactor(s) is configured for supporting the respective population of biological cells thereon.

The bioreactor system further comprises a first multi-function valve (e.g., a rotary valve) having a first at least one position for defining a first flow path through the first flow channel of each of the bioreactor(s), and a second at least one position different from the first position(s) for defining a second flow path into the first flow channel, between the first and second flow channels via the porous membrane, and out of the second flow channel of each of the bioreactor(s). In one embodiment, the first position(s) comprises a plurality of positions and/or the second position(s) comprises a plurality of positions. The bioreactor system further comprise a first pump configured for conveying media along the first flow paths of the bioreactor(s) when the first multi-function valve is in the first position(s), and for conveying media along the second flow paths of the bioreactor(s) when the multi-function valve is in the second position(s). In an optional embodiment, the bioreactor system further comprises an electric controller configured for operating the first multi-function valve and the first pump.

In one embodiment, the bioreactor system further comprises a second multi-function valve having a first at least one position for defining a third flow path through the second flow channel of each of the bioreactor(s), and a second at least one position different from the first at least one position for defining a fourth flow path into the second flow channel, between the second and first flow channels via the porous membrane, and out of the first flow channel of each of the bioreactor(s). This bioreactor system further comprises a second pump fluidly configured for conveying media along the third flow path of each of the bioreactor(s) when the second multi-function valve is in the first position(s), and configured for conveying media along the fourth flow path of each of the bioreactor(s) when the multi-function valve is in the second position(s).

In one embodiment, the bioreactor(s) is a single bioreactor. In this case, the first multi-function valve may be configured for respectively fluidly coupling at least one media source (e.g., one or more of a biological cell source, microbead source, nutrient source, reagent source, gas source, wash buffer source, and saline source) to the first flow channel of the single bioreactor while fluidly coupling a media destination (e.g., a biological cell collection destination or a waste destination) to the first flow channel of the single bioreactor when the first multi-function valve is respectively in the first position(s), thereby defining the first flow path for the single bioreactor. In this embodiment, the first multi-function valve may be further configured for respectively fluidly coupling the media source(s) to the first flow channel of the single bioreactor while fluidly coupling no media destination to the first flow channel of the single bioreactor when the first multi-function valve is respectively in the second position(s), thereby defining the second flow path for the single bioreactor. The media source(s) may comprise a plurality of media sources, in which case, the first position(s) may comprise a first plurality of positions, and the second position(s) may comprise a second plurality of positions. In this embodiment, the first multi-function valve may have a third at least one position configured for fluidly coupling no media source and no media destination to the first flow channel of the single bioreactor.

In another embodiment, the bioreactor(s) comprises a plurality of bioreactor units. In this case, the first position(s) may comprise a first plurality of positions, and the second position(s) may comprise a second plurality of positions. In this embodiment, the first multi-function valve may be configured for respectively fluidly coupling a media source to the first flow channels of the plurality of bioreactor units while fluidly coupling a media destination to the first flow channels of the plurality of bioreactor units when the first multi-function valve is respectively in the first plurality of positions, thereby respectively defining the first flow paths for the plurality of bioreactor units. The first multi-function valve may be further configured for respectively fluidly coupling the media source to the first flow channels of the plurality of bioreactor units while fluidly coupling no media destination to the first flow channels of the plurality of bioreactor units when the first multi-function valve is respectively in the second plurality of positions, thereby respectively defining the second flow paths for the plurality of bioreactor units. In this embodiment, the first multi-function valve may have a third position configured for fluidly coupling no media source and no media destination to the first flow channels of the plurality of bioreactor units.

In accordance with a sixth aspect of the present inventions, a bioreactor system comprises at least one bioreactor, each configured for containing a population of biological cells and comprising a first flow channel, a second flow channel, a porous membrane disposed between the first and second flow channels, and a first port and a second port associated with the first flow channel. In one embodiment, the porous membrane of each of the bioreactor(s) is configured for supporting the respective population of biological cells thereon.

The bioreactor system further comprises a first multi-function valve comprising a common port fluidly coupled to the first port of one of the bioreactor(s), a return port fluidly coupled to the second port of the one bioreactor, a plurality of selector ports configured for respectively being fluidly coupled to a first plurality of media sources and/or media destinations (e.g., (e.g., one or more of a biological cell source, microbead source, nutrient source, reagent source, gas source, wash buffer source, saline source, and biological cell collection destination), and an output port configured for being fluidly coupled to a first media destination. In one embodiment, the output port is the only output port of the first multi-function valve. The first multi-function valve has a first plurality of positions for respectively fluidly coupling the plurality of selector ports to the common port, while fluidly coupling the output port to the return port, and a second plurality of positions different from the first plurality of positions for respectively fluidly coupling the plurality of selector ports to the common port, while fluidly decoupling the output port from the return port. In one embodiment, the first multi-function valve comprises a first valve portion that respectively fluidly couples the plurality of selector ports to the common port when the first multi-function valve is respectively in the first plurality of positions or the second plurality of positions, and a second valve portion that fluidly couples the output port to the return port when the multi-function valve is in any of the first plurality of positions, and fluidly decouples the output port from the return port when the first multi-function valve is in any of the second plurality of positions. The bioreactor system further comprises a first pump fluidly coupled to one of the common port and the return port of the first multi-function valve. In an optional embodiment, the bioreactor system further comprises an electric controller configured for operating the first multi-function valve and the first pump.

In one embodiment, the first multi-function valve has a third position different from the first plurality of positions and the second plurality of positions for fluidly decoupling all of the plurality of selector ports from the common port, for fluidly decoupling the output port from the return port, and for fluidly decoupling the return port from the common port. In another embodiment, the first multi-function valve has a third position different from the first plurality of positions and the second plurality of positions for fluidly decoupling all of the plurality of selector ports from the common port, while fluidly decoupling the output port from the return port, and while fluidly coupling the return port to the common port.

In still another embodiment, each of the bioreactor(s) further comprises a third port and a fourth port associated with the second flow channel, in which case, the bioreactor system further comprises a second multi-function valve comprising a common port fluidly coupled to the third port of the one bioreactor, a return port fluidly coupled to the fourth port of the one bioreactor, a plurality of selector ports configured for respectively being fluidly coupled to a second plurality of media sources and/or media destinations, and an output port configured for being fluidly coupled to a second media destination (e.g., a waste destination). In this embodiment, the second multi-function valve has a first plurality of positions for respectively fluidly coupling the plurality of selector ports to the common port, while fluidly coupling the output port to the return port, and a second plurality of positions different from the first plurality of positions for respectively fluidly coupling the plurality of selector ports to the common port, while fluidly decoupling the output port from the return port. This bioreactor system further comprises a second pump fluidly coupled to one of the common port and the return port of the second multi-function valve. In this embodiment, at least some of the first plurality of media sources and/or media destinations may be respectively the same as at least some of the second plurality of media sources and/or media destinations and/or the first media destination and the second media destination may be the same.

In yet another embodiment, the bioreactor(s) comprises a plurality of bioreactor units, with the one bioreactor unit being a base bioreactor unit within the plurality of bioreactor units. This bioreactor system further comprises a second multi-function valve comprising a common port fluidly coupled to the common port of the first multi-function valve, one or more selector ports respectively fluidly coupled to the first ports of one or more of the plurality of bioreactor units different from the base bioreactor unit, one or more return ports respectively fluidly coupled to the second ports of the bioreactor(s), and an output port fluidly coupled to the first port of the base bioreactor unit. The second multi-function valve has first one or more positions for respectively fluidly coupling the selector port(s) to the common port, while fluidly coupling the return port(s) to the output port. The second multi-function valve further has second one or more positions different from the first one or more positions for respectively fluidly coupling the selector port(s) to the common port, while fluidly decoupling all of the return port(s) from the output port. The second multi-function valve further comprises a third position for respectively fluidly coupling the output port to the common port, while fluidly decoupling the selector port(s) from the common port, and while fluidly decoupling the return port(s) from the output port. In this embodiment, the bioreactor(s) may comprise two or more bioreactor units, in which case, the selector port(s) may comprises two or more selector ports, the return port(s) may comprise two or more return ports, the first position(s) may comprises two or more positions, and the second position(s) may comprises two or more positions.

In yet another embodiment, the first multi-function valve is a rotary valve. In this embodiment, the rotary valve may comprise a stator in which the common port, the return port, the plurality of selector ports, and the output port are affixed, and a rotor configured for being rotated relative to the stator to the first plurality of positions and the second plurality of positions. In one embodiment, the bioreactor system further comprises a motor configured for rotating the rotor relative to the stator. The bioreactor system may further comprise a shaft having one end affixed to the motor, and another end magnetically coupled to the rotor. The rotor comprises a first open channel and a second open channel. When the rotor is rotated relative to the stator to the first plurality of positions, the first open channel respectively fluidly couples the plurality of selector ports to the common port, while the second open channel fluidly couples the output port to the return port. For example, when the rotor is rotated relative to the stator to each of the first plurality of positions, a respective one of the plurality of selector ports and the common port may concurrently coincide with the first open channel, while the return port and the output port may concurrently coincide with the second open channel. When the rotor is rotated relative to the stator to the second plurality of positions, the first open channel respectively fluidly couples the plurality of selector ports to the common port, while the second open channel does not fluidly couple the output port to the return port. For example, when the rotor is rotated relative to the stator to each of the second plurality of positions, a respective one of the plurality of selector ports and the common port may concurrently coincide with the first open channel, while the return port and the output port may not concurrently coincide with the second open channel.

The rotor may additionally be rotated relative to the stator to a third position different from the first plurality of positions and the second plurality of positions, such that the first open channel does not fluidly couple any of the plurality of selector ports to the common port, the second open channel does not fluidly couple the output port to the return port, and the first open channel does not fluidly couple the return port to the common port. For example, when the rotor is rotated relative to the stator in the third position, the plurality of selector ports and the common port may not concurrently coincide with the first open channel, the return port and the output port may not concurrently coincide with the second open channel, and the return port and the common port may not concurrently coincide with the first open channel.

The rotor may additionally be rotated relative to the stator to a fourth position different from the first plurality of positions, the second plurality of positions, and the third position, such that the first open channel does not fluidly couple any of the plurality of selector ports to the common port, the second open channel does not fluidly couple the output port to the return port, and the first open channel fluidly couples the return port to the common port. For example, when the rotor is rotated relative to the stator to the fourth position, the plurality of selector ports and the common port may not concurrently coincide with the first open channel, the return port and the output port may not concurrently coincide with the second open channel, and the return port and the common port may concurrently coincide with the first open channel.

In accordance with a seventh aspect of the present inventions, a bioreactor system comprises a plurality of bioreactor units, each configured for containing a population of biological cells and comprising a first flow channel, a second flow channel, a porous membrane disposed between the first and second flow channels, and a first port and a second port associated with the first flow channel. In one embodiment, the porous membrane of each of the plurality of bioreactor units is configured for supporting the respective population of biological cells thereon.

The bioreactor system further comprises a first multi-function valve comprising a common port configured for being fluidly coupled to one of a first media source and a first media destination (e.g., one or more of a biological cell source, microbead source, nutrient source, reagent source, gas source, wash buffer source, saline source, and biological cell collection destination), a plurality of selector ports respectively fluidly coupled to the first ports of the plurality of bioreactor units, a plurality of return ports respectively fluidly coupled to the second ports of the plurality of bioreactor units, and an output port configured for being fluidly coupled to a second media destination (e.g., a waste destination). The first multi-function valve has a first plurality of positions for respectively fluidly coupling the plurality of selector ports to the common port, while respectively fluidly coupling the plurality of return ports to the output port. The first multi-function valve further has a second plurality of positions for respectively fluidly coupling the plurality of selector ports to the common port, while respectively fluidly decoupling all of the plurality of return ports from the output port. In one embodiment, the first multi-function valve comprises a first valve portion that respectively fluidly couples the plurality of selector ports to the common port when the first multi-function valve is respectively in the first plurality of positions or the second plurality of positions, and a second first valve portion that respectively fluidly couples the plurality of return ports to the output port when the multi-function valve is in the first plurality of positions, and fluidly respectively decouples the plurality of return ports from the output port when the first multi-function valve is in the second plurality of positions. The bioreactor system further comprise a first pump fluidly coupled to one of the common port and the output port of the first multi-function valve. In one embodiment, the first pump is fluidly coupled between the first multi-function valve and the first port of the one bioreactor. In an optional embodiment, the bioreactor system further comprises an electric controller configured for operating the first multi-function valve and the first pump.

In another embodiment, the first multi-function valve has a third position different from the first plurality of positions and the second plurality of positions for fluidly decoupling all of the plurality of selector ports from the common port, fluidly decoupling all of the plurality of return ports from the output port, and for fluidly decoupling the output port from the common port. In still another embodiment, the first multi-function valve has a third position different from the first plurality of positions and the second plurality of positions for fluidly coupling the output port to the common port, while fluidly decoupling all of the plurality of selector ports from the common port, and fluidly decoupling all of the plurality of return ports from the output port.

In yet another embodiment, each of the plurality of bioreactor units further comprises a third port and a fourth port associated with the second flow channel, in which case, the bioreactor system further comprises a second multi-function valve comprising a common port configured for being fluidly coupled to the one of the first media source and the first media destination, and a plurality of selector ports respectively fluidly coupled to the third ports of the plurality of bioreactor units, a plurality of return ports respectively fluidly coupled to the fourth ports of the plurality of bioreactor units, and an output port configured for being fluidly coupled to the second media destination. In this embodiment, the first multi-function valve has a first plurality of positions for respectively fluidly coupling the plurality of selector ports to the common port, while respectively fluidly coupling the plurality of return ports to the output port. The first multi-function valve further has a second plurality of positions for respectively fluidly coupling the plurality of selector ports to the common port, while respectively fluidly decoupling all of the plurality of return ports from the output port. In this embodiment, the bioreactor system further comprises a second pump fluidly coupled to one of the common port and the output port of the second multi-function valve.

In yet another embodiment, the bioreactor system further comprises a base bioreactor unit different from the plurality of bioreactor units. In this embodiment, the bioreactor system further comprising a second multi-function valve comprising a common port fluidly coupled to the common port of the first multi-function valve, a return port fluidly coupled to the second port of the base bioreactor unit, a plurality of selector ports configured for respectively being fluidly coupled to a plurality of media sources and/or media destinations, and an output port configured for being fluidly coupled to the second media destination. In this embodiment, the first multi-function valve has a first plurality of positions for respectively fluidly coupling the plurality of selector ports to the common port, such that one of the plurality of media sources and/or media destinations is selected as the one of the first media source and the first media destination that is fluidly coupled to the common port of the first multi-function valve, while fluidly coupling the output port to the return port. The first multi-function valve further has a second plurality of positions different from the first plurality of positions for respectively fluidly coupling the plurality of selector ports to the common port, such that one of the plurality of media sources is selected as the one of the first media source and the first media destination that is fluidly coupled to the common port of the first multi-function valve, while fluidly decoupling the output port from the return port.

In yet another embodiment, the first multi-function valve is a rotary valve. In this embodiment, the rotary valve may comprise a stator in which the common port, the plurality of selector ports, the plurality of return ports, and the output port are affixed, and a rotor configured for being rotated relative to the stator to the first plurality of positions and the second plurality of positions. In this embodiment, the bioreactor system may further a motor configured for rotating the rotor relative to the stator. The bioreactor system may further comprise a shaft having one end affixed to the motor, and another end magnetically coupled to the rotor. The rotor comprises a first open channel and a second open channel. When the rotor is rotated relative to the stator to the first plurality of positions, the first open channel respectively fluidly couples the plurality of selector ports to the common port, while the second open channel respectively fluidly couples the plurality of return ports to the output port. For example, when the rotor is rotated relative to the stator to each of the first plurality of positions, a respective one of the plurality of selector ports and the common port concurrently coincide with the first open channel, while a respective one of the plurality of return ports and the output port concurrently coincide with the second open channel. When the rotor is rotated relative to the stator to the second plurality of positions, the first open channel respectively fluidly couples the plurality of selector ports to the common port, while the second open channel does not fluidly couple any of the plurality of return ports to the output port. For example, when the rotor is rotated relative to the stator to each of the second plurality of positions, a respective one of the plurality of selector ports and the common port concurrently coincide with the first open channel, while none of the plurality of return ports and the output port concurrently coincide with the second open channel.

The rotor may additionally be rotated relative to the stator to a third position different from the first plurality of positions and the second plurality of positions, such that the first open channel does not fluidly couple any of the plurality of selector ports to the common port, the second open channel does not fluidly couple any of the plurality of return ports to the output port, and the first open channel does not fluidly couple the output port to the common port. For example, when the rotor is rotated relative to the stator in the third position, the plurality of selector ports and the common port do not concurrently coincide with the first open channel, the plurality of return ports and the output port do not concurrently coincide with the second open channel, and the output port and the common port do not concurrently coincide with the first open channel.

The rotor may additionally be rotated relative to the stator to a fourth position different from the first plurality of positions, the second plurality of positions, and the third position, such that the first open channel does not fluidly couple any of the plurality of selector ports to the common port, the second open channel does not fluidly couple any of the plurality of return ports to the output port, and the first open channel fluidly couples the output port to the common port. For example, when the rotor is rotated relative to the stator to the fourth position, the plurality of selector ports and the common port do not concurrently coincide with the first open channel, the plurality of return ports and the output port do not concurrently coincide with the second open channel, and the output port and the common port concurrently coincide with the first open channel.

In accordance with an eighth aspect of the present inventions, a bioreactor system comprises a plurality of bioreactor units, each configured for containing a population of biological cells and comprising a first flow channel, a second flow channel, a porous membrane disposed between the first and second flow channels, and a first port and a second port associated with the first flow channel. The plurality of bioreactor units comprise a base bioreactor unit and at least one supplemental bioreactor unit. In one embodiment, the porous membrane of each of the plurality of bioreactor units is configured for supporting the respective population of biological cells thereon.

The bioreactor system further comprises a first multi-function valve comprising a common port, a return port fluidly coupled to the second port of the base bioreactor unit, at least one selector port configured for respectively being fluidly coupled to a first at least one media source and/or media destination (e.g., one of a biological cell source, a microbead source, a nutrient source, a reagent source, a gas source, a wash buffer source, a saline source, and a biological cell collection destination), and an output port configured for being fluidly coupled to a first media destination (e.g., a waste destination). The first multi-function valve has a first at least one position for respectively fluidly coupling the selector port(s) to the common port, while fluidly coupling the output port to the return port, and a second at least one position different from the first position(s) for respectively fluidly coupling the selector port(s) to the common port, while fluidly decoupling the output port from the return port. In one embodiment, the first multi-function valve comprises a first valve portion that respectively fluidly couples the selector port(s) to the common port when the first multi-function valve is respectively in the first position(s) or the second position(s), and a second valve portion that fluidly couples the output port to the return port when the multi-function valve is in any of the first position(s), and fluidly decouples the output port from the return port when the first multi-function valve is in any of the second position(s).

The bioreactor system further comprises a second multi-function valve comprising a common port fluidly coupled to the common port of the first multi-function valve, and at least one selector port respectively fluidly coupled to the first ports of the supplemental bioreactor unit(s), at least one return port respectively fluidly coupled to the second ports of the supplemental bioreactor unit(s), and an output port configured for being fluidly coupled to the first port of the base bioreactor unit. The second multi-function valve has a first at least one position for respectively fluidly coupling the selector port(s) to the common port, while respectively fluidly coupling the return port(s) to the output port, and a second at least one position for respectively fluidly coupling the selector port(s) to the common port, while respectively fluidly decoupling all of the return port(s) from the output port. In one embodiment, second multi-function valve comprises a first valve portion that respectively fluidly couples the selector port(s) to the common port when the second multi-function valve is respectively in the first position(s) or the second position(s), and a second valve portion that respectively fluidly couples the return port(s) to the output port when the second multi-function valve is in the first position(s), and fluidly decouples all of the return port(s) from the output port when the second multi-function valve is in the second position(s).

The bioreactor system further comprises a first pump fluidly coupled between the common port of the first multi-function valve and the common port of the second multi-function valve, or between the output port of the second multi-function valve and the first port of the base bioreactor unit, or between the second port of the base bioreactor unit and the return port of the first multi-function valve. In an optional embodiment, the bioreactor system further comprises an electric controller configured for operating the first multi-function valve, the second multi-function valve, and the first pump.

In one embodiment, the first media source(s) and/or media destination(s) comprises a first plurality of media sources and/or media destinations, the selector port(s) of the first multi-function valve comprises a plurality of selector ports, the supplemental bioreactor unit(s) comprises a plurality of supplemental bioreactor units, the selector port(s) of the second multi-function valve comprises a plurality of selector ports, and the return port(s) of the second multi-function valve comprises a plurality of return ports.

In another embodiment, the first multi-function valve has a third position different from the first position(s) and the second position(s) for fluidly decoupling all of the selector port(s) from the common port, for fluidly decoupling the output port from the return port, and for fluidly decoupling the return port from the common port, and second multi-function valve has a third position different from the first position(s) and the second position(s) for fluidly decoupling all of the selector port(s) from the common port, fluidly decoupling all of the return port(s) from the output port, and for fluidly decoupling the output port from the common port. In still another embodiment, the first multi-function valve has a third position different from the first position(s) and the second position(s) for fluidly coupling the return port to the common port, while fluidly decoupling all of the selector port(s) from the common port, and while fluidly decoupling the output port from the return port. In yet another embodiment, the first multi-function valve has a third position different from the first position(s) and the second position(s) for fluidly coupling the output port to the common port, while fluidly decoupling all of the plurality of selector ports from the common port, and fluidly decoupling all of the plurality of return ports from the output port.

In yet another embodiment, each of the plurality of bioreactor units further comprises a third port and a fourth port associated with the second flow channel, in which case, the bioreactor system further comprises a third multi-function valve and a fourth multi-function valve. The third multi-function valve comprises a common port, a return port fluidly coupled to the fourth port of the one bioreactor, at least one selector port configured for respectively being fluidly coupled to a second at least one media source and/or media destination, and an output port configured for being fluidly coupled to a second media destination. The third multi-function valve has a first at least one position for respectively fluidly coupling the selector port(s) to the common port, while fluidly coupling the output port to the return port, and a second at least one position different from the third position for respectively fluidly coupling the selector port(s) to the common port, while fluidly decoupling the output port from the return port. The fourth multi-function valve comprises a common port fluidly coupled to the common port of the third multi-function valve, and at least one selector port respectively fluidly coupled to the third ports of the supplemental bioreactor unit(s), at least one return port respectively fluidly coupled to the fourth ports of the supplemental bioreactor unit(s), and an output port configured for being fluidly coupled to the third port of the base bioreactor unit. The fourth multi-function valve has a first at least one position for respectively fluidly coupling the selector port(s) to the common port, while respectively fluidly coupling the return port(s) to the output port, and a second at least one position for respectively fluidly coupling the selector port(s) to the common port, while respectively fluidly decoupling all of the return port(s) from the output port. This bioreactor system further comprises a second pump fluidly coupled between the common port of the third multi-function valve and the common port of the fourth multi-function valve, or between the output port of the fourth multi-function valve and the third port of the base bioreactor unit, or between the fourth port of the base bioreactor unit and the return port of the third multi-function valve. In this embodiment, at least some of the first at least one media source and/or media destination may be respectively the same as at least some of the second at least one media source and/or media destination and/or the first media destination and the second media destination may be the same

In yet another embodiment, the first multi-function valve is a first rotary valve, and the second multi-function valve is a second rotary valve. In this embodiment, each of the first and second rotary valves comprises a stator in which the common port, the return port, the selector port(s), and the output port are affixed, and a rotor configured for being rotated relative to the stator to the first position(s) and the second position(s). In this embodiment, the bioreactor system may further comprise a first motor configured for rotating the rotor relative to the stator of the first rotary valve, and a second motor configured for rotating the rotor relative to the stator of the second rotary valve. In this embodiment, the bioreactor system may further comprise a first shaft having one end affixed to the first motor, and another end magnetically coupled to the rotor of the first rotary valve, and a second shaft having one end affixed to the second motor, and another end magnetically coupled to the rotor of the second rotary valve.

In accordance with a ninth aspect of the present inventions, a rotary valve comprises a stator comprising a common port, a plurality of selector ports, an output port, and at least one return port. The rotary valve further comprises a rotor configured for being rotated relative to the stator to a first plurality of positions that respectively fluidly couples the plurality of selector ports to the common port, while respectively fluidly coupling the at least one return port to the output port, and for being rotated relative to the stator to a second plurality of positions different from the first plurality of positions that fluidly couples the plurality of selector ports to the common port, while fluidly decoupling all of the at least one return port from the output port.

In one embodiment, the return port(s) is a single return port. In this embodiment, the rotor may be configured for being rotated relative to the stator to a third position different from the first plurality of positions and the second plurality of positions that fluidly decouples all of the plurality of selector ports from the common port, fluidly decouples the single return port from the output port, and fluidly decouples the single return port from the common port. In another embodiment, the rotor may be configured for being rotated relative to the stator to a third position different from the first plurality of positions and the second plurality of positions that fluidly couples the single return port to the common port, while fluidly decoupling all of the plurality of selector ports from the common port, and fluidly decoupling the single return port from the output port.

In another embodiment, the return port(s) is a plurality of return ports. In this embodiment, the rotor may be configured for being rotated relative to the stator to a third position different from the first plurality of positions and the second plurality of positions that fluidly decouples all of the plurality of selector ports from the common port, fluidly decouples all of the plurality of return ports from the output port, and fluidly decouples the output port from the common port. In this embodiment, the rotor may be configured for being rotated relative to the stator to a fourth position different from the first plurality of positions, the second plurality of positions, and the third position, that fluidly couples the output port to the common port, while fluidly decoupling all of the plurality of selector ports from the common port, and fluidly decoupling all of the plurality of return ports from the output port.

In still another embodiment, the rotor has a first open channel that respectively fluidly couples the plurality of selector ports to the common port when the rotor is rotated relative to the stator respectively in the first plurality of positions or the second plurality of positions, and a second open channel that respectively fluidly couples the return port(s) to the output port when the rotor is rotated relative to the stator to the first plurality of positions, and respectively fluidly decouples all of the return port(s) from the output port when the rotor is rotated relative to the stator to the second plurality of positions. For example, when the rotor is rotated relative to the stator to each of the first plurality of positions, a respective one of the plurality of selector ports and the common port concurrently coincide with the first open channel, while a respective one of the at least one return port and the output port concurrently coincide with the second open channel, and when the rotor is rotated relative to the stator to each of the second plurality of positions, a respective one of the plurality of selector ports and the common port concurrently coincide with the first open channel, while none of the at least one return port and the output port concurrently coincide with the second open channel.

In this embodiment, the rotor may be rotated relative to the stator to a third position different from the first plurality of positions and the second plurality of positions, such that the first open channel does not fluidly couple any of the plurality of selector ports to the common port, the second open channel does not fluidly couple any of the at least one return port to the output port, and the first open channel does not fluidly couple the return port or the output port to the common port. For example, when the rotor is rotated relative to the stator in a third position different from the first plurality of positions and the second plurality of positions, the plurality of selector ports and the common port do not concurrently coincide with the first open channel, the return port(s) and the output port do not concurrently coincide with the second open channel, and the return port, the output port, and the common port do not concurrently coincide with the first open channel.

In this embodiment, the rotor may be rotated relative to the stator to a fourth position different from the first plurality of positions, the second plurality of positions, and the third position, such that the first open channel does not fluidly couple the plurality of selector ports to the common port, the second open channel does not fluidly couple any of the return port(s) to the output port, and the first open channel fluidly couples either one of the return port(s) or the output port to the common port. For example, when the rotor is rotated relative to the stator to a fourth position different from the first plurality of positions, the second plurality of positions, and the third position, the plurality of selector ports and the common port do not concurrently coincide with the first open channel, the return port(s) and the output port do not concurrently coincide with the second open channel, and either the return port(s) or the output port and the common port concurrently coincide with the first open channel. If the return port is a single return port, the first open channel does not fluidly couple the single return port to the common port when the rotor is rotated relative to the stator to the third position, and fluidly couples the single return port to the common port when the rotor is rotated relative to the stator to the fourth position. If the return port is a plurality of return ports, the first open channel does not fluidly couple any of the plurality of return ports to the common port when the rotor is rotated relative to the stator to the third position, and fluidly couples the output to the common port when the rotor is rotated relative to the stator to the fourth position.

In one embodiment, the first open channel has a circumferential portion with which the common port always coincides when the rotor is rotated relative to the stator to the first plurality of positions and the second plurality of positions, an axial portion extending from the circumferential portion, and a circumferential spur portion extending from the axial portion. In this case, the plurality of selector ports respectively coincide with one of the axial portion and the circumferential spur portion (e.g., the circumferential spur portion) of the first open channel when the rotor is rotated relative to the stator to the first plurality of positions, and respectively coincide with the other of the axial portion and the circumferential spur portion (e.g., the axial portion) of the first open channel when the rotor is rotated relative to the stator to the second plurality of positions. The second open channel has a circumferential portion with which the output port always coincides when the rotor is rotated relative to the stator to the first plurality of positions and the second plurality of positions, and at least one axial spur portion extending from the circumferential portion and with which one of the return port(s) coincides when the rotor is rotated relative to the stator to the first plurality of positions, and with which none of the return port(s) coincides when the rotor is rotated relative to the stator to the second plurality of positions.

If the return port(s) is a single return port, the axial spur portion(s) comprises a plurality of axial spur portions with which the single return port respectively coincides when the rotor is rotated relative to the stator to the first plurality of positions, and with which the single return port does not coincide when the rotor is rotated relative to the stator to the second plurality of positions. If the return port(s) is a plurality of return ports, the axial spur portion(s) is a single axial spur portion with which the plurality of return ports respectively coincide when the rotor is rotated relative to the stator to the first plurality of positions, and with which the plurality of return ports respectively do not coincide when the rotor is rotated relative to the stator to the second plurality of positions.

In this embodiment, the rotor may be rotated relative to the stator to a third position different from the first plurality of positions and the second plurality of positions, none of the plurality of selector ports coincide with the axial spur portion or the circumferential spur portion of the first open channel, none of the return port(s) coincides with the axial spur portion of the second open channel, and none of the return output port(s) and the output port coincides with the axial portion of the first open channel. In this embodiment, the rotor may be rotated relative to the stator to a fourth position different from the first plurality of positions, the second plurality of positions, and the third position, such that none of the plurality of selector ports coincide with the axial spur portion or the circumferential spur portion of the first open channel, none of the plurality of the return ports coincide with the axial spur portion of the second open channel, and either one of the return port(s) or the output port coincides with the axial portion of the first open channel. If the return port(s) is a single return port, it coincides with the axial portion of the first open channel when the rotor is rotated relative to the stator to the fourth position. If the return port(s) is a plurality of return ports, the output port coincides with the axial portion of the first open channel when the rotor is rotated relative to the stator to the fourth position.

Other and further aspects and features of embodiments will become apparent from the ensuing detailed description in view of the accompanying figures.

### BRIEF DESCRIPTION OF THE DRAWINGS

The drawings illustrate the design and utility of preferred embodiments of the disclosed inventions, in which similar elements are referred to by common reference numerals. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. Further, an aspect or an advantage described in conjunction with a particular embodiment of the disclosed inventions is not necessarily limited to that embodiment and can be practiced in any other embodiments even if not so illustrated.

In order to better appreciate how the above-recited and other advantages and objects of the disclosed inventions are obtained, a more particular description of the disclosed inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a block diagram of one embodiment of a bioreactor system constructed in accordance with the present inventions;

FIG. 2 is a block diagram of one embodiment of a stacked bioreactor assembly that can be used in the bioreactor system of FIG. 1;

FIGS. 3A-3D are block diagrams illustrating activation or deactivation of selected ones of flow paths extending through the stacked bioreactor assembly of FIG. 2;

FIGS. 4A-4J are block diagrams illustrating the activation different flow sequences of bioreactor units in the stacked bioreactor assembly of FIG. 2;

FIG. 5 is a perspective view of one embodiment of a bioreactor unit that can be used in the bioreactor assembly of FIG. 2;

FIG. 6 is a cross-sectional view of the bioreactor unit of FIG. 5;

FIGS. 7A-7C are plan views illustrating the manner in which flow sequences of bioreactor units of FIG. 6 can be added to different flow paths between a media source and a media destination;

FIG. 8 is a cross-sectional view of a stacked bioreactor assembly of bioreactor units of FIG. 6 that can be used in the bioreactor assembly of FIG. 2;

FIG. 9 is a flow diagram illustrating one method of operating the stacked bioreactor assembly of FIG. 8 to expand multiple populations of biological cells;

FIGS. 10A-10U illustrates a sequence of stages during operation of the stacked bioreactor assembly of FIG. 8 to expand the multiple populations of biological cells in accordance with the method of FIG. 9;

FIG. 11 is a flow diagram illustrating one method of incrementally seeding the bioreactor units of the stacked bioreactor assembly of FIG. 8 with multiple seed populations of biological cells;

FIGS. 12A-12E illustrates a sequence of stages during operation of the stacked bioreactor assembly of FIG. 8 to incrementally seed the bioreactor units in accordance with the method of FIG. 11;

FIG. 13 is a cross-sectional view of another embodiment of a bioreactor unit that can be used in the bioreactor assembly of FIG. 2;

FIGS. 14A-14C are plan views illustrating the manner in which flow sequences of bioreactor units of FIG. 13 can be added to different flow paths between a media source and a media destination;

FIG. 15 is a cross-sectional view of a stacked bioreactor assembly of bioreactor units of FIG. 13 that can be used in the bioreactor assembly of FIG. 2;

FIG. 16 is a flow diagram illustrating one method of operating the stacked bioreactor assembly of FIG. 15 to expand multiple populations of biological cells;

FIGS. 17A-17AR illustrates a sequence of stages during operation of the stacked bioreactor assembly of FIG. 15 to expand the multiple populations of biological cells in accordance with the method of FIG. 16;

FIG. 18 is another embodiment of a bioreactor system constructed in accordance with the present inventions;

FIGS. 19A-19H are block diagrams of the bioreactor system of FIG. 18, particularly illustrating a sequence of different flow paths through a single dynamic bioreactor of the bioreactor system;

FIG. 20 is a block diagram of still another bioreactor system constructed in accordance with the present inventions;

FIGS. 21A-21C are block diagrams of the bioreactor system of FIG. 20, particularly illustrating a flow path through a sequence of a stacked bioreactor assembly of the bioreactor system;

FIG. 22 is a block diagram of yet another embodiment of a bioreactor system constructed in accordance with the present inventions;

FIG. 23 is a table illustrating a sequence of valve positions for defining different flow paths through a single bioreactor of the bioreactor system of FIG. 22;

FIGS. 24A-24H are block diagrams of the bioreactor system of FIG. 22, particularly illustrating a sequence of different flow paths through the single bioreactor in accordance with the sequence of valve positions illustrated in FIG. 23;

FIG. 25 is a front view of one embodiment of a motorized valve assembly that can be used in the bioreactor system of FIG. 23;

FIG. 26 is a top perspective view of the motorized valve assembly of FIG. 25;

FIG. 27 is a top perspective view of a rotary valve of the motorized valve assembly of FIG. 25;

FIG. 28 is a bottom perspective view of a motor of the motorized valve assembly of FIG. 25;

FIG. 29 is a bottom perspective view of two polymagnets used to create a magnetic coupling between the motor and the rotary valve of the motorize valve assembly of FIG. 25;

FIG. 30 is a front view of the rotary valve of FIG. 27;

FIG. 31 is a bottom view of the rotary valve of FIG. 27;

FIGS. 32A-32E are profile views of a rotor of the rotary valve of FIG. 27;

FIGS. 33A-33J are topological views of the rotary valve of FIG. 27, particularly illustrating the fluid connections between different ports of the stator and open channels of the rotary in accordance with the sequence of valve positions illustrated in FIG. 23;

FIG. 34 is a block diagram of yet embodiment of a bioreactor system constructed in accordance with the present inventions;

FIG. 35 is a table illustrating a sequence of valve positions for defining a flow path through different combinations of bioreactor units of the bioreactor system of FIG. 34, particularly illustrating a flow path through a sequence of different combinations of the stacked bioreactor units;

FIGS. 36A-36H are block diagrams of the bioreactor system of FIG. 34, particularly illustrating a sequence of different flow paths through a base bioreactor unit in accordance with the sequence of valve positions illustrated in FIG. 23 and the sequence of valve positions illustrated in FIG. 35;

FIGS. 37A-37H are block diagrams of the bioreactor system of FIG. 34, particularly illustrating a sequence of different flow paths through a first supplemental bioreactor unit in accordance with the sequence of valve positions illustrated in FIG. 23 and the sequence of valve positions illustrated in FIG. 35;

FIGS. 38A-38H are block diagrams of the bioreactor system of FIG. 34, particularly illustrating a sequence of different flow paths through a second supplemental bioreactor unit in accordance with the sequence of valve positions illustrated in FIG. 23 and the sequence of valve positions illustrated in FIG. 35;

FIGS. 39A-39D are profile views of a rotor used in an alternative embodiment of a motorized valve assembly for use in the bioreactor system of FIG. 34; and

FIGS. 40A-40F are topological views of an alternative embodiment of a rotary valve for use in a motorized valve assembly of the bioreactor system of FIG. 34, particularly illustrating the fluid connections between different ports of the stator and open channels of the rotary in accordance with the sequence of valve positions illustrated in FIG. 35.

### DETAILED DESCRIPTION OF THE ILLUSTRATED EMBODIMENTS

The bioreactor systems described herein provide a physiologically relevant environment for expanding a population of biological cells (i.e., increasing the number of biological cells), and in particular, immunological cells, such as T lymphocytes (e.g., endogenous T-cells (ETCs), chimeric antigen receptor (CAR) T-cells, or engineered T-cells), natural killer (NK) cells, and/or other immune cells, in a cell therapy manufacturing system (CTMS). Alternatively, or in addition to, such population of biological cells may be, e.g., hematopoietic progenitor cells or stem cells, such as embryonic stem cells (ESCs), mesenchymal stem cells (MSCs), induced pluripotent stem cells (iPSCs), or the like.

Significantly, the bioreactor systems described herein are scalable in that they comprise bioreactor units that can be added to (either physically or brought into operation) or removed from (either physically or taken out of operation) a bioreactor assembly as needed for a desired yield of biological cells. For example, such bioreactor units may be stacked on each other (i.e., along the direction of gravity) to create a stacked bioreactor assembly. Each of the bioreactor units comprises at least one flow path along which media can be flowed to seed the bioreactor assembly with a population of biological cells, incubate the seeded biological cells, thereby expanding the population of biological cells, and/or harvest the expanded population of biological cells from bioreactor assembly. Such flow paths of the bioreactor can be fluidly coupled together either statically (e.g., physically) or dynamically (e.g., via a valve assembly) to add or remove bioreactor units to or from the bioreactor assembly.

The bioreactor systems described herein can be automated or semi-automated, thereby further maximizing, or at least increasing, the yield of biological cells. The bioreactor systems described herein may be incorporated into cell therapy workflow that may perform functions other than biological cell expansion, e.g., sample collection, cell sorting, cell modification, quality control assaying, formulation and filling, treatment administration, etc. The bioreactor assemblies of such bioreactor systems described herein may serve as stand-alone units may be integrated (e.g., as a cartridge or cassette) within the CTMS, which can receive, controllably manipulate, and monitor the cartridge or cassette. The cartridge or cassette may have a sealed enclosure, which may be sterile and/or hermetically sealed, and one or more inlet and/or outlet ports for fluidly accessing the sealed enclosure, e.g., the bioreactor assembly within the cartridge or cassette. In addition to a bioreactor assembly, such cartridge or cassette may include other components, regions for cell monitoring and/or assaying, reagent reservoirs, and the like. Further details of an exemplary CTMS are set forth in PCT Application Ser. No. PCT/US2022/012194, entitled “Systems, Apparatuses, and Methods for Cellular Therapeutics Manufacture,” which is expressly incorporated herein by reference.

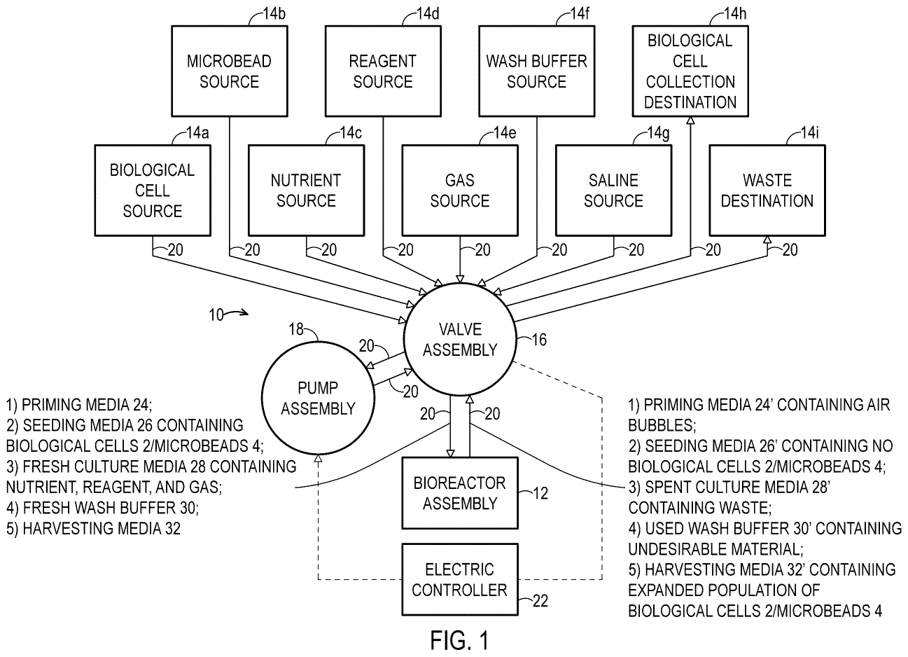

Referring first to FIG. 1, one embodiment of a bioreactor system 10 constructed in accordance with the present inventions will be described. The bioreactor system 10 generally comprises a bioreactor assembly 12, a plurality of media sources/media destinations 14, a valve assembly 16, a pump assembly 18, an arrangement of fluid conduits 20, and an electric controller 22.

The bioreactor assembly 12 is generally configured for incubating and expanding a seed population of cells, as will be described in further detail below. The arrangement of fluid conduits 20 fluidly couple the bioreactor assembly 12, media sources/media destinations 14, valve assembly 16, and pump assembly 18 together to facilitate the flow of different media between these components. The arrangement of fluid conduits 20 may, e.g., take the form of flexible plastic tubing and/or rigid channels formed within a monolithic structure.

The media sources/media destinations comprise a biological cell source 14a, a microbead source 14b, a nutrient source 14c (e.g., carbohydrates, lipids, proteins, and nucleic acids), a reagent source 14d (e.g., cytokines, such as Interleukin-2 (IL-2)), a gas source 14e (e.g., air), a wash buffer source 14f, a saline source 14g (e.g., Phosphate Buffered Saline (PBS)), a biological cell collection destination 14h, and a waste destination 14i. Each of the media sources 14a-14d and 14f-14g may, e.g., take the form of a bag or syringe supplied with the respective component (i.e., biological cells, microbeads, nutrient, reagent, wash buffer, and media), and each of the media destinations 14h-14i may take the form of a receptacle (e.g., a bag, a walled chamber, a well of a well-plate, a syringe, and the like) for subsequently collecting the respective component (i.e., an expanded population of biological cells and waste). The gas source 14e may, e.g., take the form of an ambient environment, although in alternative embodiments, the gas source may take the form of a gas chamber. The biological cells, microbeads, nutrients, and reagents may be combined with an aqueous media to facilitate their flow within the bioreactor system 10. In alternative embodiments, any of the media sources/media destinations 14a-14i may take form of rigid chambers, e.g., formed within a monolithic structure. In other alternative embodiments, some of the media sources can be combined or consolidated. For example, the biological cell source 14a and microbead source 14b can be combined into a single biological cell/micro-object source. As another example, the nutrient source 14c, reagent source 14d, and/or gas source 14e may be combined into a single nutrient/reagent/gas source.

The valve assembly 16 is configured for selectively defining the flow paths (shown by arrows) between various ones of the media sources/media destinations 14 and the bioreactor assembly 12, while the pump assembly 18 is configured for providing the necessary fluid pressure for conveying various media along the flow paths defined by the valve assembly 16, such that different media can be flowed between the media sources/media destinations 14 and the bioreactor assembly 12 for the purpose of priming the bioreactor assembly 12 (i.e., removing all air within the bioreactor assembly 12), seeding the bioreactor assembly 12 with a population of biological cells (with or without microbeads), culturing the biological cells, such that the population of biological cells are expanded, washing the biological cells, and harvesting the expanded population of biological cells from the bioreactor assembly 12. Although both the valve assembly 16 and pump assembly 18 are illustrated in FIG. 1 as being single components, it should be appreciated that each of the valve assembly 16 and pump assembly 18 can comprise multiple components (i.e., multiple valves or multiple pumps) that are fluidly coupled directly to each other or indirectly to each other via other components of the bioreactor system 10 via the arrangement of fluid conduits 20. Furthermore, although the valve assembly 16 is illustrated as being external to the bioreactor assembly 12, in some embodiments, the valve assembly 16 may be internal to the bioreactor assembly 12. For example, the bioreactor assembly 12 and the valve assembly 16 may be packaged as a cartridge or a portion of a cartridge.

In one embodiment, the valve assembly 16 may be configured for selectively directing: (a) priming media 24 from the saline source 14g to the bioreactor assembly 12, and from the bioreactor assembly 12 to the waste destination 14i, thereby priming the bioreactor assembly 12; (b) seeding media 26 containing a seed population of biological cells 2 or microbeads 4, or both, from the biological cell source 14a and/or microbead source 14b to the bioreactor assembly 12; and directing seeding media 26′ without the biological cells 2 or microbeads 4 from the bioreactor assembly 12 to the waste destination 14i, thereby seeding the bioreactor assembly 12 with a population of biological cells 2 or microbeads 4, or both; (c) directing fresh culture media 28 with nutrient, reagent, and gas from the nutrient source 14c, reagent source 14d, and gas source 14e, respectively, (or alternatively directing gas separately from the fresh culture media 28 from the gas source 14e) to the bioreactor assembly 12; and directing spent culture media 28′ containing waste from the population of biological cells 2 contained within the bioreactor assembly 12 to the waste destination 14i, thereby culturing and facilitating expansion of the seed population of biological cells 2 contained within the bioreactor assembly 12; (d) directing fresh wash buffer 30 from the wash buffer source 14f to the bioreactor assembly 12, and directing used wash buffer 30′ containing undesirable material (e.g., unbound molecules, debris, dead cells, or other undesirable molecules) from the bioreactor assembly 12 to the waste destination 14i, thereby washing the population of biological cells 2 or microbeads 4, or both; and/or (e) directing harvesting media 32 without biological cells 2 (or microbeads 4) from the saline source 14g to the bioreactor assembly 12, and directing the harvesting media 32′ with the expanded population of biological cells 2 and microbeads 4 from the bioreactor assembly 12 to the biological cell collection destination 14h, thereby harvesting the expanded population of biological cells 2 (or microbeads 4) from the bioreactor assembly 12. As will be described in further detail below, the valve assembly 16 is further configured for defining flow paths through the bioreactor assembly 12 to facilitate any combination of the aforementioned functions (a)-(e).

The electric controller 22 is configured for controlling the operation of the valve assembly 16 and pump assembly 18 in accordance with one or more programs stored within memory (not shown) to facilitate the various flow processes described herein, including priming the bioreactor assembly 12, seeding the bioreactor assembly 12 with the population of biological cells 2 and/or microbeads 4, culturing the population of biological cells 2, washing the population of biological cells 2 and/or microbeads 4, and once sufficiently expanded, harvesting the expanded population of biological cells 2 from the bioreactor assembly 12. The electric controller 22 can comprise, e.g., a processor, application specific integrated circuit (ASIC), field programmable gate array (FPGA), or other type of circuitry.

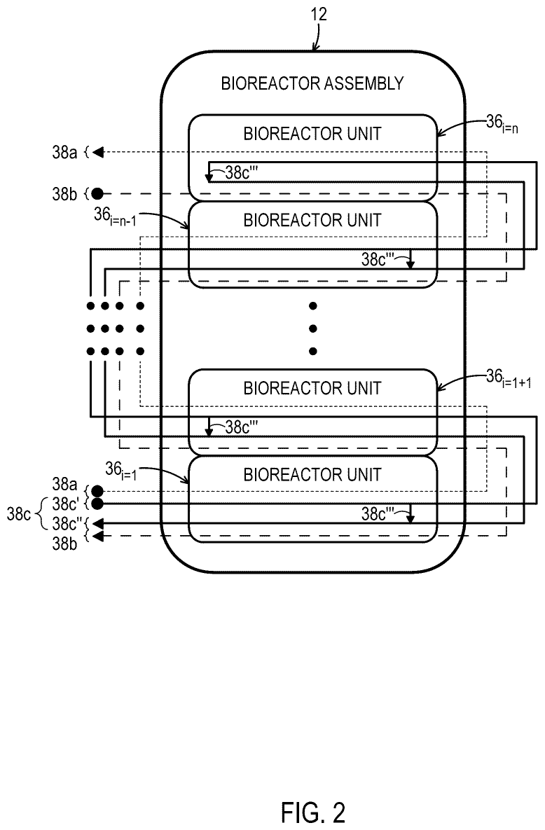

Referring now to FIG. 2, one generalized embodiment of a bioreactor assembly 12 will now be described. The bioreactor assembly 12 comprises an assembly of bioreactor units 36, each of which, at a minimum, is capable of being seeded with a population of biological cells 2, and optionally microbeads 4, culturing the population of biological cells 2, thereby facilitating the expansion of the seed population of biological cells 2, and allowing the expanded population of biological cells 2 to be harvested. As will be described in further detail below, the bioreactor units 36 may be, e.g., integrated into a single housing or may have separate housings that are then integrated together.

In the illustrated embodiment, the bioreactor units 36 are stacked on each other (i.e., along the direction of gravity, such that the ith stacked bioreactor unit 36ᵢ is directly adjacent to the ith+1 stacked bioreactor unit 36ᵢ₊₁. In alternative embodiments, the bioreactor units 36 may be arranged along a single plane, although stacking the bioreactor units 36 along gravity is particularly advantageous in that such arrangement minimizes the footprint of the bioreactor assembly 12 (e.g., by packaging the bioreactor assembly 12 as a cartridge or a portion of a cartridge) for facilitating incorporation into the bioreactor system 10.

Significantly, to facilitate the scalability of the bioreactor system 10, the bioreactor assembly 12 further comprises at least one flow path 38 capable of extending through each of the bioreactor units 36 for effecting various functions. In the illustrated embodiment, the bioreactor units 36 are fluidly coupled together via the flow path(s) 38. As will be described in further detail below, the bioreactor units 36 may be fluidly coupled internally within the housing or housings or externally outside of the housing or housings. In the illustrated embodiment, three different flow paths 38a-38c extend within each of the operational bioreactor units 36, although it should be appreciated that less than three different flow paths 38 (e.g., two or even one flow path) or more than three different flow paths 38 can extend within each of the operational bioreactor units 36. As one example, the first and second flow paths 38a, 38b may be used to prime the bioreactor assembly 12; the third flow path 38c may be used to seed the bioreactor assembly 12 with biological cells 2 and/or microbeads 4 and wash the biological cells 2 and/or microbeads 4; the second flow path 38b may be used to culture the biological cells 2; and the first flow path 38a may be used to harvest biological cells 2 and/or microbeads 4 from the bioreactor assembly 12.

When all of the bioreactor units 36 are operational (i.e., the flow paths 38 include all of the bioreactor units 36), the first flow path 38a sequentially extends through the last stacked bioreactor units 36 from the first stacked bioreactor unit 36₁ to the last stacked bioreactor unit 36ₙ; the second flow path 38b sequentially extends through the stacked bioreactor units 36 from the last stacked bioreactor unit 36ₙ to the first stacked bioreactor unit 36₁; a forward portion 38c′ of the third flow path 38c sequentially extends through the last stacked bioreactor units 36 from the first stacked bioreactor unit 36₁ to the last stacked bioreactor unit 36ₙ, and a return portion 38c″ of the third flow path 38c sequentially extends through the last stacked bioreactor units 36 from the last stacked bioreactor unit 36ₙ to the first stacked bioreactor unit 36₁, and bypass portions 38c″ of the third flow path 38c coupling the forward portion 38c′ and return portion 38c″ with each of the bioreactor units 36. It should be appreciated that other types of flow paths may be envisioned, and may depend on the nature of the bioreactor unit 36.

It should also be appreciated that, although the flow paths 38a-38c are illustrated as extending through all of the bioreactor units 36 in a sequence that matches the sequence in which the bioreactor units 36 are physically arranged; i.e., the flow sequence of the bioreactor units 36 matches the physical sequence of the bioreactor units 36 relative to all of the flow paths 38a-38c (e.g., the flow paths 38a-38c extend from the first stacked bioreactor unit 36₁, to the second stacked bioreactor unit 36₂, to the third stacked bioreactor unit 36₃, and so forth, or from the last stacked bioreactor unit 36ₙ, to the second to last stacked bioreactor unit 36ₙ₋₁, to the third to last stacked bioreactor unit 36ₙ₋₂, and so forth), any of the flow paths 38a-38c may sequentially extend through the last stacked bioreactor units 36 in an order that does not match the order in which the bioreactor units 36 are physically arranged; i.e., the flow sequence of the bioreactor units 36 for a particular one of the flow paths 38a-38c does not match the physical sequence of the bioreactor units 36 (e.g., the flow path 38a may sequentially extend from the first stacked bioreactor unit 36₁, to the second to last stacked bioreactor unit 36ₙ₋₁, back to the second stacked bioreactor unit 36₂, to the last stacked bioreactor unit 36ₙ, etc.).

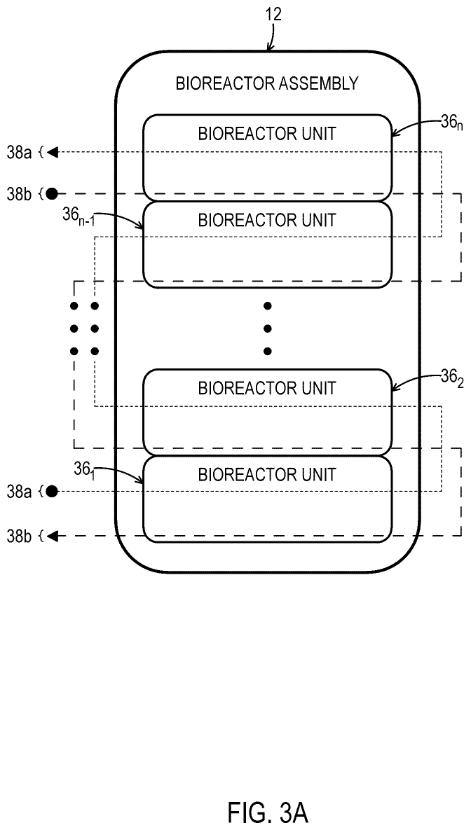

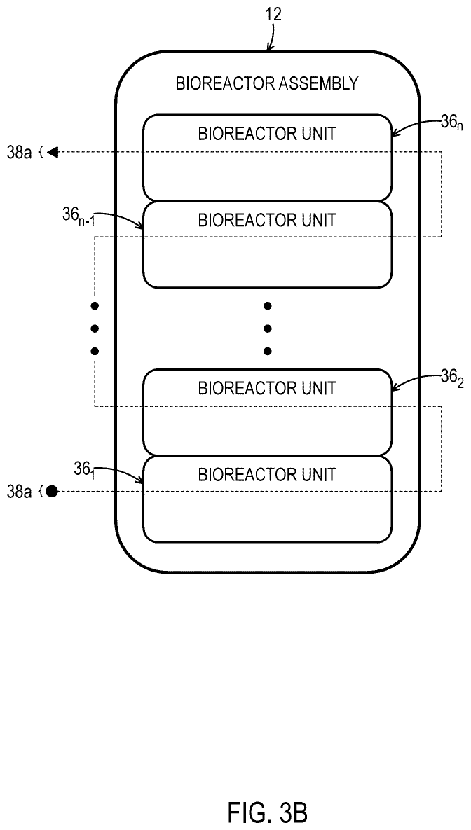

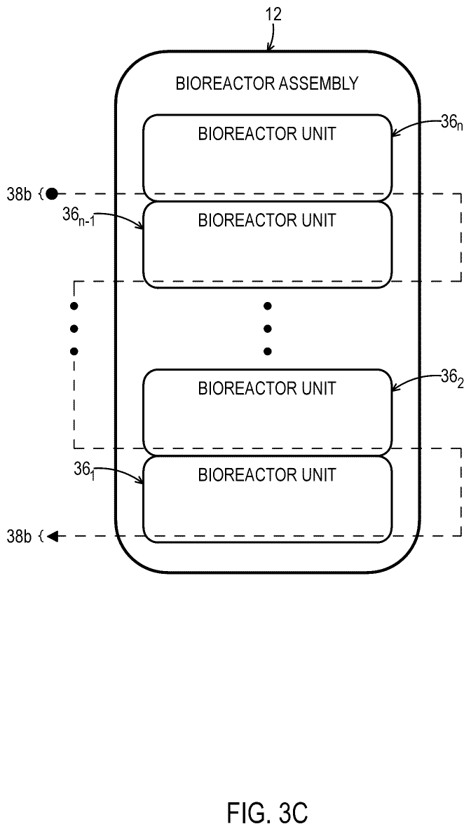

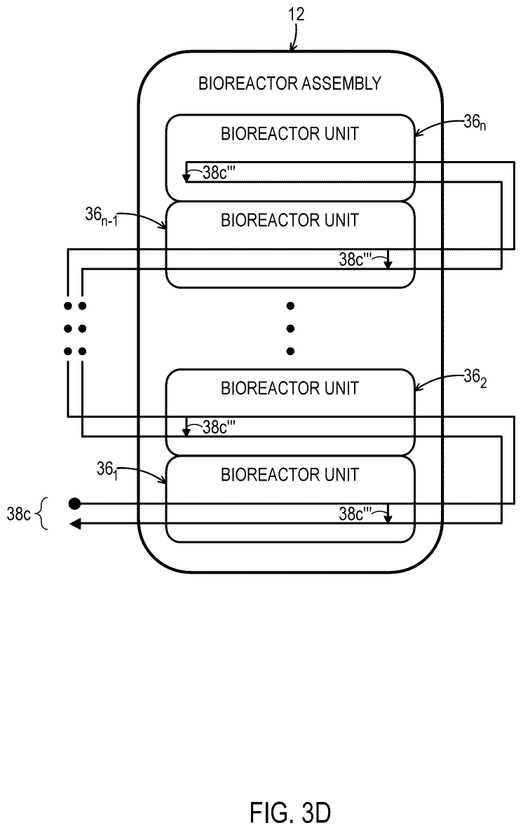

It should also be appreciated that not all the flow paths 38a-38c may be simultaneously active. In one embodiment, the valve assembly 16 may be operated to activate (allowing flow) or deactivate (preventing flow) selected ones of the flow paths 38a-38c. For example, the valve assembly 16 may be operated to activate both the flow paths 38a, 38b, while deactivating the third flow path 38c (see FIG. 3A); may be operated to activate only the first flow path 38a, while deactivating both the flow paths 38b, 38c (see FIG. 3B); may be operated to activate only the second flow path 38b, while deactivating both the flow paths 38a, 38c (see FIG. 3C); may be operated to activate only the third flow path 38c, while deactivating both the flow paths 38a, 38b (see FIG. 3D); etc.

Although all bioreactor units 36 have been discussed as being operational in the bioreactor assembly 12, in one advantageous embodiment, the valve assembly 16 (shown in FIG. 1) may be operated to add or remove selected ones of the n number of bioreactor units 36 (bioreactor unit 36_{i>1} to bioreactor unit 36ₙ) to or from the flow paths 38a-38c; i.e., by bringing or taking out the selected ones of the n number of bioreactor unit 36 into or out of operation. In this manner, the bioreactor system 10 may be dynamically scaled.

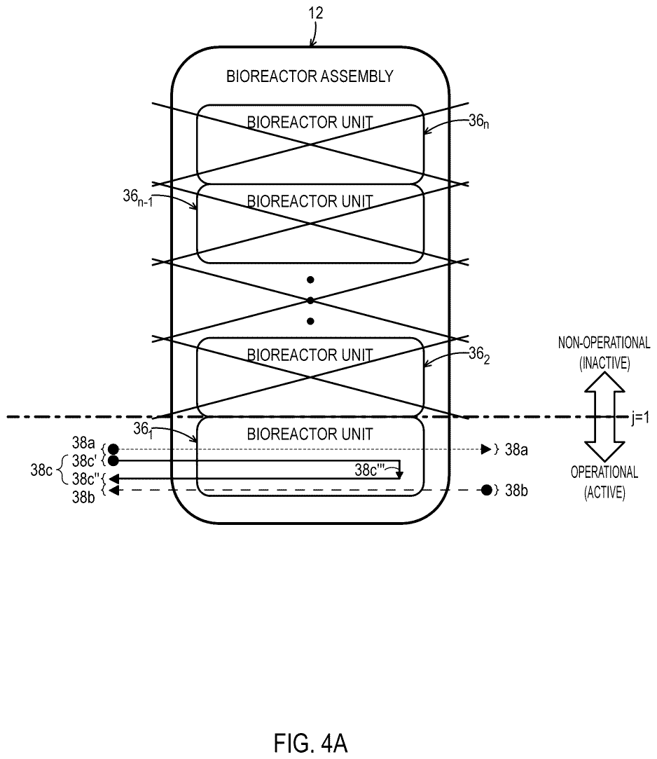

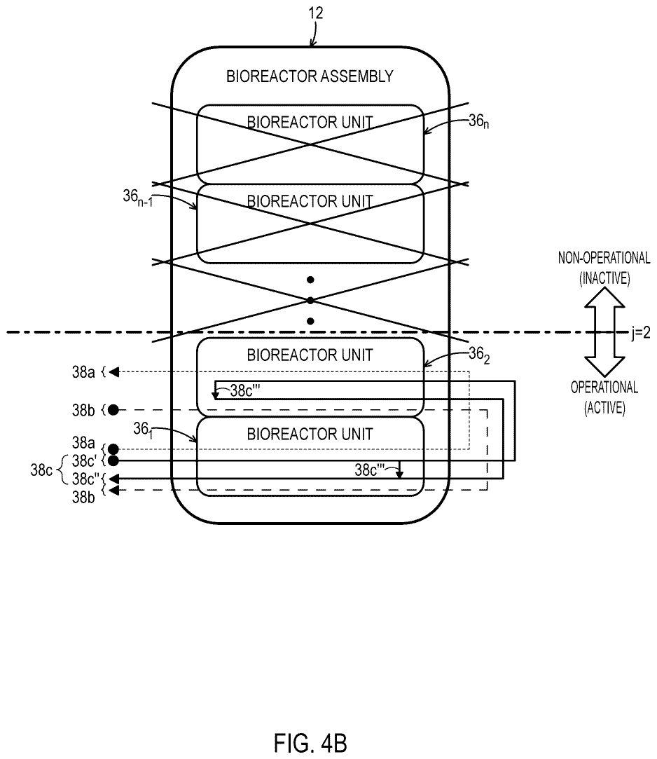

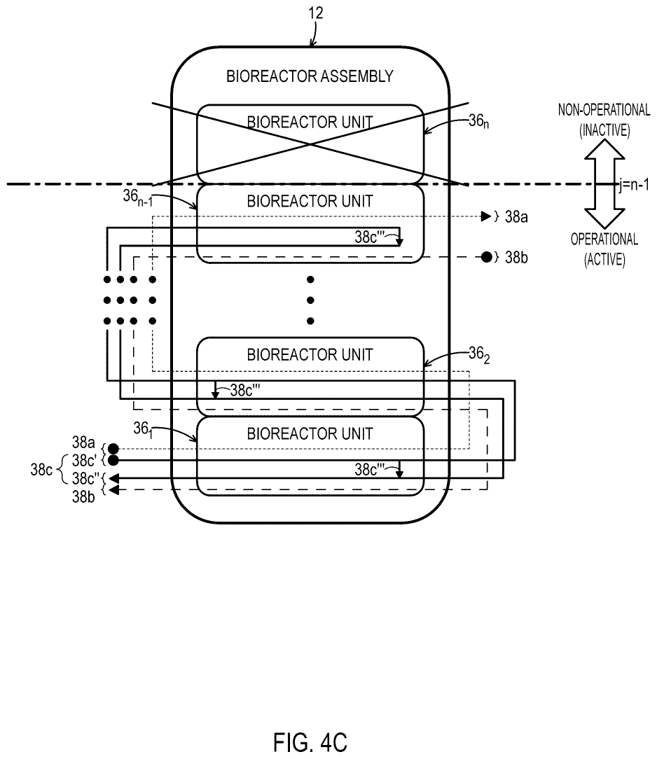

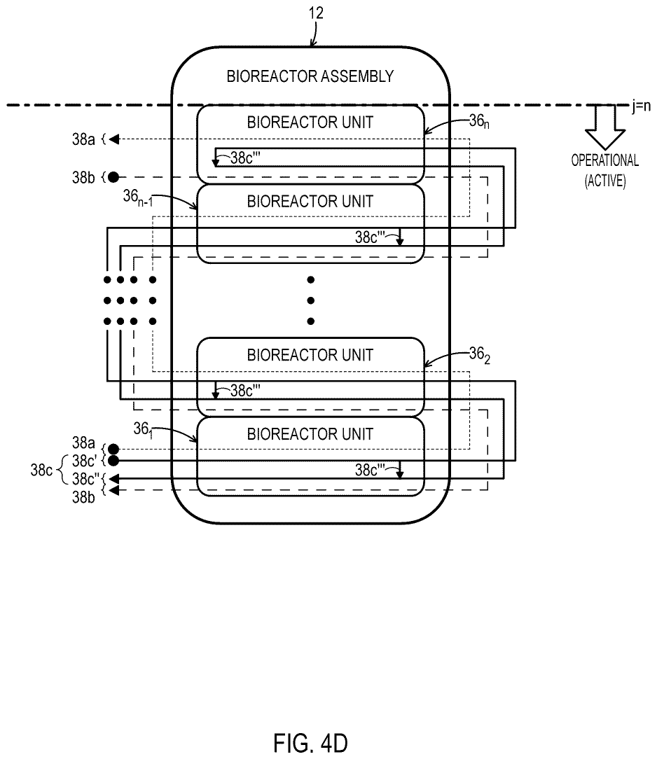

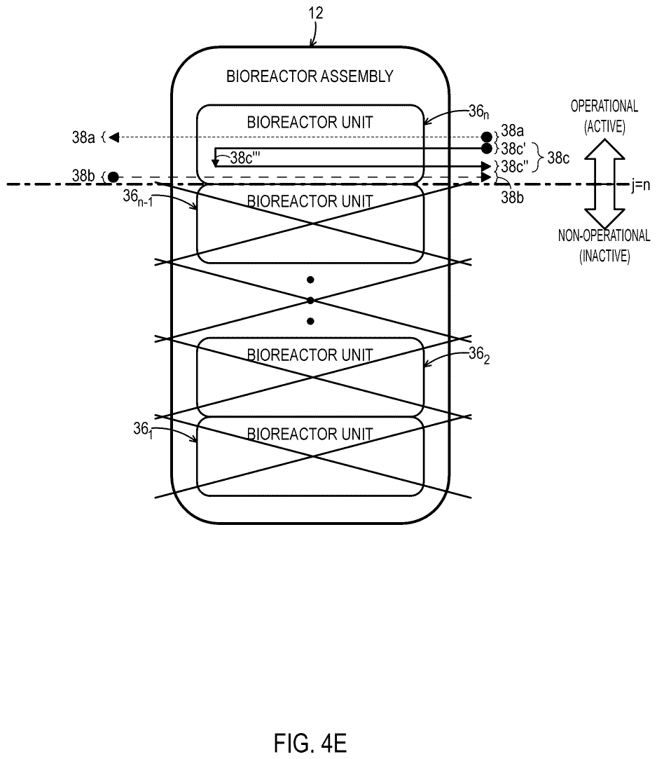

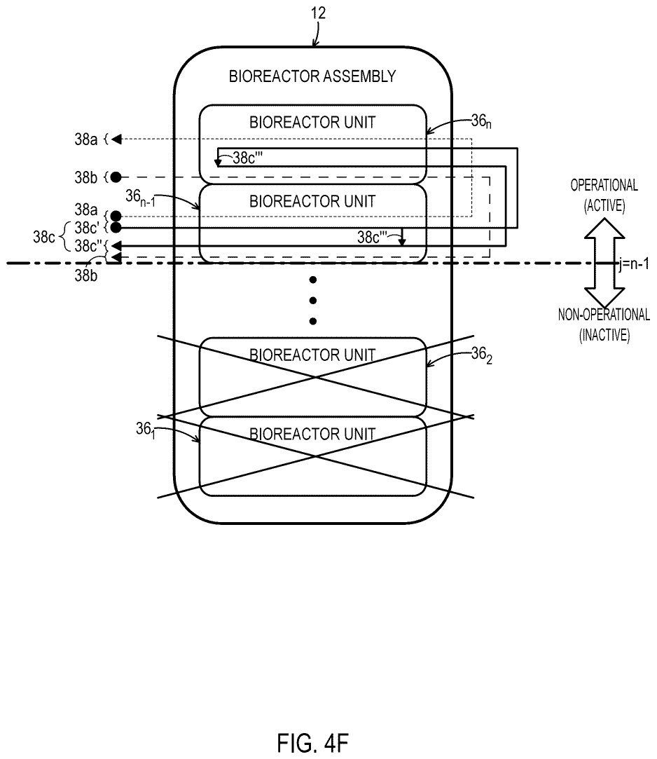

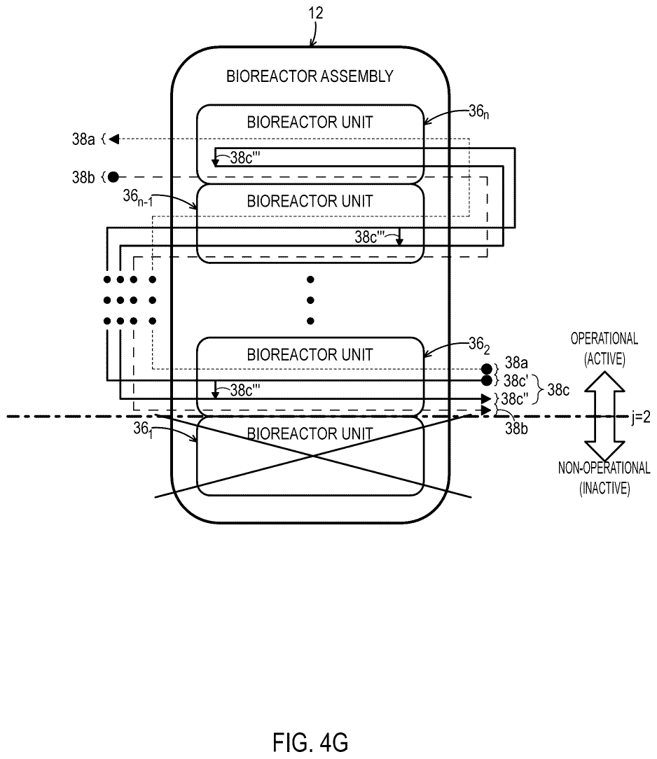

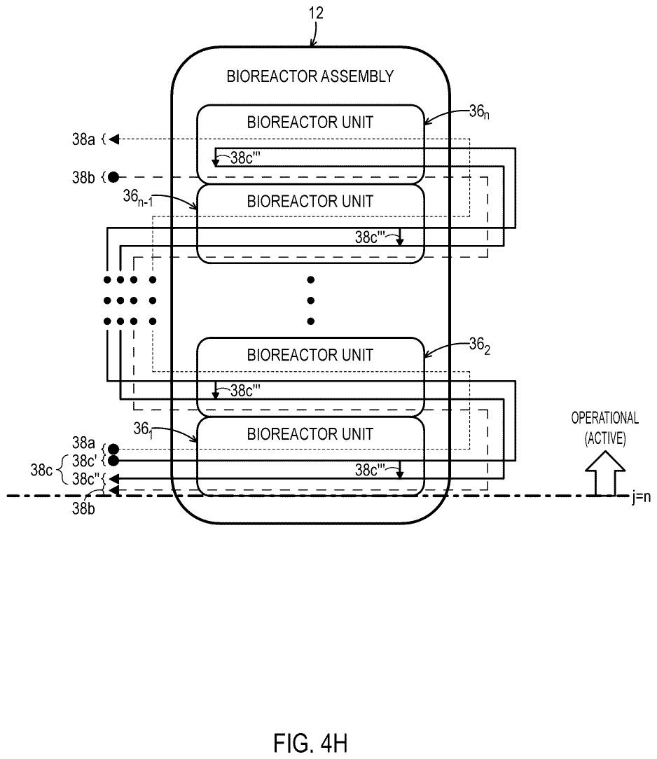

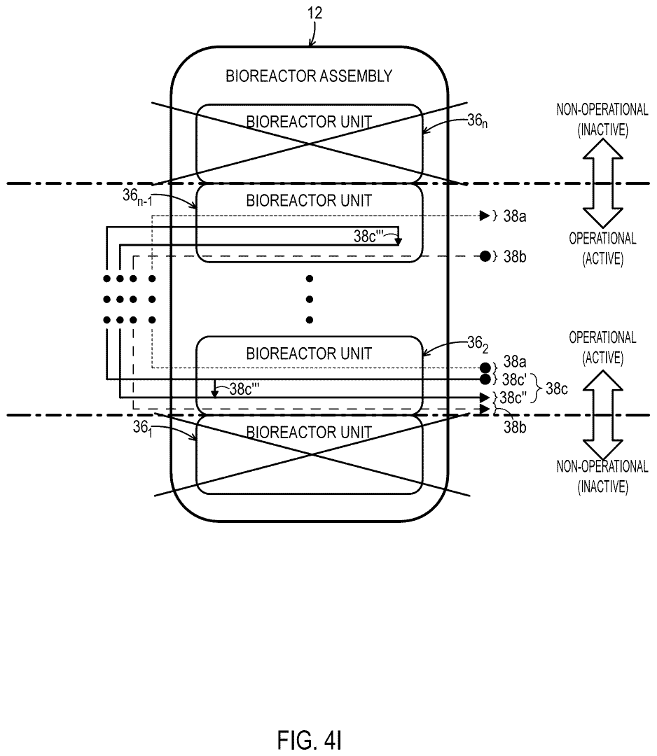

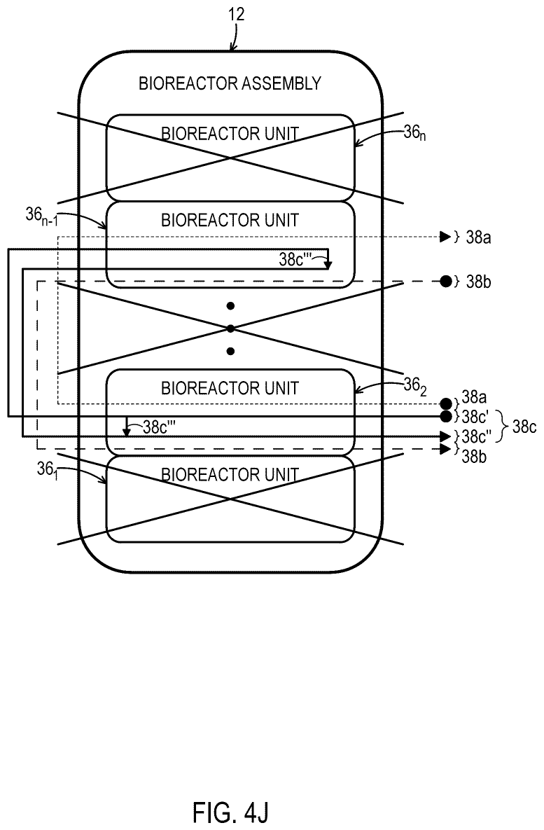

For example, in one embodiment illustrated in FIGS. 4A-4D, the valve assembly 16 may be operated (via the electric controller 22 shown in FIG. 1) to sequentially add bioreactor units 36 in time that pre-exist within the bioreactor assembly 12 to the flow paths 38a-38c. In this embodiment, the bioreactor units 36 may be sequentially added to the flow paths 38a-38c in the order that the bioreactor units 36 are stacked from bottom to top, such that all bioreactor units 36 below the last operational bioreactor unit 36ⱼ (i.e., the first stacked bioreactor unit 36₁ to the bioreactor unit 36ⱼ₋₁ immediately below the last operational bioreactor unit 36ⱼ) in the stack of bioreactor units 36 will likewise be operational (i.e., active), while all bioreactor units 36 above the last operational bioreactor unit 36ⱼ (i.e., the stacked bioreactor unit 36ⱼ₊₁ immediately above the last operational bioreactor unit 36ⱼ to the last stacked bioreactor unit 36ₙ) in the stack of bioreactor units 36 will be non-operational (i.e., inactive). Thus, the first stacked bioreactor unit 36₁ to the bioreactor unit 36ⱼ will be activated to add them to the flow paths 38a-38c, while the stacked bioreactor unit 36ⱼ and all stacked bioreactor units 36ⱼ₊₁ to 36ₙ above the bioreactor unit 36ⱼ will be deactivated to remove them from the flow paths 38a-38c.

Thus, the valve assembly 16 may be operated to activate the first stacked bioreactor unit 36₁ (i.e., added to the flow paths 38a-38c), and deactivate the second stacked bioreactor unit 36₂ to the last bioreactor unit 36ₙ (i.e., removed from the flow paths 38a-38c) (j=1) (FIG. 4A). The valve assembly 16 may then be operated to activate the first stacked bioreactor unit 36₁ and the second stacked bioreactor unit 36₂, and deactivate the third stacked bioreactor unit 36₃ to the last bioreactor unit 36ₙ (j=2) (FIG. 4B). The valve assembly 16 may then be operated to activate the first stacked bioreactor unit 36₁ to the third stacked bioreactor unit 36₃ and deactivate the fourth stacked bioreactor unit 364 to the last stacked bioreactor unit 36ₙ, and so forth until the first stacked bioreactor unit 36₁ to the third to last stacked bioreactor unit 36ₙ₋₂ are activated, and the second to last stacked bioreactor unit 36ₙ₋₁ and the last stacked bioreactor unit 36ₙ are deactivated (not shown). The valve assembly 16 may then be operated to activate the first stacked bioreactor unit 36₁ to the second to last stacked bioreactor unit 36ₙ₋₁, and deactivate the last stacked bioreactor unit 36ₙ (j=n−1) (FIG. 4C). The valve assembly 16 may then be operated to activate the first stacked bioreactor unit 36₁ to the last stacked bioreactor unit 36ₙ (j=n) (FIG. 4D).

Although the valve assembly 16 has been described as being operated to sequentially add in time the bioreactor units 36 in the order that the bioreactor units 36 are stacked from bottom to top, the valve assembly 16 may be operated (via the electric controller 22 shown in FIG. 1) to sequentially add in time from top to the bottom, as illustrated in FIGS. 4E-4H. Thus, the valve assembly 16 may be operated to activate the last stacked bioreactor unit 36ₙ (i.e., added to the flow paths 38a-38c), and deactivate the second to last stacked bioreactor unit 36ₙ₋₁ to the first stacked bioreactor unit 36₁ (i.e., removed from the flow paths 38a-38c) (j=n) (FIG. 4E). The valve assembly 16 may then be operated to activate the last stacked bioreactor unit 36ₙ and the second to last stacked bioreactor unit 36ₙ₋₁, and deactivate the third to last stacked bioreactor unit 36ₙ₋₂ to the first bioreactor unit 36₁ (j=n−1) (FIG. 4F). The valve assembly 16 may then be operated to activate the last stacked bioreactor unit 36ₙ to the third to last stacked bioreactor unit 36ₙ₋₂ and deactivate the fourth to the last stacked bioreactor unit 36ₙ₋₃ to the last stacked bioreactor unit 36ₙ, and so forth until the first stacked bioreactor unit 36₁ to the third to last stacked bioreactor unit 36ₙ₋₂ are activated, and the second to last stacked bioreactor unit 36ₙ₋₁ and the last stacked bioreactor unit 36ₙ are deactivated (not shown). The valve assembly 16 may then be operated to activate the last stacked bioreactor unit 36ₙ to the second stacked bioreactor unit 36₂, and deactivate the first stacked bioreactor unit 36₁ (j=2) (FIG. 4G). The valve assembly 16 may then be operated to activate the last stacked bioreactor unit 36ₙ to the first stacked bioreactor unit 36₁ (j=1) (FIG. 4H).

Although the valve assembly 16 has been described as being operated to generate flow sequences of bioreactor units 36 that either include the first stacked bioreactor unit 36₁, the last stacked bioreactor unit 36ₙ, or both, the valve assembly 16 may be operated to generate flow sequences of bioreactor units 36 that do not include the first stacked bioreactor unit 36₁ and the last stacked bioreactor unit 36ₙ (e.g., activating the second stacked bioreactor unit 36₂ to the second to last stacked bioreactor unit 36ₙ₋₁ (FIG. 4I)). Furthermore, although the valve assembly 16 has been described as being operated to generate flow sequences of bioreactor units 36 that are consistent with the sequence in which the bioreactor units 36 are physically arranged, the valve assembly 16 may be operated to generate flow sequences of bioreactor units 36 that are not consistent with the sequence in which the bioreactor units 36 are physically arranged (e.g., activating only the second stacked bioreactor unit 36₂ and the second to last stacked bioreactor unit 36ₙ₋₁ (see FIG. 4J).

It should be appreciated that although the valve assembly 16 has been described and illustrated as fully activating or fully deactivating various ones of the bioreactor units 36 (i.e., a particular bioreactor unit 36 is either removed from all of the flow paths 38a-38c or added to all of the flow paths 38a-38c), the valve assembly 16 may be operated to partially activate or partially deactivate a particular bioreactor unit 36 (i.e., a particular bioreactor unit 36 is removed from less than all of the flow paths 38a-38c) or added in less than all of the flow paths 38a-38c). For example, the valve assembly 16 may be operated to partially activate or deactivate a particular bioreactor unit 36 by removing the particular bioreactor unit 36 from the flow paths 38a, 38b, while the particular bioreactor unit 36 is added to the third flow path 38c.

Each of the bioreactor units 36 in the bioreactor assembly 12 may take the form of any suitable bioreactor capable of expanding a population of biological cells; for example, any of the bioreactors illustrated and described in U.S. Provisional Application Ser. No. 63/485,865, entitled “Bioreactor for Cellular Therapeutics,” filed Feb. 17, 2023, and PCT Application Ser. No. PCT/US2022/012194, entitled “Systems, Apparatuses, and Methods for Cellular Therapeutics Manufacture,” PCT/US2023/69944, entitled “Bioreactor for Cellular Therapeutics,” filed Jul. 11, 2023, which are expressly incorporated herein by reference.

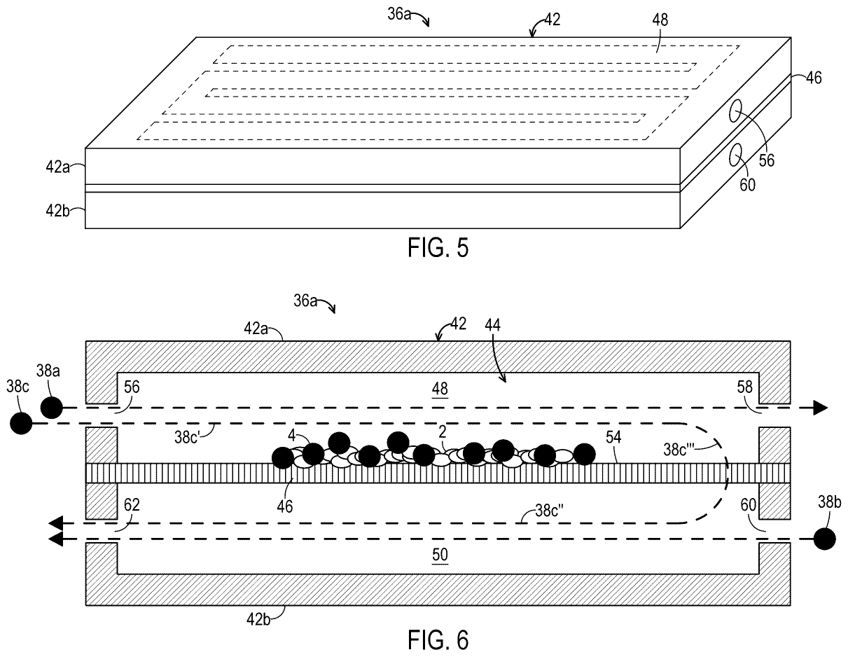

Referring to FIGS. 5 and 6, one exemplary embodiment of a bioreactor unit 36a will now be described.

The bioreactor unit 36a generally comprises a housing 42 having an interior cavity 44. The outer periphery of the housing 42, and thus the interior cavity 44, may be planar in nature (i.e., the length (x-dimension) and width (y-dimension) are much greater than the height or thickness (z-dimension)) to minimize the volume of media that flows through the bioreactor unit 36a, as will be described in further detail below. Furthermore, the outer periphery of the housing 42, and thus the interior cavity 44, has a suitable size that promotes the expansion of a population of biological cells 2 within the bioreactor unit 36a, e.g., sufficiently small enough to provide the necessary density for exhibiting cross-signaling within the population of biological cells 2, but large enough to avoid overcrowding within the population of biological cells 2.

In the illustrated embodiment, the housing 42 comprises a first (upper) hollow housing portion 42a and a second (lower) hollow housing portion 42b affixed together using suitable means (e.g., ultrasonic welding, adhesive, screws, or bolts) to form the interior cavity 44 therein. The upper and lower housing portions 42a, 42b may be composed of a suitable rigid material capable of withstanding fluid pressure within the interior cavity 44. As one example, the upper and lower housing portions 42a, 42b can be composed of a biocompatible material that is easy to manufacture, transparent, non-autofluorescent, and suitable for bonding to other plastics or elastomers. For example, the upper and lower housing portions 42a, 42b may be composed of a rigid biocompatible polymer material, such as, e.g., polycarbonate, polystyrene, cyclic olefin copolymer (COC), cyclic olefin polymer (COP), etc. In the preferred embodiment, the outer periphery of the lower housing portion 42b may have the same size as the outer periphery of the upper housing portion 42a, although in alternative embodiments, the outer periphery of the lower housing portion 42b may differ from the outer periphery of the upper housing portion 42a. In one example, the dimensions of the outer periphery of each of the upper and lower housing portions 42a, 42b may be in the range of 5 cm (width)×5 cm (length)×0.1 cm (height or thickness) to 30 cm (width)×30 cm (length)×30 cm (height or thickness). In one exemplary embodiment, the dimensions of the outer periphery of each of the upper and lower housing portions 42a, 42b is 10 cm (width)×10 cm (length)×1 cm (height or thickness).

The bioreactor unit 36a further comprises a membrane 46 affixed within the housing 42, such that the interior cavity 44 is divided into an upper flow channel 48 and a lower flow channel 50 through which different types of media can be flowed.

In the illustrated embodiment, the membrane 46 is affixed between the upper housing portion 42a and the lower housing portion 42b, such that membrane 46 is sandwiched between the housing portions 42a, 42b when they are affixed to each other. As a result, the upper flow channel 48 is formed between the upper housing portion 42a and the top surface of the membrane 46, whereas the lower flow channel 50 is formed between the lower housing portion 42b and the bottom surface of the membrane 46. The membrane 46 may be suitably bonded between the upper and lower housing portions 42a, 42b to ensure that media does not laterally leak out of the bioreactor unit 36a at the interfaces between the membrane 46 and housing portions 42a, 42b. The flow channels 48, 50 may be suitably formed, as portions of the interior cavity 44, into the respective housing portions 42a, 42b in any suitable manner, e.g., machining or injection molding.

In a similar manner as the outer peripheries of the housing 42, and thus the upper and lower housing portions 42a, 42b, described above, each of the flow channels 48, 50 may be planar in nature (i.e., the length (x-dimension) and width (y-dimension) are much greater than the height or thickness (z-dimension)) to minimize the volume of media that flows within the respective flow channels 48, 50. Furthermore, in a similar manner as the outer periphery of the upper housing portion 42a described above, the upper flow channel 48 has a suitable size and volume that promotes the expansion of biological cells, e.g., sufficiently small enough to provide the necessary density for exhibiting cross-signaling within the population of biological cells 2, but large enough to avoid overcrowding within the population of biological cells 2. By extension, the lower flow channel 50 may have the same size and volume as the upper flow channel 48.

For example, the dimensions of each of the flow channels 48, 50 may be in the range of 0.5 mm to 5 mm deep, 5 mm to 10 mm wide, and 500 mm-2500 mm long, and may have a volume in the range of 2 mL to 100 mL. In one exemplary embodiment, the dimensions of each of the flow channels 48, 50 may be 1 mm deep and 4 mm wide, and may have a volume of 4 mL. Although, in the illustrated embodiment, the dimensions and volumes of the flow channels 48, 50 are identical, the dimensions and volumes of the flow channels 48, 50 may differ from each other. In the illustrated embodiment, each of the flow channels 48, 50 is patterned (i.e., do not extend in a single straight line), and in particular, are serpentine in nature, as best illustrated in FIG. 5 (only upper flow channel 48 shown in phantom), thereby providing a more even flow across the surface area of the flow channels 48, 50, and allowing better clearance of bubbles trapped within the flow channels 48, 50, as well as more uniform nutrient distribution and cell homogeneity. In the serpentine pattern illustrated in FIG. 5, the upper flow channel 48 has a first segment that extends in a first direction for a first distance, then a second segment that extends from the first segment in second direction (e.g., perpendicular to the first segment) for a second distance smaller than the first distance; then a third segment that extends from the second segment in a third direction opposite and generally parallel to the first segment and for a third distance; then a fourth segment that extends from the third segment in a fourth direction (e.g., perpendicular to the third segment) for a fourth distance smaller than the third distance, and so forth as depicted in FIG. 5. Although, in the illustrated embodiment, the first and third distances are the same, in alternative embodiments, the first and third distances may differ from each other. In other embodiments, the different segments of the upper flow channel 48 may each extend in different directions and at different distances than that depicted in FIG. 5 to facilitate the flow of media.

The bioreactor unit 36a further comprises a cell culture surface 54 on which the population of biological cells 2 may be disposed. In the illustrated embodiment, the cell culture surface 54 is formed on the top surface of the membrane 46. In this manner, gravity urges the population of biological cells 2 to rest on the cell culture surface 54. The cell culture surface 54 may be smooth, such that after expansion of the seed population of biological cells 2, the cell culture surface 54 does not trap, and therefore easily releases, the biological cells 2, so as to not hinder harvesting of the expanded population of biological cells 2 from the upper flow channel 48. In alternative embodiments, the cell culture surface 54 may be chemically functionalized (e.g., with T cell activating agents (e.g., for antigen-specific or non-antigen-specific activation), or with surface blocking ligands and/or biocompatible polymers), biologically functionalized (e.g., biologically derived materials, such as primary and co-activating molecules), and/or structurally functionalized (e.g., with a plurality of concave features, such as dimples or grooves), as described in PCT Application Ser. No. PCT/US2022/012194, which has been previously incorporated herein by reference.

In an optional embodiment, microbeads 4 (e.g., polystyrene beads, glass beads, amorphous solid substrates, Luminex™ beads, or the like) may be disposed in the upper flow channel 48 to place the microbeads 4 in contact with the population of biological cells 2. The microbeads 4 may be liposome-coated or may include moieties/molecules covalently or non-covalently attached, such as fluorescent labels, proteins (including receptor molecules) carbohydrates, antigens, small molecule signaling moieties, or other chemical/biological species capable of use in an assay. In some embodiments, the microbeads 4 may serve as a synthetic surface for activating the biological cells 2 and may be magnetically manipulatable for sorting the population of biological cells 2 without the need for removing the population of biological cells 2 from the upper flow channel 48 until harvest.

As will be described in further detail, seeding media 26 containing a seed population of biological cells 2 (and optionally microbeads 4) can be flowed into the upper flow channel 48 to seed the bioreactor unit 36a with a population of biological cells 2 (and optionally microbeads 4), and fresh culture media 28 with nutrient, reagent, and gas can be flowed (continuously or intermittently) through the lower flow channel 50, thereby providing nutrient, reagent, and gas to, and extracting waste from, the population of biological cells 2 residing in the upper flow channel 48 via the action of diffusion through the membrane 46. As a result, expansion of the seed population of biological cells 2 is facilitated. Optionally, fresh wash buffer 30 may be flowed into the upper flow channel 48 during incubation of the population of biological cells 2 to remove undesirable material (e.g., unbound molecules, debris, dead cells, or other undesirable molecules). Further details on the use of microbeads and wash buffer are described in PCT Application Ser. No. PCT/US2022/012194, which has been expressly incorporated herein by reference.

Significantly, the membrane 46 is substantially permeable to the nutrient, reagent, and gas contained in the fresh culture media 28 and waste from the population of biological cells 2, such that the gas, nutrient, and reagent, may diffuse from the lower flow channel 50 into the upper flow channel 48 for take-up by the population of biological cells 2, and the waste may diffuse from the upper flow channel 48 into the lower flow channel 50 and away from the population of biological cells 2, thereby facilitating the expansion of the seed population of biological cells 2. Preferably, the membrane 46 provides a short diffusion path for the nutrient, gas, reagents, and waste between the flow channels 48, 50. Thus, the membrane 46 preferably has a relatively small thickness, e.g., in the range of 10-100 microns. In one specific embodiment, the thickness of the membrane 46 is 10 microns. For the purposes of this specification, the terms “diffuse” or “diffusion” may be defined as a process resulting from random motion of atoms, ions, or molecules (solid, liquid, or gas) by which there is a net flow of matter from a region of higher concentration to a region of lower concentration (i.e., across a chemical gradient).

Significantly, the diffusion of nutrient, reagent, gas, and waste across the membrane 46 creates a low flow between the upper flow channel 48 and the lower flow channel 50 that does not significantly disturb the population of biological cells 2. As a result, the population of biological cells 2 may remain statically on the cell culture surface 54, and in this illustrated case, on the membrane 46. Thus, the cell culture surface 54 may not need to be structurally functionalized to facilitate retention of the population of biological cells 2, thereby reducing or minimizing the chances that biological cells 2 will be trapped on the cell culture surface 54 when the expanded population of biological cells 2 are harvested from the upper flow channel 48.

In the preferred embodiment, the membrane 46 is also substantially permeable to the seeding media 26′ containing no biological cells 2 and microbeads 4 (and optionally, used wash buffer 30′ containing undesirable material), but is substantially impermeable to the biological cells 2 (and optionally, microbeads 4), such that, when the upper flow channel 48 is under pressure, the population of biological cells 2 (and optionally, microbeads 4) are substantially retained within the upper flow channel 48 while the seeding media 26′ containing no biological cells 2 and microbeads 4 (and optionally, used wash buffer 30′ containing undesirable material) perfuses through the membrane 46 from the upper flow channel 48 into the lower flow channel 50. In essence, the membrane 46 serves to filter the biological cells 2 (and optionally, microbeads 4) from the seeding media 26′ containing no biological cells 2 and microbeads 4 (and optionally, used wash buffer 30′ containing undesirable material) as the seeding media 26′ containing no biological cells 2 and microbeads 4 (and optionally, used containing undesirable’ containing undesirable material) perfuses through the membrane 46 from the upper flow channel 48 into the lower flow channel 50. To facilitate perfusion of the seeding media 26′ containing no biological cells 2 and microbeads 4 (and optionally, used wash buffer 30′ containing undesirable material) through the membrane 46 between the flow channels 48, 50, the membrane 46 is preferably porous, meaning that membrane 46 has a solid matrix with defined pores that have diameters ranging from 0.01 microns to 20 microns. In one example, the porous membrane 46 may have a pore diameter in the range of 0.05-0.4 microns, and a porosity in the range of 1%-20%. In one specific embodiment, the porous membrane 46 has a pore diameter of 0.2 microns, and a porosity of 10%. For the purposes of this specification the terms “perfuse” or “perfusion” may be defined as the bulk movement of a liquid from a region of higher pressure to a region of lower pressure (i.e., across a pressure gradient). In one example, the porous membrane 46 is substantially permeable to a particular component if more than 90 percent of that component can pass through the porous membrane 46 between the flow channels 48, 50, and substantially impermeable to a particular component if less than 10 percent of that component can pass through the porous membrane 46 from the upper flow channel 48 into the lower flow channel 50. Preferably, the porous membrane 46 resists flexing in the presence of a dynamic pressure differential between the flow channels 48, 50. To this end, the porous membrane 46 may be composed of any suitable durable biocompatible material, such as, e.g., polycarbonate.

The bioreactor unit 36a may optionally comprise at least one permeable support structure (not shown) configured for reducing a lateral flex of the porous membrane 46 in response to a pressure differential between the flow channels 48 that may otherwise disturb the population of biological cells 2 resting on the top surface of the porous membrane 46, resulting in less expansion of the seed population of biological cells 2. Further details discussing permeable support structures are described in U.S. Provisional Application Ser. No. 63/485,865, entitled “Bioreactor for Cellular Therapeutics,” filed Feb. 17, 2023, and PCT Application Ser. No. PCT/US2022/012194, entitled “Systems, Apparatuses, and Methods for Cellular Therapeutics Manufacture,” PCT/US2023/69944, entitled “Bioreactor for Cellular Therapeutics,” filed Jul. 11, 2023, which have been expressly incorporated herein by reference.

The bioreactor unit 36a has an arrangement of inlet and outlet ports to facilitate the flow of the priming media 24, seeding media 26, fresh culture media 28, fresh wash buffer 30, and harvesting media 32 through the interior cavity 44 of the bioreactor unit 36a.

In particular, the bioreactor unit 36a comprises an inlet port 56 and outlet port 58 associated with the upper flow channel 48, and an inlet port 60 and outlet port 62 associated with the lower flow channel 50. Although the inlet port 56 and the inlet port 60 are illustrated as being located on opposite sides (respectively, the left and right sides in FIG. 6) of the bioreactor unit 36a, and the outlet port 58 and outlet port 62 as being located on opposite sides (respectively, the right and left sides in FIG. 6) of the bioreactor unit 36a, the inlet port 56 and the inlet port 60 may be located on the same side (e.g., both on the left side in FIG. 6) of the bioreactor unit 36a, and the outlet port 58 and outlet port 62 may be located on the same side (e.g., on the right side in FIG. 6) of the bioreactor unit 36a. Furthermore, although the inlet port 56 and the outlet port 58 are illustrated as being located on opposite sides (respectively the left and right sides in FIG. 6) of the bioreactor unit 36a, and the inlet port 60 and outlet port 62 are illustrated as being located on opposite sides (respectively the right and left sides in FIG. 6) of the bioreactor unit 36a, the inlet port 56 and the outlet port 58 may be located on same side (e.g., left side) of the bioreactor unit 36a, while the inlet port 60 and outlet port 62 may be located on same side (e.g., the right side) of the bioreactor unit 36a. Notably, although the ports 56-62 are illustrated as external ports to which external conduits can be actively connected to and disconnected from, one or more of the ports 56-62 may be internal ports to which internal flow channels may be fluidly coupled.

In one embodiment, the afore-described flow paths 38a-38c may extend through the bioreactor unit 36a. In particular, the flow path 38a may extend in through the inlet port 56, through the upper flow channel 48, and out through the outlet port 58; the flow path 38b may extend in through the inlet port 60, through the lower flow channel 50, and out through the outlet port 60; and the forward portion 38c′ of the flow path 38c may extend in through the inlet port 56 and into the upper flow channel 48, the bypass portion 38c″ of the flow path 38c may cross or traverse the porous membrane 46 from the upper flow channel 48 into the lower flow channel 50, and the return portion 38c″ of the flow path 38c may exit the lower flow channel 50 out through the outlet port 62. In an alternative embodiment, the forward portion 38c′ of the flow path 38c (or another flow path) may extend in through the inlet port 60 and into the lower flow channel 50, the bypass portion 38c″ of the flow path 38c (or the other flow path) may cross or traverse the porous membrane 46 from the lower flow channel 50 into the upper flow channel 48, and the return portion 38c″ of the flow path 38c (or the other flow path) may exit the upper flow channel 48 out through the outlet port 58.

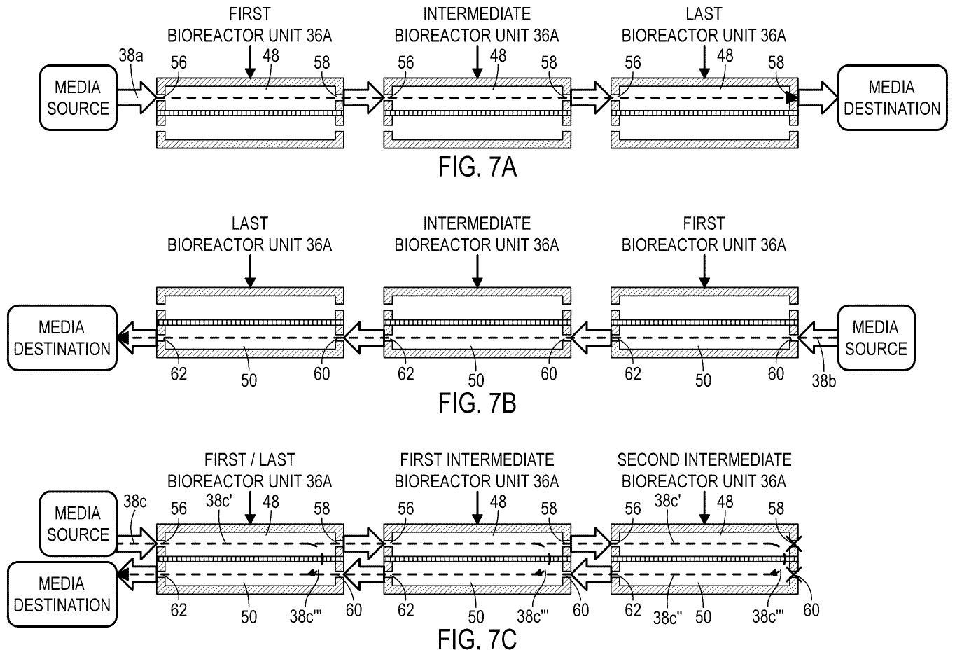

Referring further to FIGS. 7A-7C, examples illustrating the manner in which a flow sequence of bioreactor units 36a is added to each of the flow paths 38a-38c between a media source and a media destination will now be described. Depending on the nature of the flow path, the sequence of bioreactor units 36a may be added in series or added in parallel with a particular flow path. As a general rule, a first bioreactor unit in a flow sequence means the bioreactor unit having an input closest in the particular flow path to the media source, while the last bioreactor unit in a flow sequence means the bioreactor unit having an input furthest in a particular flow path to the media source. A preceding bioreactor unit in a flow sequence means the bioreactor unit upstream in a particular flow path (or portion thereof) to the media source relative to another bioreactor unit, while a succeeding bioreactor unit in a flow sequence means the bioreactor unit downstream in a particular flow path (or portion thereof) relative to another bioreactor unit. It should be appreciated that a particular bioreactor unit may be considered both a “preceding bioreactor unit” and “succeeding bioreactor unit” with respect to different flow paths (or portions thereof). Thus, a bioreactor unit 36a may be a preceding bioreactor unit with respect to the first flow path 38a, but a subsequent bioreactor unit with respect to the second flow path 38b. It should also be appreciated that a flow sequence of bioreactor units 36a does not necessarily mean that such flow sequenced bioreactor units 36a are physically located relative to each other in a series, but rather means that the bioreactor units 36a are fluidly coupled in a sequence notwithstanding that all of such bioreactor units 36a may not be physically located in series relative to each other.

As illustrated in FIG. 7A, a flow sequence of bioreactor units 36a can be added in series to the first flow path 38a by fluidly coupling a media source (e.g., one or more of the wash buffer source 14f, and saline source 14g (shown in FIG. 1)) to the inlet port 56 of the first bioreactor unit 36a in the flow sequence; fluidly coupling a media destination (e.g., one of the biological cell collection destination 14h and waste destination 14i (shown in FIG. 1)) to the outlet port 58 of the last bioreactor unit 36a in the flow sequence; and fluidly coupling the outlet port 58 of each preceding bioreactor unit 36a to the inlet port 56 of the immediately succeeding bioreactor unit 36a (in this case, the outlet port 58 of the first bioreactor unit 36a to the inlet port 56 of the intermediate bioreactor unit 36a, and the outlet port 58 of the intermediate bioreactor unit 36a to the inlet port 56 of the last bioreactor unit 36a), thereby fluidly coupling the upper flow channels 48 of the bioreactor units 36a in series along the first flow path 38a between the media source and the media destination.

As illustrated in FIG. 7B, a flow sequence of bioreactor units 36a can be added in series to the second flow path 38b by fluidly coupling a media source (e.g., one or more of the nutrient source 14c, reagent source 14d, gas source 14e, wash buffer source 14f, and saline source 14g (shown in FIG. 1)) to the inlet port 60 of the first bioreactor unit 36a in the flow sequence; fluidly coupling a media destination (e.g., the waste destination 14i (shown in FIG. 1)) to the outlet port 62 of the last bioreactor unit 36a in the flow sequence; and fluidly coupling the outlet port 62 of each preceding bioreactor unit 36a to the inlet port 60 of the immediately succeeding bioreactor unit 36a (in this case, the outlet port 62 of the first bioreactor unit 36a to the inlet port 60 of the intermediate bioreactor unit 36a, and the outlet port 62 of the intermediate bioreactor unit 36a to the inlet port 60 of the last bioreactor unit 36a), thereby fluidly coupling the lower flow channels 50 of the bioreactor units 36a in series along the second flow path 38b between the media source and the media destination.

As illustrated in FIG. 7C, a flow sequence of bioreactor units 36a can be added in parallel to the third flow path 38c by fluidly coupling a media source (e.g., one or more of the biological cell source 14a, microbead source 14b, wash buffer source 14f, and saline source 14g (shown in FIG. 1)) to the inlet port 56 of the first bioreactor unit 36a in the flow sequence; fluidly coupling a media destination (e.g., the waste destination 14i (shown in FIG. 1)) to the outlet port 62 of the last bioreactor unit 36a in the flow sequence (which is the same as the first bioreactor unit in the flow sequence); fluidly coupling the outlet port 58 and inlet port 60 of each preceding bioreactor unit 36a respectively to the inlet port 56 and outlet port 62 of the immediately subsequent bioreactor unit 36a (in this case, the outlet port 58 and inlet port 60 of the first bioreactor unit 36a to the inlet port 56 and outlet port 62 of the first intermediate bioreactor unit 36a, and the outlet port 58 and inlet port 60 of the first intermediate bioreactor unit 36a to the inlet port 56 and outlet port 62 of the second intermediate bioreactor unit 36a), thereby fluidly coupling the upper flow channels 48 of the bioreactor units 36a in series along the forward portion 38c′ of the third flow path 38a between the media source and the media destination, and fluidly coupling the lower flow channels 50 of the bioreactor units 36a in series along the return portion 38c″ of the third flow path 38c between the media source and the media destination; and fluidly decoupling the outlet port 58 and inlet port 60 of the bioreactor unit 36a in the flow sequence in which the forward portion 38c′ of the third flow path 38a ends and the return portion 38c″ of the third flow patch 38a ends from any source of pressure less than the pressure required to perfuse the various media through the porous membrane 46 between the flow channels 48, 50, thereby fluidly coupling the upper flow channel 48 and lower flow channel 50 of each bioreactor unit 36a to each other.

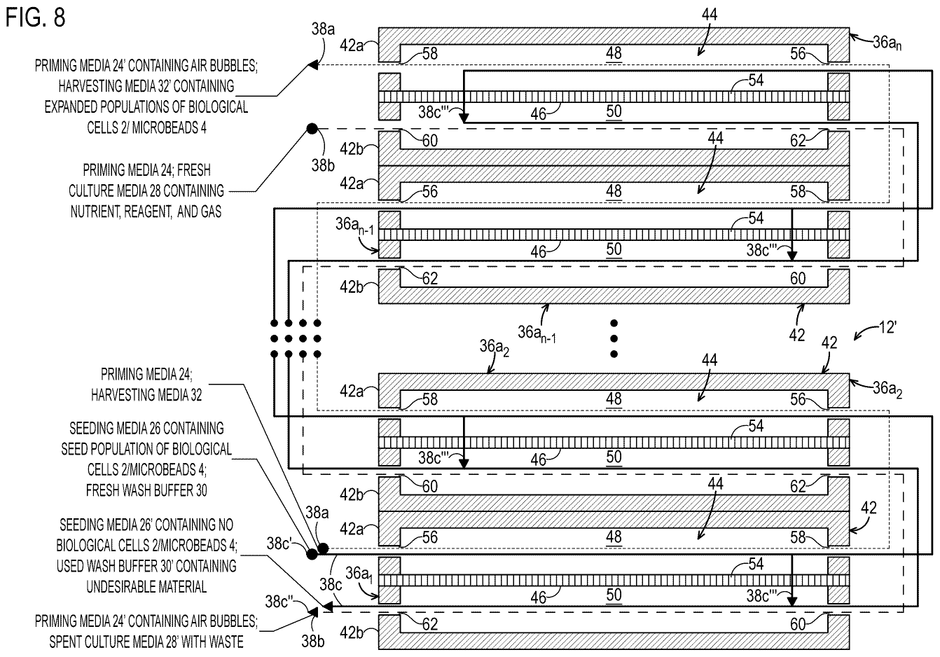

Referring now to FIG. 8, one embodiment of a bioreactor assembly 12′ that incorporates bioreactor units 36a of FIGS. 5-6 will be described. In this embodiment, the bioreactor units 36a are structurally distinct (e.g., they have separate housings), such that they can be structurally affixed to each other or removed from each other (e.g., via fasteners (not shown)) in a dynamic manner. In this manner, the bioreactor assembly 12′ can be structurally scaled up or scaled down in a dynamic manner; in essence, adding or subtracting flow channels 48, 50, as needed either prior to the beginning of the biological cell expansion process or during the biological cell expansion process. In an alternative embodiment of a bioreactor assembly, bioreactor units may be permanently structurally integrated (e.g., they have a single housing), in which case, scaling of such bioreactor assembly up or down may be accomplished during the manufacturing process, e.g., by fabricating bioreactor assemblies with different sizes and/or different numbers of bioreactor units.

Whether the bioreactor assembly includes structurally distinct bioreactor units 36a that can be dynamically affixed to each other or removed from each other or whether the bioreactor assembly has permanently integrated bioreactor units 36a, such bioreactor assembly may be functionally scaled up or down by activating (rendering operational) selected ones of the bioreactor units 36a by adding them to the flow paths 38a-38c, or deactivating (rendering non-operational) selected ones of the bioreactor units 36a by removing them from the flow paths 38a-38c; e.g., by adding a flow sequence of bioreactor units 36a to one or more of the flow paths 38a-38c between a media source and a media destination, as illustrated in FIGS. 7A-7C.

In the examples described below, the flow sequence of bioreactor units 36a added to a particular one of the flow paths 38a-38c includes all n number of stacked bioreactor units 36a₁ to 36aₙ, which are fluidly coupled in sequence in the same order as the order in which the n number of stacked bioreactor units 36a₁ to 36aₙ are physically arranged with each other (i.e., fluidly coupling the first stacked bioreactor unit 36a₁ directly to the second stacked bioreactor unit 36a₂, fluidly coupling the second stacked bioreactor unit 36a₂ directly to the third stacked bioreactor unit 36a₃, and so forth). However, it should be appreciated that the flow sequence of bioreactor units 36a added to a particular one of the flow paths 38a-38c may include less than all n number of stacked bioreactor units 36a₁ to 36aₙ, including a flow sequence that excludes the first stacked bioreactor unit 36a₁ (e.g., stacked bioreactor units 36a₂ to 36aₙ), a flow sequence that excludes the last stacked bioreactor unit 36aₙ (e.g., stacked bioreactor units 36a₁ to 36aₙ₋₁), a flow sequence that excludes any one or more of the stacked bioreactor units 36a₂-36aₙ₋₁ between the first stacked bioreactor unit 36a₁ and the last stacked bioreactor unit 36aₙ (e.g., stacked bioreactor units 36a₂ to 36aₙ₋₁), or any flow sequence that fluidly couples bioreactor units 36a in an order that does not match the order in which such bioreactor units 36a are physically arranged relative to each other (e.g., the first stacked bioreactor unit 36a₁, then the second to last stacked bioreactor unit 36aₙ₋₁, then the third stacked bioreactor unit 36a₃, etc.).

The valve assembly 16 (illustrated in FIG. 1) may be operated to selectively open or close the ports 56-62 of a flow sequence of bioreactor units 36a of the bioreactor assembly 12′ to selected ones of the flow paths 38a-38c (i.e., by actively fluidly coupling selected ones the ports 56-62 to, or actively decoupling the selected ones of ports 56-62 from, the selected flow paths 38a-38c), thereby adding (activating) the flow sequence of bioreactor units 36a to the selected ones of the flow paths 38a-38c, while selectively fluidly coupling selected ones of the media sources/media destinations 14a-14i to the selected flow paths 38a-38c, to effect different functions, e.g., any one (or any combination) of the aforementioned functions (a)-(e) described above with respect to FIG. 1.

In one example, the valve assembly 16, under control of the electric controller 22 (shown in FIG. 1), may be operated to prime the upper flow channels 48 of the bioreactor assembly 12′ by adding a flow sequence of bioreactor units 36a (in the illustrated case, all n number of stacked bioreactor units 36a₁ to 36aₙ) to the first flow path 38a between the saline source 14g and the waste destination 14i (shown in FIG. 1), with the saline source 14g serving as the media source, the waste destination 14i serving as the media destination, the first stacked bioreactor unit 36a₁ serving as a first bioreactor unit 36a in the flow sequence, the last stacked bioreactor unit 36aₙ serving as the last bioreactor unit 36a in the flow sequence, and the second stacked bioreactor unit 36a₂ to the second to last stacked bioreactor unit 36aₙ₋₁ serving as intermediate bioreactor units 36a in the flow sequence (see FIG. 7A).

In particular, the valve assembly 16 may be operated to prime the upper flow channels 48 of the bioreactor assembly 12′ of FIG. 8 by fluidly coupling the saline source 14g to the inlet port 56 of the first stacked bioreactor unit 36a₁; fluidly coupling the waste destination 14i to the outlet port 58 of the last stacked bioreactor unit 36aₙ); and fluidly coupling the outlet port 58 of each bioreactor unit 36a to the inlet port 56 of the immediately succeeding bioreactor unit 36a in the flow sequence along the second flow path 38b (i.e., the outlet port 58 of the first stacked bioreactor unit 36a₁ to the inlet port 56 of the second stacked bioreactor unit 36a₂, the outlet port 58 of the second stacked bioreactor unit 36a₂ to the inlet port 56 of the third stacked bioreactor unit 36a₃, and so forth), thereby fluidly coupling the upper flow channels 48 of the bioreactor units 36a in series between the saline source 14g and the waste destination 14i.

As a result, under the fluid pressure applied by the pump assembly 18 (shown in FIG. 1), priming media 24 flows along the first flow path 38a from the saline source 14g, in through the open inlet port 56 and through the upper flow channel 48 of the first stacked bioreactor unit 36a₁, thereby accumulating air bubbles from the upper flow channel 48, and priming media 24′ containing air bubbles then flows out through the open outlet port 58 of the first stacked bioreactor unit 36a₁; then the priming media 24′ containing air bubbles from the first stacked bioreactor unit 36a₁ flows further along the first flow path 38a in through the open inlet port 56 and through the upper flow channel 48 of the second stacked bioreactor unit 36a₂, thereby accumulating more air bubbles from the upper flow channel 48, and the priming media 24′ containing air bubbles then flows out through the open outlet port 58 of the second stacked bioreactor unit 36a₂; and so forth until the priming media 24′ containing air bubbles from all n number of bioreactor units 36a flows out of the open outlet port 58 of the last stacked bioreactor unit 36aₙ to the waste destination 14i. Thus, any or most of the air within the upper flow channels 48 of the n number of bioreactor units 36a will be completely displaced (i.e., flushed out) by the priming media 24. In this manner, the first flow path 38a (and at least the portion of the third flow path 38c that coincides with the first flow path 38a) extending through the n number of bioreactor units 36a will be consistent and well-defined.

Likewise, the valve assembly 16, under control of the electric controller 22 (shown in FIG. 1), may be operated to prime the lower flow channels 50 of the bioreactor assembly 12′ by adding a flow sequence of bioreactor units 36a (in the illustrated case, all n number of stacked bioreactor units 36a₁ to 36aₙ) to the second flow path 38b between the saline source 14g and the waste destination 14i (shown in FIG. 1), with the saline source 14g serving as the media source, the waste destination 14i serving as the media destination, the last stacked bioreactor unit 36aₙ serving as a first bioreactor unit 36a in the flow sequence, the first stacked bioreactor unit 36a₁ serving as the last bioreactor unit 36a in the flow sequence, and the second to last stacked bioreactor unit 36aₙ₋₁ to the second stacked bioreactor unit 36a₂ serving as intermediate bioreactor units 36a in the flow sequence (see FIG. 7B).

In particular, the valve assembly 16 may be operated to prime the lower flow channels 50 of the bioreactor assembly 12′ by fluidly coupling the saline source 14g to the inlet port 60 of the last stacked bioreactor unit 36aₙ; fluidly coupling the waste destination 14i to the outlet port 62 of the first stacked bioreactor unit 36a₁); and fluidly coupling the outlet port 62 of each bioreactor unit 36a to the inlet port 60 of the immediately succeeding bioreactor unit 36a in the flow sequence along the first flow path 38a (i.e., the outlet port 62 of the last stacked bioreactor unit 36aₙ to the inlet port 60 of the second to last stacked bioreactor unit 36aₙ₋₁, the outlet port 62 of the second to last stacked bioreactor unit 36aₙ₋₁ to the inlet port 60 of the third to last stacked bioreactor unit 36aₙ₋₂, and so forth), thereby fluidly coupling the lower flow channels 50 of the bioreactor units 36a in series between the saline source 14g and the waste destination 14i.

As a result, under the fluid pressure applied by the pump assembly 18 (shown in FIG. 1), priming media 24 flows along the second flow path 38b from the saline source 14g, in through the open inlet port 60 and through the lower flow channel 50 of the last stacked bioreactor unit 36aₙ, thereby accumulating air bubbles from the lower flow channel 50, and priming media 24′ containing air bubbles then flows out through the open outlet port 62 of the last stacked bioreactor unit 36aₙ; then the priming media 24′ containing air bubbles from the last stacked bioreactor unit 36aₙ flows further along the second flow path 38b in through the open inlet port 60 and through the lower flow channel 50 of the second to last stacked bioreactor unit 36aₙ₋₁, thereby accumulating more air bubbles from the lower flow channel 50, and the priming media 24′ containing air bubbles then flows out through the open outlet port 62 of the second to last stacked bioreactor unit 36aₙ₋₁; and so forth until the priming media 24′ containing air bubbles from all n number of bioreactor units 36a flows out of the open outlet port 62 of the first stacked bioreactor unit 36a₁ to the waste destination 14i. Thus, any or most of the air within the lower flow channels 50 of the respective n number of bioreactor units 36a will be completely displaced (i.e., flushed out) by the priming media 24. In this manner, the second flow path 38b (and at least the portion of the third flow path 38c that coincides with the second flow path 38b) extending through the n number of bioreactor units 36a will be consistent and well-defined.

In another example, the valve assembly 16, under control of the electric controller 22 (shown in FIG. 1), may be operated to seed the bioreactor assembly 12′ with multiple populations of biological cells 2 and/or microbeads 4 by adding a flow sequence of bioreactor units 36a (in the illustrated case, all n number of stacked bioreactor units 36a₁ to 36aₙ) to the third flow path 38c between the biological cell source 14a and/or microbead source 14b and the waste destination 14i (shown in FIG. 1), with the biological cell source 14a and/or microbead source 14b serving as the media source, the waste destination 14i serving as the media destination, the first stacked bioreactor unit 36a₁ serving as both the first bioreactor unit 36a and the last bioreactor unit 36a, and the second stacked bioreactor unit 36a₂ to the last stacked bioreactor unit 36aₙ serving as intermediate bioreactor units 36a, in the flow sequence along the forward portion 38c′ and return portion 38c″ of the third flow path 38c (see FIG. 7C).

In particular, the valve assembly 16 may be operated to seed the bioreactor assembly 12′ with the populations of biological cells 2 and/or microbeads 4 by fluidly coupling the biological cell source 14a and/or microbead source 14b to the inlet port 56 of the first stacked bioreactor unit 36a₁; fluidly coupling the waste destination 14i to the outlet port 62 of the first stacked bioreactor unit 36a₁; fluidly coupling the outlet port 58 of each bioreactor unit 36a to the inlet port 56 of the immediately succeeding bioreactor unit 36a in the flow sequence along the forward path 38c′ of the third flow path 38c (i.e., the outlet port 58 of the first stacked bioreactor unit 36a₁ to the inlet port 56 of the second stacked bioreactor unit 36a₂, the outlet port 58 of the second stacked bioreactor unit 36a₂ to the inlet port 56 of the third stacked bioreactor unit 36a₃, and so forth), thereby fluidly coupling the upper flow channels 48 of the bioreactor units 36a in series between the saline source 14g and the waste destination 14i; fluidly coupling the outlet port 62 of each bioreactor unit 36a to the inlet port 60 of the immediately succeeding bioreactor unit 36a in the flow sequence along the return path 38c″ of the third flow path 38c (i.e., the outlet port 62 of the last stacked bioreactor unit 36aₙ to the inlet port 60 of the second to last stacked bioreactor unit 36aₙ₋₁, the outlet port 62 of the second to last stacked bioreactor unit 36a₂ to the inlet port 60 of the third to last stacked bioreactor unit 36a₃, and so forth), thereby fluidly coupling the lower flow channels 50 of the bioreactor units 36a in series between the saline source 14g and the waste destination 14i; and fluidly decoupling the outlet port 58 and the inlet port 60 of the last stacked bioreactor unit 36aₙ from any source of fluid, thereby fluidly coupling the upper flow channel 48 and lower flow channel 50 of each bioreactor unit 36a to each other (in effect, removing the stacked bioreactor units 36a₁ to 36aₙ from the other flow paths 38a, 38b to increase the pressure in the upper flow channels 48 to urge fluidly coupling between the upper flow channels 48 and the lower flow channels 50.

As a result, under the fluid pressure applied by the pump assembly 18 (shown in FIG. 1), seeding media 26 containing a seed population of biological cells 2 and/or microbeads 4 flows along the forward portion 38c′ of the third flow path 38c from the biological cell source 14a and/or microbead source 14b, in through the open inlet port 56, through the upper flow channel 48, and out through the open outlet port 58, of the first stacked bioreactor unit 36a₁; then some of the seeding media 26′ without the biological cells 2 and/or microbeads 4 perfuses along the bypass portion 38c″ of the third flow path 38c through the porous membrane 46 from the upper flow channel 48 into the lower flow channel 50, of the first stacked bioreactor unit 36a₁ (due to the pressure buildup in the upper flow channel 48 caused by the closed outlet port 58 of the last stacked bioreactor unit 36aₙ creating a pressure gradient between the flow channels 48, 50 of all n number of stacked bioreactor units 36a₁ to 36aₙ); and then the perfused seeding media 26′ without the population of biological cells 2 and/or microbeads 4 flows along the return portion 38c″ of the third flow path 38c from the lower flow channel 50 and only out through the open outlet port 62, of the first stacked bioreactor unit 36a₁ (due to the closed inlet port 60 of the last stacked bioreactor unit 36aₙ preventing the flow of fluid along the second flow path 38b in the opposite direction from the lower flow channels 50 out through the inlet ports 60 of all n number of stacked bioreactor units 36a₁ to 36aₙ), and to the waste destination 14i. Significantly, while the seeding media 26′ perfuses through the porous membrane 46, the porous membrane 46 captures (filters) the biological cells 2 and/or microbeads 4, which are urged against the cell culture surface 54 on the porous membrane 46 via the action of convection, thereby seeding the upper flow channel 48 of the first stacked bioreactor unit 36a₁ with a population of biological cells 2 and/or microbeads 4.

This flow of the seeding media 26 containing the seed population of biological cells 2 and/or microbeads 4 and the seeding media 26 containing no biological cells 2 and/or microbeads 4 repeats for the second stacked bioreactor unit 36a₂ to the second to last bioreactor 36aₙ₋₁, with the seeding media 26 containing the seed population of biological cells 2 and/or microbeads 4 flowing along the forward portion 38c′ of the third flow path 38c out through the open outlet port 58 of each bioreactor unit 36a, flowing in through the open inlet port 56 of an immediately succeeding bioreactor unit 36a in the flow sequence, and the seeding media 26 containing no biological cells 2 and/or microbeads 4 that has perfused along the bypass portion 38c″ of the third flow path 38c across the porous membrane 46, flowing along the return portion 38c″ of the third flow path 38c out through the open outlet port 62 of each bioreactor unit 36a and flowing in through the open inlet port 60 of an immediately succeeding bioreactor unit 36a in the flow sequence, thereby seeding the second stacked bioreactor unit 36a₂ to the second to last bioreactor 36aₙ₋₁ with populations of biological cells 2 and/or microbeads 4.

Lastly, the seeding media 26 containing the seed population of biological cells 2 and/or microbeads 4 flows along the forward portion 38c′ of the third flow path 38c from the open outlet port 58 of the second to last stacked bioreactor unit 36aₙ₋₁, in through the open inlet port 56, and into the upper flow channel 48, of the last stacked bioreactor unit 36aₙ; then the seeding media 26′ without the biological cells 2 and/or microbeads 4 perfuses along the bypass portion 38c″ of the third flow path 38c through the porous membrane 46 from the upper flow channel 48 into the lower flow channel 50, of the last stacked bioreactor unit 36aₙ (due to the pressure buildup in the upper flow channel 48 caused by the closed outlet port 58 of the last stacked bioreactor unit 36aₙ creating a pressure gradient between the flow channels 48, 50 of all n number of stacked bioreactor units 36a₁ to 36aₙ); and then the perfused seeding media 26′ without the population of biological cells 2 and/or microbeads 4 flows along the return portion 38c″ of the third flow path 38c from the lower flow channel 50 and out through the open outlet port 62, of the last stacked bioreactor unit 36aₙ, back in through the open inlet port 60 of the second to last stacked bioreactor unit 36aₙ₋₁, thereby seeding the upper flow channel 48 of the last stacked bioreactor unit 36aₙ with a population of biological cells 2 and/or microbeads 4.

Significantly, because the biological cells 2 and/or microbeads 4 are filtered and captured by the porous membranes 46 of the respective n number of bioreactors 36a while the seeding media 26 containing the seed population of biological cells 2 and microbeads 4 are prevented from flowing out of the upper flow channel 48 of the last stacked bioreactor unit 36aₙ via the closed outlet port 58, loss of biological cells 2 and/or microbeads 4 out of the bioreactor assembly 12′ is minimized during respective seeding of the n number of bioreactor units 36a with the populations of biological cells 2 and/or microbeads 4. In this manner, seeding of the respective n number of bioreactor units 36a with populations of biological cells 2 and/or microbeads 4 may be accomplished robustly and expeditiously without concern that some of the biological cells 2 and/or microbeads 4 will be lost with the outflow of seeding media 26 from the bioreactor assembly 12′. It should be appreciated that, once flow of the seeding media 26 ceases, such that there is no perfusion through the porous membranes 46 of the bioreactor units 36a, the biological cells 2 and/or microbeads 4 will continue to be urged against the cell culture surfaces 54 on the porous membranes 46 via the force of gravity. Although, in an alternative embodiment, the seeding media 26 with the biological cells 2 and/or microbeads 4 may be flowed entirely through the upper flow channels 48, e.g., by opening both the inlet ports 56 and outlet ports 58 of all the bioreactor units 36a (including the last stacked bioreactor unit 36aₙ), essentially adding the n number of bioreactor units 36a to the first flow path 38a, care must be taken to ensure that a significant amount of biological cells 2 and/or microbeads 4 do not escape out of the bioreactor assembly 12′ with the outflow of the seeding media 26.

In still another example, the valve assembly 16, under control of the electric controller 22 (shown in FIG. 1), may be operated to cultivate and expand the populations of biological cells 2 by adding a flow sequence of bioreactor units 36a (in the illustrated case, all n number of stacked bioreactor units 36a₁ to 36aₙ) to the second flow path 38a between the nutrient source 14c, reagent source 14d, and gas source 14e and the waste destination 14i (shown in FIG. 1), with the nutrient source 14c, reagent source 14d, and gas source 14e serving as the media source, the waste destination 14i serving as the media destination, the last stacked bioreactor unit 36aₙ serving as a first bioreactor unit 36a in the flow sequence, the first stacked bioreactor unit 36a₁ serving as the last bioreactor unit 36a in the flow sequence, and the second to last stacked bioreactor unit 36aₙ₋₁ to the second stacked bioreactor unit 36a₂ serving as intermediate bioreactor units 36a in the flow sequence (see FIG. 7B).

In particular, the valve assembly 16 may be operated to cultivate and expand the populations of biological cells 2 by fluidly coupling the nutrient source 14c, reagent source 14d, and gas source 14e to the inlet port 60 of the last stacked bioreactor unit 36aₙ; fluidly coupling the waste destination 14i to the outlet port 62 of the first stacked bioreactor unit 36a₁); and fluidly coupling the outlet port 62 of each bioreactor unit 36a to the inlet port 60 of the immediately succeeding bioreactor unit 36a in the flow sequence along the second flow path 38b (i.e., the outlet port 62 of the last stacked bioreactor unit 36aₙ to the inlet port 60 of the second to last stacked bioreactor unit 36aₙ₋₁, the outlet port 62 of the second to last stacked bioreactor unit 36a₂ to the inlet port 60 of the third to last stacked bioreactor unit 36a₃, and so forth), thereby fluidly coupling the lower flow channels 50 of the bioreactor units 36a in series between the nutrient source 14c, reagent source 14d, and gas source 14e and the waste destination 14i.

As a result, under the fluid pressure applied by the pump assembly 18 (shown in FIG. 1), fresh culture media 28 flows along the second flow path 38b from the nutrient source 14c, reagent source 14d, and gas source 14e, in through the open inlet port 60, through the lower flow channel 50, and out through the open outlet port 62, of the last stacked bioreactor unit 36aₙ; then in through the open inlet port 60, through the lower flow channel 50, and out through the open outlet port 62, of the second to last stacked bioreactor unit 36aₙ₋₁; and so forth until spent culture media 28′ flows out of the open outlet port 58 of the first stacked bioreactor unit 36a₁ to the waste destination 14i. Thus, nutrient, reagent, and gas is delivered to the environment surrounding the populations of biological cells 2 residing in the n number of bioreactor assemblies 36a, and waste is extracted from the environment surrounding the populations of biological cells 2 residing the n number of bioreactor assemblies 36a, and in particular, by allowing the nutrient, reagent, and gas within the fresh culture media 28 to diffuse through the porous membranes 46 from the lower flow channels 50 into the upper flow channels 48, of the respective n number of bioreactor units 36a, and allowing the waste to diffuse through the porous membranes 46 from the upper flow channels 48 into the lower flow channels 50, of the respective n number of bioreactor units 36a, thereby facilitating expansion of the seed populations of biological cells 2.

In yet another example, the valve assembly 16, under control of the electric controller 22 (shown in FIG. 1), may be operated to wash the populations of biological cells 2 and/or microbeads 4 by adding a flow sequence of bioreactor units 36a (in the illustrated case, all n number of stacked bioreactor units 36a₁ to 36aₙ) to the third flow path 38c between the wash buffer source 14f and the waste destination 14i (shown in FIG. 1), with the wash buffer source 14f serving as the media source, the waste destination 14i serving as the media destination, the first stacked bioreactor unit 36a₁ serving as both the first bioreactor unit 36a and the last bioreactor unit 36a, and the second stacked bioreactor unit 36a₂ to the last stacked bioreactor unit 36aₙ serving as intermediate bioreactor units 36a, in the flow sequence along the forward portion 38c′ and return portion 38c″ of the third flow path 38c (see FIG. 7C).

In particular, the valve assembly 16 may be operated to wash the populations of biological cells 2 and/or microbeads 4 by fluidly coupling the wash buffer source 14f to the inlet port 56 of the first stacked bioreactor unit 36a₁; fluidly coupling the waste destination 14i to the outlet port 62 of the first stacked bioreactor unit 36a₁; fluidly coupling the outlet port 58 of each bioreactor unit 36a to the inlet port 56 of the immediately succeeding bioreactor unit 36a in the flow sequence along the forward path 38c′ of the third flow path 38c (i.e., the outlet port 58 of the first stacked bioreactor unit 36a₁ to the inlet port 56 of the second stacked bioreactor unit 36a₂, the outlet port 58 of the second stacked bioreactor unit 36a₂ to the inlet port 56 of the third stacked bioreactor unit 36a₃, and so forth), thereby fluidly coupling the upper flow channels 48 of the bioreactor units 36a in series between the wash buffer source 14f and the waste destination 14i; fluidly coupling the outlet port 62 of each bioreactor unit 36a to the inlet port 60 of the immediately subsequent bioreactor unit 36a in the flow sequence along the return path 38c″ of the third flow path 38c (i.e., the outlet port 62 of the last stacked bioreactor unit 36aₙ to the inlet port 60 of the second to last stacked bioreactor unit 36aₙ₋₁, the outlet port 62 of the second to last stacked bioreactor unit 36a₂ to the inlet port 60 of the third to last stacked bioreactor unit 36a₃, and so forth), thereby fluidly coupling the lower flow channels 50 of the bioreactor units 36a in series between the wash buffer source 14f and the waste destination 14i; and fluidly decoupling the outlet port 58 and the inlet port 60 of the last stacked bioreactor unit 36aₙ from any source of fluid, thereby fluidly coupling the upper flow channel 48 and lower flow channel 50 of each bioreactor unit 36a to each other (in effect, removing the stacked bioreactor units 36a₁ to 36aₙ from the other flow paths 38a, 38b to increase the pressure in the upper flow channels 48 to urge fluidly coupling between the upper flow channels 48 and the lower flow channels 50.

As a result, under the fluid pressure applied by the pump assembly 18 (shown in FIG. 1), fresh wash buffer 30 flows along the forward portion 38c′ of the third flow path 38c from the wash buffer source 14f, in through the open inlet port 56, through the upper flow channel 48, and out through the open outlet port 58, of the first stacked bioreactor unit 36a₁; while used wash buffer 30′ containing undesirable material (e.g., unbound molecules, debris, dead cells, or other undesirable molecules) perfuses along the bypass portion 38c′″ of the third flow path 38c through the porous membrane 46 from the upper flow channel 48 into the lower flow channel 50, of the first stacked bioreactor unit 36a₁ (due to the pressure buildup in the upper flow channel 48 caused by the closed outlet port 58 of the last stacked bioreactor unit 36aₙ creating a pressure gradient between the flow channels 48, 50 of all n number of stacked bioreactor units 36a₁ to 36aₙ); and then the perfused used wash buffer 30′ containing the undesirable material flows along the return portion 38c″ of the third flow path 38c from the lower flow channel 50 and only out through the open outlet port 62, of the first stacked bioreactor unit 36a₁ (due to the closed inlet port 60 of the last stacked bioreactor unit 36aₙ preventing the flow of fluid along the second flow path 38b in the opposite direction from the lower flow channels 50 out through the inlet ports 60 of all n number of stacked bioreactor units 36a₁ to 36aₙ), and to the waste destination 14i. Significantly, while the used wash buffer 30′ containing the undesirable material perfuses through the porous membrane 46, the porous membrane 46 retains the biological cells 2 and/or microbeads 4, which continue to be urged against the cell culture surface 54 on the porous membrane 46 via the action of convection, thereby washing the population of biological cells 2 and microbeads 4 residing in the first stacked bioreactor unit 36a₁.

This flow of the fresh wash buffer 30 and used wash buffer 30′ containing the undesirable material repeats for the second stacked bioreactor unit 36a₂ to the second to last bioreactor 36aₙ₋₁, with the fresh wash buffer 30 flowing along the forward portion 38c′ of the third flow path 38c out through the open outlet port 58 of each bioreactor unit 36a, then flowing in through the open inlet port 56 of the immediately succeeding bioreactor unit 36a, and the used wash buffer 30′ containing the undesirable material that has perfused along the bypass portion 38c″ of the third flow path 38c across the porous membrane 46, flowing along the return portion 38c″ of the third flow path 38c out through the open outlet port 62 of each bioreactor unit 36a and flowing in through the open inlet port 60 of an immediately succeeding bioreactor unit 36a in the flow sequence, thereby washing the population of biological cells 2 and/or microbeads 4 residing in the second stacked bioreactor unit 36a₂ to the second to last bioreactor 36aₙ₋₁.

Lastly, the fresh wash buffer 30 flows along the forward portion 38c′ of the third flow path 38c from the open outlet port 58 of the second to last stacked bioreactor unit 36aₙ₋₁, in through the open inlet port 56, and into the upper flow channel 48, of the last stacked bioreactor unit 36aₙ; then the used wash buffer 30′ containing the undesirable material perfuses along the bypass portion 38c″ of the third flow path 38c through the porous membrane 46 from the upper flow channel 48 into the lower flow channel 50, of the last stacked bioreactor unit 36aₙ (due to the pressure buildup in the upper flow channel 48 caused by the closed outlet port 58 of the last stacked bioreactor unit 36aₙ creating a pressure gradient between the flow channels 48, 50 of all n number of stacked bioreactor units 36a₁ to 36aₙ); and then the perfused used wash buffer 30′ containing the undesirable material flows along the return portion 38c″ of the third flow path 38c from the lower flow channel 50 and out through the open outlet port 62, of the last stacked bioreactor unit 36aₙ, back in through the open inlet port 60 of the second to last stacked bioreactor unit 36aₙ₋₁, thereby washing the population of biological cells 2 and/or microbeads 4 residing in the last stacked bioreactor unit 36aₙ.

In yet another example, the valve assembly 16, under control of the electric controller 22 (shown in FIG. 1), may be operated to harvest the expanded populations of biological cells 2 and/or microbeads 4 from the bioreactor assembly 12′ by adding a flow sequence of bioreactor units 36a (in the illustrated case, all n number of stacked bioreactor units 36a₁ to 36aₙ) to the first flow path 38a between the saline source 14g and the biological cell collection destination 14h (shown in FIG. 1), with the saline source 14g serving as the media source, the biological cell collection destination 14h serving as the media destination, the first stacked bioreactor unit 36a₁ serving as a first bioreactor unit 36a in the flow sequence, the last stacked bioreactor unit 36aₙ serving as the last bioreactor unit 36a in the flow sequence, and the second stacked bioreactor unit 36a₂ to the second to last stacked bioreactor unit 36aₙ₋₁ serving as intermediate bioreactor units 36a in the flow sequence (see FIG. 7A).

In particular, the valve assembly 16 may be operated to harvest the expanded populations of biological cells 2 and/or microbeads 4 from the bioreactor assembly 12′ by fluidly coupling the saline source 14g to the inlet port 56 of the first stacked bioreactor unit 36a₁; fluidly coupling the biological cell collection destination 14h to the outlet port 58 of the last stacked bioreactor unit 36aₙ); and fluidly coupling the outlet port 58 of each bioreactor unit 36a to the inlet port 56 of the immediately succeeding bioreactor unit 36a in the flow sequence along the first flow path 38a (i.e., the outlet port 58 of the first stacked bioreactor unit 36a₁ to the inlet port 56 of the second stacked bioreactor unit 36a₂, the outlet port 58 of the second stacked bioreactor unit 36a₂ to the inlet port 56 of the third stacked bioreactor unit 36a₃, and so forth), thereby fluidly coupling the upper flow channels 48 of the bioreactor units 36a in series between the saline source 14g and the waste destination 14i.

As a result, under the fluid pressure applied by the pump assembly 18 (shown in FIG. 1), harvesting media 32 flows along the first flow path 38a from the saline source 14g, in through the open inlet port 56 of the first stacked bioreactor unit 36a₁, and through the upper flow channel 48, thereby suspending the expanded population of biological cells 2 and microbeads 4 from the upper flow channel 48 within the harvesting media 32, and harvesting media 32′ with the suspended expanded population of biological cells 2 and microbeads 4 then flows out through the open outlet port 58 of the first stacked bioreactor unit 36a₁; then the harvesting media 32′ with the suspended expanded population of biological cells 2 and microbeads 4 from the first stacked bioreactor unit 36a₁ flows further along the first flow path 38a in through the open inlet port 56 of the second stacked bioreactor unit 36a₂, and through the upper flow channel 48, thereby suspending the expanded population of biological cells 2 and microbeads 4 from the upper flow channel 48 within the harvesting media 32′ then flows out through the open outlet port 58 of the second stacked bioreactor unit 36a₂; and so forth until the harvesting media 32′ with the suspended expanded population of biological cells 2 and microbeads 4 from all n number of bioreactor units 36a flows out of the open outlet port 58 of the last stacked bioreactor unit 36aₙ to the biological cell collection destination 14h. Thus, expanded populations of the biological cells 2 and microbeads 4 will be harvested from the upper flow channels 48 of the respective n number of bioreactor units 36a.

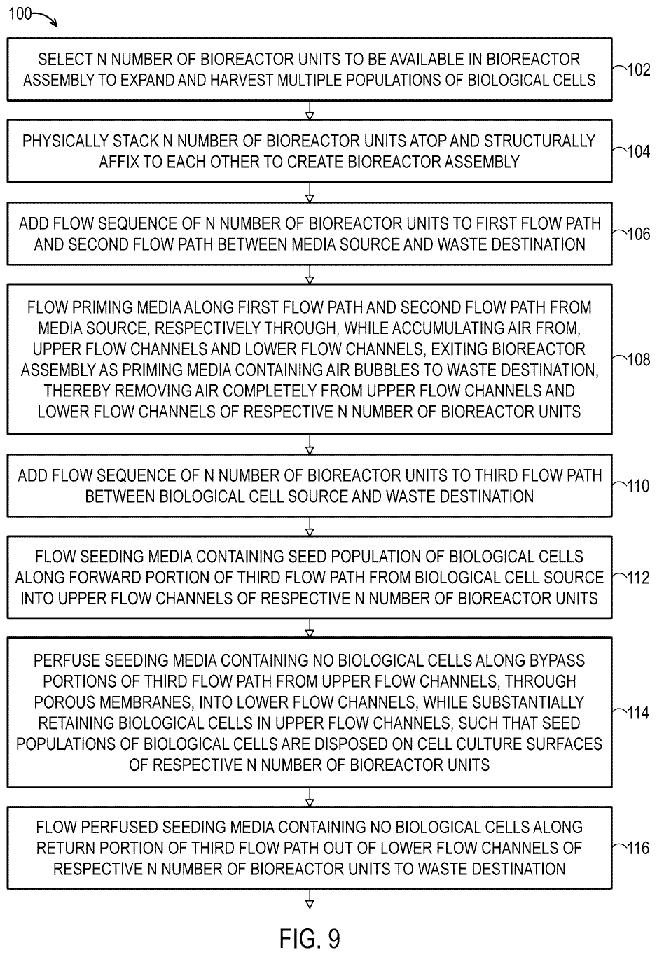

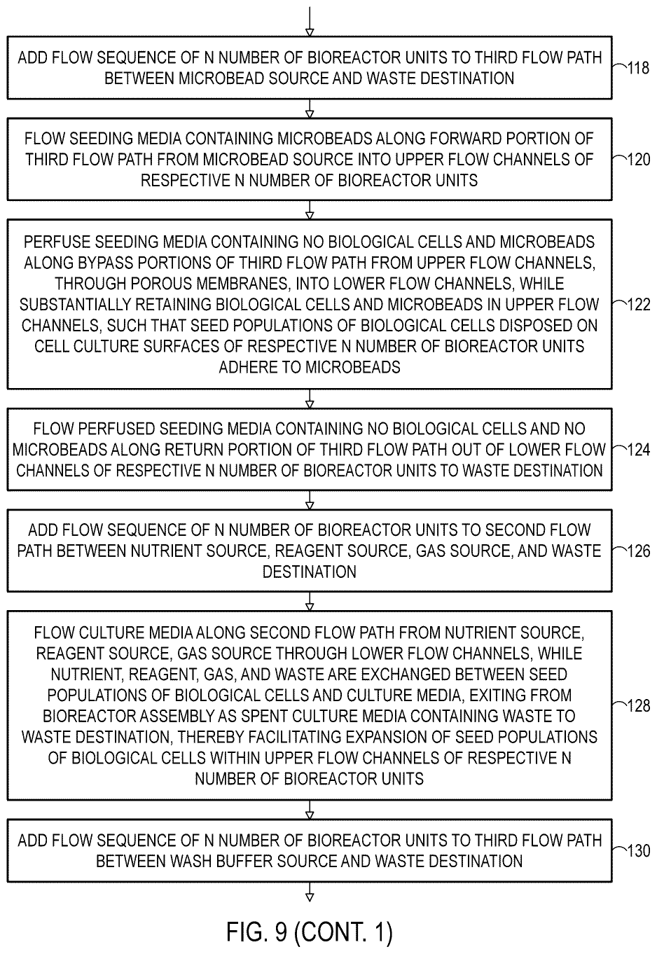

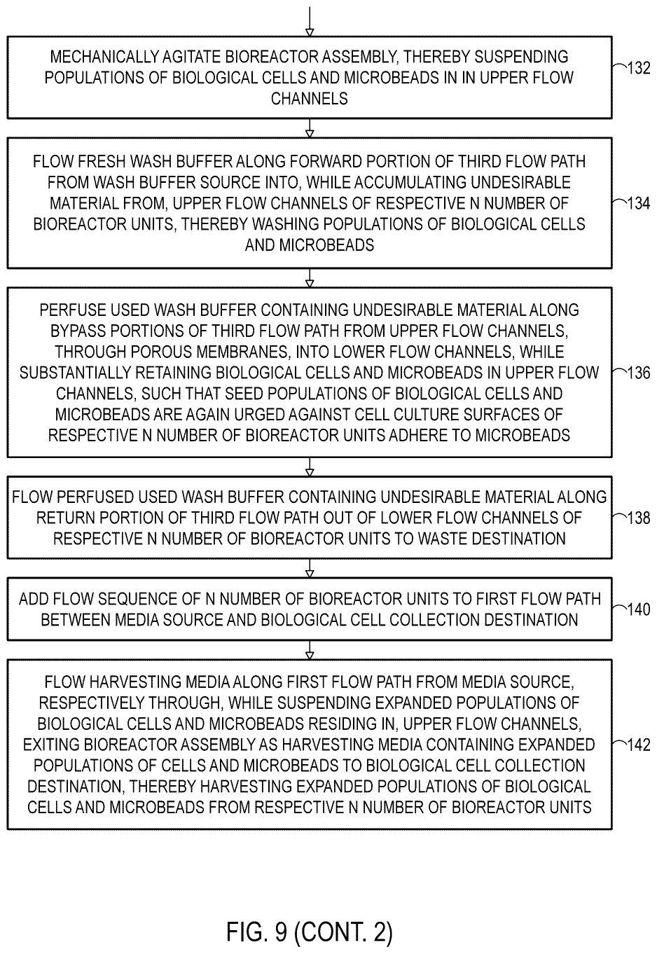

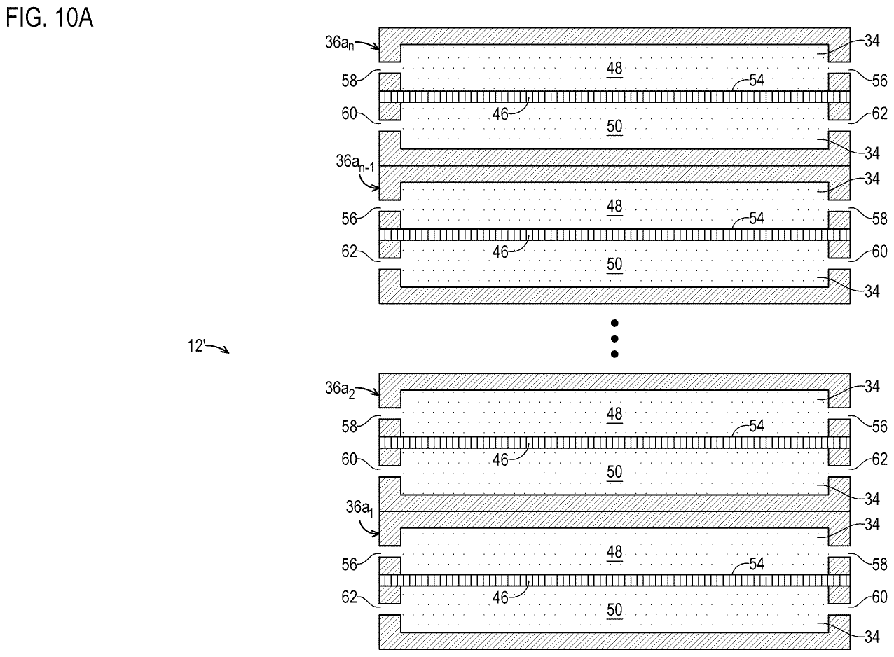

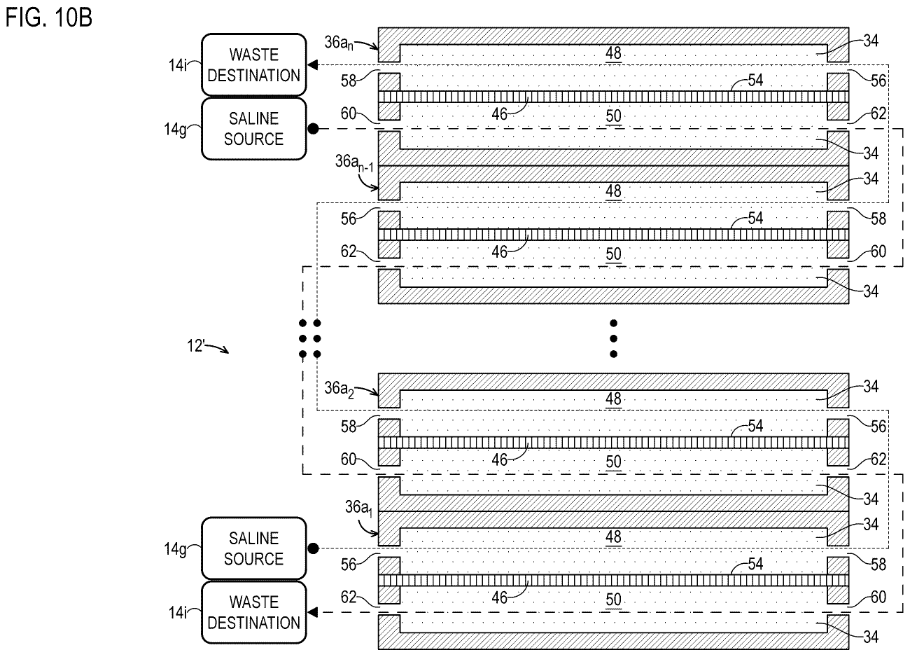

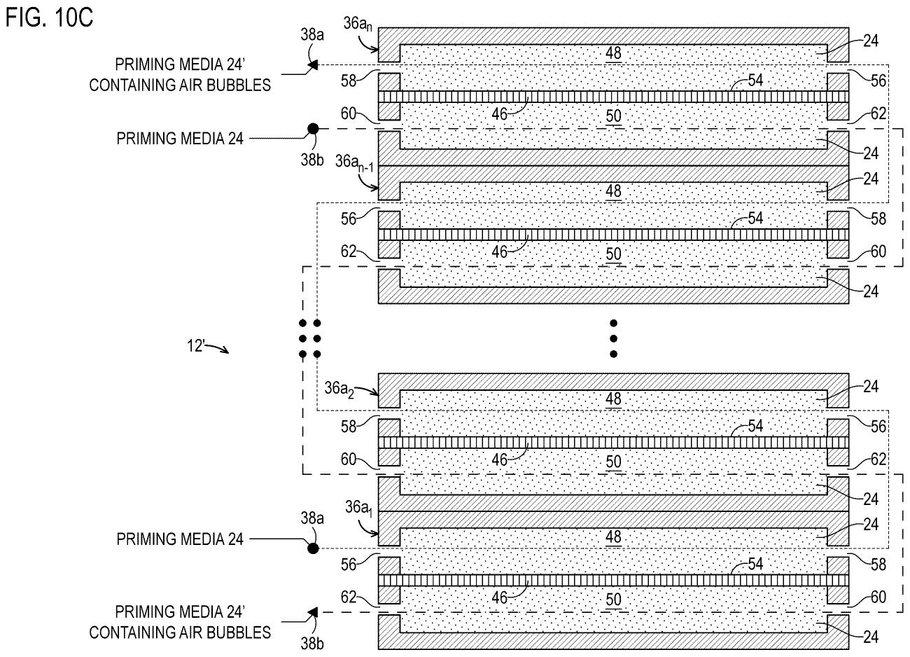

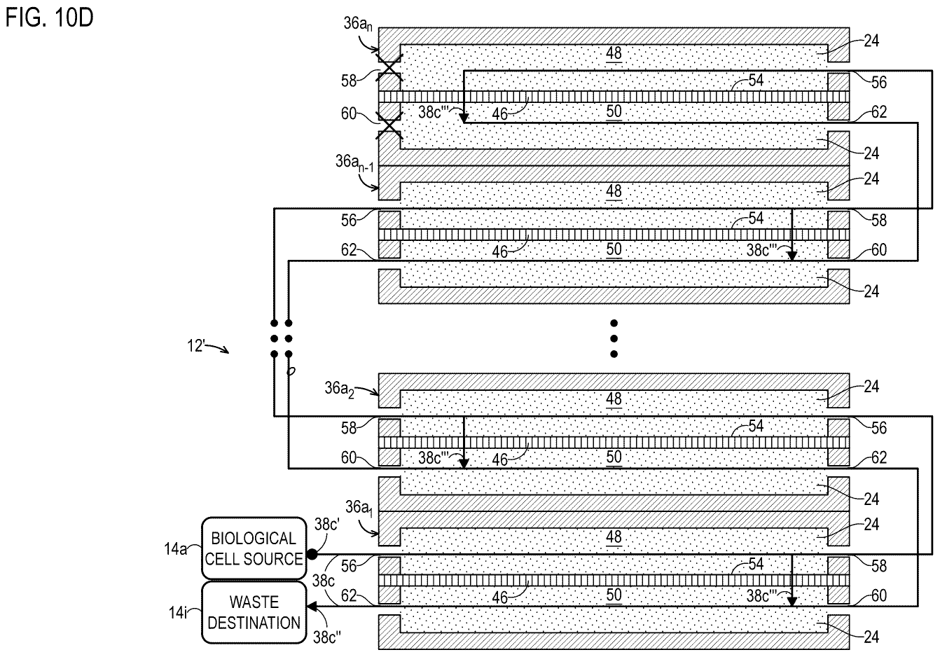

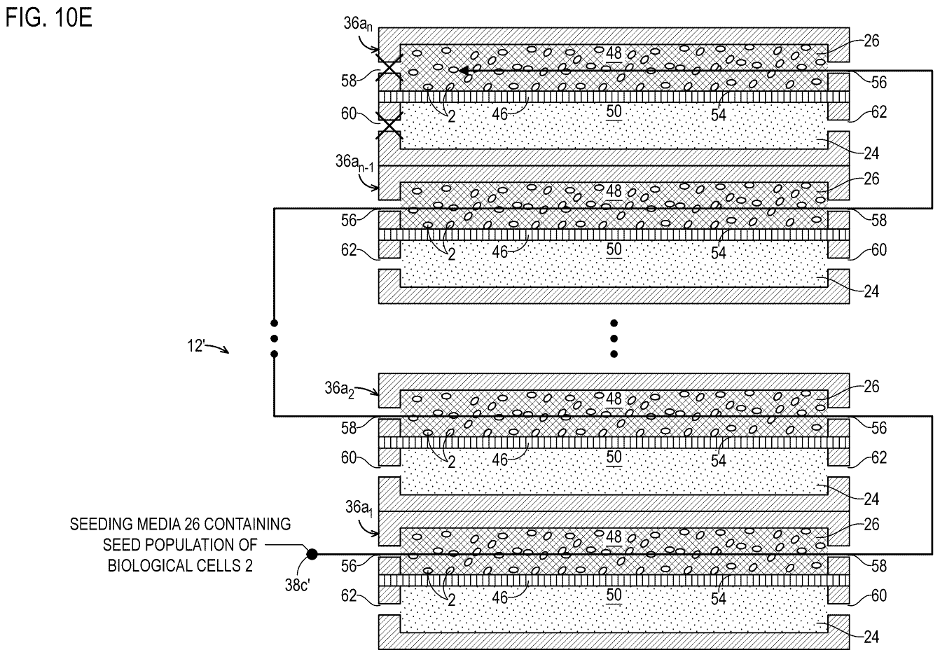

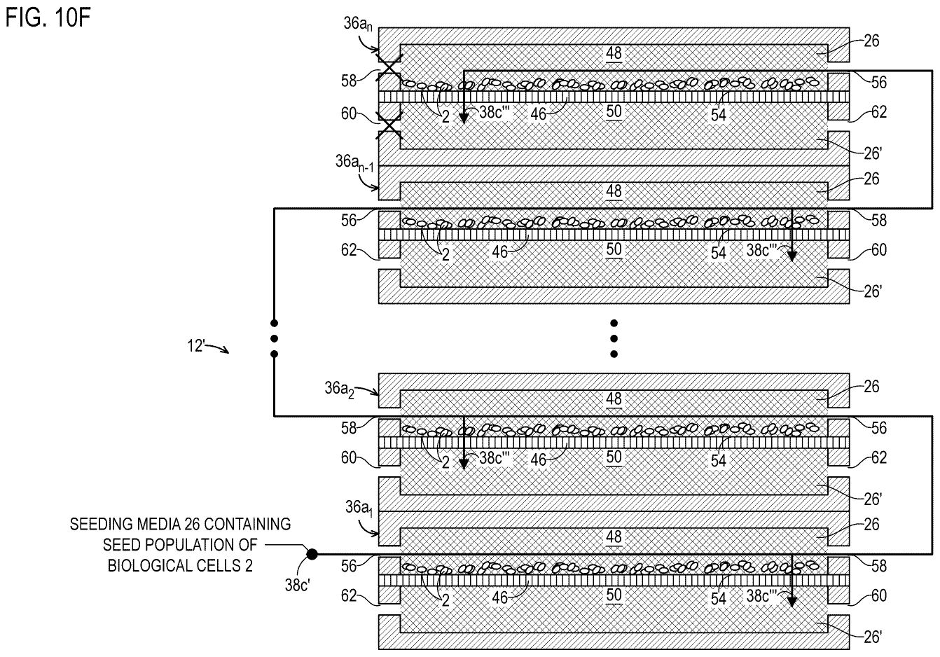

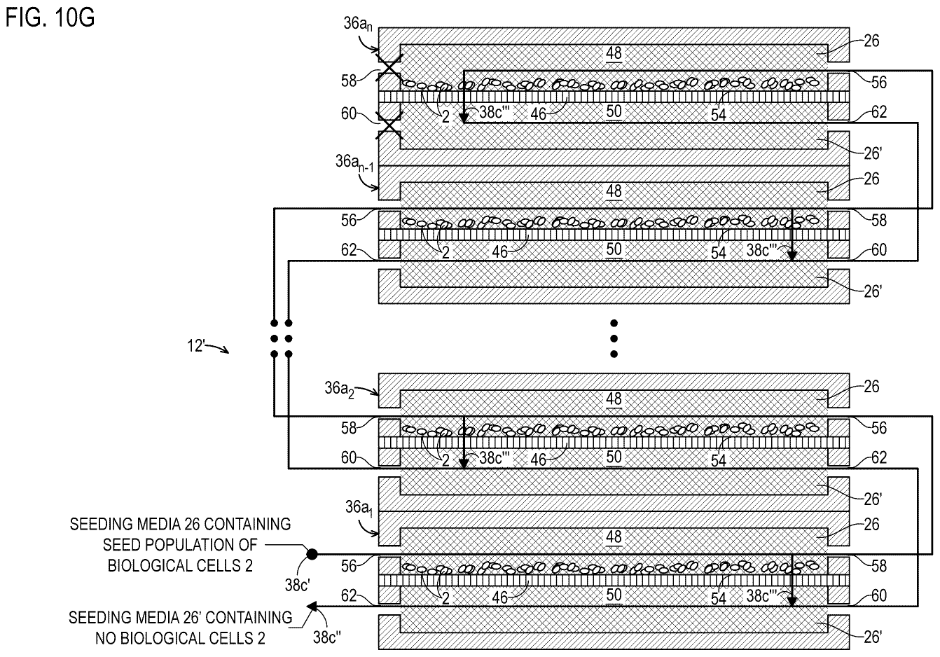

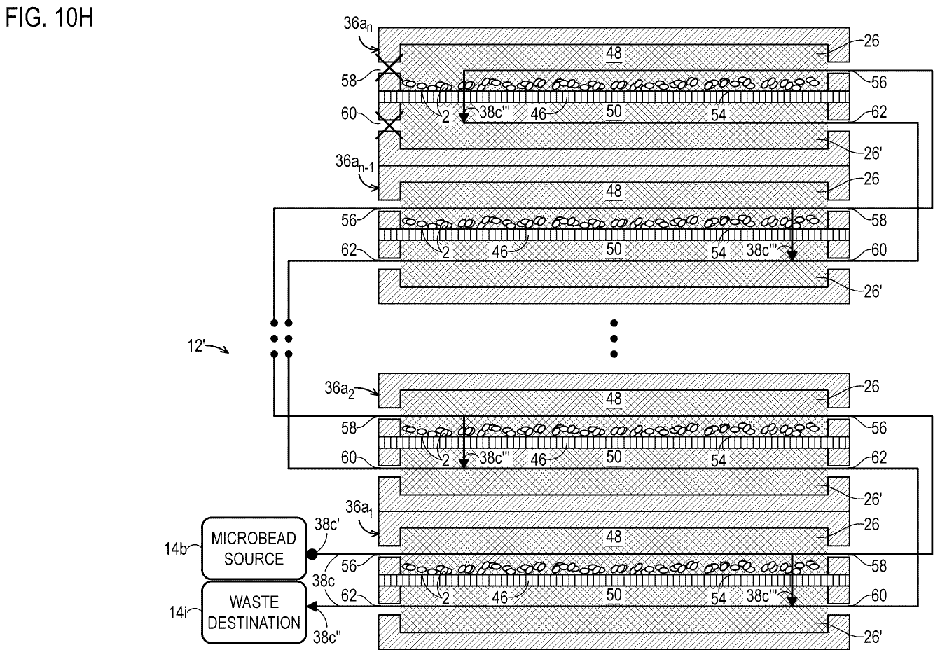

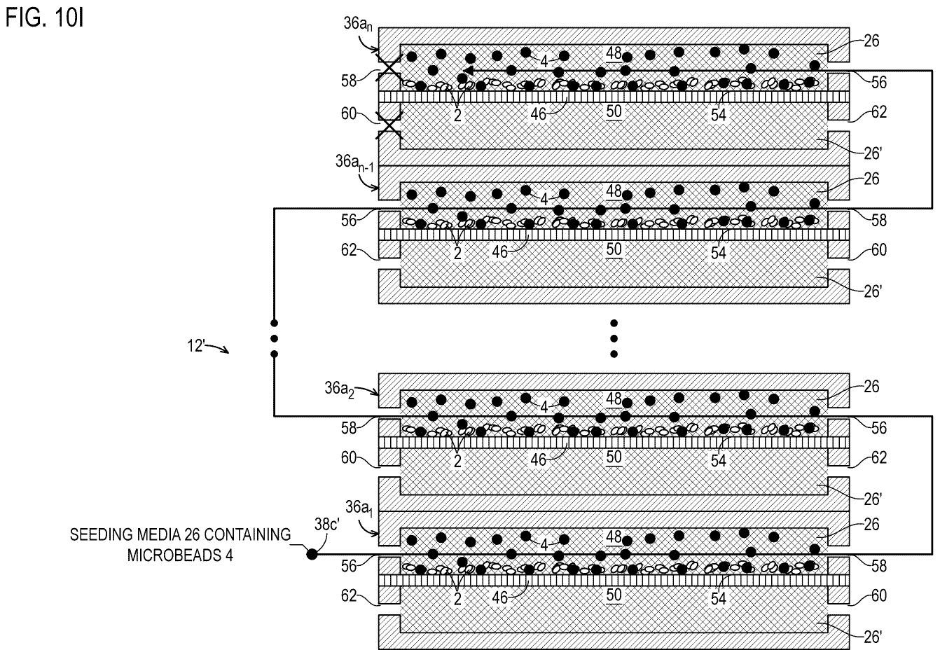

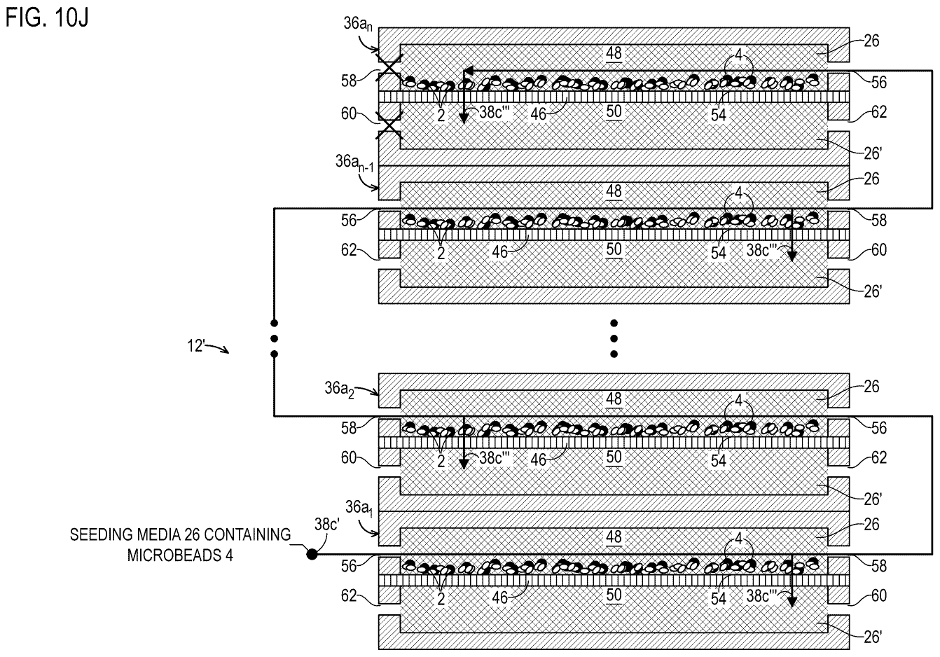

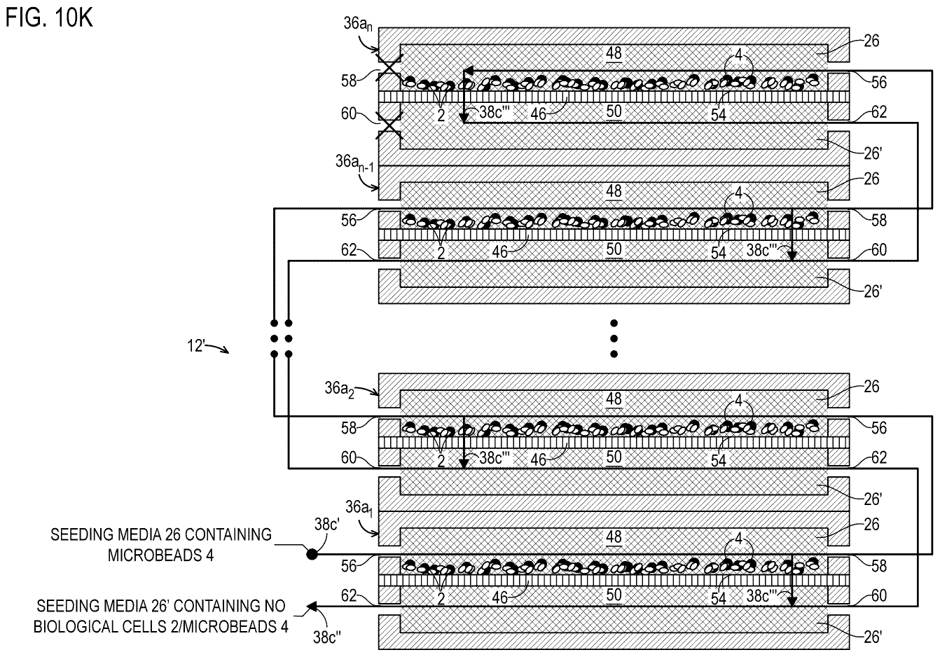

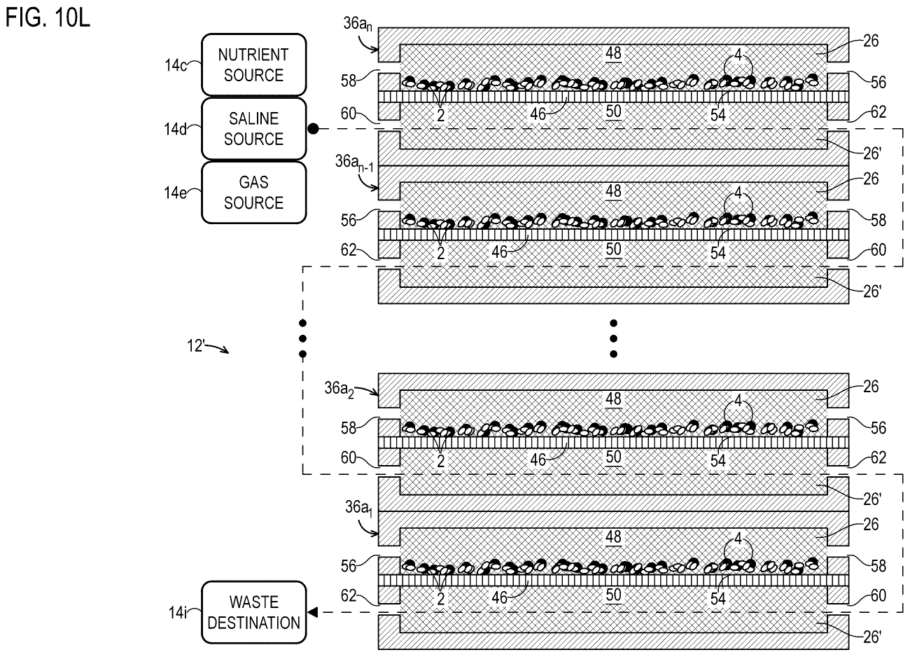

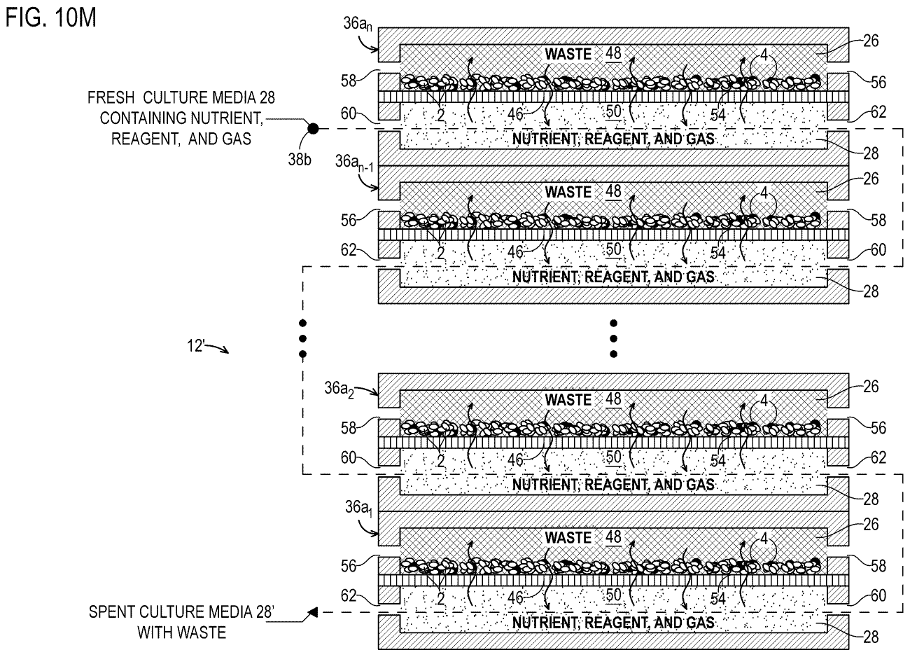

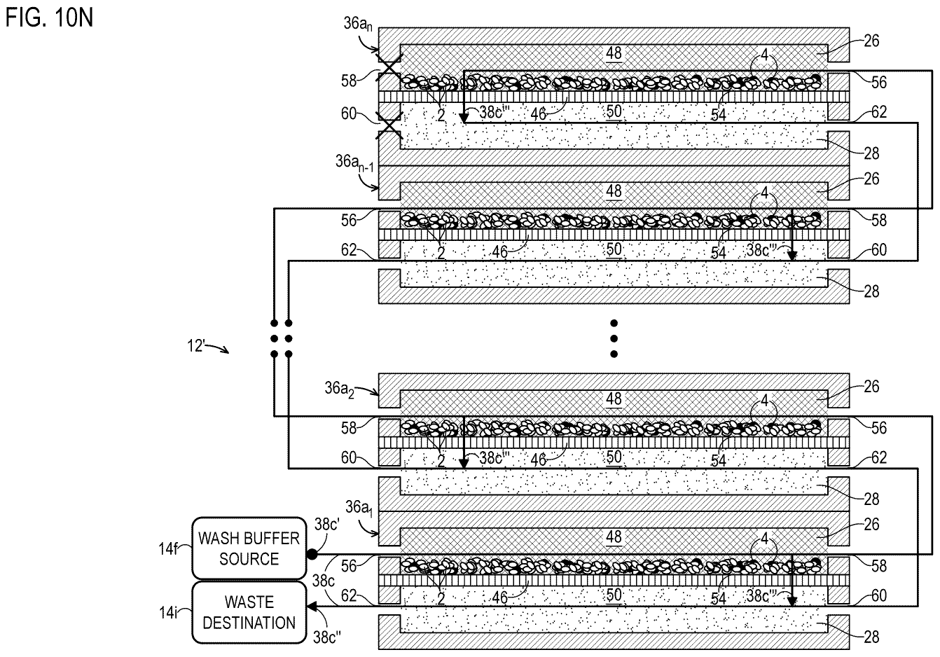

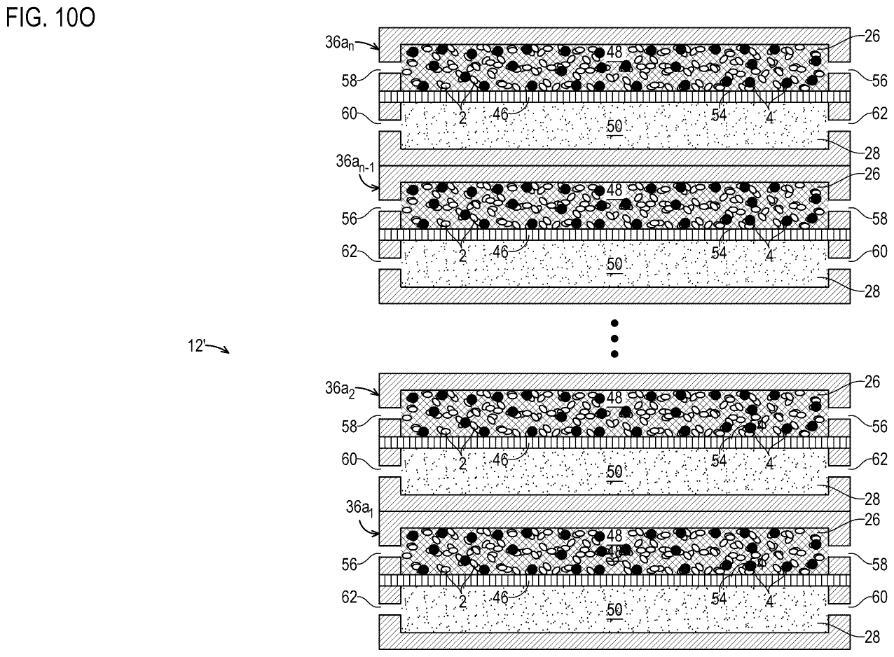

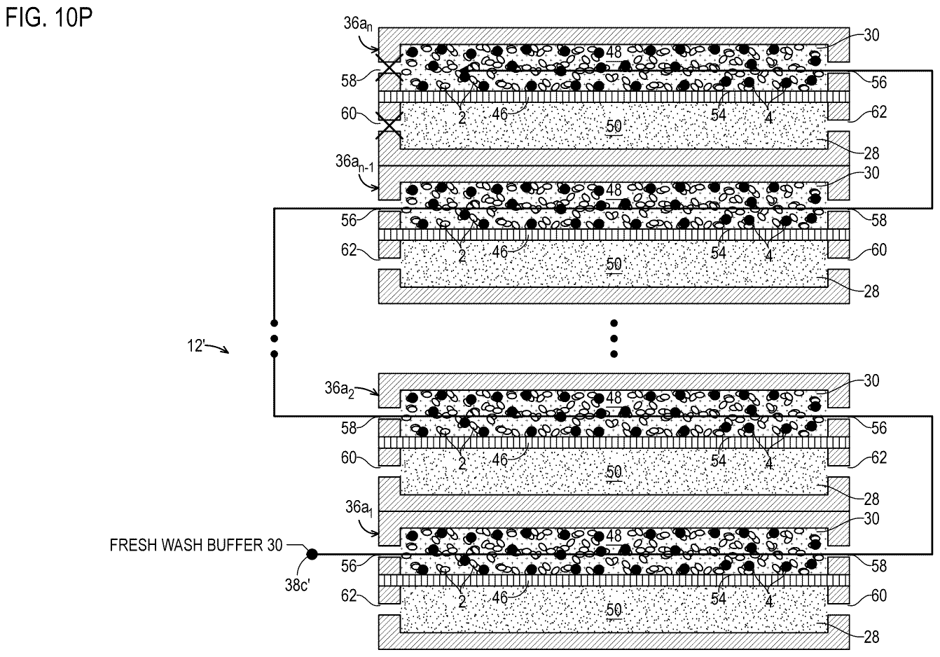

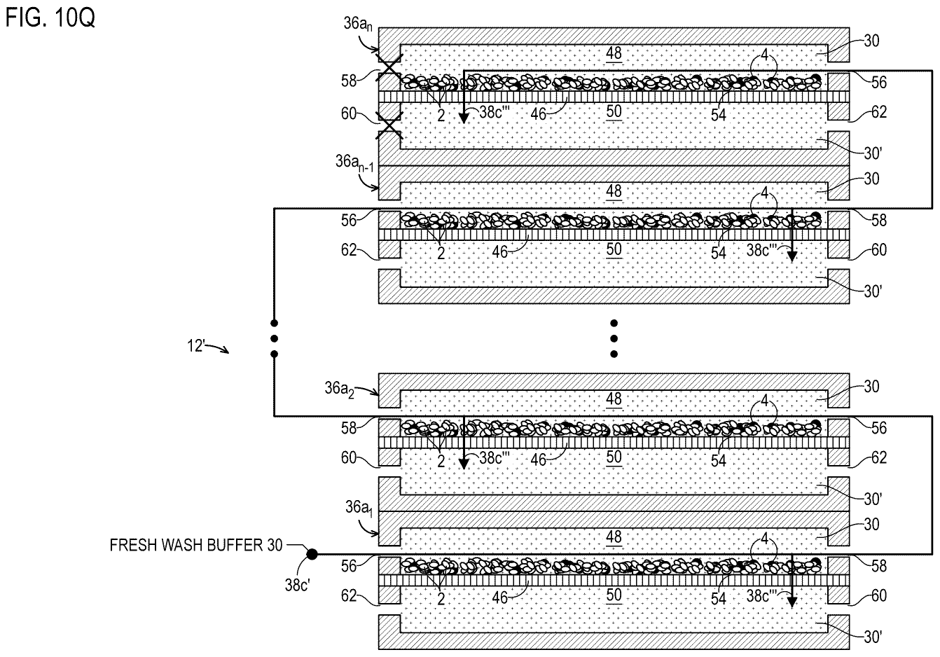

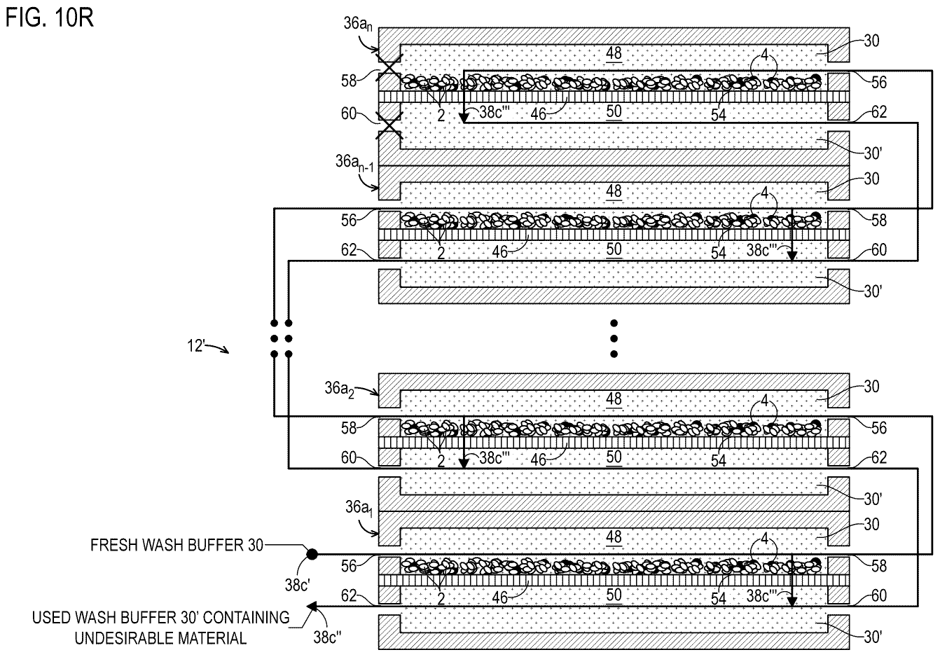

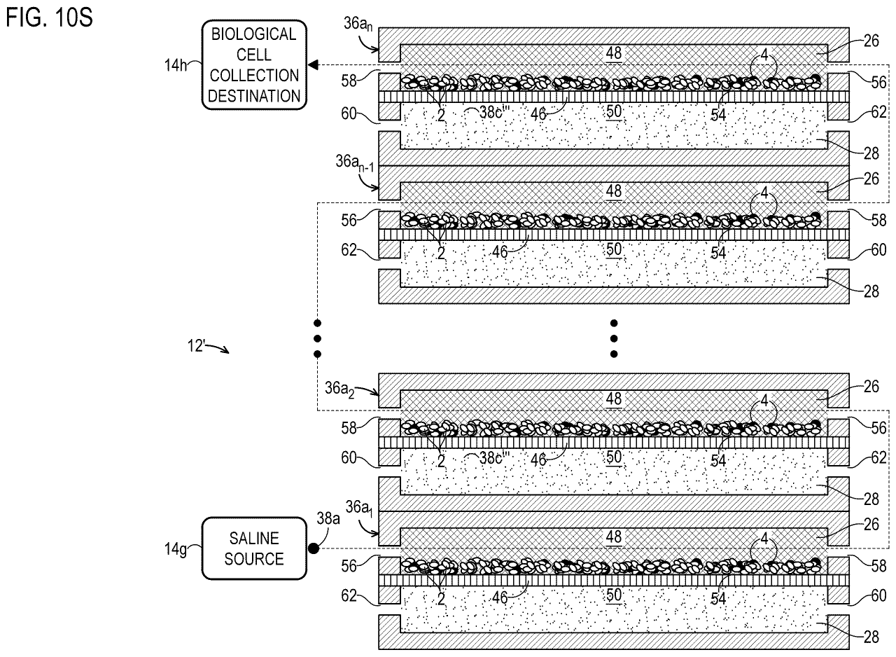

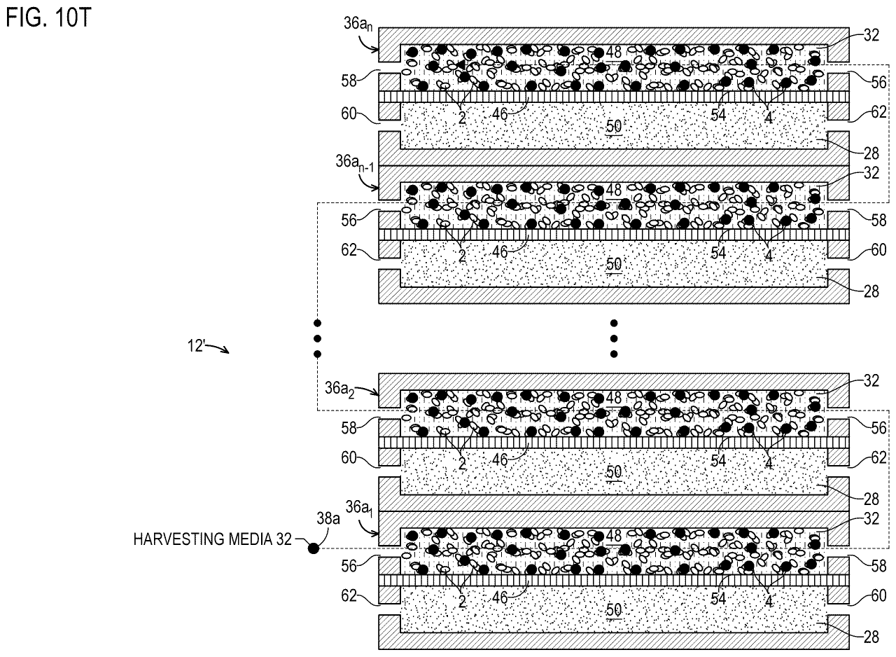

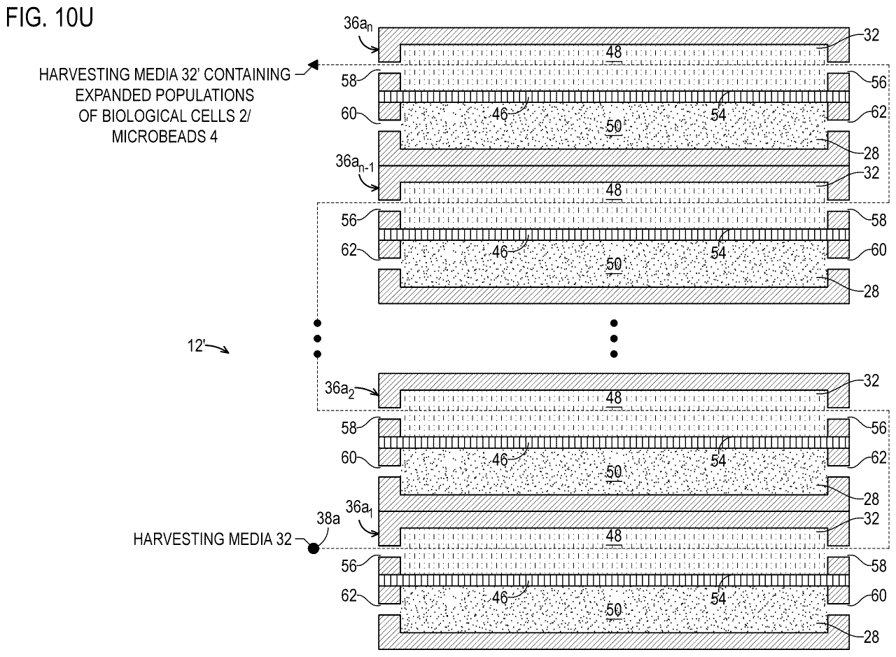

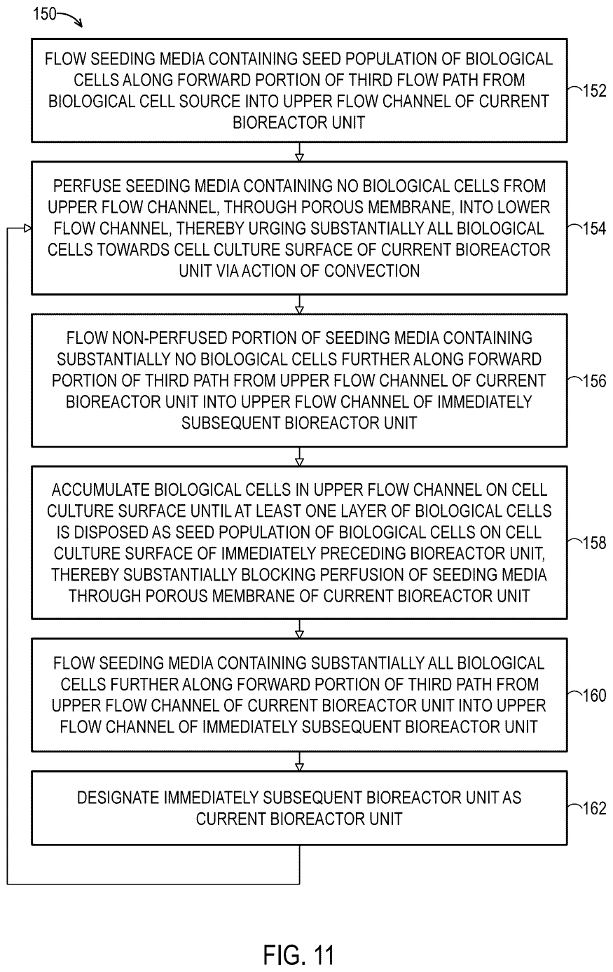

Having described the structure and function of the bioreactor system 10 illustrated in FIG. 1, one method 100 of operating the bioreactor assembly 12′ with the bioreactor units 36a illustrated in FIG. 8 to expand and harvest multiple populations of biological cells 2 (e.g., T lymphocytes (T-cells)) will now be described with reference to FIGS. 9 and 10A-10U.

The n number of bioreactor units 36a to be available in the bioreactor assembly 12′ to expand and harvest multiple populations of biological cells 2 is selected depending on the desired quantity of biological cells 2 to be harvested (step 102). In this manner, the bioreactor system 10 may be scaled up or down to meet the desired quantity of biological cells 2 to be harvested. In the illustrated method 100, the n number of bioreactor units 36a to be structurally used in the bioreactor assembly 12′ to expand and harvest multiple populations of biological cells 2 is pre-selected; that is, selected prior to the initiation of the seeding and incubation process. However, in alternative methods, the n number of bioreactor units 36a structurally used in the bioreactor assembly 12′ may be dynamically selected throughout the seeding and incubation process; that is, as additional expansion of populations of biological cells 2 is needed during the seeding and incubation process, one or more additional bioreactor units 36a may be selected.

In the illustrated method 100, the bioreactor units 36a are separate and distinct, in which case, such bioreactor system 10 may be structurally scaled up or down at the user level and at the resolution of a single bioreactor unit 36a. In alternative methods, the bioreactor assembly 12′ has permanently integrated bioreactor units 36a, in which case, a bioreactor assembly 12′ with a suitable n number of bioreactor units 36a appropriate for the desired quantity of biological cells 2 to be harvested may be used to structurally scale the bioreactor system 10 up or down at the manufacturing level.

However, if bioreactor assemblies 12 with different n numbers of permanently integrated bioreactor units 36a (e.g., a bioreactor assembly 12′ with three permanently integrated bioreactor units 36a, a bioreactor assembly 12′ with six permanently integrated bioreactor units 36a, a bioreactor assembly 12′ with twelve permanently integrated bioreactor units 36a, etc.) are manufactured, the bioreactor system 10 may be effectively structurally scaled up or down at the user level by selecting one of the bioreactor assemblies 12 with the n number of integrated bioreactor units 36a that best matches the desired quantity of biological cells 2 to be harvested. Alternatively, if the bioreactor assembly 12′ has permanently integrated bioreactor units 36a, the bioreactor system 10 may be may be structurally scaled up or down at the user level and at the resolution of the n number of bioreactor units 36a in the bioreactor assembly 12′ by combining multiple bioreactor assemblies 12 together. For example, multiple bioreactor assemblies 12, each with three permanently integrated bioreactor units 36a, may be selected at the user level and at a resolution of three bioreactor units 36a to structurally scale the bioreactor system 10 up or down (e.g., three bioreactor units 36a, six bioreactor units 36a, nine bioreactor units 36a, etc.).

Notwithstanding that the n number of bioreactor units 36a to be used in the method 100 may be structurally selected depending on the desired quantity of biological cells 2 to be harvested in order to structurally scale the bioreactor assembly 12′ up or down, the n number of bioreactor units 36a to be used in the method 100 may also be functionally selected (by activating/deactivating selected ones of the bioreactor units 36a) depending on the desired quantity of biological cells 2 to be harvested, thereby functionally scaling the bioreactor assembly 12′ up or down at the user level and at the resolution of a single bioreactor unit 36a by selectively activating/deactivating the physically available of bioreactor units 36a (i.e., by adding/removing selected ones of the bioreactor units 36a to or from the flow paths 38a-38c) to generate a flow sequence of bioreactor units 36a, as will be described in further detail below. Thus, while the physical selection of the n number of bioreactor units 36a sets the maximum yield of biological cells 2 that can be harvested from the bioreactor system 10, the bioreactor system 10 may be functionally scaled up to or down from the maximum yield of the harvested biological cells 2.

Next, the selected n number of stacked bioreactor units 36a₁ to 36aₙ are physically stacked atop and structurally affixed to each other to create bioreactor assembly 12′ (step 104) (see FIG. 10A). In the illustrated method 100, the bioreactor units 36a are separate and distinct, in which case, the user may stack and structural affix the selected n number of bioreactor units 36a atop each other. In alternative methods, the bioreactor units 36a are permanently integrated within the bioreactor assembly 12′, in which case, the stacking and structural affixation of the n number of bioreactor units 36a atop each other is performed at the manufacturing level and/or structural affixation of the selected n number of bioreactor units 36a atop each may be performed by stacking and affixing multiple bioreactor assemblies 12 atop each other by the user. Although, stacking of the n number of stacked bioreactor units 36a₁ to 36aₙ atop each other minimizes the footprint of the resulting bioreactor assembly 12′ (or bioreactor assemblies 12), it should be appreciated that the n number of stacked bioreactor units 36a₁ to 36aₙ may be alternatively arranged relative to each other in a single plane, in which case, physical stacking and affixation of the n number of stacked bioreactor units 36a₁ to 36aₙ need not be performed.

Once the n number of stacked bioreactor units 36a₁ to 36aₙ are physically stacked atop and structurally affixed to each other, flow sequences of the respective n number of stacked bioreactor units 36a₁ to 36aₙ may be created by adding the stacked bioreactor units 36a₁ to 36aₙ to selected ones of the flow paths 38a-38c between different combinations of the media sources (biological cell source 14a, microbead source 14b, nutrient source 14c, reagent source 14d, gas source 14e, wash buffer source 14f, and saline source 14g) and the media destinations (biological cell collection destination 14h and waste destination 14i) to effect the aforementioned functions (a)-(e) described above with respect to FIG. 1. In the method 100, the flow sequences include all n number of stacked bioreactor units 36a₁ to 36aₙ in the same order in which the n number of bioreactor units 36a are physically arranged with each other, although in alternative methods, the flow sequences may include any selected ones of the n number of stacked bioreactor units 36a₁ to 36aₙ in the same order as, or a different order from, the order in which the n number of stacked bioreactor units 36a₁ to 36aₙ are physically arranged with each other. In the method 100, the flow sequences including all n number of stacked bioreactor units 36a₁ to 36aₙ are created prior to the beginning of the performance of the aforementioned functions (a)-(e), although it should be appreciated that the flow sequences of the respective n number of bioreactor units may be increased over time by incrementally adding at least one succeeding bioreactor unit 36a to the flow paths 38a-38c.

First, the n number of stacked bioreactor units 36a₁ to 36aₙ are primed.

In particular, a flow sequence of the n number of stacked bioreactor units 36a₁ to 36aₙ is added (e.g., via operation of the valve assembly 16) to the first flow path 38a and the second flow path 38b between the saline source 14g and the waste destination 14i (step 106) (see FIG. 10B), and in this case, by fluidly coupling the upper flow channels 48 and the lower flow channels 50 of the respective n number of stacked bioreactor units 36a₁ to 36aₙ in series along the first flow path 38a and the second flow path 38b between the saline source 14g and the waste destination 14i via opening/closing of the ports 56-62 of each of the n number of stacked bioreactor units 36a₁ to 36aₙ in a similar manner described with respect to FIGS. 7A-7B.

Then, priming media 24 is flowed along the first flow path 38a and the second flow path 38b from the saline source 14g, respectively through, while accumulating air bubbles from, the upper flow channels 48 and lower flow channels 50, exiting the bioreactor assembly 12′ as priming media 24′ containing air bubbles to the waste destination 14i, thereby removing air 34 (see FIG. 10B) completely or mostly from the upper flow channels 48 and lower flow channels 50 of the respective n number of stacked bioreactor units 36a₁ to 36aₙ (step 108) (see FIG. 10C). It should be noted that, although the upper flow channels 48 and the lower flow channels 50 of the respective n number of stacked bioreactor units 36a₁ to 36aₙ, have been illustrated and described as being concurrently primed, the upper flow channels 48 and the lower flow channels 50 of the respective n number of stacked bioreactor units 36a₁ to 36aₙ may be sequentially primed at different times; that is, all of the upper flow channels 48 of the respective n number of stacked bioreactor units 36a₁ to 36aₙ are primed, and then all of the lower flow channels 50 of the respective n number of stacked bioreactor units 36a₁ to 36aₙ are primed, or vice versa.

Next, the n number of stacked bioreactor units 36a₁ to 36aₙ are respectively seeded with populations of biological cells 2.

In particular, a flow sequence of the n number of stacked bioreactor units 36a₁ to 36aₙ is added (e.g., via operation of the valve assembly 16) to the third flow path 38c between the biological cell source 14a and the waste destination 14i (step 110) (see FIG. 10D), and in this case, by fluidly coupling the upper flow channels 48 of the respective n number of stacked bioreactor units 36a₁ to 36aₙ in series along the forward portion 38c′ of the third flow path 38a between the biological cell source 14a and the waste destination 14i; fluidly coupling the lower flow channels 50 of the respective n number of stacked bioreactor units 36a₁ to 36aₙ in series along the return portion 38c″ of the third flow path 38c between the waste destination 14i and the biological cell source 14a; and fluidly coupling the upper flow channel 48 and lower flow channel 50 of each of the n number of stacked bioreactor units 36a₁ to 36aₙ to each other via opening/closing of the ports 56-62 of each of the n number of stacked bioreactor units 36a₁ to 36aₙ in a similar manner described with respect to FIG. 7C.

Then, the upper flow channels 48 of the respective n number of stacked bioreactor units 36a₁ to 36aₙ are respectively seeded with populations of biological cells 2. In particular, seeding media 26 containing a seed population of biological cells 2 is flowed from the biological cell source 14a along the forward portion 38c′ of the third flow path 38c into the upper flow channels 48 of the respective n number of stacked bioreactor units 36a₁ to 36aₙ (step 112) (see FIG. 10E); seeding media 26′ containing no biological cells 2 is perfused along bypass portions 38c″ of the third flow path 38c from the upper flow channels 48, through the porous membranes 46, and into the lower flow channels 50, of the respective n number of stacked bioreactor units 36a₁ to 36aₙ, while substantially retaining the biological cells 2 within the upper flow channels 48 of the respective n number of stacked bioreactor units 36a₁ to 36aₙ, such that the populations of biological cells 2 are disposed on the cell culture surfaces 54 of the respective n number of stacked bioreactor units 36a₁ to 36aₙ (step 114) (see FIG. 10F); and the perfused seeding media 26′ containing no biological cells 2 is flowed along the return portion 38c″ of the third flow path 38c out of the lower flow channels 50 of the respective n number of stacked bioreactor units 36a₁ to 36aₙ to the waste destination 14i (step 116) (see FIG. 10G).

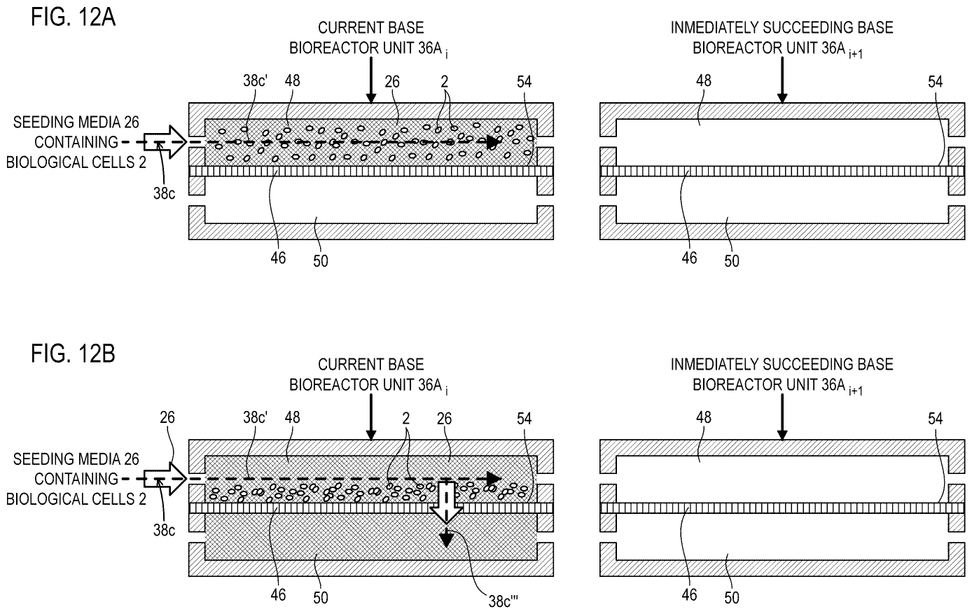

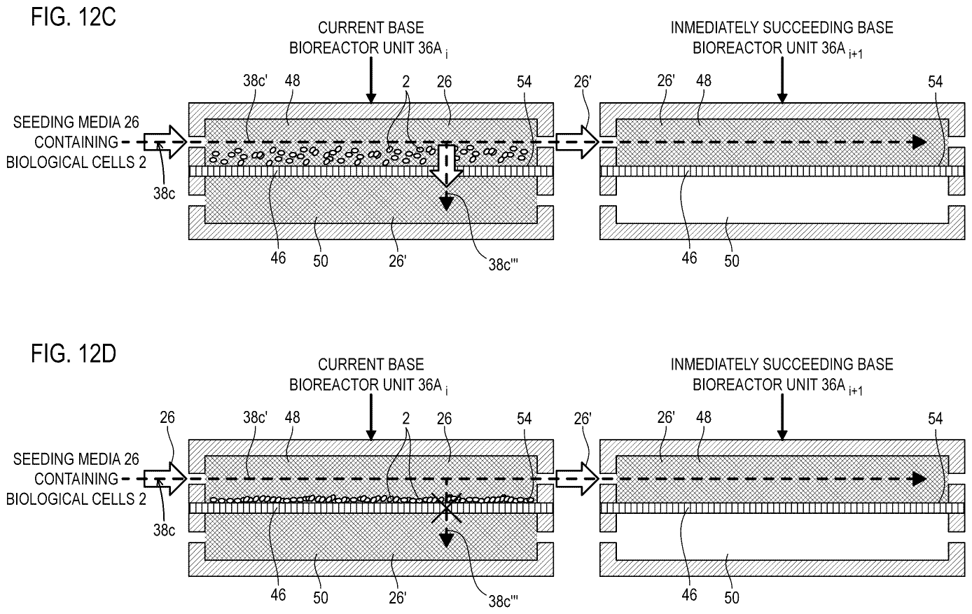

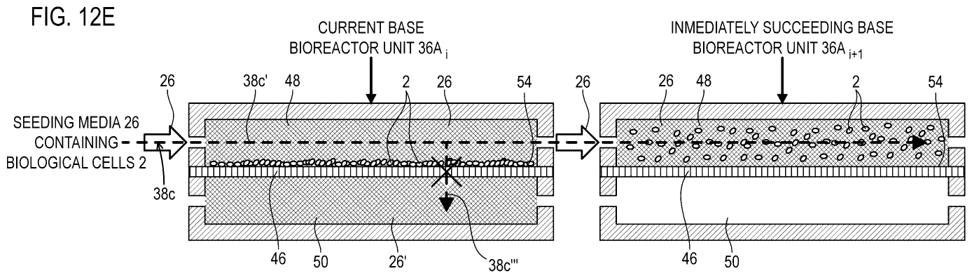

In one advantageous method 150 illustrated in FIGS. 11 and 12A-12E, each of the steps 112-116 are incrementally performed in each of the n number of stacked bioreactor units 36a₁ to 36aₙ in the order of the flow sequence, such that a monolayer of biological cells 2, as a seed population of biological cells 2, is incrementally disposed on the cell culture surfaces 54 in the upper flow channels 48 of the respective n number of stacked bioreactor units 36a₁ to 36aₙ. In this manner, seed populations of biological cells 2 can be more uniformly and efficiently disposed on the cell culture surfaces 54 in the upper flow channels 48 of the respective n number of stacked bioreactor units 36a₁ to 36aₙ.

For example, in one method 150 of incrementally seeding the n number of stacked bioreactor units 36a₁ to 36aₙ, seeding media 26 containing a seed population of biological cells 2 is first flowed along the forward portion 38c′ of the third flow path 38c from the biological cell source 14a into the upper flow channel 48 of a current bioreactor unit 36aᵢ preceding the last bioreactor unit 36a in the flow sequence (step 152) (see FIG. 12A). Notably, if the current bioreactor unit 36aᵢ is not the first stacked bioreactor unit 36a₁ in the flow sequence, the seeding media 26 containing the seed population of biological cells 2 will be flowed along the forward portion 38c′ of the third flow path 38c from an immediately preceding bioreactor unit 36aᵢ₋₁.

Next, seeding media 26′ containing no biological cells 2 is initially perfused along the bypass portion 38c″ of the third flow path 38c from the upper flow channel 48, through the porous membrane 46, and into the lower flow channel 50, thereby urging substantially all of the biological cells 2 contained in the seeding media 26 that has flowed into the upper flow channel 48 (e.g., at least 90 percent of the biological cells 2 contained in the seeding media 26 that has been flowed into the upper flow channel 48 of the current bioreactor unit 36aᵢ) towards the cell culture surface 54 of the current bioreactor unit 36aᵢ via the action of convection (step 154) (see FIG. 12B).

As a result, a non-perfused portion of the seeding media 26′ that has not perfused from the upper flow channel 48, through the porous membrane 46, and into the lower flow channel 50, will flow further along the forward portion 38c′ of the third flow path 38c out of the upper flow channel 48 of the current bioreactor unit 36aᵢ and into the upper flow channel 48 of the immediately succeeding bioreactor unit 36aᵢ₊₁ in the flow sequence (step 156) (see FIG. 12C). However, such portion of the seeding media 26′ that flows into the upper flow channel 48 of the immediately succeeding bioreactor unit 36aᵢ₊₁ will contain substantially no biological cells 2 (e.g., less than 10 percent of the biological cells 2 contained in the seeding media 26 that has been flowed into the upper flow channel 48 of the current bioreactor unit 36aᵢ), since substantially all of the biological cells 2 have been urged towards the towards the cell culture surface 54 on the porous membrane 46 of the current bioreactor unit 36aᵢ via the action of convection.

The biological cells 2 that are urged towards the cell culture surface 54 on the porous membrane 46 of the current bioreactor unit 36aᵢ accumulate on the cell culture surface 54 until at least one layer of biological cells 2 is disposed as a seed population on the cell culture surface 54 of the current bioreactor unit 36aᵢ, thereby substantially blocking perfusion of the seeding media 26′ through the porous membrane 46 of the current bioreactor unit 36aᵢ, such that the current bioreactor unit 36aᵢ is effectively removed (fluidly decoupled) from the bypass portion 38c′″ of the third flow path 38c (e.g., less than 20 percent of the seeding media 26 that has been flowed into the upper flow channel 48 of the current bioreactor unit 36aᵢ) (step 158) (see FIG. 12D).

As a result of the cessation of the action of convection that previously urged substantially all of the biological cells 2 towards the cell culture surface 54 of the current bioreactor unit 36aᵢ, seeding media 26 containing substantially all of the biological cells 2 (e.g., at least 90 percent of the biological cells 2 contained in the seeding media 26 that has been flowed into the upper flow channel 48 of the current bioreactor unit 36aᵢ) will flow further along the forward portion 38c′ of the third flow path 38c out of the upper flow channel 48 of the current bioreactor unit 36aᵢ and into the upper flow channel 48 of the immediately succeeding bioreactor unit 36aᵢ₊₁ (step 160) (see FIG. 12E). The immediately succeeding bioreactor unit 36aᵢ₊₁ will then be designated as the current bioreactor unit 36aᵢ (step 162), and steps 154-162 are then repeated until the last bioreactor unit 36a in the flow sequence (in this case, the last stacked bioreactor unit 36aₙ) has been seeded with a population of biological cells 2. Notably, since there is no immediately succeeding bioreactor unit 36a after the last stacked bioreactor unit 36aₙ, no seeding media 26 will flow directly out of the upper flow channel 48 of the last stacked bioreactor unit 36aₙ as per steps 156 and 160. Instead, once at least one layer of biological cells 2 is disposed as a seed population of biological cells 2 on the cell culture surface 54 of the last stacked bioreactor unit 36aₙ, the pressure build-up in the upper flow channels 48 of the respective n number of stacked bioreactor units 36a₁ to 36aₙ will cause the seeding media 26′ to perfuse from the upper flow channels 48, through the porous membranes 46, and into the lower flow channels 50 of all n number of stacked bioreactor units 36a₁ to 36aₙ.

It should be appreciated that the method 150 advantageously allows incremental seeding of the n number of stacked bioreactor units 36a₁ to 36aₙ using a simple valve assembly 16 that need only be operated to fluidly couple the n number of stacked bioreactor units 36a₁ to 36aₙ to the third flow path 38c between the biological cell source 14a and the waste destination 14i at the beginning of the seeding process, as opposed to requiring a more complicated valve assembly 16 that would be operated to incrementally add bioreactor units 36a to the third flow path 38c that may be required to incrementally seed the n number of stacked bioreactor units 36a₁ to 36aₙ.

Referring now back to FIG. 9, microbeads 4 are optionally introduced into the n number of stacked bioreactor units 36a₁ to 36aₙ, such that the respective seed populations of biological cells 2 adhere to the microbeads 4.

In particular, a flow sequence of the n number of stacked bioreactor units 36a₁ to 36aₙ is added (e.g., via operation of the valve assembly 16) to the third flow path 38c between the microbead source 14b and the waste destination 14i (step 118) (see FIG. 10H), and in this case, by fluidly coupling the upper flow channels 48 of the respective n number of stacked bioreactor units 36a₁ to 36aₙ in series along the forward portion 38c′ of the third flow path 38a between the microbead source 14b and the waste destination 14i; fluidly coupling the lower flow channels 50 of the respective n number of stacked bioreactor units 36a₁ to 36aₙ in series along the return portion 38c″ of the third flow path 38c between the waste destination 14i and the microbead source 14b; and fluidly coupling the upper flow channel 48 and lower flow channel 50 of each of the n number of stacked bioreactor units 36a₁ to 36aₙ to each other via opening/closing of the ports 56-62 of each of the n number of stacked bioreactor units 36a₁ to 36aₙ in a similar manner described with respect to FIG. 7C.

Then, the microbeads 4 are introduced into the upper flow channels 48 of the respective n number of stacked bioreactor units 36a₁ to 36aₙ. In particular, the seeding media 26 containing microbeads 4 is flowed along the forward portion 38c′ of the third flow path 38c into the upper flow channels 48 of the respective n number of stacked bioreactor units 36a₁ to 36aₙ (step 120) (see FIG. 10I); seeding media 26′ containing no biological cells 2 and no microbeads 4 is perfused along bypass portions 38c″ of the third flow path 38c from the upper flow channels 48, through the porous membranes 46, and into the lower flow channels 50, of the respective n number of stacked bioreactor units 36a₁ to 36aₙ, while substantially retaining the biological cells 2 and microbeads 4 within the upper flow channels 48 of the respective n number of stacked bioreactor units 36a₁ to 36aₙ, such that the populations of biological cells 2 disposed on the cell culture surfaces 54 of the respective n number of stacked bioreactor units 36a₁ to 36aₙ adhere to the microbeads 4 (step 122) (see FIG. 10J); and the perfused seeding media 26′ containing no biological cells 2 and no microbeads 4 is flowed along the return portion 38c″ of the third flow path 38c out of the lower flow channels 50 of the respective n number of stacked bioreactor units 36a₁ to 36aₙ to the waste destination 14i (step 124) (see FIG. 10K).

While the microbeads 4 have been described as being delivered separately from the biological cells 2, into the upper flow channels 48 of the respective n number of stacked bioreactor units 36a₁ to 36aₙ, it should be appreciated that the microbeads 4 may be concurrently delivered with the biological cells 2, into the upper flow channels 48 of the respective n number of stacked bioreactor units 36a₁ to 36aₙ; e.g., by adding a flow sequence of the n number of stacked bioreactor units 36a₁ to 36aₙ to the third flow path 38c between the both the biological cell source 14a and microbead source 14b and the waste destination 14i. In such a case, the microbeads 4 may be incrementally introduced with the biological cells 2 into the upper flow channels 48 of the respective n number of stacked bioreactor units 36a₁ to 36aₙ in accordance with the method 150 illustrated in FIGS. 11 and 12A-12E.

Next, nutrient, reagent, and gas are delivered to the populations of biological cells 2, and waste is extracted from the populations of biological cells 2, thereby facilitating the expansion of the seed populations of biological cells 2 within the upper flow channels 48 of the respective n number of stacked bioreactor units 36a₁ to 36aₙ.

In particular, a flow sequence of the n number of stacked bioreactor units 36a₁ to 36aₙ is added (e.g., via operation of the valve assembly 16) to the second flow path 38b between the nutrient source 14c, reagent source 14d, and gas source 14e and the waste destination 14i, and in this case, by fluidly coupling the lower flow channels 50 of the respective n number of stacked bioreactor units 36a₁ to 36aₙ in series along the second flow path 38b between the nutrient source 14c, reagent source 14d, and gas source 14e and the waste destination 14i via opening/closing of the ports 56-62 of each of the n number of stacked bioreactor units 36a₁ to 36aₙ in a similar manner described with respect to FIG. 7B (step 126) (see FIG. 10L).

Then, fresh culture media 28 is flowed (continually or intermittently) along the second flow path 38b from the nutrient source 14c, reagent source 14d, and gas source 14e, respectively through the lower flow channels 50 of the respective n number of stacked bioreactor units 36a₁ to 36aₙ, while nutrient, reagent, gas, and waste are exchanged between the populations of biological cells 2 and fresh culture media 28 (i.e., the nutrient, reagent, and gas are provided to, while the waste is extracted from, the populations of biological cells 2, with the nutrient, reagent, and gas diffusing from the lower flow channels 50, through the porous membranes 46, and into the upper flow channels 48, and the waste diffusing from the upper flow channels 48, through the porous membranes 46, and into the lower flow channels 50, of the respective n number of stacked bioreactor units 36a₁ to 36aₙ), exiting the bioreactor assembly 12′ as spent culture media 28′ with the waste, thereby facilitating the expansion of the seed populations of biological cells 2 within the upper flow channels 48 of the respective n number of stacked bioreactor units 36a₁ to 36aₙ (step 128) (see FIG. 10M).

It should be appreciated that, over a period of time if the fresh culture media 28 is not replaced within the lower flow channels 50 (either continuously or intermittently), the seeding media 26 within the upper flow channels 48 and the culture media 28 within the lower flow channels 50 will become a homogenous solution with diffusion of nutrient, gas, reagents, and waste occurring across the porous membranes 46 between the flow channels 48, 50 ceasing upon achieving homogeneity between the seeding media 26 and the culture media 28. Thus, it is preferred that culture media 28 be replaced within the lower flow channels 50 with fresh culture media 28 before the culture media 28 becomes completely spent; i.e., prior to the seeding media 26 (or any other media) and the culture media 28 achieving homogeneity. Thus, the continual or intermittent flow of culture media 28 will maintain the chemical gradient between the flow channels 48, 50 necessary to effect the diffusion of nutrient, gas reagent, and waste through the porous membranes 46 (i.e., will maintain the concentration of nutrient, reagent, and gas in the culture media 28 at a higher level than the previously diffused nutrient, reagent, and gas remaining in the upper flow channels 48, and will maintain the concentration of previously diffused waste in the culture media 28 at a lower level than the remaining waste in the upper flow channels 48).

Anytime during incubation, the populations of biological cells 2 and microbeads 4 in the upper flow channels 48 of the respective n number of stacked bioreactor units 36a₁ to 36aₙ may be washed.

In particular, a flow sequence of the n number of stacked bioreactor units 36a₁ to 36aₙ is added (e.g., via operation of the valve assembly 16) to the third flow path 38c between the wash buffer source 14f and the waste destination 14i, and in this case, by fluidly coupling the upper flow channels 48 of the respective n number of stacked bioreactor units 36a₁ to 36aₙ in series along the forward portion 38c′ of the third flow path 38a between the wash buffer source 14f and the waste destination 14i; fluidly coupling the lower flow channels 50 of the respective n number of stacked bioreactor units 36a₁ to 36aₙ in series along the return portion 38c″ of the third flow path 38c between the waste destination 14i and the wash buffer source 14f; and fluidly coupling the upper flow channel 48 and lower flow channel 50 of each of the n number of stacked bioreactor units 36a₁ to 36aₙ to each other via opening/closing of the ports 56-62 of each of the n number of stacked bioreactor units 36a₁ to 36aₙ in a similar manner described with respect to FIG. 7C (step 130) (see FIG. 10N).

Then, biological cells 2 residing in the upper flow channels 48 of the respective n number of stacked bioreactor units 36a₁ to 36aₙ are washed. In particular, the bioreactor assembly 12′ is mechanically agitated, thereby suspending the populations of biological cells 2 and microbeads 4 in the upper flow channels 48, such that the populations of biological cells 2 will be maximally exposed to the fresh wash buffer 30 and will not block perfusion of the used wash buffer 30 containing undesirable material (e.g., unbound molecules, debris, dead cells, or other undesirable molecules) through the porous membranes 46, of the respective n number of stacked bioreactor units 36a₁ to 36aₙ (step 132) (see FIG. 10O); the fresh wash buffer 30 is flowed along the forward portion 38c′ of the third flow path 38c into, while accumulating the undesirable material from, the upper flow channels 48 of the respective n number of stacked bioreactor units 36a₁ to 36aₙ, thereby washing the populations of biological cells 2 and microbeads 4 (step 134) (see FIG. 10P); used wash buffer 30′ containing the undesirable material, is perfused along bypass portions 38c″ of the third flow path 38c from the upper flow channels 48, through the porous membranes 46, and into the lower flow channels 50, of the respective n number of stacked bioreactor units 36a₁ to 36aₙ, while substantially retaining the biological cells 2 and microbeads 4 within the upper flow channels 48, such that the populations of biological cells 2 and microbeads 4 are again urged against the cell culture surfaces 54 of the respective n number of stacked bioreactor units 36a₁ to 36aₙ, of the respective n number of stacked bioreactor units 36a₁ to 36aₙ (step 136) (see FIG. 10Q); and the used wash buffer 30′ containing the undesirable material is flowed along the return portion 38c″ of the third flow path 38c out of the lower flow channels 50 of the respective n number of stacked bioreactor units 36a₁ to 36aₙ to the waste destination 14i (step 138) (see FIG. 10R). After the washing steps, nutrient, reagent, and gas may be delivered to the populations of biological cells 2, and waste may be extracted from the populations of biological cells 2, thereby facilitating further expansion of the seed populations of biological cells 2 within the upper flow channels 48 of the respective n number of stacked bioreactor units 36a₁ to 36aₙ in accordance with steps 126 and 128.

Next, the expanded populations of biological cells 2 are harvested from the respective n number of stacked bioreactor units 36a₁ to 36aₙ.

In particular, a flow sequence of the n number of stacked bioreactor units 36a₁ to 36aₙ is added (e.g., via operation of the valve assembly 16) to the first flow path 36a between the saline source 14g and the biological cell collection destination 14h, and in this case, by fluidly coupling the upper flow channels 48 of the respective n number of stacked bioreactor units 36a₁ to 36aₙ in series along the first flow path 38a between the saline source 14g and the biological cell collection destination 14h via opening/closing of the ports 56-62 of each of the n number of stacked bioreactor units 36a₁ to 36aₙ in a similar manner described with respect to FIG. 7A (step 140) (see FIG. 10S).

Then, harvesting media 32 is flowed along the first flow path 38a from the saline source 14g, respectively through, while suspending expanded populations of biological cells 2 and microbeads 4 residing in, upper flow channels 48 (see FIG. 10T), exiting the bioreactor assembly 12′ as harvesting media 32′ with the suspended expanded populations of biological cells 2 and microbeads 4 to the biological cell collection destination 14h, thereby harvesting the expanded populations of the biological cells 2 and microbeads 4 from the upper flow channels 48 of the respective n number of stacked bioreactor units 36a₁ to 36aₙ (step 142) (see FIG. 10U).

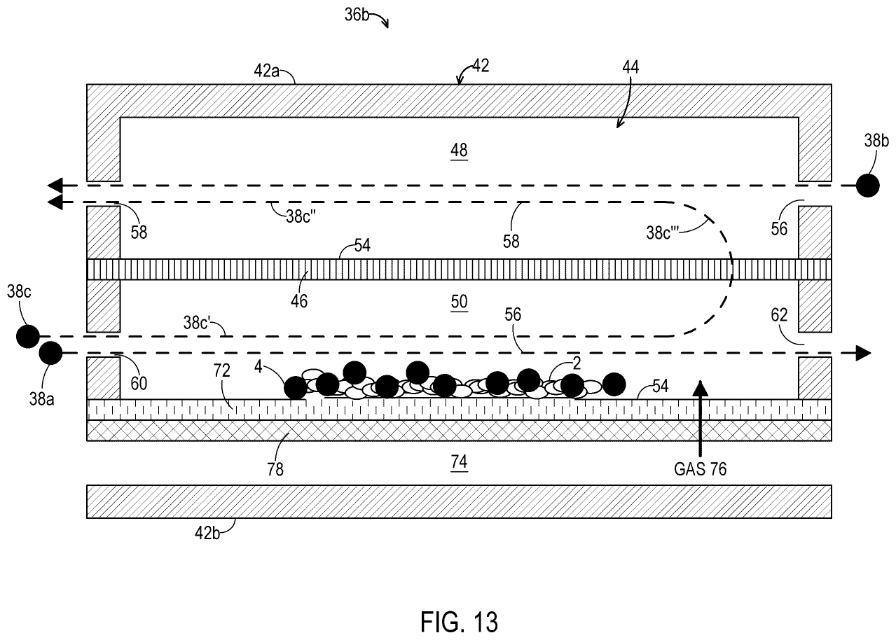

Referring to FIG. 13, another exemplary embodiment of a bioreactor unit 36b will now be described. The bioreactor unit 36b is similar to the bioreactor unit 36a illustrated in FIGS. 5-6, with the exception that the cell culture surface 54 is not formed on the porous membrane 46, but rather is separated and spaced a predetermined distance from the porous membrane 46. In the embodiment illustrated in FIG. 13, the cell culture surface 54 is located in the lower flow channel 50 and formed on a wall of the housing 42, and in particular, on the bottom wall of the housing 42 (or a layer disposed upon the bottom wall of the housing 42). In this manner, gravity urges the population of biological cells 2 to rest on the cell culture surface 54.

In this embodiment, the seeding media 26 with biological cells 2 (and optionally microbeads 4) can be flowed into the lower flow channel 50 to seed the bioreactor unit 36b with a population of biological cells 2 (and optionally microbeads 4); fresh culture media 28 with nutrient and reagent can be flowed (continuously or intermittently) through the upper flow channel 48, thereby providing nutrient, and reagent to, and extracting waste from, the population of biological cells 2 residing in the lower flow channel 50 via the action of diffusion through the porous membrane 46, thereby facilitating expansion of the seed population of biological cells 2; fresh wash buffer 30 may be flowed into the lower flow channel 50 during incubation of the population of biological cells 2 to remove undesirable material (e.g., unbound molecules, debris, dead cells, or other unwanted molecules); and harvesting media 32 can be flowed into the lower flow channel 50 to extract the expanded population of biological cells 2 from the bioreactor unit 36b.

Notably, by locating the cell culture surface 54 on which the population of biological cells 2 rest away from the porous membrane 46, the population of biological cells 2 will not be disturbed by any shear force that would otherwise result from the lateral flexing of the porous membrane 46 in response to a significant pressure differential between the flow channels 48, 50. Since the cell culture surface 54 is located on the rigid wall of the housing 42, a very stable platform on which the population of biological cells 2 rest is provided. It is preferred that the cell culture surface 54 be spaced away from the porous membrane 46 a low or minimal distance (e.g., at least 1 mm), such that the population of biological cells 2 resting on the cell culture surface 54 do not come into contact with the porous membrane 46. Not only does sufficient spacing between the population of biological cells 2 and the porous membrane 46 reduce or minimize any disturbance of the population of biological cells 2 when the porous membrane 46 lateral flexes in response to a significant pressure differential between the flow channels 48, 50, clogging of the pores within the porous membrane 46 with biological cells 2 will be avoided. As such, perfusion of the seeding media 26 and optional fresh wash buffer 30, as well as diffusion of nutrient, reagent, gas, and waste, across the membrane 46 will be more efficient. Furthermore, to the extent that the porous membrane 46 is not completely impermeable to the biological cells 2, maintaining the population of the biological cells 2 on the cell culture surface 54 away from the porous membrane 46 will prevent any loss (or reduce loss) of biological cells 2 (e.g., via creeping or suction) through the porous membrane 46.

The bioreactor unit 36b further comprises another membrane 72 affixed within the housing 42 at the bottom of the lower flow channel 50 spaced apart from and opposite the porous membrane 46, and on which the cell culture surface 54 is located. An ambient environment 74 is located below the other membrane 72, such that gas 66 (in this case, air) may be delivered through the other membrane 72 to the population of biological cells 2 via diffusion, while the nutrient and reagent from the fresh culture media 28 and waste from the population of biological cells 2 diffuses across the porous membrane 46 between the flow channels 48, 50, as discussed above. In an alternative embodiment, a gas chamber (not shown) with an inlet port and an outlet port may be located below the other membrane 72, such that the gas 66 may be flowed within the gas chamber in a controlled manner.

To this end, the other membrane 72 is substantially permeable to gas, such that the gas 76 may diffuse from the ambient environment 74 into the lower flow channel 50 for take-up by the population of biological cells 2, but substantially impermeable to liquid (e.g., the priming media 24, seeding media 26, culture media 28, washer buffer 30, or harvesting media 32) in the lower flow channel 50. The other membrane 72 is preferably a dense membrane (i.e., a membrane presenting no detectable pores at the limits of electron microscopy) that is composed of a low friction biocompatible material, e.g., a polymer, such as silicone, polytetrafluoroethylene (PTFE), polyether ether ketone (PEEK), etc., to prevent the population of biological cells 2 from adhering to the cell culture surface 54. Preferably, the dense membrane 72 provides a short diffusion path for the gas between the ambient environment 74 and the lower flow channel 50. For example, the dense membrane 72 may have a relatively small thickness, e.g., in the range of 50-250 microns. In one specific embodiment, the thickness of the dense membrane 56 is 100 microns. Notably, the use of a dedicated gas flow channel 58 separate from the fresh culture media 28 improves gas delivery to the population of biological cells 2. In an alternative embodiment, the other membrane 72 may be a porous, but hydrophobic, membrane. The bubble point of the membrane 72 should be relatively high (preferably, several times higher than the maximum pressure differential between the flow channels 48, 50).

It is preferred that the dense membrane 72, and thus the cell culture surface 54, be spaced away from the porous membrane 46 a minimal distance (e.g., at least 1 mm), such that the population of biological cells 2 resting on the cell culture surface 54 do not come into contact with the porous membrane 46. Not only does sufficient spacing between the population of biological cells 2 and the porous membrane 46 reduce or minimize any disturbance of the population of biological cells 2 when the porous membrane 46 lateral flexes in response to a significant pressure differential between the flow channels 48, 50, clogging of the pores within the porous membrane 46 with biological cells 2 will be avoided. As such, diffusion of nutrient, reagent, and waste across the porous membrane 46 between the flow channels 48, 50 will be more efficient. Furthermore, to the extent that the porous membrane 46 is not completely impermeable to the biological cells 2, maintaining the population of the biological cells 2 on the cell culture surface 54 away from the porous membrane 46 will prevent any loss of biological cells 2 (e.g., via creeping or suction) through the porous membrane 46.

The bioreactor unit 36b additionally comprises a permeable support structure 78 configured for reducing a lateral flex of the dense membrane 72 in response to a pressure differential between the lower flow channel 50 and the ambient environment 74. Because, the lower flow channel 50 will generally have a higher pressure than the pressure of the ambient environment 74, the permeable support structure 78 is affixed to the lower housing portion 42b between the bottom surface of the dense membrane 72 and the ambient environment 74 (e.g., via heat staking, thermal or ultrasonic welding, laminating, or simply being pinched in an interference fit) to reduce or minimize downward lateral flexing of the dense membrane 72 into the ambient environment 74 in response to a pressure differential resulting from the flow of seeding media 26, culture media 28, washer buffer 30, and harvesting media 32 through the lower flow channel 50 (i.e., the fluid pressure in the lower flow channel 50 becomes higher than the fluid pressure of the ambient environment 74. Thus, the population of biological cells 2 will not be disturbed by any shear force that would otherwise result from the lateral flexing of the dense membrane 72 in response to a significant pressure differential between the lower flow channel 50 and the ambient environment 74. As a result, the expansion of the seed population of biological cells 2 is improved. The permeable support structure 78 is highly porous so as to not unduly restrict the diffusion of gas through the dense membrane 72 between the lower flow channel 48 and the ambient environment 74. For example, the permeable support structure 78 may comprise a honeycomb or grid structure (e.g., composed stainless steel or a polymer, such as, e.g., polycarbonate, polystryrene, COC, COP, polypropylene, etc.). The permeable support structure 78 may have a suitable thickness, e.g., in the range of 0.1-2 mm, and a high porosity, e.g., in the range of 10-90%. Although the bioreactor unit 36b is described and illustrated as comprising a single permeable support structure 78, the bioreactor unit 36b may alternatively comprise two permeable support structures between which the dense membrane 72 is sandwiched.

Like the bioreactor unit 36a illustrated in FIG. 6, the bioreactor unit 36b has an arrangement of inlet and outlet ports to facilitate the flow of the priming media 24, seeding media 26, fresh culture media 28, fresh wash buffer 30, and harvesting media 32 through the interior cavity 44 of the bioreactor unit 36b, and in particular, comprises an inlet port 56 and outlet port 58 associated with the upper flow channel 48, and an inlet port 60 and outlet port 62 associated with the lower flow channel 50. Like the flow paths 38a-38c extending through the bioreactor unit 36a, the flow paths 38a-38c may extend through the bioreactor unit 36b. However, the flow paths 38a-38c are routed ones of the inlet port 56, outlet port 58, inlet port 60, and outlet port 62 that are opposite to the inlet port 56, outlet port 58, inlet port 60, and outlet port 62 of the bioreactor unit 36a through which the flow paths 38a-38c are routed. In particular, the flow path 38a may extend in through the inlet port 60, through the lower flow channel 50, and out through the outlet port 62; the flow path 38b may extend in through the inlet port 56, through the upper flow channel 48, and out through the outlet port 58; and the forward portion of the flow path 38c′ may extend in through the inlet port 60 and into the lower flow channel 50, the bypass portion of the flow path 38c″ may cross or traverse the porous membrane 46 from the lower flow channel 50 into the upper flow channel 48, and the return portion of the flow path 38c″ may exit the upper flow channel 50 out through the outlet port 58.

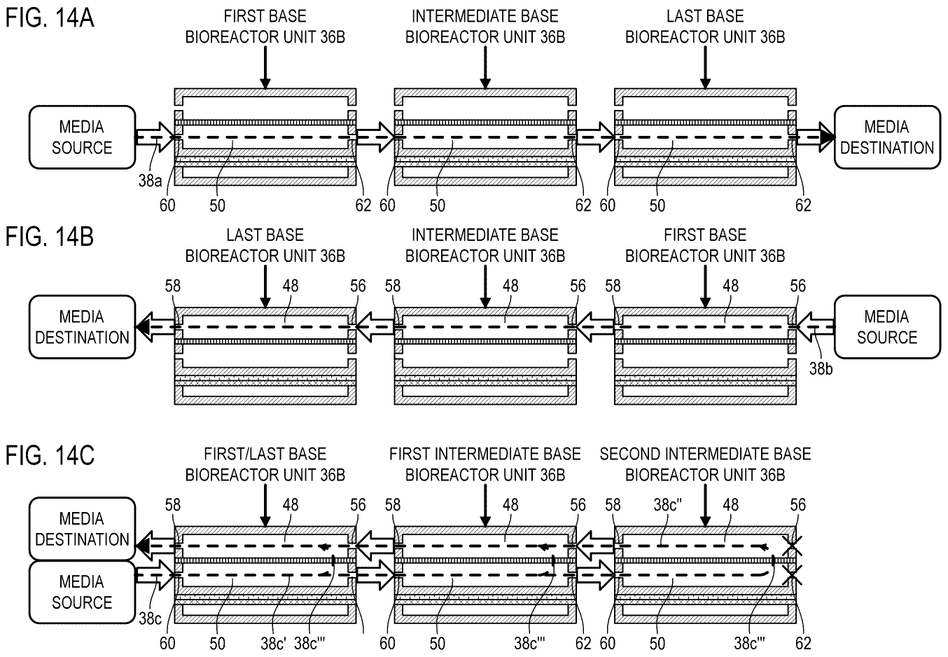

Referring further to FIGS. 14A-14C, examples illustrating the manner in which a flow sequence of bioreactor units 36b is added to each of the flow paths 38a-38c between a media source and a media destination will now be described. The manner in which flow sequences of bioreactor units 36b are added to each of the flow paths 38a-38c is similar to the manner in which flow sequences of bioreactor units 36a are added to each of the flow paths 38a-38c between a media source and a media destination described above with respect to FIGS. 7A-7C, with the exception that media source and media destination are fluidly coupled to different ones of the ports 56-62.

As illustrated in FIG. 14A, a flow sequence of bioreactor units 36b can be added in series to the first flow path 38a by fluidly coupling a media source (e.g., one or more of the wash buffer source 14f, and saline source 14g (shown in FIG. 1)) to the inlet port 60 of the first bioreactor unit 36b in the flow sequence; fluidly coupling a media destination (e.g., one of the biological cell collection destination 14h and waste destination 14i (shown in FIG. 1)) to the outlet port 62 of the last bioreactor unit 36b in the flow sequence; and fluidly coupling the outlet port 62 of each preceding bioreactor unit 36b to the inlet port 60 of the immediately succeeding bioreactor unit 36b (in this case, the outlet port 62 of the first bioreactor unit 36b to the inlet port 60 of the intermediate bioreactor unit 36b, and the outlet port 62 of the intermediate bioreactor unit 36b to the inlet port 60 of the last bioreactor unit 36b), thereby fluidly coupling the lower flow channels 50 of the bioreactor units 36b in series along the first flow path 38a between the media source and the media destination.

As illustrated in FIG. 7B, a flow sequence of bioreactor units 36b can be added in series to the second flow path 38b by fluidly coupling a media source (e.g., one or more of the nutrient source 14c, reagent source 14d, gas source 14e, wash buffer source 14f, and saline source 14g (shown in FIG. 1)) to the inlet port 56 of the first bioreactor unit 36b in the flow sequence; fluidly coupling a media destination (e.g., the waste destination 14i (shown in FIG. 1)) to the outlet port 58 of the last bioreactor unit 36b in the flow sequence; and fluidly coupling the outlet port 58 of each preceding bioreactor unit 36b to the inlet port 56 of the immediately succeeding bioreactor unit 36b (in this case, the outlet port 58 of the first bioreactor unit 36b to the inlet port 56 of the intermediate bioreactor unit 36b, and the outlet port 58 of the intermediate bioreactor unit 36b to the inlet port 56 of the last bioreactor unit 36b), thereby fluidly coupling the upper flow channels 48 of the bioreactor units 36b in series along the second flow path 38b between the media source and the media destination.

As illustrated in FIG. 7C, a flow sequence of bioreactor units 36b can be added in parallel to the third flow path 38c by fluidly coupling a media source (e.g., one or more of the biological cell source 14a, microbead source 14b, wash buffer source 14f, and saline source 14g (shown in FIG. 1)) to the inlet port 60 of the first bioreactor unit 36b in the flow sequence; fluidly coupling a media destination (e.g., the waste destination 14i (shown in FIG. 1)) to the outlet port 58 of the last bioreactor unit 36b in the flow sequence (which is the same as the first bioreactor unit in the flow sequence); fluidly coupling the outlet port 62 and inlet port 56 of each preceding bioreactor unit 36b respectively to the inlet port 60 and outlet port 58 of the immediately subsequent bioreactor unit 36b (in this case, the outlet port 62 and inlet port 56 of the first bioreactor unit 36b to the inlet port 60 and outlet port 58 of the first intermediate bioreactor unit 36b, and the outlet port 62 and inlet port 56 of the first intermediate bioreactor unit 36b to the inlet port 60 and outlet port 58 of the second intermediate bioreactor unit 36b), thereby fluidly coupling the lower flow channels 50 of the bioreactor units 36b in series along the forward portion 38c′ of the third flow path 38a between the media source and the media destination, and fluidly coupling the upper flow channels 48 of the bioreactor units 36b in series along the return portion 38c″ of the third flow path 38c between the media source and the media destination; and fluidly decoupling the outlet port 62 and inlet port 56 of the bioreactor unit 36b in the flow sequence in which the forward portion 38c′ of the third flow path 38a ends and the return portion 38c″ of the third flow patch 38a ends from any source of pressure less than the pressure required to perfuse the various media through the porous membrane 46 between the flow channels 48, 50, thereby fluidly coupling the upper flow channel 48 and lower flow channel 50 of each bioreactor unit 36b to each other.

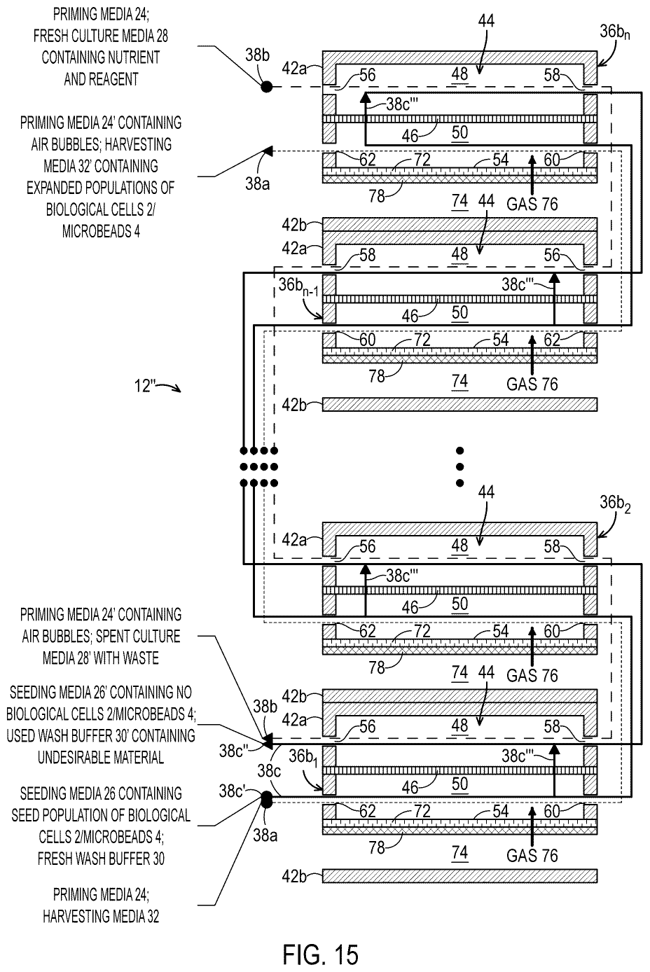

Referring now to FIG. 15, another embodiment of a bioreactor assembly 12″ that incorporates bioreactor unit 36b of FIG. 13 will be described. In this embodiment, the bioreactor units 36b are structurally distinct (e.g., they have separate housings), such that they can be structurally affixed to each other or removed from each other (e.g., via fasteners (not shown)) in a dynamic manner. In this manner, the bioreactor assembly 12″ can be structurally scaled up or scaled down in a dynamic manner; in essence, adding or subtracting flow channels 48, 50, as needed either prior to the beginning of the biological cell expansion process or during the biological cell expansion process. In an alternative embodiment of a bioreactor assembly, bioreactor units may be permanently structurally integrated (e.g., they have a single housing), in which case, scaling of such bioreactor assembly up or down may be accomplished during the manufacturing process, e.g., by fabricating bioreactor assemblies with different sizes and/or different numbers of bioreactor units.

Whether the bioreactor assembly includes structurally distinct bioreactor units 36b that can be dynamically affixed to each other or removed from each other or whether the bioreactor assembly has permanently integrated bioreactor units 36b, such bioreactor assembly may be functionally scaled up or down by activating (rendering operational) selected ones of the bioreactor units 36a by adding them to the flow paths 38a-38c, or deactivating (rendering non-operational) selected ones of the bioreactor units 36a by removing them from the flow paths 38a-38c; e.g., by adding a flow sequence of bioreactor units 36a to one or more of the flow paths 38a-38c between a media source and a media destination, as illustrated in FIGS. 14A-14C.

In the examples described below, the flow sequence of bioreactor units 36b added to a particular one of the flow paths 38a-38c includes all n number of stacked bioreactor units 36b₁ to 36bₙ, which are fluidly coupled in sequence in the same order as the order in which the n number of stacked bioreactor units 36b₁ to 36bₙ are physically arranged with each other (i.e., fluidly coupling the first stacked bioreactor unit 36b₁ directly to the second stacked bioreactor unit 36b₂, fluidly coupling the second stacked bioreactor unit 36b₂ directly to the third stacked bioreactor unit 36b₃, and so forth). However, it should be appreciated that the flow sequence of stacked bioreactor units 36b added to a particular one of the flow paths 38a-38c may include less than all n number of stacked bioreactor units 36b₁ to 36bₙ, including a flow sequence that excludes the first stacked bioreactor unit 36b₁ (e.g., stacked bioreactor units 36b₂ to 36bₙ), a flow sequence that excludes the last stacked bioreactor unit 36bₙ (e.g., stacked bioreactor units 36b₁ to 36bₙ₋₁), a flow sequence that excludes any one or more of the stacked bioreactor units 36b₂-36bₙ₋₁ between the first stacked bioreactor unit 36b₁ and the last stacked bioreactor unit 36bₙ (e.g., stacked bioreactor units 36b₂ to 36bₙ₋₁), or any flow sequence that fluidly couples stacked bioreactor units 36b in an order that does not match the order in which such bioreactor units 36b are physically arranged relative to each other (e.g., bioreactor unit 36b₁, then bioreactor unit 36bₙ₋₁, then bioreactor unit 36b₃, etc.).

The valve assembly 16 (illustrated in FIG. 1) may be operated to selectively open or close the ports 56-62 of a flow sequence of bioreactor units 36b of the bioreactor assembly 12″ illustrated in FIG. 15 to selected ones of the flow paths 38a-38c (i.e., by actively fluidly coupling selected ones the ports 56-62 to, or actively decoupling the selected ones of ports 56-62 from, the selected flow paths 38a-38c), thereby adding (activating) the flow sequence of bioreactor units 36b to the selected ones of the flow paths 38a-38c, while selectively fluidly coupling selected ones of the media sources/media destinations 14a-14i to the selected flow paths 38a-38c, to effect different functions, e.g., any of the aforementioned functions (a)-(e) described above with respect to FIG. 1.

In one example, the valve assembly 16, under control of the electric controller 22 (shown in FIG. 1), may be operated to prime the lower flow channels 50 of the bioreactor assembly 12″ by adding a flow sequence of bioreactor units 36b (in the illustrated case, all n number of stacked bioreactor units 36b₁ to 36bₙ) to the first flow path 38a between the saline source 14g and the waste destination 14i (shown in FIG. 1), with the saline source 14g serving as the media source, the waste destination 14i serving as the media destination, the first stacked bioreactor unit 36b₁ serving as a first bioreactor unit 36b in the flow sequence, the last stacked bioreactor unit 36bₙ serving as the last bioreactor unit 36b in the flow sequence, and the second stacked bioreactor unit 36b₂ to the second to last stacked bioreactor unit 36bₙ₋₁ serving as intermediate bioreactor units 36b in the flow sequence (see FIG. 14B).

In particular, the valve assembly 16 may be operated to prime the lower flow channels 50 of the bioreactor assembly 12″ by fluidly coupling the saline source 14g to the inlet port 60 of the first stacked bioreactor unit 36b₁; fluidly coupling the waste destination 14i to the outlet port 62 of the last stacked bioreactor unit 36bₙ); and fluidly coupling the outlet port 62 of each bioreactor unit 36b to the inlet port 60 of the immediately succeeding bioreactor unit 36b in the flow sequence along the first flow path 38a (i.e., the outlet port 62 of the first stacked bioreactor unit 36b₁ to the inlet port 60 of the second stacked bioreactor unit 36b₂, the outlet port 62 of the second stacked bioreactor unit 36b₂ to the inlet port 60 of the third stacked bioreactor unit 36b₃, and so forth), thereby fluidly coupling the lower flow channels 50 of the bioreactor units 36b in series between the saline source 14g and the waste destination 14i.

As a result, under the fluid pressure applied by the pump assembly 18 (shown in FIG. 1), priming media 24 flows along the first flow path 38a from the saline source 14g, in through the open inlet port 60 and through the lower flow channel 50 of the first stacked bioreactor unit 36b₁, thereby accumulating air bubbles from the lower flow channel 50, and priming media 24′ containing air bubbles then flows out through the open outlet port 62 of the first stacked bioreactor unit 36b₁; then the priming media 24′ containing air bubbles from the first stacked bioreactor unit 36b₁ flows further along the first flow path 38a in through the open inlet port 60 and through the lower flow channel 50 of the second stacked bioreactor unit 36b₂, thereby accumulating more air bubbles from the lower flow channel 50, and the priming media 24′ containing air bubbles then flows out through the open outlet port 62 of the second stacked bioreactor unit 36b₂; and so forth until the priming media 24′ containing air bubbles from all n number of bioreactor units 36b flows out of the open outlet port 62 of the last stacked bioreactor unit 36bₙ to the waste destination 14i. Thus, any or most of the air within the lower flow channels 48 of the respective n number of bioreactor units 36b will be completely displaced (i.e., flushed out) by the priming media 24. In this manner, the first flow path 38 (and at least the portion of the third flow path 38c that coincides with the first flow path 38a) extending through the n number of bioreactor units 36b will be consistent and well-defined.

Likewise, the valve assembly 16, under control of the electric controller 22 (shown in FIG. 1), may be operated to prime the upper flow channels 48 of the bioreactor assembly 12″ by adding a flow sequence of bioreactor units 36b (in the illustrated case, all n number of stacked bioreactor units 36b₁ to 36bₙ) to the second flow path 38b between the saline source 14g and the waste destination 14i (shown in FIG. 1), with the saline source 14g serving as the media source, the waste destination 14i serving as the media destination, the last stacked bioreactor unit 36bₙ serving as a first bioreactor unit 36b in the flow sequence, the first stacked bioreactor unit 36b₁ serving as the last bioreactor unit 36b in the flow sequence, and the second to last stacked bioreactor unit 36bₙ₋₁ to the second stacked bioreactor unit 36b₂ serving as intermediate bioreactor units 36b in the flow sequence (see FIG. 14A).

In particular, the valve assembly 16 may be operated to prime the upper flow channels 48 of the bioreactor assembly 12″ by fluidly coupling the saline source 14g to the inlet port 56 of the last stacked bioreactor unit 36bₙ; fluidly coupling the waste destination 14i to the outlet port 58 of the first stacked bioreactor unit 36b₁); and fluidly coupling the outlet port 58 of each bioreactor unit 36b to the inlet port 56 of the immediately succeeding bioreactor unit 36b in the flow path along the second flow path 38b (i.e., the outlet port 58 of the last stacked bioreactor unit 36bₙ to the inlet port 56 of the second to last stacked bioreactor unit 36bₙ₋₁, the outlet port 58 of the second to last stacked bioreactor unit 36bₙ₋₁ to the inlet port 56 of the third to last stacked bioreactor unit 36bₙ₋₂, and so forth), thereby fluidly coupling the lower flow channels 48 of the bioreactor units 36b in series between the saline source 14g and the waste destination 14i.

As a result, under the fluid pressure applied by the pump assembly 18 (shown in FIG. 1), priming media 24 flows along the second flow path 38b from the saline source 14g, in through the open inlet port 56 and through the upper flow channel 48 of the last stacked bioreactor unit 36bₙ, thereby accumulating air bubbles from the upper flow channel 48, and priming media 24′ containing air bubbles then flows out through the open outlet port 58 of the last stacked bioreactor unit 36bₙ; then the priming media 24′ containing air bubbles from the last stacked bioreactor unit 36bₙ flows further along the second flow path 38b in through the open inlet port 56 and through the upper flow channel 48 of the second to last stacked bioreactor unit 36bₙ₋₁, thereby accumulating more air bubbles from the upper flow channel 48, and the priming media 24′ containing air bubbles then flows out through the open outlet port 58 of the second to last stacked bioreactor unit 36bₙ₋₁; and so forth until the priming media 24′ containing air bubbles from all n number of bioreactor units 36b flows out of the open outlet port 58 of the first stacked bioreactor unit 36b₁ to the waste destination 14i. Thus, any or most of the air within the upper flow channels 48 of the respective n number of bioreactor units 36b will be completely displaced (i.e., flushed out) by the priming media 24. In this manner, the second flow path 38b (and at least the portion of the third flow path 38c that coincides with the second flow path 38b) extending through the n number of bioreactor units 36b will be consistent and well-defined.

In another example, the valve assembly 16, under control of the electric controller 22 (shown in FIG. 1), may be operated to seed the bioreactor assembly 12″ with multiple populations of biological cells 2 and/or microbeads 4 by adding a flow sequence of bioreactor units 36b (in the illustrated case, all n number of stacked bioreactor units 36b₁ to 36bₙ) to the third flow path 38c between the biological cell source 14a and/or microbead source 14b and the waste destination 14i (shown in FIG. 1), with the biological cell source 14a and/or microbead source 14b serving as the media source, the waste destination 14i serving as the media destination, the first stacked bioreactor unit 36b₁ serving as both the first bioreactor unit 36b and the last bioreactor unit 36b, and the second stacked bioreactor unit 36b₂ to the last stacked bioreactor unit 36bₙ serving as intermediate bioreactor units 36b, in the flow sequence along the forward portion 38c′ and return portion 38c″ of the third flow path 38c (see FIG. 14C).

In particular, the valve assembly 16 may be operated to seed the bioreactor assembly 12″ with the populations of biological cells 2 and/or microbeads 4 by fluidly coupling the biological cell source 14a and/or microbead source 14b to the inlet port 60 of the first stacked bioreactor unit 36b₁; fluidly coupling the waste destination 14i to the outlet port 58 of the first stacked bioreactor unit 36b₁; fluidly coupling the outlet port 62 of each bioreactor unit 36b to the inlet port 60 of the immediately succeeding bioreactor unit 36b in the flow sequence along the forward path 38c′ of the third flow path 38c (i.e., the outlet port 62 of the first stacked bioreactor unit 36b₁ to the inlet port 60 of the second stacked bioreactor unit 36b₂, the outlet port 62 of the second stacked bioreactor unit 36b₂ to the inlet port 60 of the third stacked bioreactor unit 36b₃, and so forth), thereby fluidly coupling the lower flow channels 50 of the bioreactor units 36b in series between the saline source 14g and the waste destination 14i; fluidly coupling the outlet port 58 of each bioreactor unit 36b to the inlet port 56 of the immediately succeeding bioreactor unit 36b in the flow sequence along the return path 38c″ of the third flow path 38c (i.e., the outlet port 58 of the last stacked bioreactor unit 36bₙ to the inlet port 56 of the second to last stacked bioreactor unit 36bₙ₋₁, the outlet port 58 of the second to last stacked bioreactor unit 36b₂ to the inlet port 56 of the third to last stacked bioreactor unit 36b₃, and so forth), thereby fluidly coupling the upper flow channels 50 of the bioreactor units 36b in series between the saline source 14g and the waste destination 14i; and fluidly decoupling the outlet port 62 and the inlet port 56 of the last stacked bioreactor unit 36bₙ from any source of fluid, thereby fluidly coupling the upper flow channel 48 and lower flow channel 50 of each bioreactor unit 36b to each other (in effect, removing the stacked bioreactor units 36b₁ to 36bₙ from the other flow paths 38a, 38b to increase the pressure in the lower flow channels 48 to urge fluidly coupling between the upper flow channels 48 and the lower flow channels 50.

As a result, under the fluid pressure applied by the pump assembly 18 (shown in FIG. 1), seeding media 26 containing a seed population of biological cells 2 and/or microbeads 4 flows along the forward portion 38c′ of the third flow path 38c from the biological cell source 14a and/or microbead source 14b, in through the open inlet port 60, through the lower flow channel 50, and out through the open outlet port 62, of the first stacked bioreactor unit 36b₁; then some of the seeding media 26′ without the biological cells 2 and/or microbeads 4 perfuses along the bypass portion 38c″ of the third flow path 38c through the porous membrane 46 from the lower flow channel 50 into the upper flow channel 48, of the first stacked bioreactor unit 36b₁ (due to the pressure buildup in the lower flow channel 50 caused by the closed outlet port 62 of the last stacked bioreactor unit 36bₙ creating a pressure gradient between the flow channels 48, 50 of all n number of stacked bioreactor units 36b₁ to 36bₙ); and then the perfused seeding media 26′ without the population of biological cells 2 and/or microbeads 4 flows along the return portion 38c″ of the third flow path 38c from the upper flow channel 48 and only out through the open outlet port 58, of the first stacked bioreactor unit 36b₁ (due to the closed inlet port 56 of the last stacked bioreactor unit 36bₙ preventing the flow of fluid along the second flow path 38b in the opposite direction from the upper flow channels 50 out through the inlet ports 56 of all n number of stacked bioreactor units 36b₁ to 36bₙ), and to the waste destination 14i. Significantly, while the seeding media 26′ perfuses through the porous membrane 46, the porous membrane 46 captures (filters) the biological cells 2 and/or microbeads 4 via the action of convection, which captured biological cells 2 will then settle downward in the lower flow channel 50 via gravity to the cell culture surface 54 at the bottom of the lower flow channels 50, thereby seeding the upper flow channel 48 of the first stacked bioreactor unit 36b₁ with a population of biological cells 2 and/or microbeads 4.

This flow of the seeding media 26 containing the seed population of biological cells 2 and/or microbeads 4 and the seeding media 26 containing no biological cells 2 and/or microbeads 4 repeats for the second stacked bioreactor unit 36b₂ to the second to last bioreactor 36bₙ₋₁, with the seeding media 26 containing the seed population of biological cells 2 and/or microbeads 4 flowing along the forward portion 38c′ of the third flow path 38c out through the open outlet port 62 of each bioreactor unit 36b, flowing in through the open inlet port 60 of an immediately succeeding bioreactor unit 36b in the flow sequence, and the seeding media 26 containing no biological cells 2 and/or microbeads 4 that has perfused along the bypass portion 38c″ of the third flow path 38c across the porous membrane 46, flowing along the return portion 38c″ of the third flow path 38c out through the open outlet port 58 of each bioreactor unit 36b and flowing in through the open inlet port 56 of an immediately succeeding bioreactor unit 36b in the flow sequence, thereby seeding the second stacked bioreactor unit 36b₂ to the second to last bioreactor 36bₙ₋₁ with populations of biological cells 2 and/or microbeads 4.

Lastly, the seeding media 26 containing the seed population of biological cells 2 and/or microbeads 4 flows along the forward portion 38c′ of the third flow path 38c from the open outlet port 62 of the second to last stacked bioreactor unit 36bₙ₋₁, in through the open inlet port 60, and into the lower flow channel 50, of the last stacked bioreactor unit 36bₙ; then the seeding media 26′ without the biological cells 2 and/or microbeads 4 perfuses along the bypass portion 38c″ of the third flow path 38c through the porous membrane 46 from the lower flow channel 50 into the upper flow channel 48, of the last stacked bioreactor unit 36bₙ (due to the pressure buildup in the lower flow channel 48 caused by the closed outlet port 62 of the last stacked bioreactor unit 36bₙ creating a pressure gradient between the flow channels 48, 50 of all n number of stacked bioreactor units 36b₁ to 36bₙ); and then the perfused seeding media 26′ without the population of biological cells 2 and/or microbeads 4 flows along the return portion 38c″ of the third flow path 38c from the upper flow channel 48 and out through the open outlet port 58, of the last stacked bioreactor unit 36bₙ, back in through the open inlet port 56 of the second to last stacked bioreactor unit 36bₙ₋₁, thereby seeding the lower flow channel 50 of the last stacked bioreactor unit 36bₙ with a population of biological cells 2 and/or microbeads 4.

Significantly, because the biological cells 2 and/or microbeads 4 are filtered and captured by the porous membranes 46 of the respective n number of bioreactors 36b while the seeding media 26 containing the seed population of biological cells 2 and microbeads 4 are prevented from flowing out of the upper flow channel 48 of the last stacked bioreactor unit 36bₙ via the closed outlet port 58, loss of biological cells 2 and/or microbeads 4 out of the bioreactor assembly 12″ is minimized during respective seeding of the respective n number of bioreactor units 36b with the populations of biological cells 2 and/or microbeads 4. In this manner, seeding of the respective n number of bioreactor units 36b with populations of biological cells 2 and/or microbeads 4 may be accomplished robustly and expeditiously without concern that some of the biological cells 2 and/or microbeads 4 will be lost with the outflow of seeding media 26 from the bioreactor assembly 12″. Although, in an alternative embodiment, the seeding media 26 with the biological cells 2 and/or microbeads 4 may be flowed entirely through the lower flow channels 50, e.g., by opening both the inlet ports 60 and outlet ports 62 of all the bioreactor units 36b (including the last stacked bioreactor unit 36bₙ), essentially adding the n number of bioreactor units 36b to the first flow path 38a, care must be taken to ensure that a significant amount of biological cells 2 and/or microbeads 4 do not escape out of the bioreactor assembly 12″ with the outflow of the seeding media 26.

In still another example, the valve assembly 16, under control of the electric controller 22, may be operated to cultivate and expand the populations of biological cells 2 by adding a flow sequence of bioreactor units 36b (in the illustrated case, all n number of stacked bioreactor units 36b₁ to 36bₙ) to the second flow path 38a between the nutrient source 14c and reagent source 14d, and the waste destination 14i (shown in FIG. 1), with the nutrient source 14c and reagent source 14d, serving as the media source, the waste destination 14i serving as the media destination, the last stacked bioreactor unit 36bₙ serving as a first bioreactor unit 36b in the flow sequence, the first stacked bioreactor unit 36b₁ serving as the last bioreactor unit 36b in the flow sequence, and the second to last stacked bioreactor unit 36bₙ₋₁ to the second stacked bioreactor unit 36b₂ serving as intermediate bioreactor units 36b in the flow sequence (see FIG. 14A).

In particular, the valve assembly 16 may be operated to cultivate and expand the populations of biological cells 2 by fluidly coupling the nutrient source 14c and reagent source 14d to the inlet port 56 of the last stacked bioreactor unit 36bₙ; fluidly coupling the waste destination 14i to the outlet port 58 of the first stacked bioreactor unit 36b₁); and fluidly coupling the outlet port 58 of each bioreactor unit 36b to the inlet port 56 of the immediately succeeding bioreactor unit 36b in the flow sequence along the second flow path 38b (i.e., the outlet port 58 of the last stacked bioreactor unit 36bₙ to the inlet port 56 of the second to last stacked bioreactor unit 36bₙ₋₁, the outlet port 58 of the second to last stacked bioreactor unit 36b₂ to the inlet port 56 of the third to last stacked bioreactor unit 36b₃, and so forth), thereby fluidly coupling the upper flow channels 48 of the bioreactor units 36b in series between the nutrient source 14c and reagent source 14d and the waste destination 14i.

As a result, under the fluid pressure applied by the pump assembly 18 (shown in FIG. 1), fresh culture media 28 flows along the second flow path 38b from the nutrient source 14c and reagent source 14d, in through the open inlet port 56, through the upper flow channel 48, and out through the open outlet port 58, of the last stacked bioreactor unit 36bₙ; then in through the open inlet port 56, through the upper flow channel 48, and out through the open outlet port 58, of the second to last stacked bioreactor unit 36bₙ₋₁; and so forth until spent culture media 28′ flows out of the open outlet port 62 of the first stacked bioreactor unit 36b₁ to the waste destination 14i. Thus, nutrient and reagent is delivered to the environment surrounding the populations of biological cells 2 residing in the n number of bioreactor assemblies 36b, and waste is extracted from the environment surrounding the populations of biological cells 2 residing the n number of bioreactor assemblies 36b, and in particular, by allowing the nutrient and reagent within the fresh culture media 28 to diffuse through the porous membranes 46 from the upper flow channels 48 into the lower flow channels 50, of the respective n number of bioreactor units 36b, and allowing the waste to diffuse through the porous membranes 46 from the lower flow channels 50 into the upper flow channels 48, of the respective n number of bioreactor units 36b, thereby facilitating expansion of the seed populations of biological cells 2.

In yet another example, the valve assembly 16, under control of the electric controller 22 (shown in FIG. 1), may be operated to wash the populations of biological cells 2 and/or microbeads 4 by adding a flow sequence of bioreactor units 36b (in the illustrated case, all n number of stacked bioreactor units 36b₁ to 36bₙ) to the third flow path 38c between the wash buffer source 14f and the waste destination 14i (shown in FIG. 1), with the wash buffer source 14f serving as the media source, the waste destination 14i serving as the media destination, the first stacked bioreactor unit 36b₁ serving as both the first bioreactor unit 36b and the last bioreactor unit 36b, and the second stacked bioreactor unit 36b₂ to the last stacked bioreactor unit 36bₙ serving as intermediate bioreactor units 36b, in the flow sequence along the forward portion 38c′ and return portion 38c″ of the third flow path 38c (see FIG. 14C).

In particular, the valve assembly 16 may be operated to wash the populations of biological cells 2 and/or microbeads 4 by fluidly coupling the wash buffer source 14f to the inlet port 60 of the first stacked bioreactor unit 36b₁; fluidly coupling the waste destination 14i to the outlet port 58 of the first stacked bioreactor unit 36b₁; fluidly coupling the outlet port 62 of each bioreactor unit 36b to the inlet port 60 of the immediately succeeding bioreactor unit 36b in the flow sequence along the forward path 38c′ of the third flow path 38c (i.e., the outlet port 62 of the first stacked bioreactor unit 36b₁ to the inlet port 60 of the second stacked bioreactor unit 36b₂, the outlet port 62 of the second stacked bioreactor unit 36b₂ to the inlet port 60 of the third stacked bioreactor unit 36b₃, and so forth), thereby fluidly coupling the lower flow channels 50 of the bioreactor units 36b in series between the wash buffer source 14f and the waste destination 14i; fluidly coupling the outlet port 58 of each bioreactor unit 36b to the inlet port 56 of the immediately subsequent bioreactor unit 36b in the flow sequence along the return path 38c″ of the third flow path 38c (i.e., the outlet port 58 of the last stacked bioreactor unit 36bₙ to the inlet port 56 of the second to last stacked bioreactor unit 36bₙ₋₁, the outlet port 58 of the second to last stacked bioreactor unit 36b₂ to the inlet port 56 of the third to last stacked bioreactor unit 36b₃, and so forth), thereby fluidly coupling the lower flow channels 50 of the bioreactor units 36b in series between the wash buffer source 14f and the waste destination 14i; and fluidly decoupling the outlet port 62 and the inlet port 56 of the last stacked bioreactor unit 36bₙ from any source of fluid, thereby fluidly coupling the upper flow channel 48 and lower flow channel 50 of each bioreactor unit 36b to each other (in effect, removing the stacked bioreactor units 36b₁ to 36bₙ from the other flow paths 38a, 38b to increase the pressure in the lower flow channels 48 to urge fluidly coupling between the upper flow channels 48 and the lower flow channels 50.

As a result, under the fluid pressure applied by the pump assembly 18 (shown in FIG. 1), fresh wash buffer 30 flows along the forward portion 38c′ of the third flow path 38c from the wash buffer source 14f, in through the open inlet port 60, through the lower flow channel 50, and out through the open outlet port 62, of the first stacked bioreactor unit 36b₁; while used wash buffer 30′ containing undesirable material (e.g., unbound molecules, debris, dead cells, or other undesirable molecules) perfuses along the bypass portion 38c′″ of the third flow path 38c through the porous membrane 46 from the lower flow channel 50 into the upper flow channel 48, of the first stacked bioreactor unit 36b₁ (due to the pressure buildup in the lower flow channel 50 caused by the closed outlet port 62 of the last stacked bioreactor unit 36bₙ creating a pressure gradient between the flow channels 48, 50 of all n number of stacked bioreactor units 36b₁ to 36bₙ); and then the perfused used wash buffer 30′ containing the undesirable material flows along the return portion 38c″ of the third flow path 38c from the upper flow channel 48 and only out through the open outlet port 58, of the first stacked bioreactor unit 36b₁ (due to the closed inlet port 56 of the last stacked bioreactor unit 36bₙ preventing the flow of fluid along the second flow path 38b in the opposite direction from the upper flow channels 48 out through the inlet ports 56 of all n number of stacked bioreactor units 36b₁ to 36bₙ), and to the waste destination 14i. Significantly, while the used wash buffer 30′ containing the undesirable material perfuses through the porous membrane 46, the porous membrane 46 retains the biological cells 2 and/or microbeads 4, thereby washing the population of biological cells 2 and microbeads 4 residing in the first stacked bioreactor unit 36b₁.

This flow of the fresh wash buffer 30 and used wash buffer 30′ containing the undesirable material repeats for the second stacked bioreactor unit 36b₂ to the second to last bioreactor 36bₙ₋₁, with the fresh wash buffer 30 flowing along the forward portion 38c′ of the third flow path 38c out through the open outlet port 62 of each bioreactor unit 36b, then flowing in through the open inlet port 60 of the immediately succeeding bioreactor unit 36b, and the used wash buffer 30′ containing the undesirable material that has perfused along the bypass portion 38c″ of the third flow path 38c across the porous membrane 46, flowing along the return portion 38c″ of the third flow path 38c out through the open outlet port 58 of each bioreactor unit 36b and flowing in through the open inlet port 56 of an immediately succeeding bioreactor unit 36b in the flow sequence, thereby washing the population of biological cells 2 and/or microbeads 4 residing in the second bioreactor unit 36b₂ to the second to last bioreactor 36bₙ₋₁.

Lastly, the fresh wash buffer 30 flows along the forward portion 38c′ of the third flow path 38c from the open outlet port 62 of the second to last stacked bioreactor unit 36bₙ₋₁, in through the open inlet port 60, and into the lower flow channel 50, of the last stacked bioreactor unit 36bₙ; then the used wash buffer 30′ containing the undesirable material perfuses along the bypass portion 38c″ of the third flow path 38c through the porous membrane 46 from the lower flow channel 50 into the upper flow channel 48, of the last stacked bioreactor unit 36bₙ (due to the pressure buildup in the lower flow channel 50 caused by the closed outlet port 62 of the last stacked bioreactor unit 36bₙ creating a pressure gradient between the flow channels 48, 50 of all n number of stacked bioreactor units 36b₁ to 36bₙ); and then the perfused used wash buffer 30′ containing the undesirable material flows along the return portion 38c″ of the third flow path 38c from the upper flow channel 48 and out through the open outlet port 58, of the last stacked bioreactor unit 36bₙ, back in through the open inlet port 56 of the second to last stacked bioreactor unit 36bₙ₋₁, thereby washing the population of biological cells 2 and/or microbeads 4 residing in the last stacked bioreactor unit 36bₙ.

In yet another example, the valve assembly 16, under control of the electric controller 22 (shown in FIG. 1), may be operated to harvest the expanded populations of biological cells 2 and/or microbeads 4 from the bioreactor assembly 12″ by adding a flow sequence of bioreactor units 36b (in the illustrated case, all n number of stacked bioreactor units 36b₁ to 36bₙ) to the first flow path 38a between the saline source 14g and the biological cell collection destination 14h (shown in FIG. 1), with the saline source 14g serving as the media source, the biological cell collection destination 14h serving as the media destination, the first stacked bioreactor unit 36b₁ serving as a first bioreactor unit 36b in the flow sequence, the last stacked bioreactor unit 36bₙ serving as the last bioreactor unit 36b in the flow sequence, and the second stacked bioreactor unit 36b₂ to the second to last stacked bioreactor unit 36bₙ₋₁ serving as intermediate bioreactor units 36b in the flow sequence (see FIG. 14B).

In particular, the valve assembly 16 may be operated to harvest the expanded populations of biological cells 2 and/or microbeads 4 from the bioreactor assembly 12″ by fluidly coupling the saline source 14g to the inlet port 60 of the first stacked bioreactor unit 36b₁; fluidly coupling the biological cell collection destination 14h to the outlet port 62 of the last stacked bioreactor unit 36bₙ); and fluidly coupling the outlet port 62 of each bioreactor unit 36b to the inlet port 60 of the immediately succeeding bioreactor unit 36b in the flow sequence along the first flow path 38a (i.e., the outlet port 62 of the first stacked bioreactor unit 36b₁ to the inlet port 60 of the second stacked bioreactor unit 36b₂, the outlet port 62 of the second stacked bioreactor unit 36b₂ to the inlet port 60 of the third stacked bioreactor unit 36b₃, and so forth), thereby fluidly coupling the lower flow channels 50 of the bioreactor units 36b in series between the saline source 14g and the waste destination 14i.

As a result, under the fluid pressure applied by the pump assembly 18 (shown in FIG. 1), harvesting media 32 flows along the first flow path 38a from the saline source 14g, in through the open inlet port 60 of the first stacked bioreactor unit 36b₁, and through the lower flow channel 50, thereby suspending the expanded population of biological cells 2 and microbeads 4 from the lower flow channel 50 within the harvesting media 32, and harvesting media 32′ with the suspended expanded population of biological cells 2 and microbeads 4 then flows out through the open outlet port 62 of the first stacked bioreactor unit 36b₁; then the harvesting media 32′ with the suspended expanded population of biological cells 2 and microbeads 4 from the first stacked bioreactor unit 36b₁ flows further along the first flow path 38a in through the open inlet port 60 of the second stacked bioreactor unit 36b₂, and through the lower flow channel 50, thereby suspending the expanded population of biological cells 2 and microbeads 4 from the lower flow channel 50 within the harvesting media 32′ then flows out through the open outlet port 62 of the second stacked bioreactor unit 36b₂; and so forth until the harvesting media 32′ with the suspended expanded population of biological cells 2 and microbeads 4 from all n number of bioreactor units 36b flows out of the open outlet port 62 of the last stacked bioreactor unit 36bₙ to the biological cell collection destination 14h. Thus, expanded populations of the biological cells 2 and microbeads 4 will be harvested from the lower flow channels 50 of the respective n number of bioreactor units 36b.

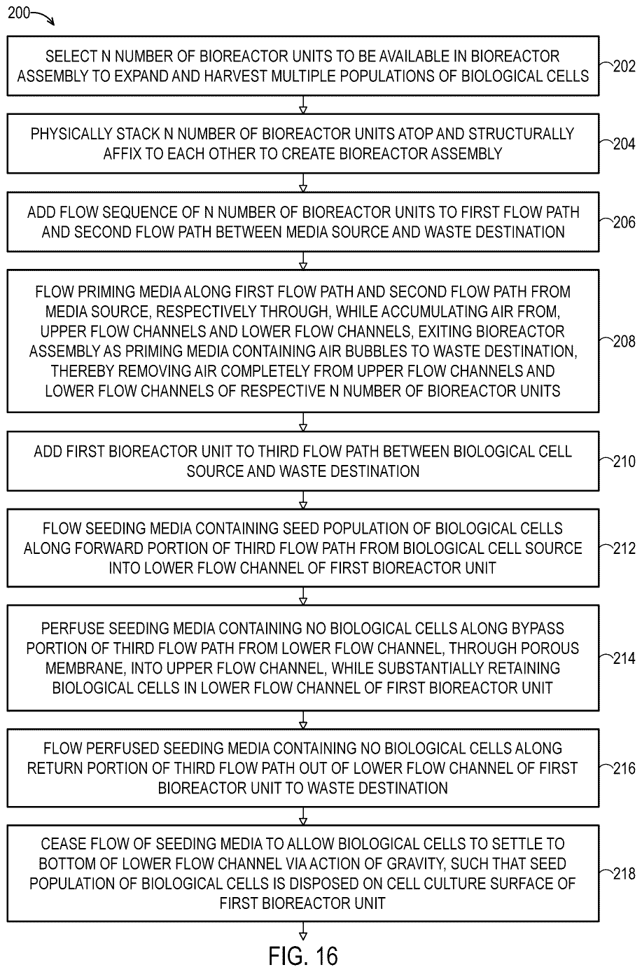

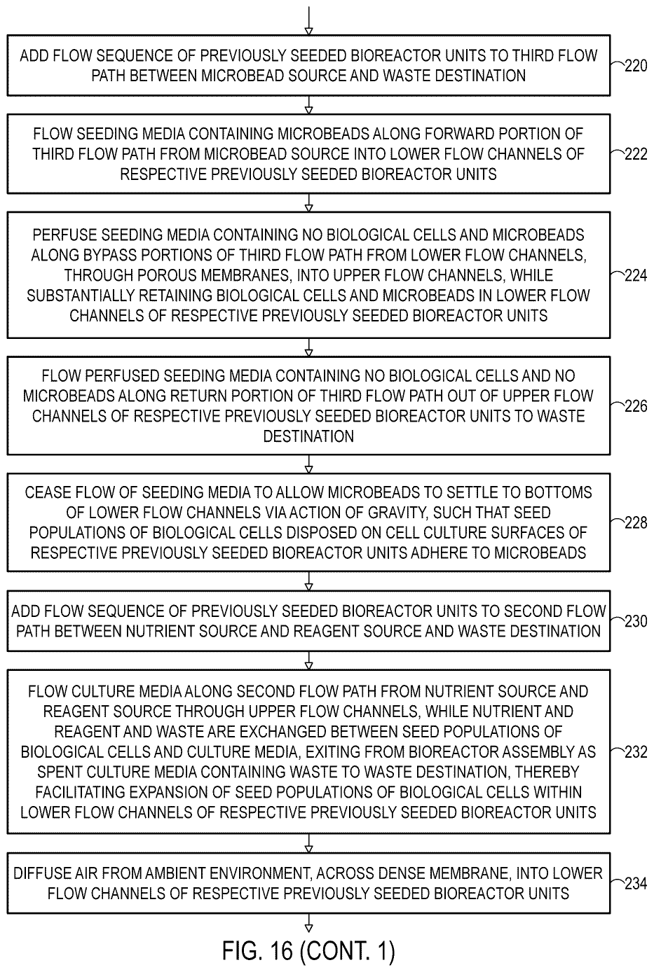

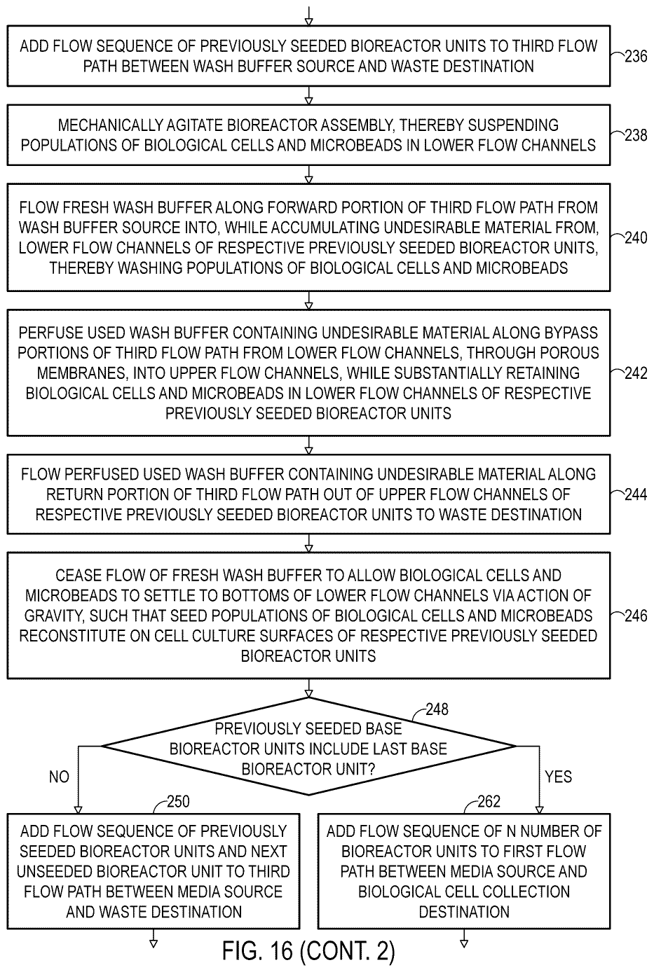

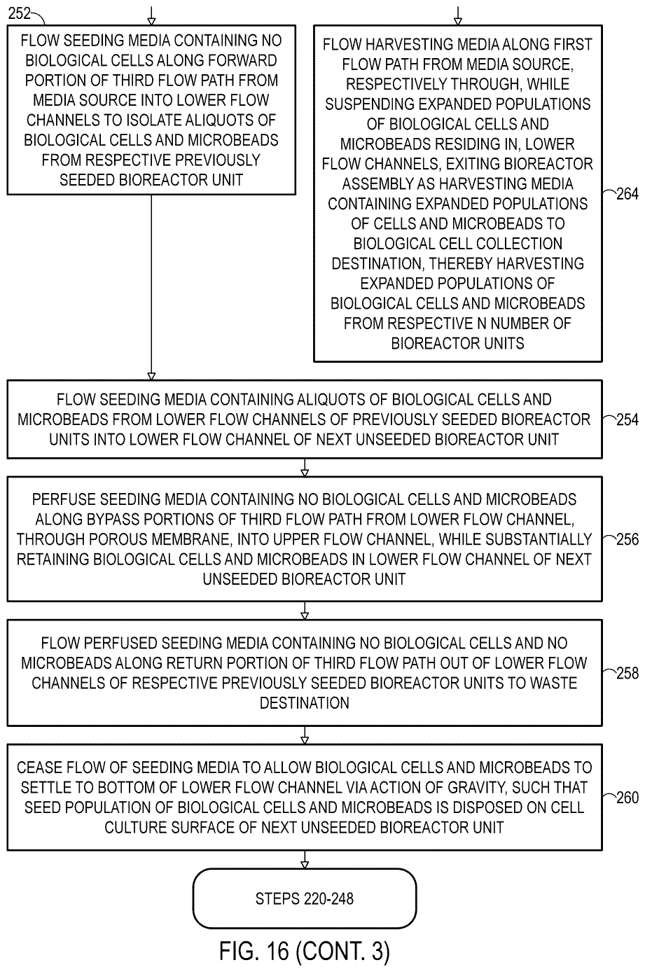

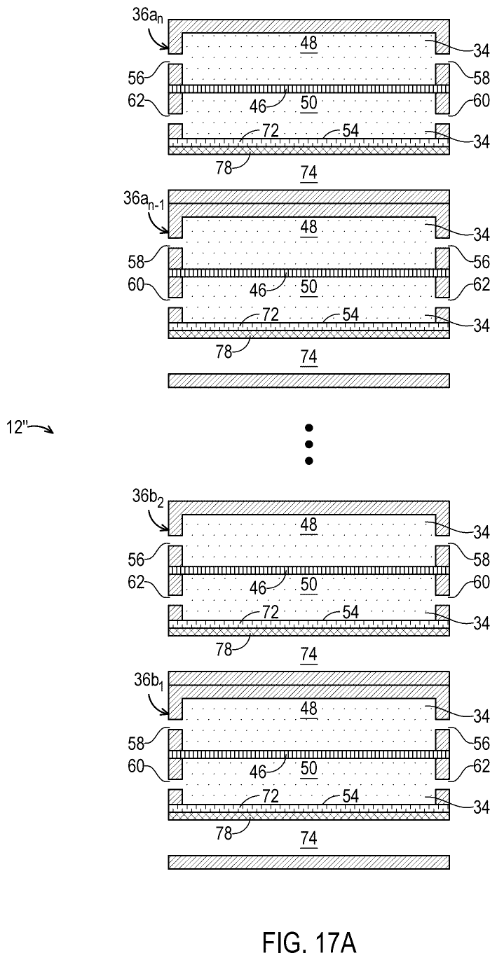

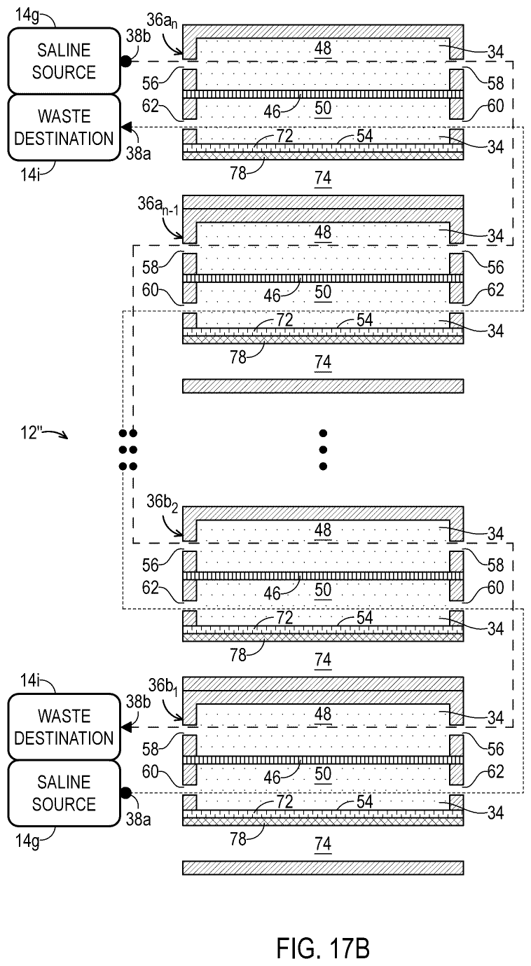

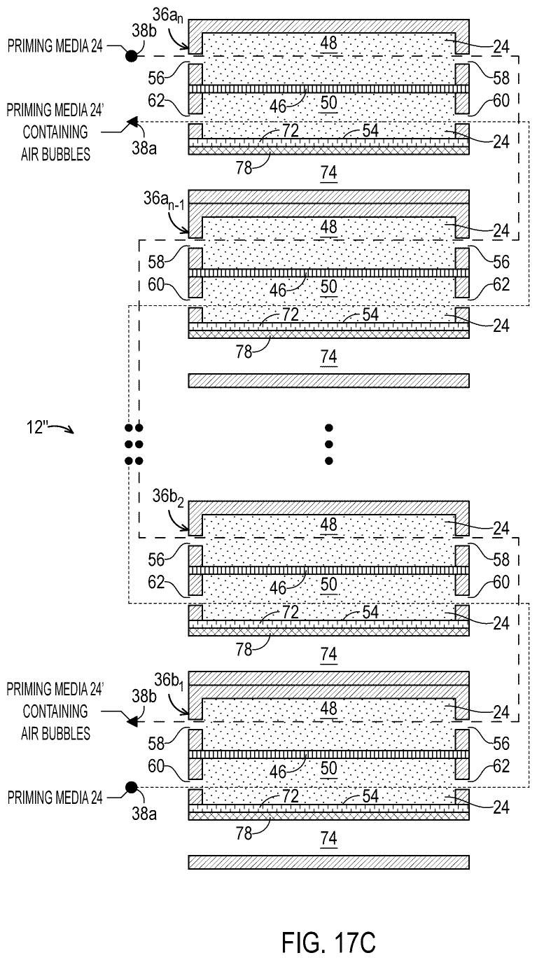

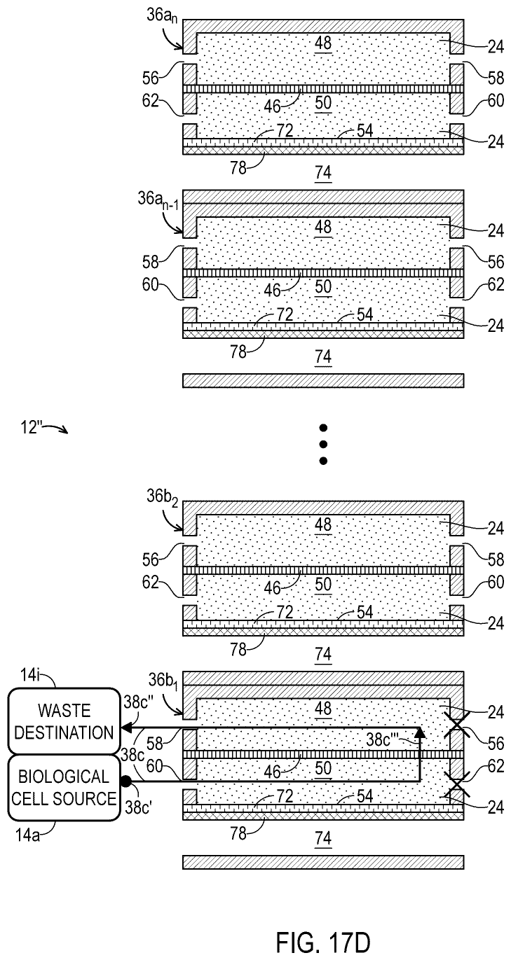

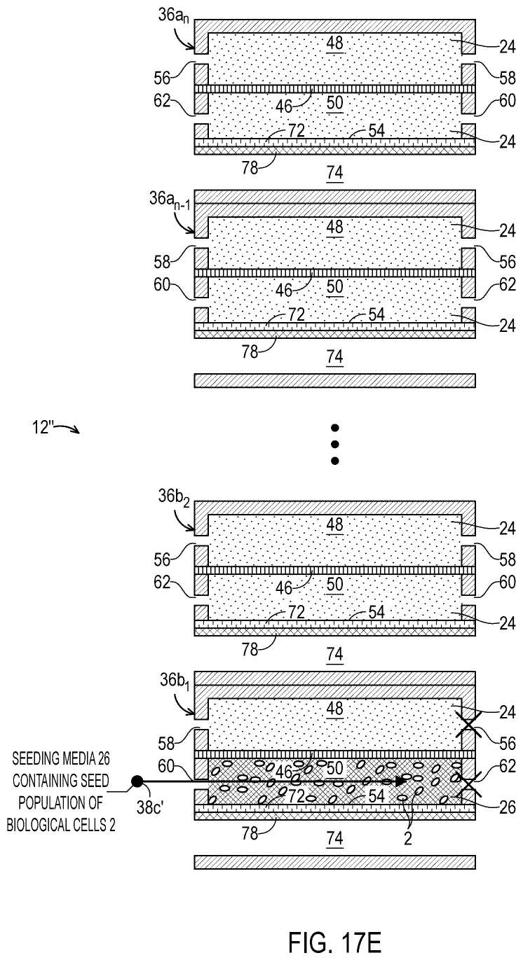

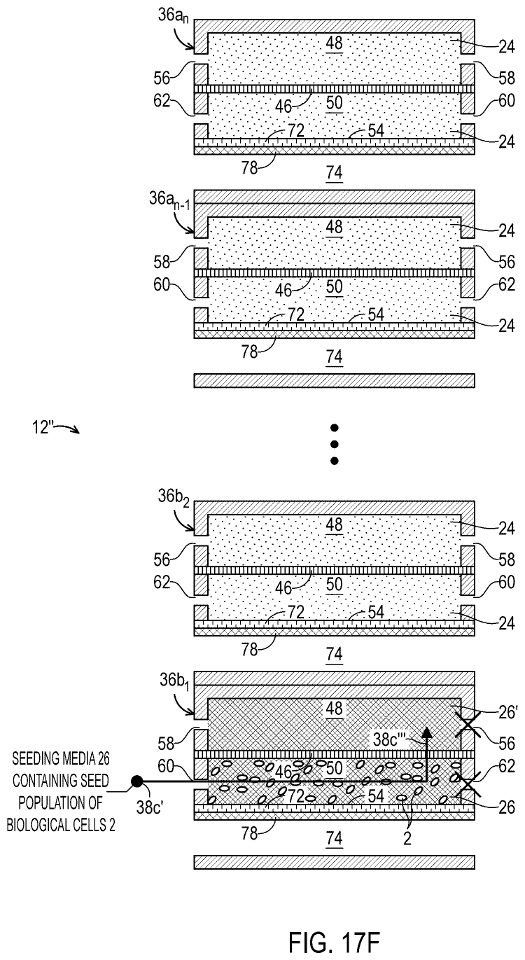

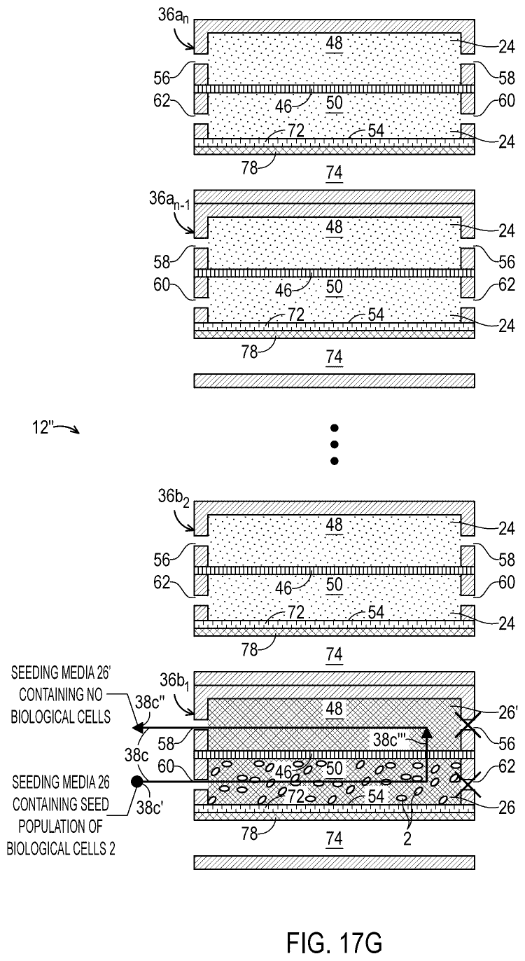

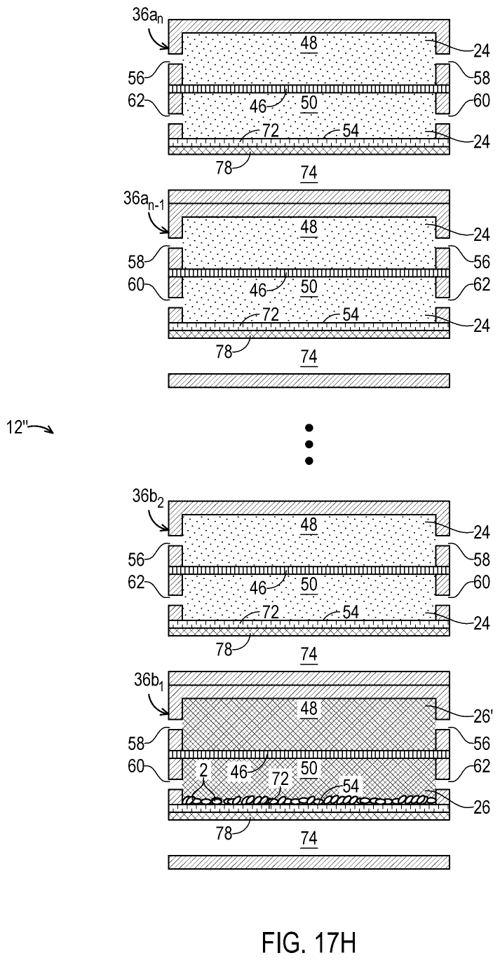

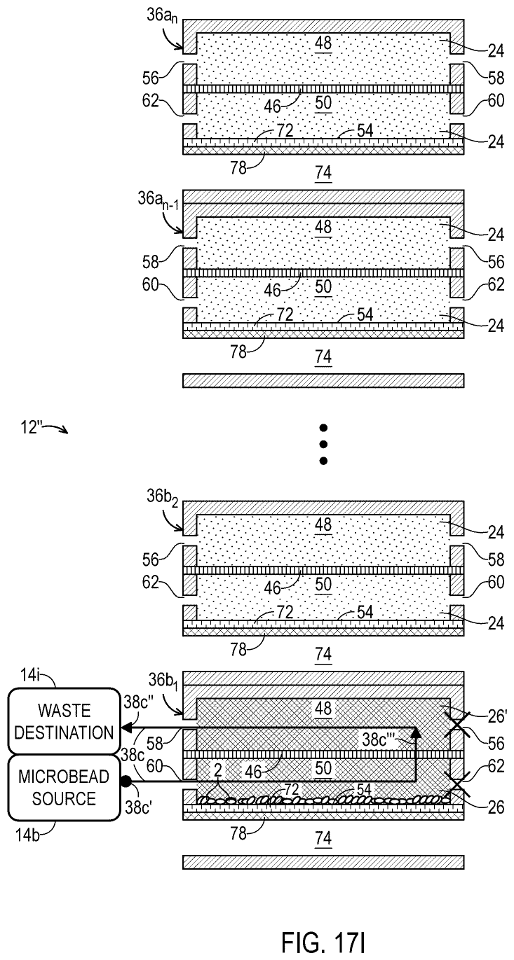

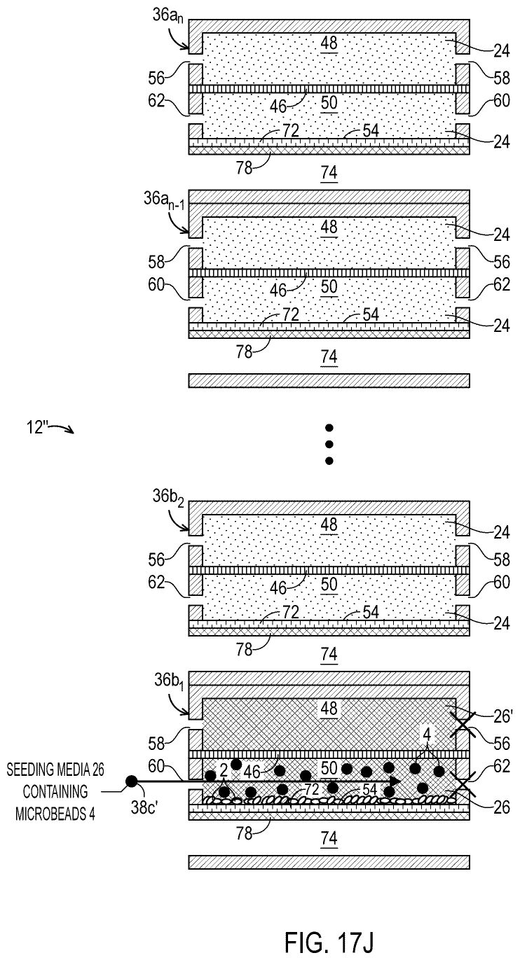

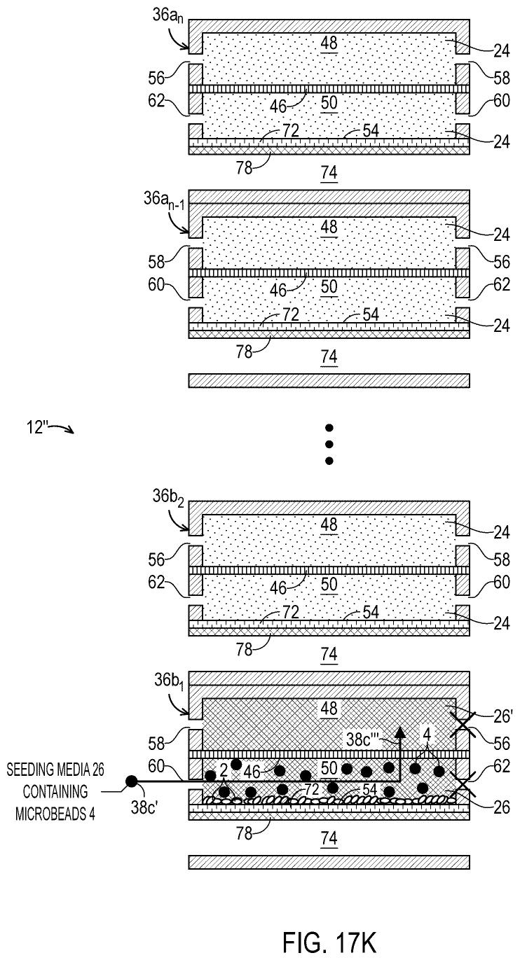

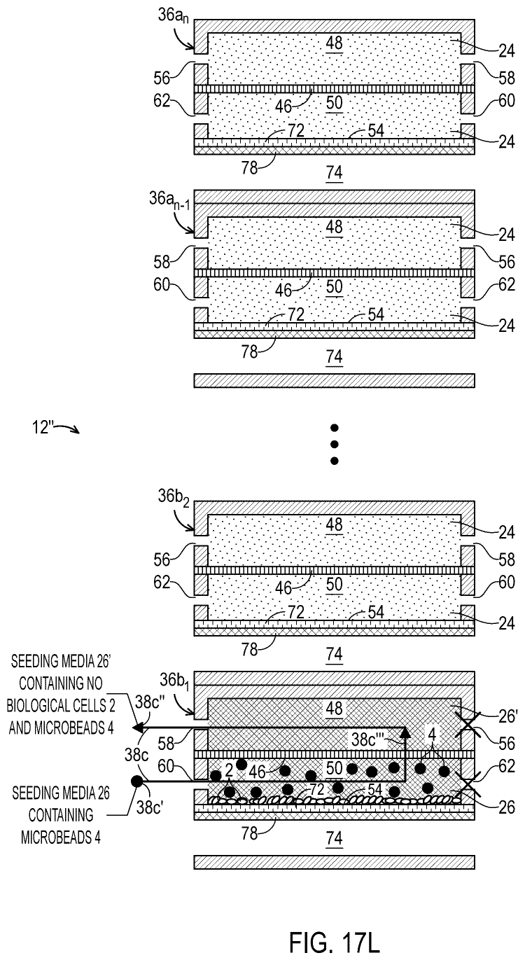

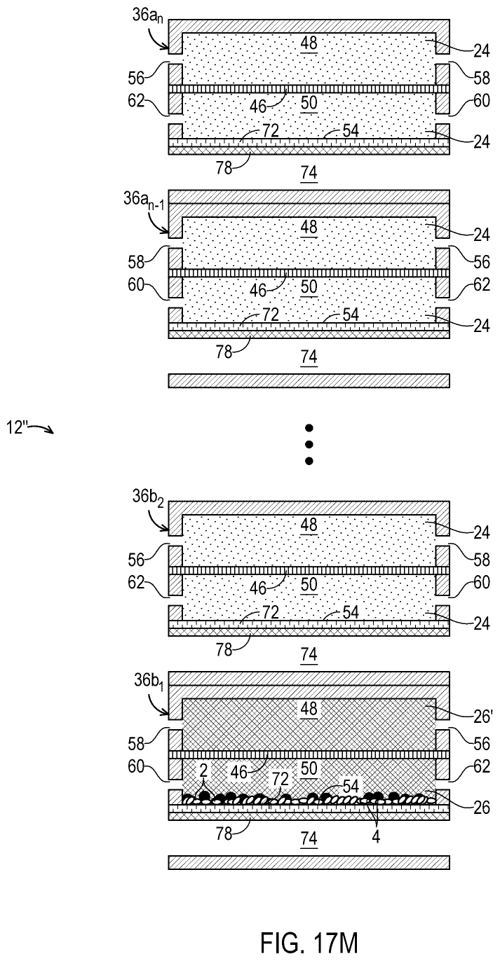

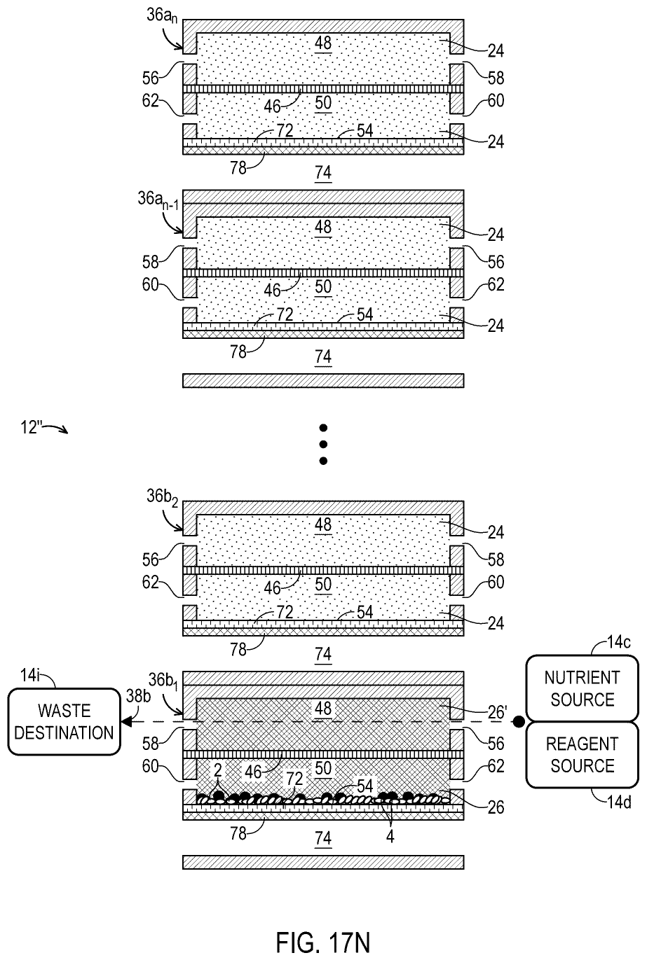

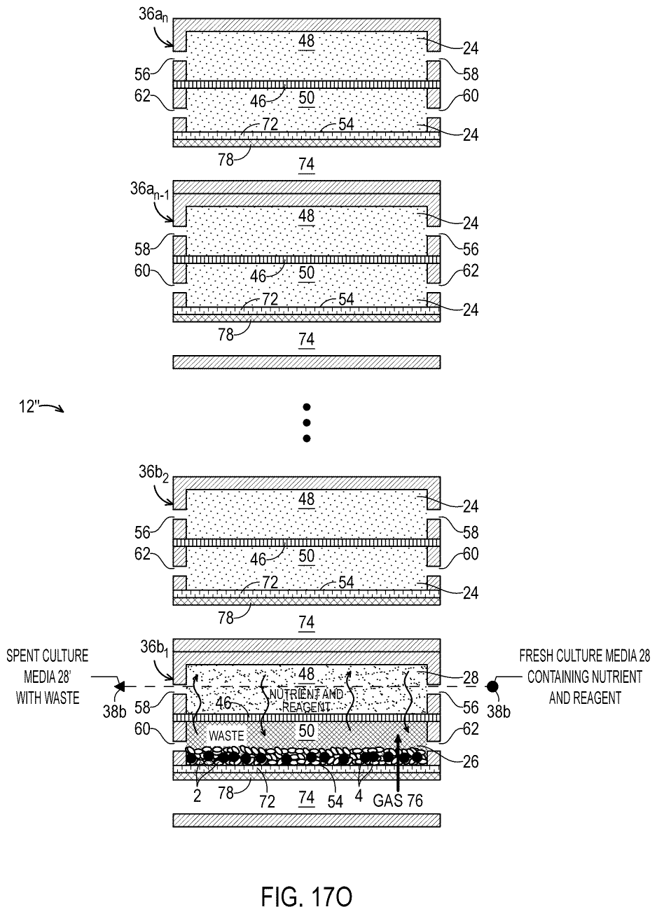

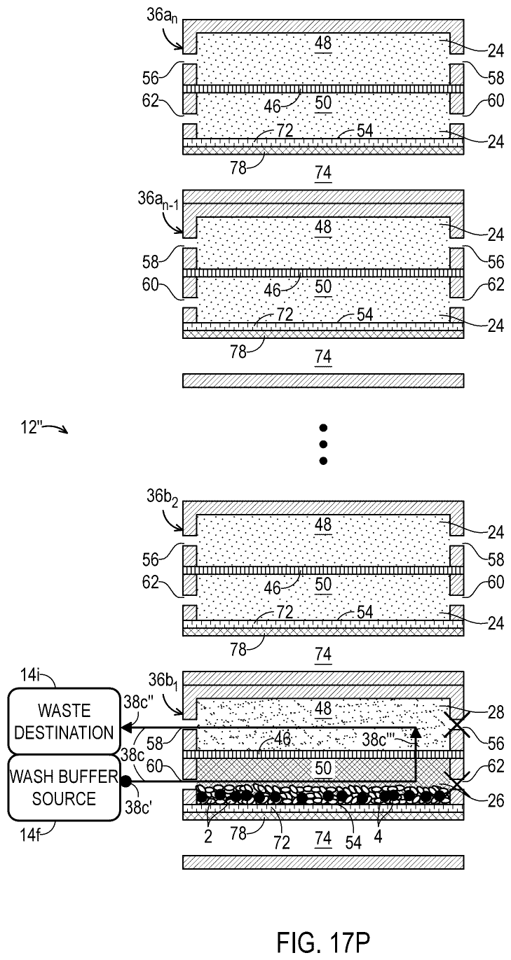

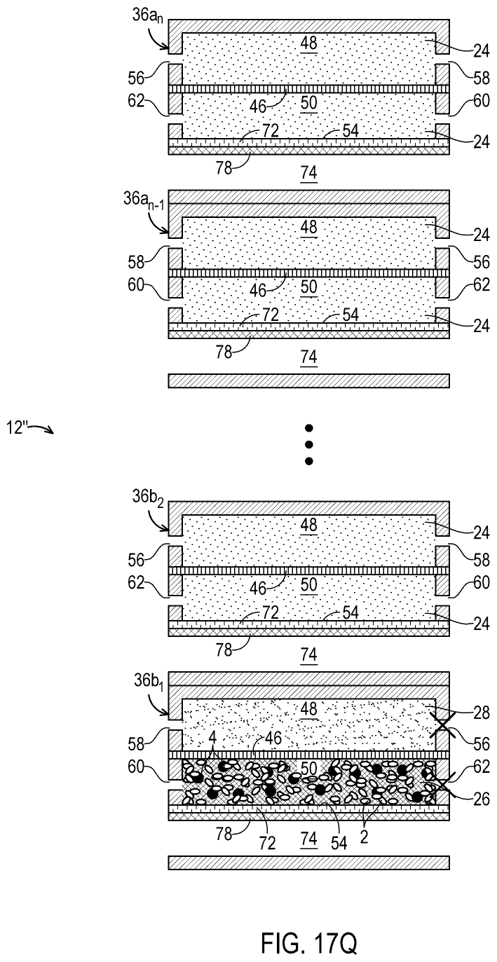

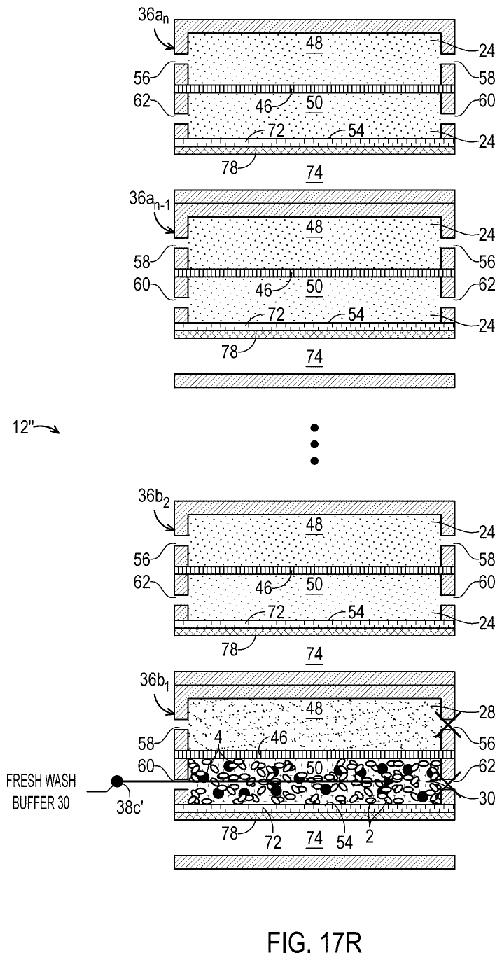

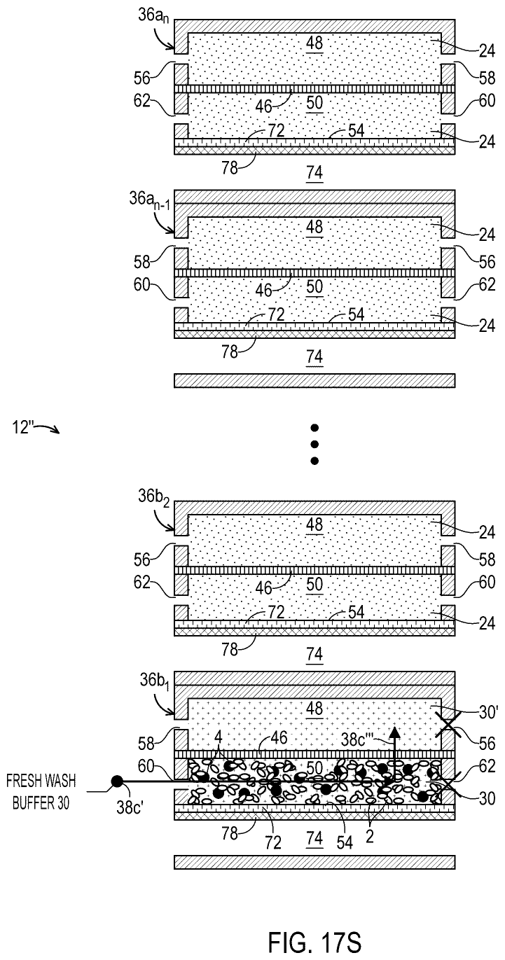

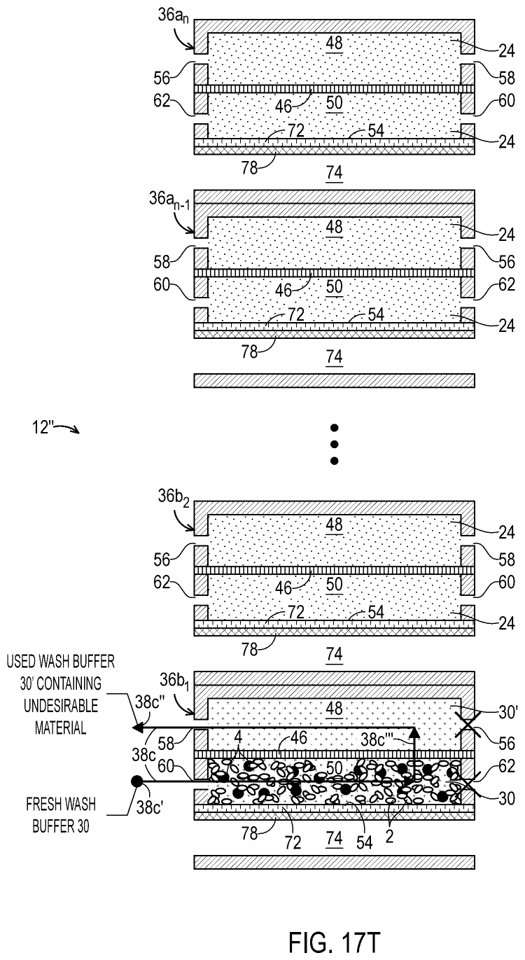

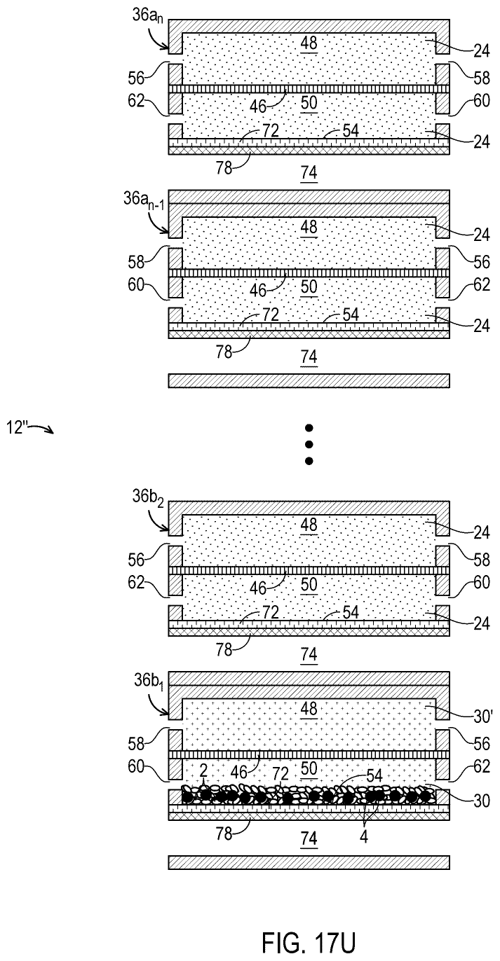

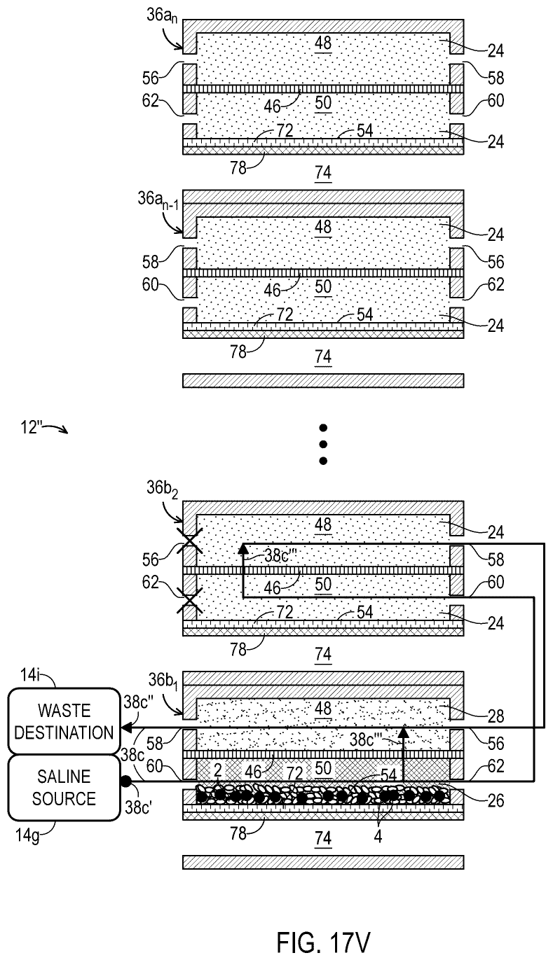

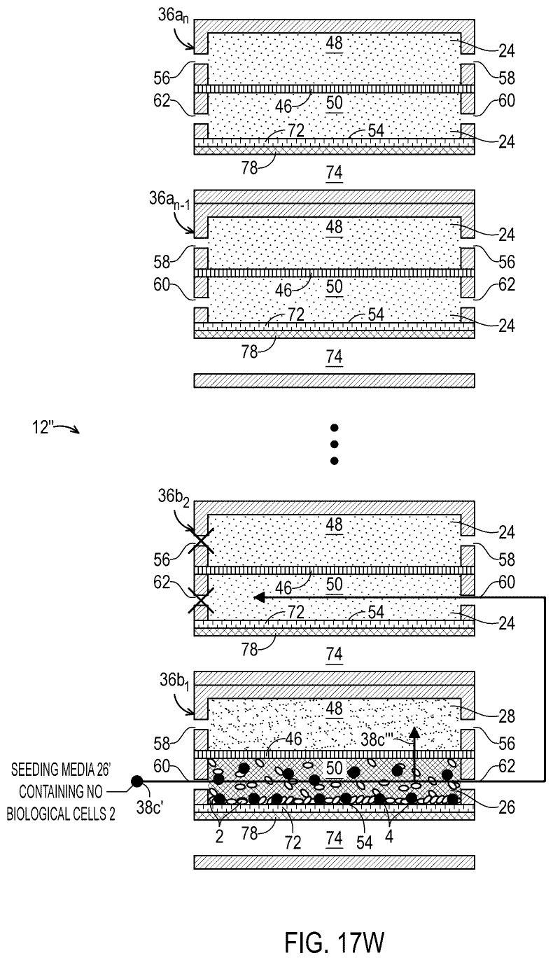

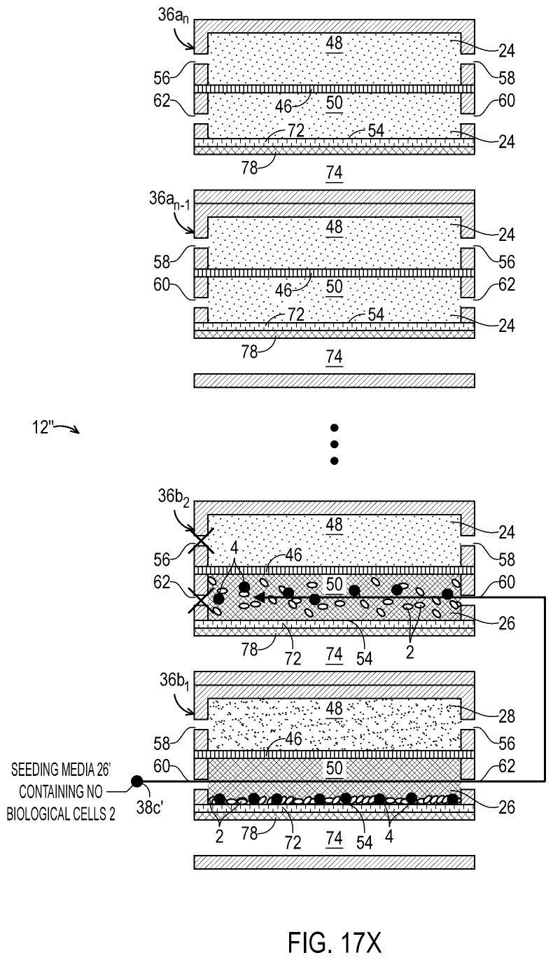

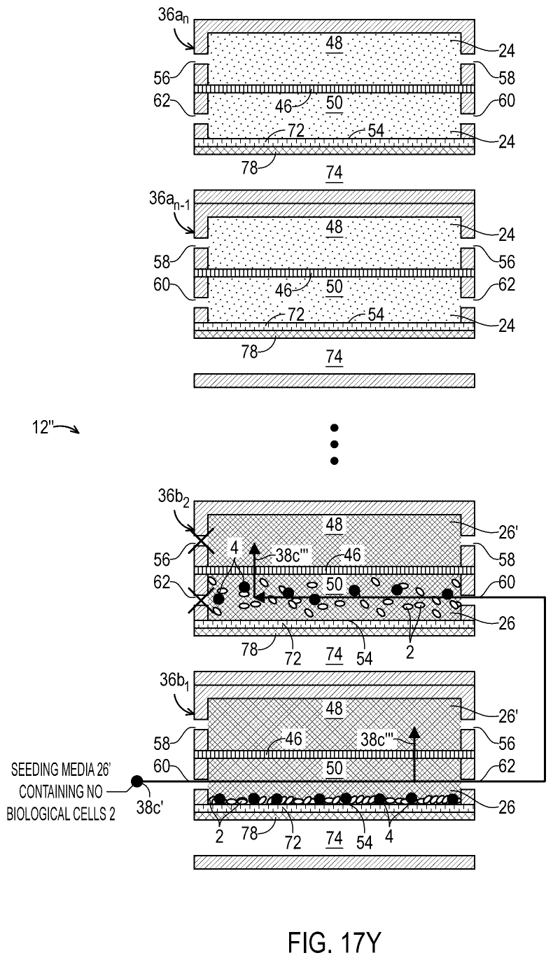

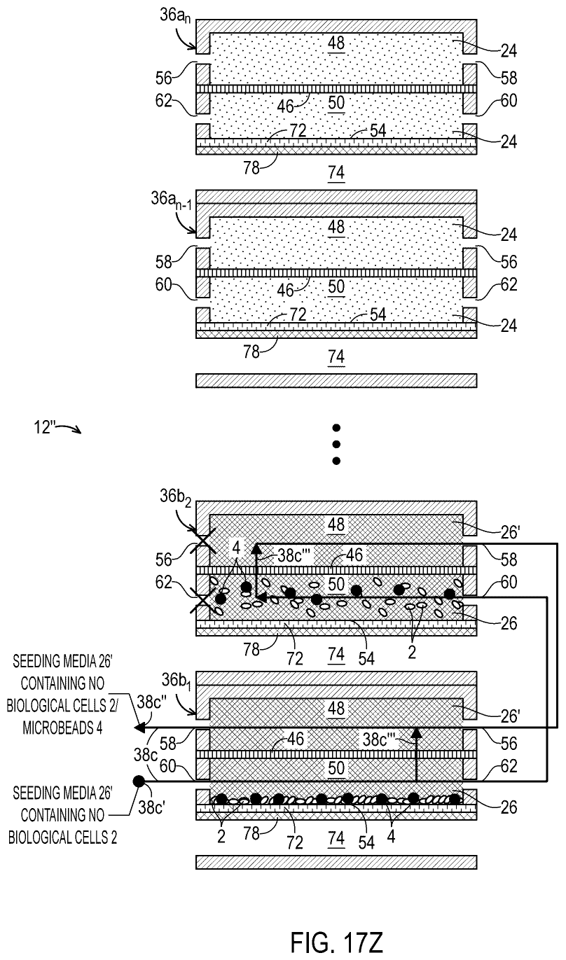

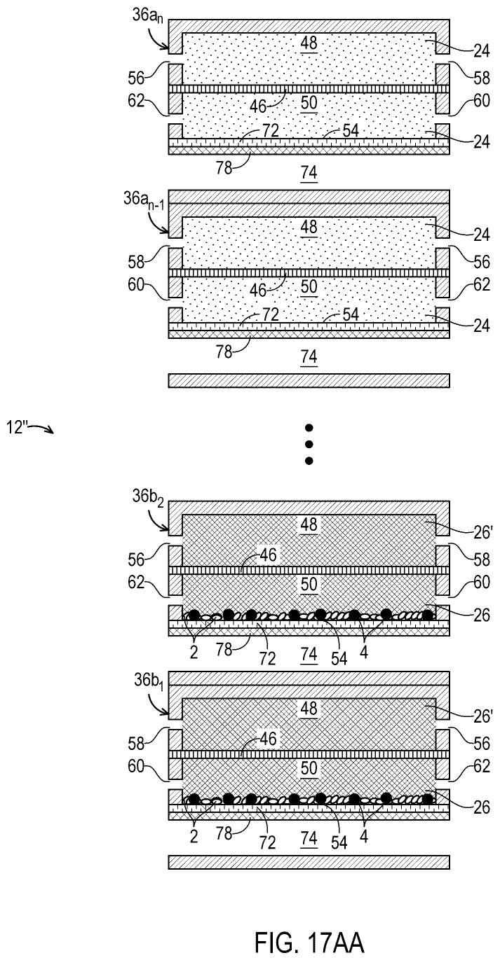

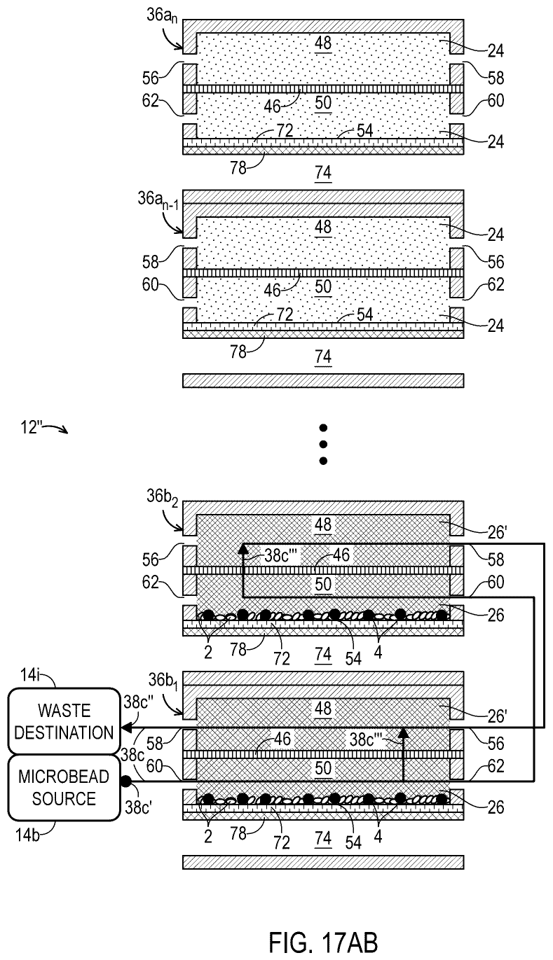

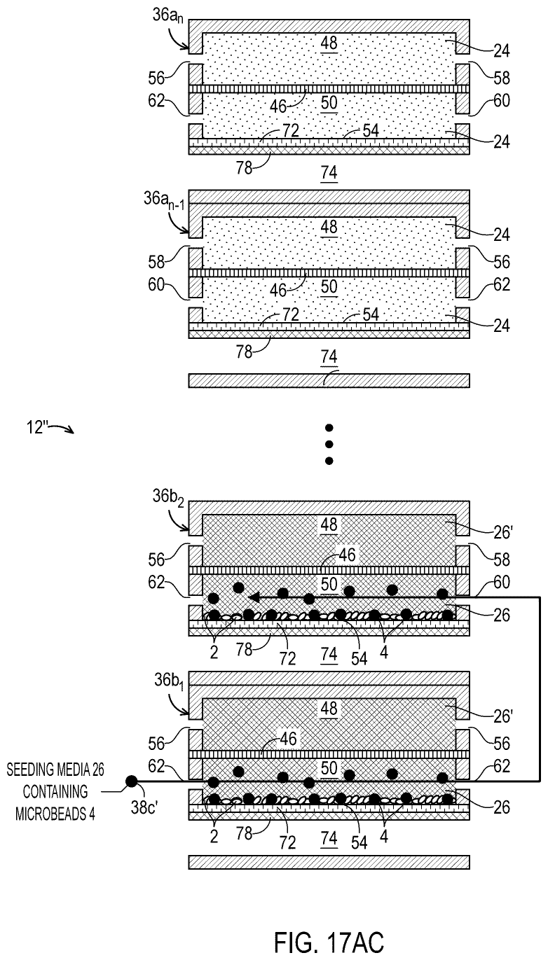

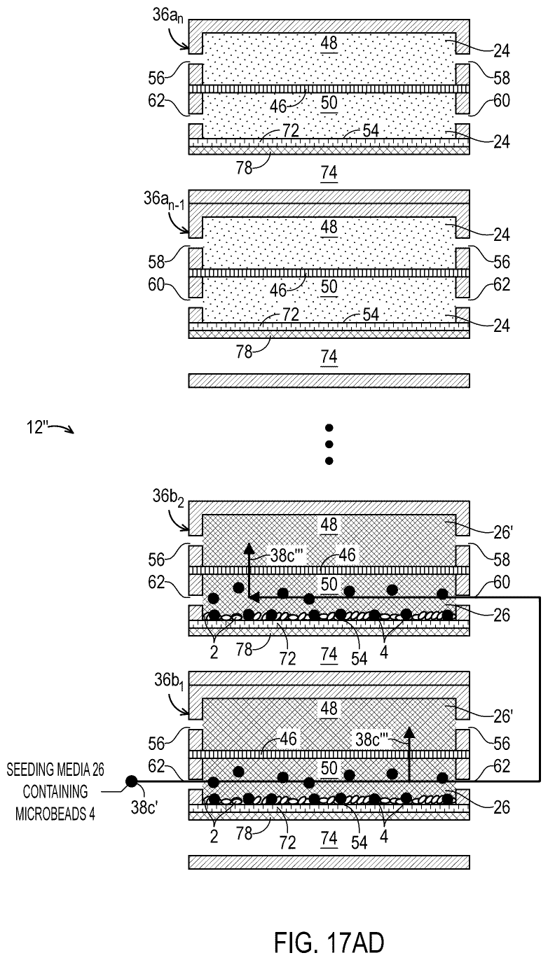

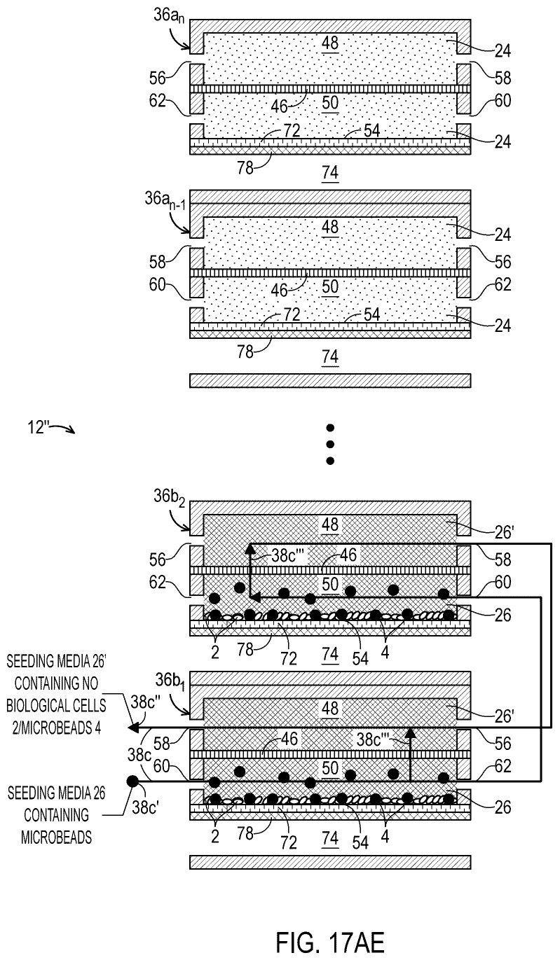

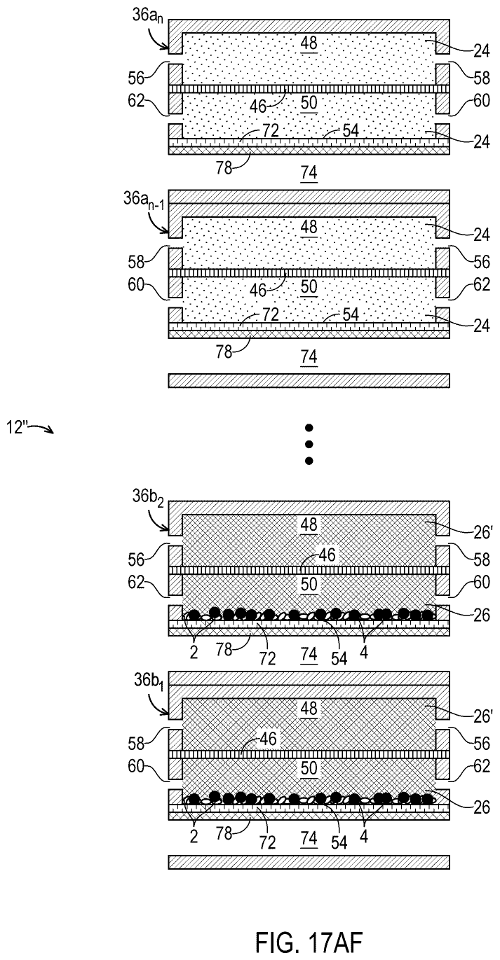

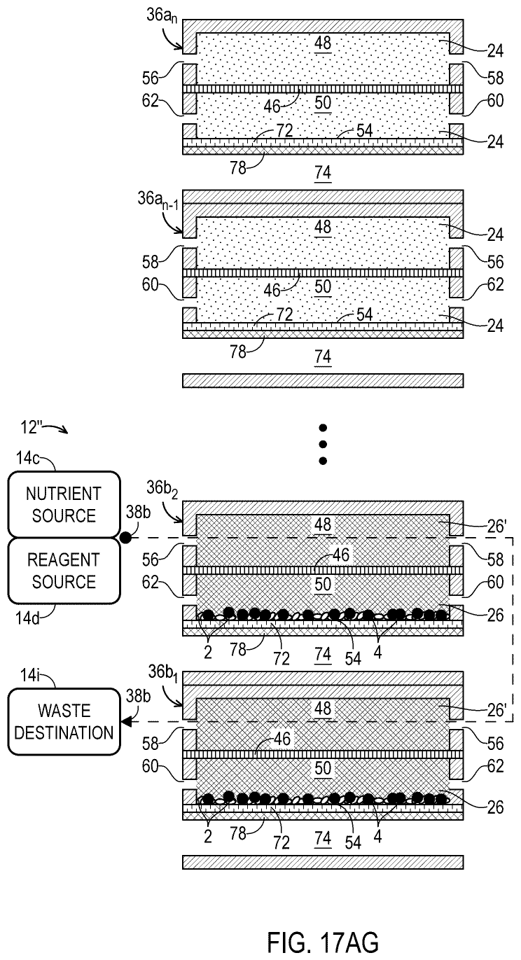

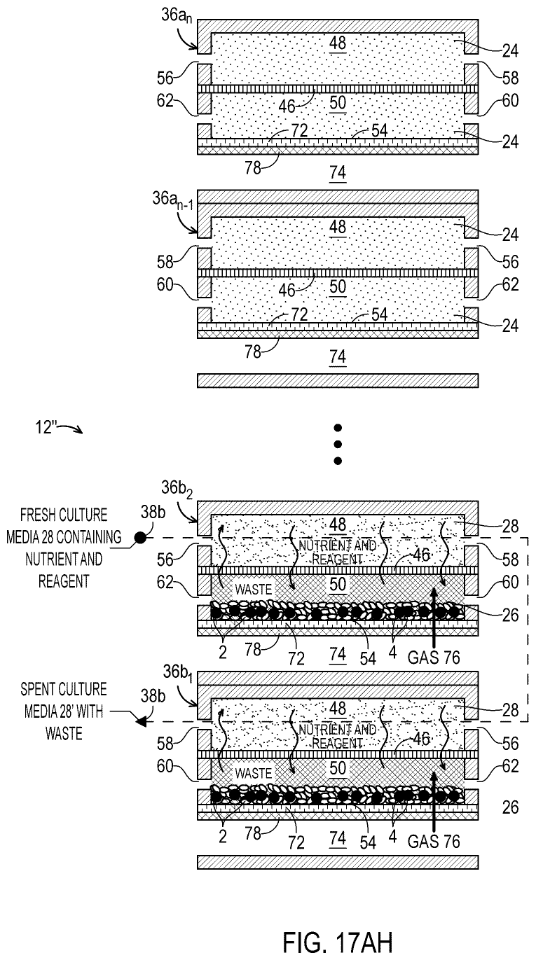

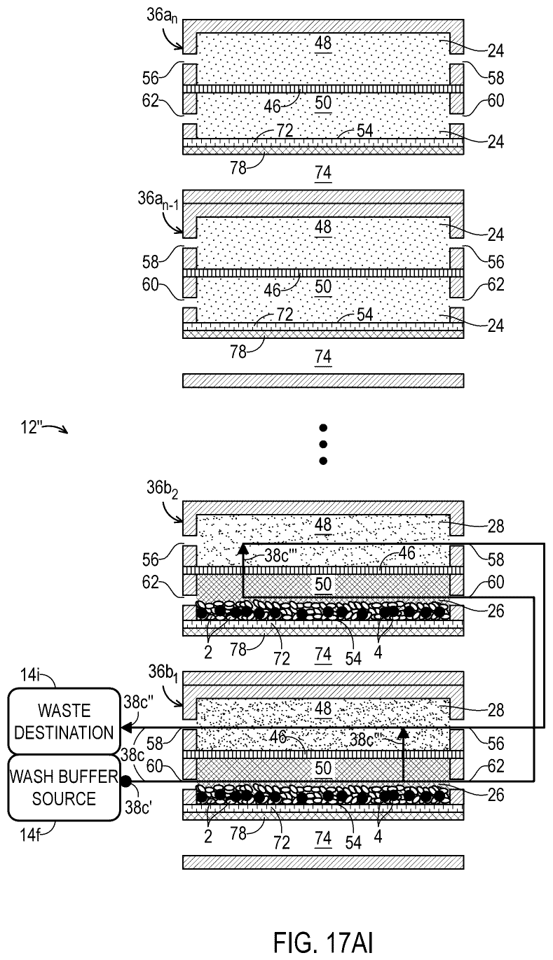

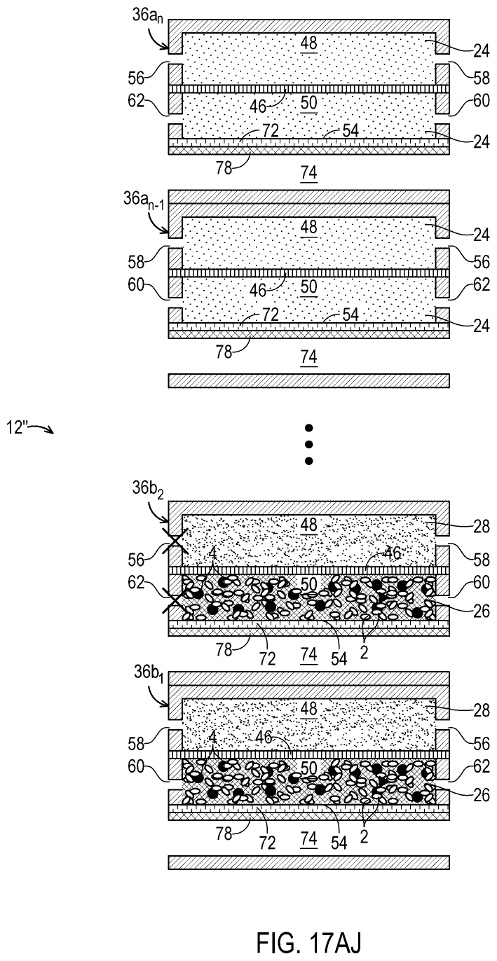

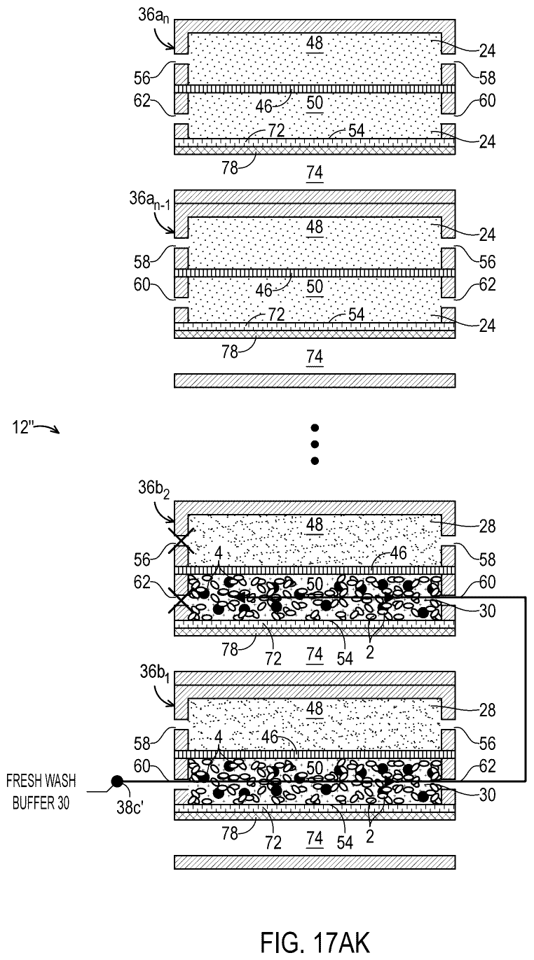

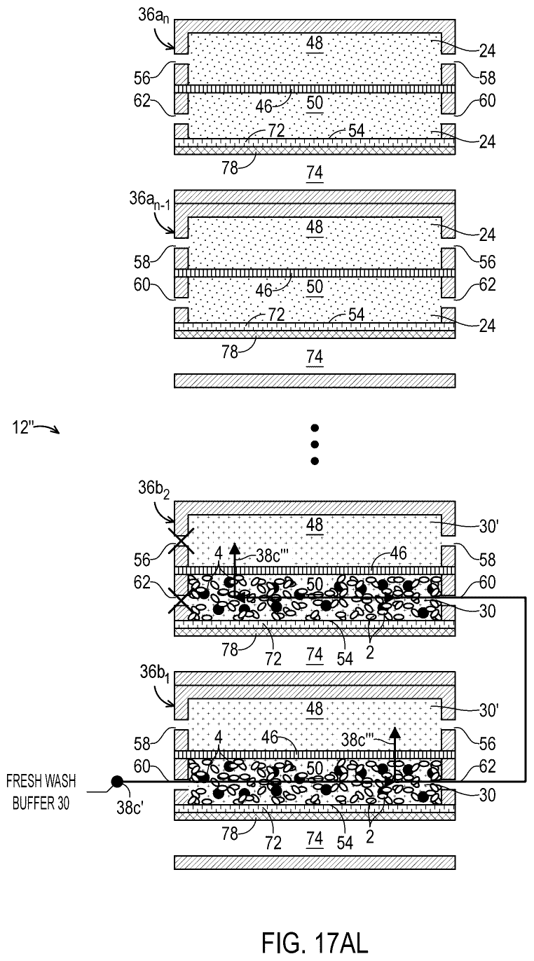

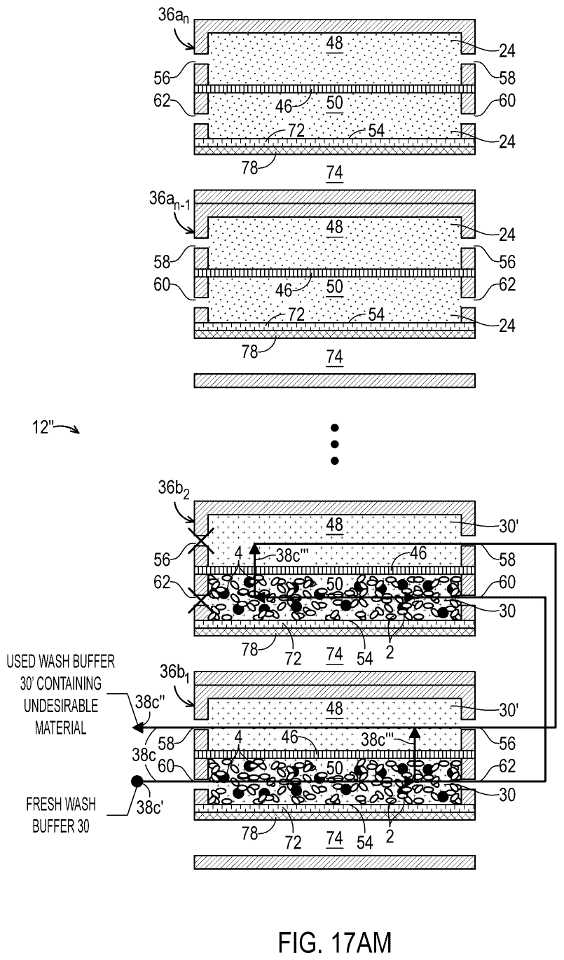

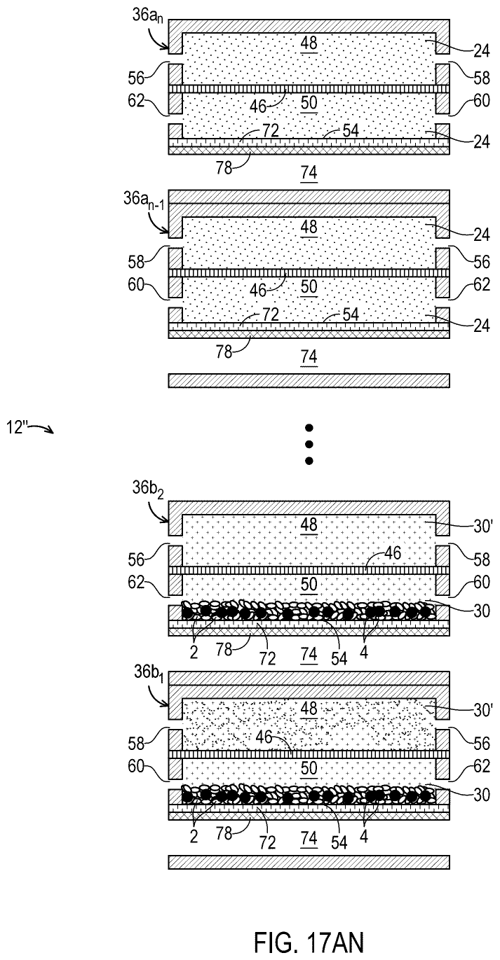

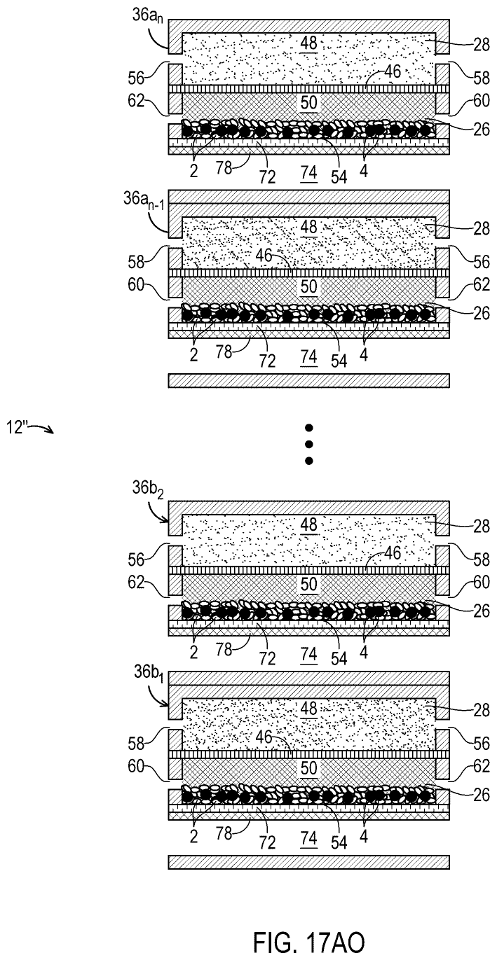

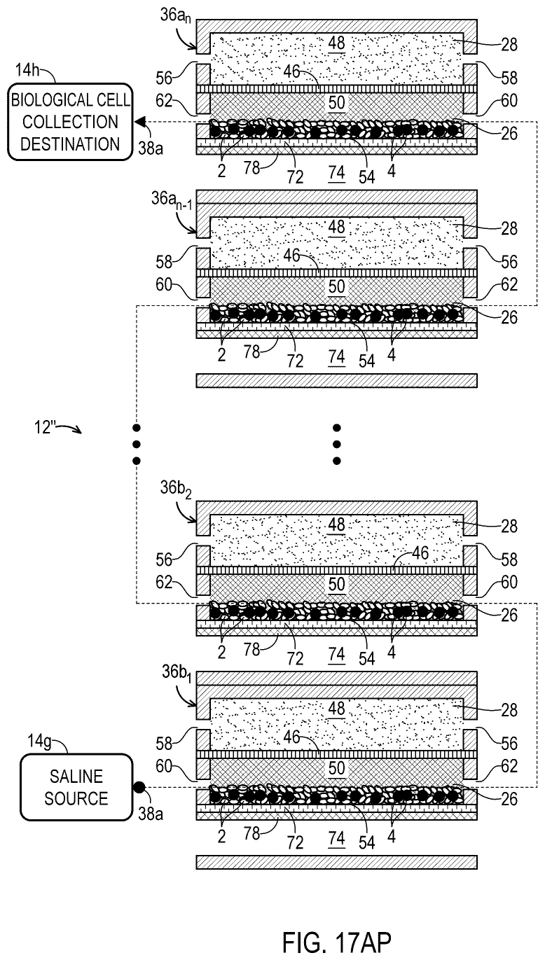

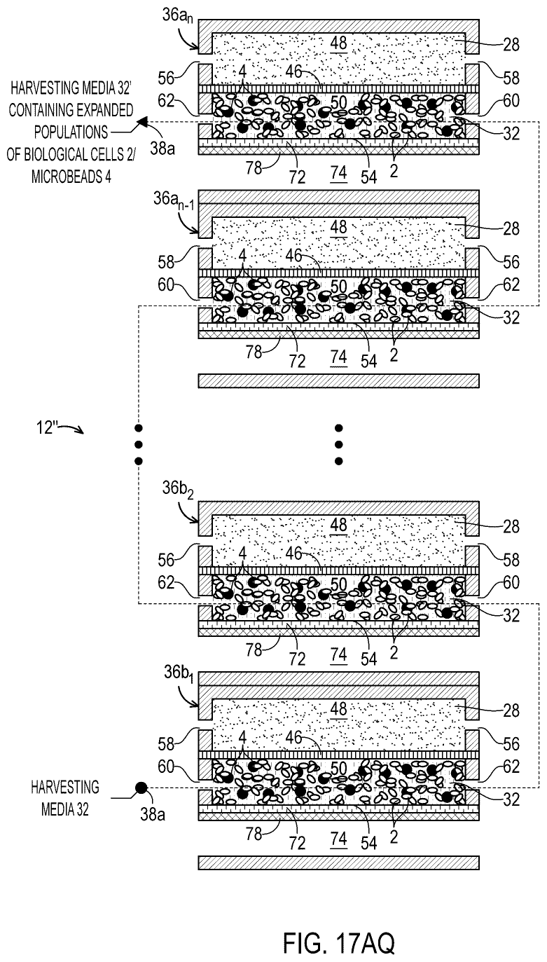

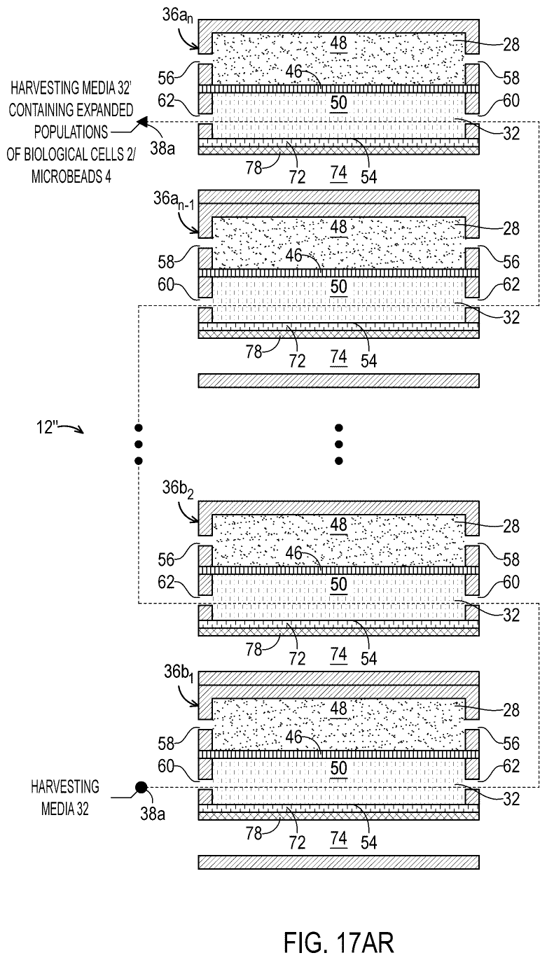

One method 200 of operating the bioreactor assembly 12″ with the bioreactor units 36b illustrated in FIG. 13 to expand and harvest multiple populations of biological cells 2 (e.g., T lymphocytes (T-cells)) will now be described with reference to FIGS. 16 and 17A-17R. The method 200 is similar to the method 100 described above with respect to FIGS. 9 and 10A-10U, with the exception that the seeding media 26, culture media 28, washer buffer 30, and harvesting media 32 are flowed through opposite flow channels 48, 50 than that of the method 100, and gas 76 is diffused into the lower flow channels 50 separately from the culture media 28.

Furthermore, in this method 200, the bioreactor units 36b are incrementally seeded with populations of biological cells 2 over time as needed during the incubation and expansion process, as opposed to being concurrently seeded at the beginning of the incubation and expansion process. In the illustrated method 200, the bioreactor units 36b are incrementally seeded with populations of biological cells 2 over time one-by-one; that is, one bioreactor unit 36b is seeded with a population of biological cells 2 and then incubated to expand the population of biological cells 2 therein, and then the next bioreactor unit 36b is seeded with another population of biological cells 2 and then incubated to expand the other population of biological cells 2, and so forth. In the illustrated method 200, the first seeded bioreactor unit 36b is seeded with biological cells 2 originating directly from the biological cell source 14a, whereas any bioreactor unit 36b to be subsequently seeded is seeded with aliquots of biological cells 2 taken from the expanded populations of biological cells 2 incubated in previously seeded bioreactor units 36b, as will be described in further detail below.

In the same manner as steps 102 and 104 of the method 100, the n number of bioreactor units 36b to be available in the bioreactor assembly 12″ to expand and harvest multiple populations of biological cells 2 may be selected depending on the desired quantity of biological cells 2 to be harvested, and the selected n number of bioreactor units 36b may be physically stacked atop and structurally affixed to each other to create bioreactor assembly 12″.

Thus, in the same manner discussed above with steps 102-104 of the method 100, the n number of bioreactor units 36b to be available in the bioreactor assembly 12″ to expand and harvest multiple populations of biological cells 2 is selected depending on the desired quantity of biological cells 2 to be harvested (step 202), and the selected n number of stacked bioreactor units 36b₁ to 36bₙ are physically stacked atop and structurally affixed to each other to create bioreactor assembly 12″ (step 204) (see FIG. 17A). Once the n number of stacked bioreactor units 36b₁ to 36bₙ are physically stacked atop and structurally affixed to each other, flow sequences of the n number of stacked bioreactor units 36b₁ to 36bₙ may be created by adding the stacked bioreactor units 36b₁ to 36bₙ to selected ones of the flow paths 38a-38c between different combinations of the media sources (biological cell source 14a, microbead source 14b, nutrient source 14c, reagent source 14d, wash buffer source 14f, and saline source 14g) and the media destinations (biological cell collection destination 14h and waste destination 14i) to effect the aforementioned functions (a)-(e) described above with respect to FIG. 1.

First, the n number of stacked bioreactor units 36b₁ to 36bₙ are primed. In this method, all n number of bioreactor units 36b₁ to 36bₙ are primed prior to the beginning of the incremental seeding process, although in alternative methods, the bioreactor units 36b₁ to 36bₙ may be incrementally primed over time (e.g., if the n number of bioreactor units 36b₁ to 36bₙ are structurally added incrementally to the flow paths 38a-38c over time). However, by priming all n number of stacked bioreactor units 36b₁ to 36bₙ prior to the beginning of the incremental seeding process, any chance that populations of biological cells 2 that are currently being incubated will be disturbed during the priming process will be eliminated.

In particular, the n number of stacked bioreactor units 36a₁ to 36aₙ are added (e.g., via operation of the valve assembly 16) to the first flow path 36a and the second flow path 36b between the saline source 14g and the waste destination 14i (step 206) (see FIG. 17B), and in this case, by fluidly coupling the lower flow channels 50 and the upper flow channels 48 of the respective n number of stacked bioreactor units 36b₁ to 36bₙ in series along the first flow path 38a and the second flow path 38b between the saline source 14g and the waste destination 14i via opening/closing of the ports 56-62 of each of the n number of stacked bioreactor units 36b₁ to 36bₙ in a similar manner described with respect to FIGS. 14A-14B.

Then, priming media 24 is flowed along the first flow path 38a and the second flow path 38b from the saline source 14g, respectively through, while accumulating air bubbles from, the lower flow channels 50 and upper flow channels 48, exiting the bioreactor assembly 12′ as priming media 24′ containing air bubbles to the waste destination 14i, thereby removing air 34 (see FIG. 17B) completely or mostly from the lower flow channels 50 and upper flow channels 48 of the respective n number of stacked bioreactor units 36b₁ to 36bₙ (step 208) (see FIG. 17C). It should be noted that, although the lower flow channels 50 and the upper flow channels 48 of the respective n number of stacked bioreactor units 36b₁ to 36bₙ, have been illustrated and described as being concurrently primed, the lower flow channels 50 and the upper flow channels 48 of the respective n number of stacked bioreactor units 36b₁ to 36bₙ may be sequentially primed; that is, all of the lower flow channels 50 of the respective n number of stacked bioreactor units 36b₁ to 36bₙ are primed, and then all of the upper flow channels 48 of the respective n number of stacked bioreactor units 36b₁ to 36bₙ are primed, or vice versa.

Next, the first stacked bioreactor unit 36b₁ is initially seeded with a population of biological cells 2.

In particular, the first stacked bioreactor unit 36b₁ is added (e.g., via operation of the valve assembly 16) to the third flow path 38c between the biological cell source 14a and the waste destination 14i (step 210) (see FIG. 17D), and in this case, by fluidly coupling the lower flow channel 48 of the first stacked bioreactor unit 36b₁ in series along the forward portion 38c′ of the third flow path 38a between the biological cell source 14a and the waste destination 14i; fluidly coupling the upper flow channel 50 of the first stacked bioreactor unit 36b₁ in series along the return portion 38c″ of the third flow path 38c between the waste destination 14i and the biological cell source 14a; and fluidly coupling the upper flow channel 48 and lower flow channel 50 of each of the n number of stacked bioreactor units 36b₁ to 36bₙ to each other via opening/closing of the ports 56-62 of the first stacked bioreactor unit 36b₁ in a similar manner described with respect to FIG. 14C.

Then, the lower flow channel 50 of the first stacked bioreactor unit 36b₁ is seeded with a population of biological cells 2, and in particular, seeding media 26 containing a seed population of biological cells 2 is flowed from the biological cell source 14a along the forward portion 38c′ of the third flow path 38c into the lower flow channel 48 of the first stacked bioreactor unit 36b₁ (step 212) (see FIG. 17E); seeding media 26′ containing no biological cells 2 is perfused along bypass portion 38c″ of the third flow path 38c from the lower flow channel 50, through the porous membrane 46, and into the upper flow channel 48, of the first stacked bioreactor unit 36b₁, while substantially retaining the biological cells 2 within the lower flow channel 50 of the first stacked bioreactor unit 36b₁ (step 214) (see FIG. 17F); the perfused seeding media 26′ containing no biological cells 2 is flowed along the return portion 38c″ of the third flow path 38c out of the upper flow channel 48 of the first stacked bioreactor unit 36b₁ to the waste destination 14i (step 216) (see FIG. 17G); and, once the flow of the seeding media 26 ceases, the biological cells 2 settle to the bottom of the lower flow channel 50 via the action of gravity, such that a seed population of biological cells 2 is disposed on the cell culture surface 54 of the first stacked bioreactor unit 36b₁ (step 218) (see FIG. 17H).

Although only the first stacked bioreactor unit 36b₁ has been described as being initially seeded with populations of biological cells 2, in alternative methods, more than the first stacked bioreactor unit 36b₁ may be initially seeded with populations of biological cells 2, e.g., only the first stacked bioreactor unit 36b₁ and the second stacked bioreactor unit 36b₂ may be initially seeded with two populations of biological cells 2. Furthermore, other ones of the bioreactor units 36b₂ to 36bₙ may be initially seeded with a population of biological cells 2. Thus, the first bioreactor unit 36b that is initially seeded need not be the first in the physical sequence of stacked bioreactor units 36b₁ to 36bₙ. As will be described in further detail below, the subsequent stacked bioreactor units 36b₂ to 36bₙ may be respectively seeded with biological cells 2 from preceding bioreactor units 36b₁ to 36bₙ₋₁. In alternative methods, some of the stacked bioreactor units 36b₂ to 36bₙ may be seeded with biological cells 2 directly from the biological cell source 14a, while other ones of the stacked bioreactor units 36b₂ to 36bₙ may be respectively seeded with biological cells 2 from preceding bioreactor units 36b₁ to 36bₙ₋₁.

Once, at least one of the stacked bioreactor units 36b₁ to 36bₙ has been seeded, a flow sequence of bioreactor units 36b that have been previously seeded can be further seeded with microbeads 4, cultured, and washed. In this case, the flow sequence of bioreactor units 36b that have been previously seeded has an order that matches the physical sequence of the stacked bioreactor units 36b₁ to 36bₙ relative to all of the flow paths 38a-38c. Thus, the previously seeded bioreactor units 36b may be designed as 36b₁ to 36bⱼ (where j is the last bioreactor unit 36b in the flow sequence that has been previously seeded with a population of biological cells 2). Of course, if only the first stacked bioreactor unit 36b₁ has been seeded with a population of biological cells 2 (j=1), the previously seeded stacked bioreactor units 36b₁ to 36bⱼ will only include the first stacked bioreactor unit 36b₁.

After (or during) at least one at least one of the stacked bioreactor units 36b₁ to 36bₙ has been seeded, microbeads 4 are optionally introduced into a flow sequence of the previously seeded stacked bioreactor units 36b₁ to 36bⱼ, such that the respective seed populations of biological cells 2 adhere to the microbeads 4 in the previously seeded stacked bioreactor units 36b₁ to 36bⱼ.

In particular, a flow sequence of the previously seeded stacked bioreactor units 36b₁ to 36bⱼ is added (e.g., via operation of the valve assembly 16) to the third flow path 38c between the microbead source 14b and the waste destination 14i (step 220), and in this case, by fluidly coupling the lower flow channels 50 of the previously seeded stacked bioreactor units 36b₁ to 36bⱼ in series along the forward portion 38c′ of the third flow path 38a between the microbead source 14b and the waste destination 14i; fluidly coupling the upper flow channels 48 of the previously seeded stacked bioreactor units 36b₁ to 36bⱼ in series along the return portion 38c″ of the third flow path 38c between the waste destination 14i and the microbead source 14b; and fluidly coupling the upper flow channel 48 and lower flow channel 50 of each of the previously seeded stacked bioreactor units 36b₁ to 36bⱼ to each other via opening/closing of the ports 56-62 of each of the previously seeded stacked bioreactor units 36b₁ to 36bⱼ in a similar manner described with respect to FIG. 14C.

Then, the microbeads 4 are introduced into the lower flow channels 50 of the respective previously seeded stacked bioreactor units 36b₁ to 36bⱼ. In particular, the seeding media 26 containing microbeads 4 is flowed along the forward portion 38c′ of the third flow path 38c into the lower flow channels 50 of the respective previously seeded stacked bioreactor units 36b₁ to 36bⱼ (step 222); seeding media 26′ containing no biological cells 2 and no microbeads 4 is perfused along bypass portions 38c″ of the third flow path 38c from the lower flow channels 50, through the porous membranes 46, and into the upper flow channels 50, of the respective previously seeded stacked bioreactor units 36b₁ to 36bⱼ, while substantially retaining the biological cells 2 and microbeads 4 within the lower flow channels 50 of the respective previously seeded stacked bioreactor units 36b₁ to 36bⱼ (step 224); the perfused seeding media 26′ containing no biological cells 2 and no microbeads 4 is flowed along the return portion 38c″ of the third flow path 38c out of the upper flow channels 48 of the respective previously seeded stacked bioreactor units 36b₁ to 36bⱼ to the waste destination 14i (step 226); and, once the flow of the seeding media 26 ceases, the microbeads 4 settle to the bottom of the lower flow channels 50 via the action of gravity, such that the populations of biological cells 2 disposed on the cell culture surfaces 54 of the respective previously seeded stacked bioreactor units 36b₁ to 36bⱼ adhere to the microbeads 4 (step 228).

Of course, if the previously seeded stacked bioreactor units 36b₁ to 36bⱼ only includes the first stacked bioreactor unit 36b₁ (j=1), microbeads 4 will only be introduced into the first stacked bioreactor unit 36b₁, such that the population of biological cells 2 adhere to the microbeads 4 in the first stacked bioreactor unit 36b₁.

In particular, only the first stacked bioreactor unit 36b₁ is added (e.g., via operation of the valve assembly 16) to the third flow path 38c between the microbead source 14b and the waste destination 14i (step 220) (see FIG. 17I) by fluidly coupling the lower flow channel 50 of the first stacked bioreactor unit 36b₁ in series along the forward portion 38c′ of the third flow path 38a between the microbead source 14b and the waste destination 14i; fluidly coupling the upper flow channel 48 of the first stacked bioreactor unit 36b₁ in series along the return portion 38c″ of the third flow path 38c between the waste destination 14i and the microbead source 14b; and fluidly coupling the upper flow channel 48 and lower flow channel 50 of first stacked bioreactor unit 36b₁.

Then, the microbeads 4 are introduced into the lower flow channel 50 of the first stacked bioreactor unit 36b₁. In particular, the seeding media 26 containing microbeads 4 is flowed along the forward portion 38c′ of the third flow path 38c into the lower flow channel 50 of the first stacked bioreactor unit 36b₁ to 36bⱼ (step 222) (see FIG. 17J); seeding media 26′ containing no biological cells 2 and no microbeads 4 is perfused along bypass portions 38c″ of the third flow path 38c from the lower flow channel 48, through the porous membrane 46, and into the upper flow channel 48, of the first stacked bioreactor unit 36b₁, while substantially retaining the biological cells 2 and microbeads 4 within the lower flow channel 50 of the first stacked bioreactor unit 36b₁ (step 224) (see FIG. 17K); the perfused seeding media 26′ containing no biological cells 2 and no microbeads 4 is flowed along the return portion 38c″ of the third flow path 38c out of the upper flow channel 48 of the first stacked bioreactor unit 36b₁ to the waste destination 14i (step 226) (see FIG. 17L); and, once the flow of the seeding media 26 ceases, the microbeads 4 settle to the bottom of the lower flow channel 50 via the action of gravity, such that the population of biological cells 2 disposed on the cell culture surface 54 of the first stacked bioreactor unit 36b₁ adhere to the microbeads 4 (step 228) (see FIG. 17M).

While the microbeads 4 have been described as being delivered separately from the biological cells 2, into the lower flow channels 48 of the respective previously seeded stacked bioreactor units 36b₁ to 36bⱼ, it should be appreciated that, at least with respect to the initially seeded first stacked bioreactor unit 36b₁, the microbeads 4 may be concurrently delivered with the biological cells 2, into the upper flow channel 48 of the first stacked bioreactor unit 36b₁; e.g., by adding the first stacked bioreactor unit 36b₁ to the third flow path 38c between the both the biological cell source 14a and microbead source 14b and the waste destination 14i.

Next, nutrient, reagent, and gas are delivered to the populations of biological cells 2, and waste is extracted from the populations of biological cells 2, thereby facilitating the expansion of the seed populations of biological cells 2 within the lower flow channels 48 of the respective previously seeded stacked bioreactor units 36b₁ to 36bⱼ.

In particular, a flow sequence of the previously seeded stacked bioreactor units 36b₁ to 36bⱼ is added (e.g., via operation of the valve assembly 16) to the second flow path 36b between the nutrient source 14c and reagent source 14d and the waste destination 14i, and in this case, by fluidly coupling the upper flow channels 50 of the respective previously seeded stacked bioreactor units 36b₁ to 36bⱼ in series along the second flow path 38b between the nutrient source 14c and reagent source 14d and the waste destination 14i via opening/closing of the ports 56-62 of each of the previously seeded stacked bioreactor units 36b₁ to 36bⱼ in a similar manner described with respect to FIG. 14A (step 230).

Then, fresh culture media 28 is flowed (continually or intermittently) along the second flow path 38b from the nutrient source 14c and reagent source 14d, through the upper flow channels 50 of the respective previously seeded stacked bioreactor units 36b₁ to 36bⱼ, while nutrient, reagent, and waste are exchanged between the populations of biological cells 2 and fresh culture media 28 (i.e., the nutrient and reagent are provided to, while the waste is extracted from, the populations of biological cells 2, with the nutrient and reagent diffusing from the upper flow channels 48, through the porous membranes 46, and into the lower flow channels 50, and the waste diffusing from the lower flow channels 50, through the porous membranes 46, and into the upper flow channels 48, of the respective previously seeded stacked bioreactor units 36b₁ to 36bⱼ), exiting the bioreactor assembly 12″ as spent culture media 28′ with the waste, thereby facilitating the expansion of the seed populations of biological cells 2 within the lower flow channels 50 of the respective previously seeded stacked bioreactor units 36b₁ to 36bⱼ (step 232). As with the case in the method 100 described above, it is preferred that culture media 28 be replaced within the upper flow channels 50 with fresh culture media 28 before the culture media 28 becomes completely spent.

Simultaneously with the diffusion of the nutrient, reagent, and waste across the porous membrane 46 between the flow channels 48, 50 of the respective previously seeded stacked bioreactor units 36b₁ to 36bⱼ, gas 76 is conveyed from the ambient environment 74 into the culture media 28, and in particular, diffuses from the ambient environment 74, through the dense membrane 72, and into the lower flow channels 48, while allowing the culture media 28 to be retained within the lower flow channels 48, of the respective previously seeded stacked bioreactor units 36b₁ to 36bⱼ (step 234).

Of course, if the previously seeded stacked bioreactor units 36b₁ to 36bⱼ only includes the first stacked bioreactor unit 36b₁ (j=1), nutrient, reagent, and gas will be delivered to the population of biological cells 2, and waste will be extracted from the population of biological cells 2, thereby facilitating the expansion of the seed population of biological cells 2 within the lower flow channel 48 of only the first stacked bioreactor unit 36b₁.

In particular, the first stacked bioreactor unit 36b₁ is added (e.g., via operation of the valve assembly 16) to the second flow path 36b between the nutrient source 14c and reagent source 14d and the waste destination 14i, and in this case, by fluidly coupling the upper flow channel 50 of the first stacked bioreactor unit 36b₁ in series along the second flow path 38b between the nutrient source 14c and reagent source 14d and the waste destination 14i via opening/closing of the ports 56-62 of the first stacked bioreactor unit 36b₁ in a similar manner described with respect to FIG. 14A (step 230) (see FIG. 17N).

Then, fresh culture media 28 is flowed (continually or intermittently) along the second flow path 38b from the nutrient source 14c and reagent source 14d, through the lower flow channel 50 of the first stacked bioreactor unit 36b₁, while nutrient, reagent, and waste are exchanged between the population of biological cells 2 and fresh culture media 28 (i.e., the nutrient and reagent are provided to, while the waste is extracted from, the population of biological cells 2, with the nutrient and reagent diffusing from the upper flow channel 48, through the porous membrane 46, and into the lower flow channel 50, and the waste diffusing from the lower flow channel 50, through the porous membrane 46, and into the upper flow channel 48, of the first stacked bioreactor unit 36b₁), exiting the first stacked bioreactor unit 36b₁ as spent culture media 28′ with the waste, thereby facilitating the expansion of the seed population of biological cells 2 within the lower flow channel 50 of the first stacked bioreactor unit 36b₁ (step 232) (see FIG. 17O).

Simultaneously with the diffusion of the nutrient, reagent, and waste across the porous membrane 46 between the flow channels 48, 50 of the first stacked bioreactor unit 36b₁, gas 76 diffused from the ambient environment 74, through the dense membranes 72, and into the lower flow channel 48, while allowing liquid media to be retained within the lower flow channel 48, of the first stacked bioreactor unit 36b₁ (step 234) (see FIG. 17O).

Anytime during incubation, the populations of biological cells 2 and microbeads 4 in the lower flow channels 48 of the respective previously seeded stacked bioreactor units 36b₁ to 36bⱼ may be washed.

In particular, a flow sequence of the previously seeded stacked bioreactor units 36b₁ to 36bⱼ is added (e.g., via operation of the valve assembly 16) to the third flow path 38c between the wash buffer source 14f and the waste destination 14i, and in this case, by fluidly coupling the lower flow channels 50 of the respective previously seeded stacked bioreactor units 36b₁ to 36bⱼ in series along the forward portion 38c′ of the third flow path 38a between the wash buffer source 14f and the waste destination 14i; fluidly coupling the upper flow channels 48 of the respective previously seeded stacked bioreactor units 36b₁ to 36bⱼ in series along the return portion 38c″ of the third flow path 38c between the waste destination 14i and the wash buffer source 14f; and fluidly coupling the upper flow channel 48 and lower flow channel 50 of each of the previously seeded stacked bioreactor units 36b₁ to 36bⱼ to each other via opening/closing of the ports 56-62 of each of the previously seeded stacked bioreactor units 36b₁ to 36bⱼ in a similar manner described with respect to FIG. 14C (step 236).

Then, the bioreactor assembly 12″ is mechanically agitated, thereby suspending the populations of biological cells 2 and microbeads 4 in the lower flow channels 48, such that the populations of biological cells 2 will be maximally exposed to the fresh wash buffer 30 and will not block perfusion of the used wash buffer 30 containing undesirable material (e.g., unbound molecules, debris, dead cells, or other undesirable molecules) through the porous membranes 46, of the respective previously seeded stacked bioreactor units 36b₁ to 36bⱼ (step 238); the fresh wash buffer 30 is flowed along the forward portion 38c′ of the third flow path 38c into, while accumulating the undesirable material from, the lower flow channels 50 of the respective previously seeded stacked bioreactor units 36b₁ to 36bⱼ, thereby washing the populations of biological cells 2 and microbeads 4 (step 240); used wash buffer 30′ containing the undesirable material, is perfused along bypass portions 38c″ of the third flow path 38c from the lower flow channels 50, through the porous membranes 46, and into the upper flow channels 48, of the respective previously seeded stacked bioreactor units 36b₁ to 36bⱼ, while substantially retaining the biological cells 2 and microbeads 4 within the lower flow channels 50 (step 242); the used wash buffer 30′ containing the undesirable material is flowed along the return portion 38c″ of the third flow path 38c out of the upper flow channels 48 of the respective previously seeded stacked bioreactor units 36b₁ to 36bⱼ to the waste destination 14i (step 244); and, once the flow of the fresh wash buffer 30 ceases, the biological cells 2 and microbeads 4 settle to the bottoms of the lower flow channels 50 via the action of gravity, such that seed populations of biological cells 2 and microbeads 4 reconstitute on the cell culture surfaces 54 of the respective previously seeded stacked bioreactor units 36b₁ to 36bⱼ (step 246). After the washing steps, nutrient, reagent, and gas may be delivered to the populations of biological cells 2, and waste may be extracted from the populations of biological cells 2, thereby facilitating further expansion of the seed populations of biological cells 2 within the upper flow channels 48 of the respective previously seeded stacked bioreactor units 36b₁ to 36bⱼ in accordance with steps 230-234.

Of course, if the previously seeded stacked bioreactor units 36b₁ to 36bⱼ only includes the first stacked bioreactor unit 36b₁ (j=1), the population of biological cells 2 residing in the lower flow channel 48 of only the first stacked bioreactor unit 36b₁ will be washed.

In particular, the first stacked bioreactor unit 36b₁ is added (e.g., via operation of the valve assembly 16) to the third flow path 38c between the wash buffer source 14f and the waste destination 14i, and in this case, by fluidly coupling the lower flow channel 50 of the first stacked bioreactor unit 36b₁ in series along the forward portion 38c′ of the third flow path 38a between the wash buffer source 14f and the waste destination 14i; fluidly coupling the upper flow channel 48 of the first stacked bioreactor unit 36b₁ in series along the return portion 38c″ of the third flow path 38c between the waste destination 14i and the wash buffer source 14f; and fluidly coupling the upper flow channel 48 and lower flow channel 50 of the first stacked bioreactor unit 36b₁ to each other via opening/closing of the ports 56-62 of first stacked bioreactor unit 36b₁ in a similar manner described with respect to FIG. 14C (step 236) (see FIG. 17P).

Then, the bioreactor assembly 12″ is mechanically agitated, thereby suspending the population of biological cells 2 and microbeads 4 in the lower flow channel 48, such that the population of biological cells 2 will be maximally exposed to the fresh wash buffer 30 and will not block perfusion of the used wash buffer 30 containing the undesirable material through the porous membrane 46, of the first stacked bioreactor unit 36b₁ (step 238) (see FIG. 17Q); the fresh wash buffer 30 is flowed along the forward portion 38c′ of the third flow path 38c into, while accumulating the undesirable material from, the lower flow channel 50 of the first stacked bioreactor unit 36b₁, thereby washing the populations of biological cells 2 and microbeads 4 (step 240) (see FIG. 17R); used wash buffer 30′ containing the undesirable material, is perfused along bypass portions 38c″ of the third flow path 38c from the lower flow channel 50, through the porous membrane 46, and into the upper flow channel 48, of the first stacked bioreactor unit 36b₁, while substantially retaining the biological cells 2 and microbeads 4 within the lower flow channel 50 (step 242) (see FIG. 17S); the used wash buffer 30′ containing the undesirable material is flowed along the return portion 38c″ of the third flow path 38c out of the upper flow channel 48 of the first stacked bioreactor unit 36b₁ to the waste destination 14i (step 244) (see FIG. 17T); and, once the flow of the fresh wash buffer 30 ceases, the biological cells 2 and microbeads 4 settle to the bottom of the lower flow channel 50 via the action of gravity, such that the population of biological cells 2 reconstitutes on the cell culture surface 54 of the first stacked bioreactor unit 36b₁ (step 246) (see FIG. 17U).

If the previously seeded stacked bioreactor units 36b₁ to 36bⱼ do not include the last stacked bioreactor unit 36bₙ (step 248), the next bioreactor unit 36b that has not already been seeded with a population of biological cells 2 is seeded with a population of biological cells 2. In the illustrated case, the next bioreactor unit 36b to be seeded is the bioreactor unit 36bⱼ₊₁ physically after the last seeded bioreactor unit 36bⱼ, although the next bioreactor unit 36b to be seeded need not be the next physical bioreactor unit 36bⱼ₊₁ after the last seeded bioreactor unit 36bⱼ, and in fact, can be any bioreactor unit 36b that can be fluidly coupled in series to any previously seeded bioreactor unit 36b).

In particular, a flow sequence of previously seeded stacked bioreactor units 36b₁ to 36bⱼ (i.e., preceding bioreactor units 36b in the flow sequence) and the next unseeded stacked bioreactor unit 36bⱼ₊₁ (i.e., a succeeding bioreactor unit 36b in the flow sequence) is added (e.g., via operation of the valve assembly 16) to the third flow path 38c between the saline source 14g and the waste destination 14i (step 250), and in this case, by fluidly coupling the lower flow channels 48 of the respective previously seeded stacked bioreactor units 36b₁ to 36bⱼ and the next unseeded stacked bioreactor unit 36bⱼ₊₁ in series along the forward portion 38c′ of the third flow path 38a between the saline source 14g and the waste destination 14i; fluidly coupling the upper flow channels 50 of the respective previously seeded stacked bioreactor units 36b₁ to 36bⱼ and the next unseeded stacked bioreactor unit 36bⱼ₊₁ in series along the return portion 38c″ of the third flow path 38c between the waste destination 14i and the saline source 14g; and fluidly coupling the upper flow channels 48 and lower flow channels 50 of each of the previously seeded stacked bioreactor units 36b₁ to 36bⱼ and the next unseeded stacked bioreactor unit 36bⱼ₊₁ to each other via opening/closing of the ports 56-62 of the respective previously seeded stacked bioreactor units 36b₁ to 36bⱼ and the next unseeded stacked bioreactor unit 36bⱼ₊₁ in a similar manner described with respect to FIG. 14C.

Then, the lower flow channel 50 of the next unseeded stacked bioreactor unit 36bⱼ₊₁ is seeded with a population of biological cells 2 and microbeads 4 using aliquots isolated from the expanded populations of biological cells 2 and microbeads 4 within the respective previously seeded stacked bioreactor units 36b₁ to 36bⱼ.

In particular, seeding media 26′ containing no biological cells 2 is flowed from the saline source 14g along the forward portion 38c′ of the third flow path 38c into the lower flow channels 48 to isolate aliquots of biological cells 2 and microbeads 4 from respective previously seeded stacked bioreactor units 36b₁ to 36bⱼ (step 252). In particular, the flow of the seeding media 26′ within the lower flow channels 48 of the respective previously seeded stacked bioreactor units 36b₁ to 36bⱼ causes at least one upper layer of biological cells 2 and microbeads 4 to slough off of each of the multilayer expanded populations of biological cells 2 disposed on the cell culture surfaces 54 of the respective previously seeded stacked bioreactor units 36b₁ to 36bⱼ, thereby suspending biological cells 2 and microbeads 4 in the seeding media 26′ as isolated aliquots of biological cells 2 and microbeads 4, while a monolayer or one or more lower layers of the expanded populations of biological cells 2 remain on the cell culture surfaces 54 of the respective previously seeded stacked bioreactor units 36b₁ to 36bⱼ (step 252).

Then, the seeding media 26 containing the aliquots of biological cells 2 and microbeads 4 is flowed from the lower flow channels 48 of the respective previously seeded stacked bioreactor units 36b₁ to 36bⱼ further along the forward portion 38c′ of the third flow path 38c into the lower flow channel 48 of the next unseeded stacked bioreactor unit 36bⱼ₊₁ (step 254); seeding media 26′ containing no biological cells 2 and microbeads 4 is perfused along bypass portion 38c″ of the third flow path 38c from the lower flow channel 50, through the porous membrane 46, and into the upper flow channel 48, of the next unseeded stacked bioreactor unit 36bⱼ₊₁, while substantially retaining the biological cells 2 and microbeads 4 within the lower flow channel 50 of the next unseeded stacked bioreactor unit 36bⱼ₊₁ (step 256); the perfused seeding media 26′ containing no biological cells 2 and microbeads 4 is flowed along the return portion 38c″ of the third flow path 38c out of the upper flow channel 48 of the next unseeded stacked bioreactor unit 36bⱼ₊₁ to the waste destination 14i (step 258); and, once the flow of the seeding media 26′ ceases, the biological cells 2 and microbeads 4 settle to the bottom of the lower flow channel 50 via the action of gravity, such that a seed population of biological cells 2 and microbeads 4 is disposed on the cell culture surface 54 of the next unseeded stacked bioreactor unit 36bⱼ₊₁ (step 260). In alternative methods, more than one bioreactor unit 36b (e.g., the next unseeded stacked bioreactor unit 36bⱼ₊₁ and the next unseeded stacked bioreactor unit 36bⱼ₊₁) may be seeded with aliquots of biological cells 2 isolated from the respective previously seeded stacked bioreactor units 36b₁ to 36bⱼ.

In an alternative method, seeding media 26 containing biological cells 2 and/or microbeads 4 may be flowed from the biological cell source 14a and/or microbead source 14b along the forward portion 38c′ of the third flow path 38c into the lower flow channels 48 of the respective previously seeded stacked bioreactor units 36b₁ to 36bⱼ, and then into the lower flow channel 50 of the unseeded stacked bioreactor unit 36bⱼ₊₁, such that the unseeded stacked bioreactor unit 36bⱼ₊₁ may be seeded with both the aliquots of biological cells 2 and microbeads 4 isolated from the previously seeded stacked bioreactor units 36b₁ to 36bⱼ and with biological cells 2 and microbeads 4 obtained directly from the biological cell source 14a and microbead source 14b.

Of course, if the previously seeded stacked bioreactor units 36b₁ to 36bⱼ only includes the first stacked bioreactor unit 36b₁ (j=1), the next unseeded bioreactor unit 36bⱼ₊₁ will only be seeded with one aliquot of biological cells 2 isolated from the first stacked bioreactor unit 36b₁.

In particular, a flow sequence of first stacked bioreactor unit 36b₁ and the next unseeded stacked bioreactor unit 36bⱼ₊₁ (i.e., the second stacked bioreactor unit 36b₂) is added (e.g., via operation of the valve assembly 16) to the third flow path 38c between the saline source 14g and the waste destination 14i (step 250) (see FIG. 17V), and in this case, by fluidly coupling the lower flow channels 48 of the first stacked bioreactor unit 36b₁ and the second stacked bioreactor unit 36b₂ in series along the forward portion 38c′ of the third flow path 38a between the saline source 14g and the waste destination 14i; fluidly coupling the upper flow channels 50 of the first stacked bioreactor unit 36b₁ and the second stacked bioreactor unit 36b₂ in series along the return portion 38c″ of the third flow path 38c between the waste destination 14i and the saline source 14g; and fluidly coupling the upper flow channels 48 and lower flow channels 50 of each of the first stacked bioreactor unit 36b₁ and the second stacked bioreactor unit 36b₂ to each other via opening/closing of the ports 56-62 of the respective first stacked bioreactor unit 36b₁ and the second stacked bioreactor unit 36b₂ in a similar manner described with respect to FIG. 14C.

Then, the lower flow channel 50 of the second stacked bioreactor unit 36b₂ is seeded with a population of biological cells 2 and microbeads 4 using an aliquot isolated from the expanded population of biological cells 2 and microbeads 4 within the first stacked bioreactor unit 36b₁.

In particular, seeding media 26′ containing no biological cells 2 is flowed from the saline source 14g along the forward portion 38c′ of the third flow path 38c into the lower flow channel 48 to isolate an aliquot of biological cells 2 and microbeads 4 from the first stacked bioreactor units 36b₁ (step 252). In particular, the flow of the seeding media 26′ within the lower flow channel 48 of the first stacked bioreactor units 36b₁ causes at least one upper layer of biological cells 2 and microbeads 4 to slough off of the multilayer expanded population of biological cells 2 disposed on the cell culture surfaces 54 of the first stacked bioreactor units 36b₁, thereby suspending biological cells 2 and microbeads 4 in seeding media 26 as isolated aliquots of biological cells 2 and microbeads 4, while a monolayer or one or more lower layers of the expanded population of biological cells 2 remains on the cell culture surface 54 of the first stacked bioreactor units 36b₁ (step 252) (see FIG. 17W).

Then, the seeding media 26 containing the aliquot of biological cells 2 and microbeads 4 is flowed from the lower flow channel 48 of the first stacked bioreactor unit 36b₁ further along the forward portion 38c′ of the third flow path 38c into the lower flow channel 48 of the second stacked bioreactor unit 36b₂ (step 254) (see first stacked bioreactor unit 36b₁) (see FIG. 17X); seeding media 26′ containing no biological cells 2 and microbeads 4 is perfused along bypass portion 38c″ of the third flow path 38c from the lower flow channel 50, through the porous membrane 46, and into the upper flow channel 48, of the second stacked bioreactor unit 36b₂, while substantially retaining the biological cells 2 and microbeads 4 within the lower flow channel 50 of the second stacked bioreactor unit 36b₂ (step 256) (see FIG. 17Y); the perfused seeding media 26′ containing no biological cells 2 and microbeads 4 is flowed along the return portion 38c″ of the third flow path 38c out of the upper flow channels 48 of the first and second stacked bioreactor units 36b₂, 36b₁ to the waste destination 14i (step 258) (see FIG. 17Z); and, once the flow of the seeding media 26′ ceases, the biological cells 2 and microbeads 4 settle to the bottom of the lower flow channel 50 via the action of gravity, such that a seed population of biological cells 2 and microbeads 4 is disposed on the cell culture surface 54 of the second stacked bioreactor unit 36b₂ (step 260) (see FIG. 17AA).

The method 200 then returns to steps 220-246, whereby the populations of biological cells 2 residing in the first stacked bioreactor unit 36b₁ and second stacked bioreactor unit 36b₂, as the previously seeded stacked bioreactor units 36b₁ to 36bⱼ, are expanded (re-expanded in the case of the first stacked bioreactor unit 36b₁).

In particular, additional microbeads 4 may be optionally introduced into the first and second stacked bioreactor units 36b₁, 36b₂, such that the respective seed populations of biological cells 2 adhere to the additional microbeads 4 in the first and second stacked bioreactor units 36b₁, 36b₂.

In particular, a flow sequence of the first and second stacked bioreactor units 36b₁, 36b₂ is added (e.g., via operation of the valve assembly 16) to the third flow path 38c between the microbead source 14b and the waste destination 14i (step 220) (see FIG. 17AB), and in this case, by fluidly coupling the lower flow channels 50 of the first and second stacked bioreactor units 36b₁, 36b₂ in series along the forward portion 38c′ of the third flow path 38a between the microbead source 14b and the waste destination 14i; fluidly coupling the upper flow channels 48 of the first and second stacked bioreactor units 36b₁, 36b₂ in series along the return portion 38c″ of the third flow path 38c between the waste destination 14i and the microbead source 14b; and fluidly coupling the upper flow channel 48 and lower flow channel 50 of each of the first and second stacked bioreactor units 36b₁, 36b₂ to each other via opening/closing of the ports 56-62 of each of the first and second stacked bioreactor units 36b₁, 36b₂ in a similar manner described with respect to FIG. 14C.

Then, the microbeads 4 are introduced into the lower flow channels 50 of the respective first and second stacked bioreactor units 36b₁, 36b₂. In particular, the seeding media 26 containing microbeads 4 is flowed along the forward portion 38c′ of the third flow path 38c into the lower flow channels 50 of the respective first and second stacked bioreactor units 36b₁, 36b₂ (step 222) (see FIG. 17AC); seeding media 26′ containing no biological cells 2 and no microbeads 4 is perfused along bypass portions 38c″ of the third flow path 38c from the lower flow channels 50, through the porous membranes 46, and into the upper flow channels 50, of the respective first and second stacked bioreactor units 36b₁, 36b₂, while substantially retaining the biological cells 2 and microbeads 4 within the lower flow channels 50 of the respective first and second stacked bioreactor units 36b₁, 36b₂ (step 224) (see FIG. 17AD); the perfused seeding media 26′ containing no biological cells 2 and no microbeads 4 is flowed along the return portion 38c″ of the third flow path 38c out of the upper flow channels 48 of the respective first and second stacked bioreactor units 36b₁, 36b₂ to the waste destination 14i (step 226) (see FIG. 17AE); and, once the flow of the seeding media 26 ceases, the microbeads 4 settle to the bottom of the lower flow channels 50 via the action of gravity, such that the populations of biological cells 2 disposed on the cell culture surfaces 54 of the respective first and second stacked bioreactor units 36b₁, 36b₂ adhere to the microbeads 4 (step 228) (see FIG. 17AF).

Next, nutrient, reagent, and gas are delivered to the populations of biological cells 2, and waste is extracted from the populations of biological cells 2, thereby facilitating the expansion of the seed populations of biological cells 2 within the lower flow channels 48 of the respective first and second stacked bioreactor units 36b₁, 36b₂.

In particular, a flow sequence of the first and second stacked bioreactor units 36b₁, 36b₂ is added (e.g., via operation of the valve assembly 16) to the second flow path 36b between the nutrient source 14c and reagent source 14d and the waste destination 14i, and in this case, by fluidly coupling the upper flow channels 50 of the respective first and second stacked bioreactor units 36b₁, 36b₂ in series along the second flow path 38b between the nutrient source 14c and reagent source 14d and the waste destination 14i via opening/closing of the ports 56-62 of each of the first and second stacked bioreactor units 36b₁, 36b₂ in a similar manner described with respect to FIG. 14A (step 230) (see FIG. 17AG).

Then, fresh culture media 28 is flowed (continually or intermittently) along the second flow path 38b from the nutrient source 14c and reagent source 14d, through the upper flow channels 50 of the respective first and second stacked bioreactor units 36b₁, 36b₂, while nutrient, reagent, and waste are exchanged between the populations of biological cells 2 and fresh culture media 28 (i.e., the nutrient and reagent are provided to, while the waste is extracted from, the populations of biological cells 2, with the nutrient and reagent diffusing from the upper flow channels 48, through the porous membranes 46, and into the lower flow channels 50, and the waste diffusing from the lower flow channels 50, through the porous membranes 46, and into the upper flow channels 48, of the respective first and second stacked bioreactor units 36b₁, 36b₂), exiting the bioreactor assembly 12″ as spent culture media 28′ with the waste, thereby facilitating the expansion of the seed populations of biological cells 2 within the lower flow channels 50 of the respective first and second stacked bioreactor units 36b₁, 36b₂ (step 232) (see FIG. 17AH).

Simultaneously with the diffusion of the nutrient, reagent, and waste across the porous membrane 46 between the flow channels 48, 50 of the respective first and second stacked bioreactor units 36b₁, 36b₂, gas 76 is diffused from the ambient environment 74, through the dense membranes 72, and into the lower flow channels 48, while allowing liquid media to be retained within the lower flow channels 48, of the respective first and second stacked bioreactor units 36b₁, 36b₂ (step 234) (see FIG. 17AH).

Anytime during incubation, the populations of biological cells 2 and microbeads 4 in the lower flow channels 48 of the respective first and second stacked bioreactor units 36b₁, 36b₂ may be washed.

In particular, a flow sequence of the first and second stacked bioreactor units 36b₁, 36b₂ is added (e.g., via operation of the valve assembly 16) to the third flow path 38c between the wash buffer source 14f and the waste destination 14i, and in this case, by fluidly coupling the lower flow channels 50 of the respective first and second stacked bioreactor units 36b₁, 36b₂ in series along the forward portion 38c′ of the third flow path 38a between the wash buffer source 14f and the waste destination 14i; fluidly coupling the upper flow channels 48 of the respective first and second stacked bioreactor units 36b₁, 36b₂ in series along the return portion 38c″ of the third flow path 38c between the waste destination 14i and the wash buffer source 14f; and fluidly coupling the upper flow channel 48 and lower flow channel 50 of each of the first and second stacked bioreactor units 36b₁, 36b₂ to each other via opening/closing of the ports 56-62 of each of the first and second stacked bioreactor units 36b₁, 36b₂ in a similar manner described with respect to FIG. 14C (step 236) (see FIG. 17AI).

Then, the bioreactor assembly 12″ is mechanically agitated, thereby suspending the populations of biological cells 2 and microbeads 4 in the lower flow channels 48, such that the populations of biological cells 2 will be maximally exposed to the fresh wash buffer 30 and will not block perfusion of the used wash buffer 30 containing undesirable material (e.g., unbound molecules, debris, dead cells, or other undesirable molecules) through the porous membranes 46, of the respective first and second stacked bioreactor units 36b₁, 36b₂ (step 238) (see FIG. 17AJ); the fresh wash buffer 30 is flowed along the forward portion 38c′ of the third flow path 38c into, while accumulating the undesirable material from, the lower flow channels 50 of the respective first and second stacked bioreactor units 36b₁, 36b₂, thereby washing the populations of biological cells 2 and microbeads 4 (step 240) (see FIG. 17AK); used wash buffer 30′ containing the undesirable material, is perfused along bypass portions 38c″ of the third flow path 38c from the lower flow channels 50, through the porous membranes 46, and into the upper flow channels 48, while substantially retaining the biological cells 2 and microbeads 4 within the lower flow channels 50, of the respective first and second stacked bioreactor units 36b₁, 36b₂ (step 242) (see FIG. 17AL); the perfused used wash buffer 30′ containing the undesirable material is flowed along the return portion 38c″ of the third flow path 38c out of the upper flow channels 48 of the respective first and second stacked bioreactor units 36b₁, 36b₂ to the waste destination 14i (step 244) (see FIG. 17AM); and, once the flow of the fresh wash buffer 30 ceases, the biological cells 2 and microbeads 4 settle to the bottoms of the lower flow channels 50 via the action of gravity, such that seed populations of biological cells 2 reconstitute on the cell culture surfaces 54 of the respective first and second stacked bioreactor units 36b₁, 36b₂ (step 246) (see FIG. 17AN). After the washing steps, nutrient, reagent, and gas may be delivered to the populations of biological cells 2, and waste may be extracted from the populations of biological cells 2, thereby facilitating further expansion of the seed populations of biological cells 2 within the upper flow channels 48 of the respective previously first and second stacked bioreactor units 36b₁, 36b₂ in accordance with steps 230-234.

Steps 220-248 are then repeated until expanded populations of biological cells 2 reside in all n number of stacked bioreactor units 36b₁ to 36bₙ (see FIG. 17AO). That is, if the previously seeded stacked bioreactor units 36b₁ to 36bⱼ do include the last stacked bioreactor unit 36bₙ (step 248), the expanded populations of biological cells 2 are harvested from the respective n number of stacked bioreactor units 36a₁ to 36aₙ.

In particular, a flow sequence of the n number of stacked bioreactor units 36a₁ to 36aₙ is added (e.g., via operation of the valve assembly 16) to the first flow path 36a between the saline source 14g and the biological cell collection destination 14h, and in this case, by fluidly coupling the lower flow channels 48 of the respective n number of stacked bioreactor units 36a₁ to 36aₙ in series along the first flow path 38a between the saline source 14g and the biological cell collection destination 14h via opening/closing of the ports 56-62 of each of the n number of stacked bioreactor units 36a₁ to 36aₙ in a similar manner described with respect to FIG. 14B (step 262) (see FIG. 17AP).

Then, harvesting media 32 is flowed along the first flow path 38a from the saline source 14g, respectively through, while suspending expanded populations of biological cells 2 and microbeads 4 residing in, lower flow channels 48 (see FIG. 17AQ), exiting the bioreactor assembly 12′ as harvesting media 32′ with the suspended expanded populations of biological cells 2 and microbeads 4 to the biological cell collection destination 14h, thereby harvesting the expanded populations of the biological cells 2 and microbeads 4 from the upper flow channels 48 of the respective n number of stacked bioreactor units 36a₁ to 36aₙ (step 264) (see FIG. 17AR).

As briefly described in herein, as well as in U.S. Provisional Application Ser. No. 63/485,865, entitled “Bioreactor for Cellular Therapeutics,” filed Feb. 17, 2023, and PCT Application Ser. No. PCT/US2022/012194, entitled “Systems, Apparatuses, and Methods for Cellular Therapeutics Manufacture,” PCT/US2023/69944, entitled “Bioreactor for Cellular Therapeutics,” filed Jul. 11, 2023, which have been expressly incorporated herein by reference, the valve assembly and pump assembly used to control the flow paths within the bioreactor or bioreactors may comprise multiple valves and pumps.

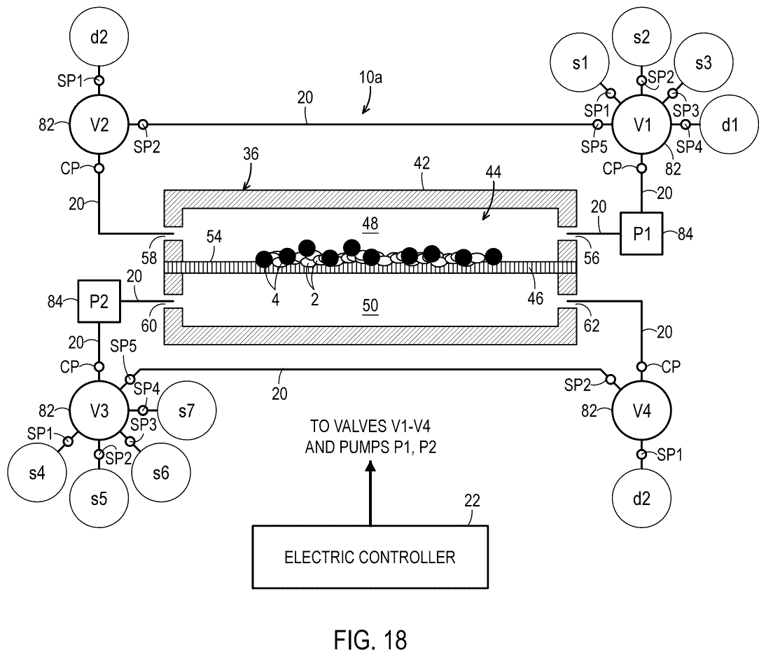

For example, as illustrated in FIG. 18, one embodiment of a bioreactor system 10a constructed in accordance with the present inventions will be described. The bioreactor system 10a generally comprises a single bioreactor 36, a plurality of media sources s1-s7, a plurality of media destinations d1-d2, four single-function valves 82 corresponding to the valve assembly 16 illustrated in FIG. 1 (in particular, a first selector valve V1, a second selector valve V2, a third selector valve V3, and a fourth selector valve V4), two pumps 84 corresponding to the pump assembly 18 illustrated in FIG. 1 (in particular, a first pump P1 and a second pump P2), the arrangement of fluid conduits 20, and the electric controller 22.

In one embodiment, the media sources s1-s7 and media destinations d1-d2 are unique relative to each other (e.g., they may respectively correspond to the media sources 14a-14g and media destinations 14h-14i illustrated in FIG. 1). In an alternative embodiment, at least some of the media sources s1-s3 may be the same as the media sources s4-s7 (e.g., both the media source s1 and s4 may be saline), whereas the media destinations d1-d2 are preferably different from each other (e.g., the media destination d1 may be a biological cell collection region or a biological cell analytical region, whereas the media destination d2 may be waste).

In the illustrated embodiment, the bioreactor 36 has a similar structure and function as the bioreactor unit 36a illustrated in FIGS. 5-6, although it should be appreciated that the bioreactor 36 may have any suitable structure and function, e.g., similar structure and function as the bioreactor unit 36b illustrated in FIG. 13. Notably, in the illustrated embodiment, the ports 56-62 are arbitrary in that media may flow in or out of a particular port depending on the configuration of the V1-V4 and pumps P1, P2. Thus, in this embodiment, the ports 56-62 are characterized as a first port 56, a second port 58, a third port 60, and a fourth port 62 without reference to the terms “inlet” or “outlet.”

As will be described in further detail below, the valves V1-V4 are configured for selectively defining different flow paths between various ones of the media sources s1-s7 and media destinations d1-d2 and the bioreactor 36, while the pumps P1, P2 are configured for providing the necessary fluid pressure for conveying various media along the flow paths defined by the single-function valves V1-V4, such that different media can be flowed between the a sources s1-s7 and media destinations d1-d2 and the bioreactor 36 for the purpose of: (1) priming the bioreactor 36 (i.e., removing all air within the bioreactor 36); (2) seeding the bioreactor 36 with a population of biological cells (with or without microbeads); (3) culturing the biological cells, such that the population of biological cells are expanded; washing the biological cells; (4) harvesting or sampling the expanded population of biological cells from the bioreactor 36; and (5) clearing the fluid conduits 20 (i.e., relocating stray biological cells from the fluid conduits 20 back into the bioreactor 36).

Each of the pumps P1, P2 can be any pump capable of applying fluid pressure to media without contaminating such media (e.g., a peristaltic pump). In the illustrated embodiment, the pumps P1, P2 are connected downstream from the respective media sources s1-s7, such that media is pulled from the media sources s1-s7 into and through the bioreactor 36. In this manner, cavitation of, and the resultant introduction of air bubbles into, the media is minimized. Alternatively, one or both of the pumps P1, P2 may be connected upstream from the respective media sources s1-s7, such that media is pushed from the media sources s1-s7 into and through the bioreactor 36.

In order to selectively define the different flow paths, as well as the particular ones of the media sources s1-s7 and media destinations d1-d2 associated with the defined flow paths, the valves V1-V4 and pumps P1, P2 may be operated in coordination with each other. In particular, the valves V1, V2 and pump P1 are functionally associated with the upper flow channel 48, and the valves V3, V4 and pump P2 are functionally associated with the lower flow channel 50.

In particular, the first valve V1 comprises a common port CP fluidly connected to the first port 56 of the bioreactor 36 via the first pump P1, and a plurality of selector ports SP1-SP5 respectively fluidly connected to the media sources s1-s3, the media destination d1. The second valve V2 comprises a common port CP fluidly connected to the second port 58 of the bioreactor 36, and a plurality of selector ports SP1-SP2 respectively fluidly connected to the media destination d2 and the first valve V1. The third valve V3 comprises a common port CP fluidly connected to the third port 60 of the bioreactor 36 via the second pump P2, and a plurality of selector ports SP1-SP5 respectively fluidly connected to the media sources s4-s7 and the fourth valve V4. The fourth single-function valve V4 comprises a common port CP fluidly connected to the fourth port 62 of the bioreactor 36, and a plurality of selector ports SP1-SP2 respectively fluidly connected to the media destination d2 and the third valve V3.

The first valve V1 may be operated to fluidly couple a selected one of the media sources s1-s3, the media destination d1, and the second port 58 of the bioreactor 36 (via the second valve V2) to (or fluidly decouple all of the media sources s1-s3, media destination d1, and second port 58 of the bioreactor 36 from) the first port 56 of the bioreactor 36. The second valve V2 may be operated to fluidly couple a selected one of the media destination d2 and the first port 56 of the bioreactor 36 (via the first valve V1) to (or fluidly decouple both of the media destination d2 and first port 56 of the bioreactor 36 from) the second port 58 of the bioreactor 36. The third valve V3 may be operated to fluidly couple a selected one of the media sources s4-s7 and fourth port 62 of the bioreactor 36 (via the fourth valve V4) to (or fluidly decouple all of the media sources s4-s7 and fourth port 62 of the bioreactor 36 from) the third port 60 of the bioreactor 36. The fourth valve V4 may be operated to fluidly couple a selected one of the media destination d2 and the third port 60 of the bioreactor 36 (via the third valve V3) to (or fluidly decouple both of the media destination d2 and third port 60 of the bioreactor 36 from) the fourth port 62 of the bioreactor 36.

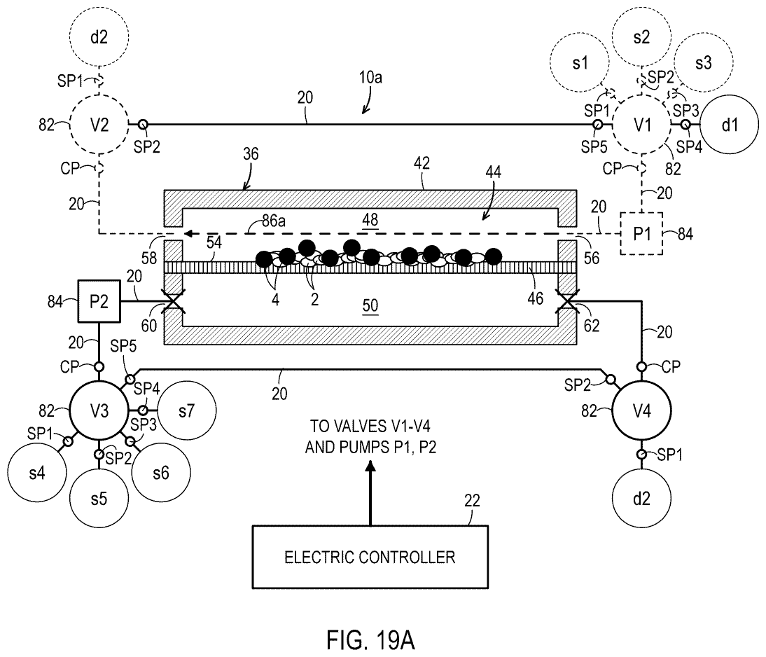

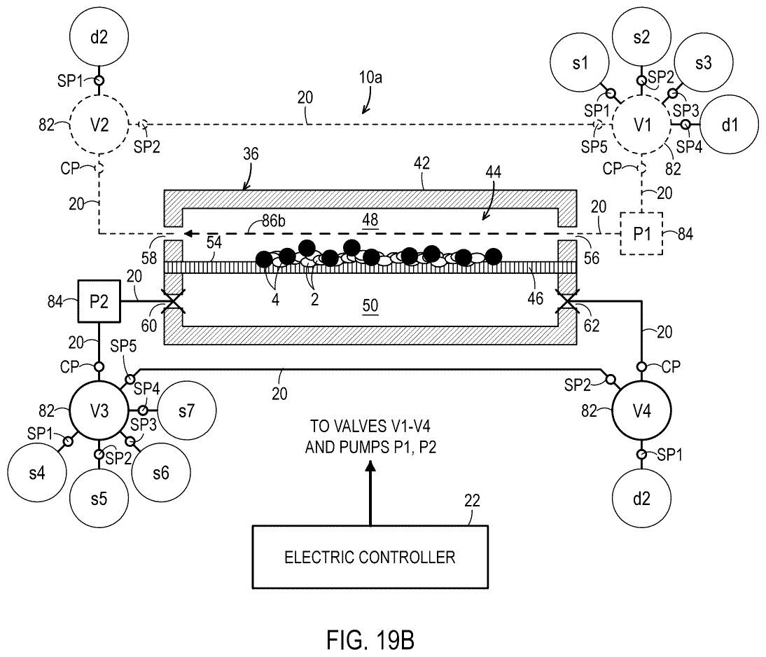

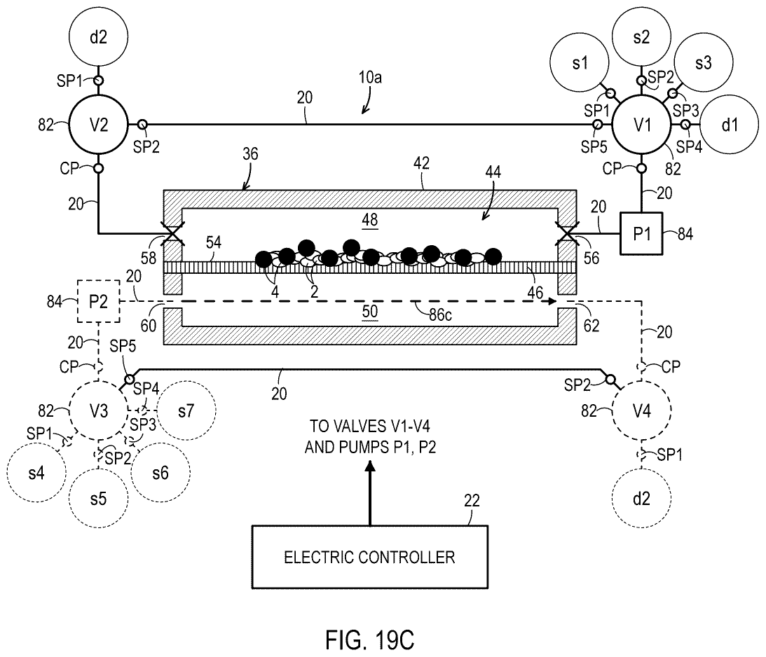

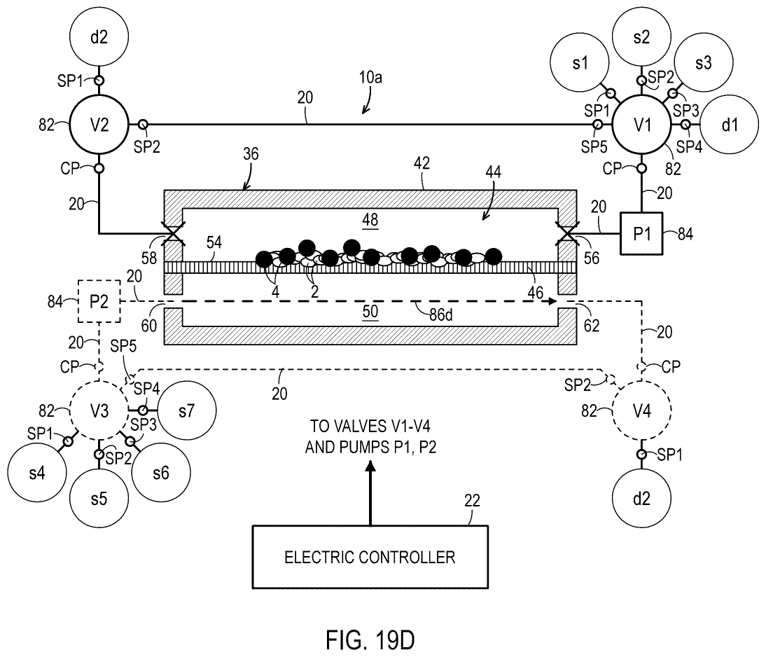

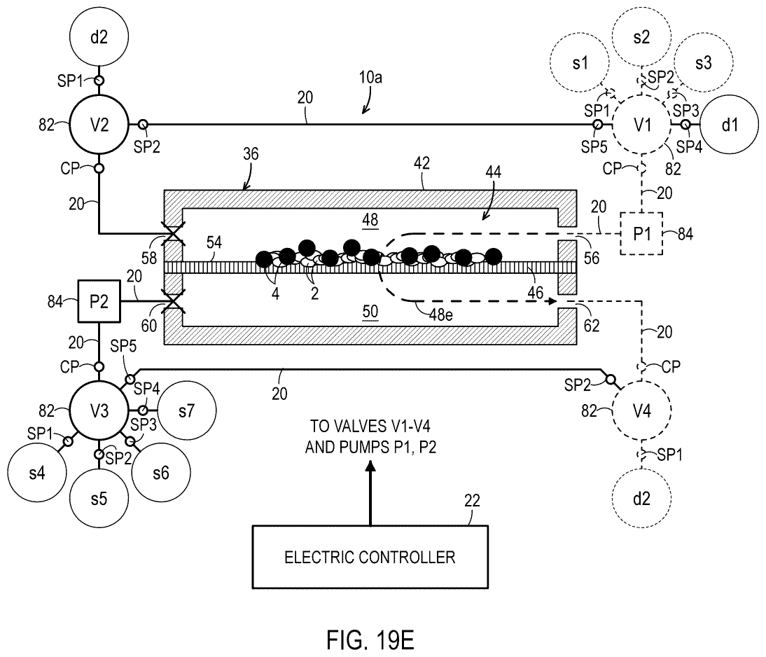

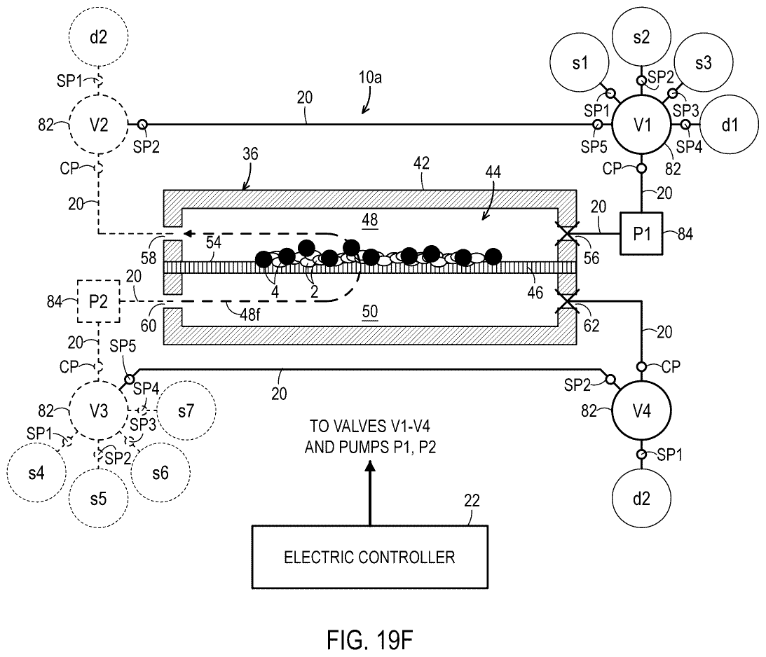

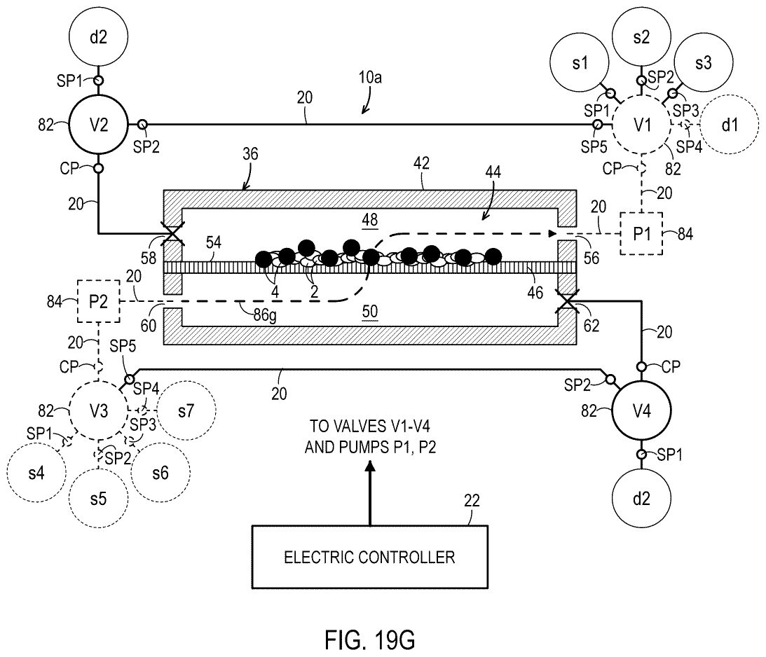

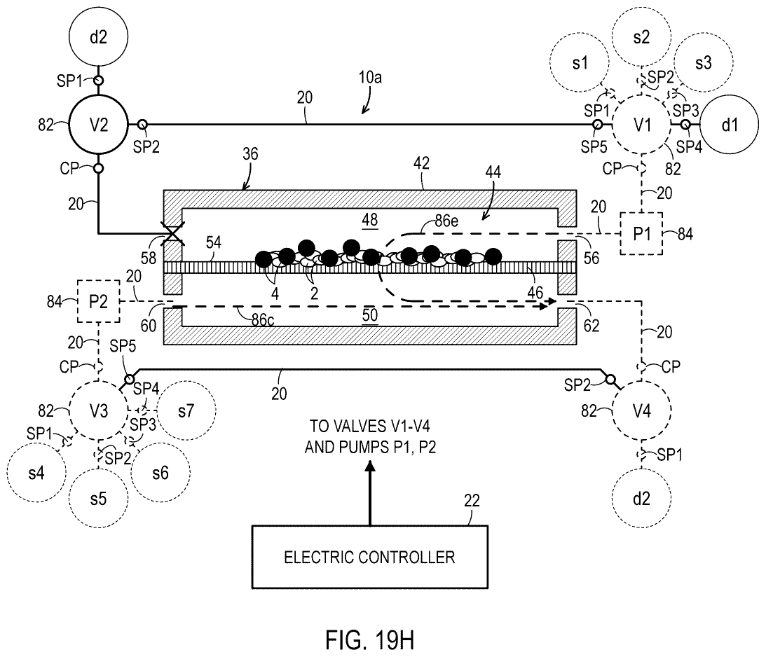

Thus, is should be appreciated that the valves V1-V4 can be collectively controlled by the electric controller 22 to define a particular flow path through the interior cavity 44 of the bioreactor 36. For example, referring to FIGS. 19A-19H, the valves V1-V4 are operated in conjunction with the media sources s1-s7 and media destinations d1-d2 in a variety of arrangements to define various flow paths 86a-86h through the bioreactor 36 to perform certain functions. Notably, the particular media sources s1-s7, media destinations d1-d2, valves V1-V4, pumps P1, P2, and fluid conduits 20 from, through, and to which media associated with a particular flow is shown dashed for purposes of illustration in FIGS. 19A-19H.

In one arrangement illustrated in FIG. 19A, the valves V1-V4 may be operated to define a first flow path 86a entirely through the upper flow channel 48 via the first and second ports 56, 58 of the bioreactor 36. In particular, the first valve V1 is operated to fluidly couple a selected one of the media sources s1-s3 to the first port 56 of the bioreactor 36, and the second valve V2 is operated to fluidly couple the media destination d2 to the second port 58 of the bioreactor 36 (effectively opening the first and second ports 56, 58 between the selected one of the media sources s1-s3 and the media destination d2), while the third valve V3 is operated to fluidly decouple all of the media sources s4-s7 and fourth valve V4 from the third port 60 of the bioreactor 36, and the fourth valve V4 is operated to fluidly decouple the media destination d2 and the third valve V3 from the fourth port 62 of the bioreactor 36 (effectively closing the third and fourth ports 60, 62). The first pump P1 is turned on, while the second pump P2 is turned off.

The first flow path 86a may be uni-directional in that media can flow along the first flow path 86a in only one direction, and in this case, from the first port 56 to the second port 58. Under fluid pressure applied by the first pump P1, media flows in this direction from a selected one of the media sources s1-s3 along the first flow path 86a entirely through the upper flow channel 48 via the first and second ports 56, 58 to the media destination d2. The first flow path 86a can be used to perform such functions as, e.g., priming of the bioreactor 36. For example, one of the media sources s1-s3 selected by the first valve V1 may be saline, and the media destination d2 may be waste, such that the saline (as priming media) may flow along the first flow path 86a entirely through the upper flow channel 48 to waste. Thus, any air within the upper flow channel 48 will be displaced (i.e., flushed out) by the saline. In this manner, when the upper flow channel 48 is pressurized, any media flowing through the upper flow channel 48 will be consistent and well-defined.

In another arrangement illustrated in FIG. 19B, the valves V1-V4 may be operated to define a second flow path 86b entirely through the upper flow channel 48 via the first and second ports 56, 58 of the bioreactor 36 that effectively bypasses the media sources s1-s3 and media destinations d1-d2. In particular, the first and second valves V1, V2 are operated to fluidly couple to each other (effectively opening the first and second ports 56, 58 to each other), while the third valve V3 is operated to fluidly decouple all of the media sources s1-s3 and fourth valve V4 from the third port 60 of the bioreactor 36, and the fourth valve V4 is operated to fluidly decouple the media destination d2 and the third valve V3 from the fourth port 62 of the bioreactor 36 (effectively closing the third and fourth ports 60, 62). The first pump P1 is turned on, while the second pump P2 is turned off.

The second flow path 86b may be bi-directional in that media can flow along the second flow path 86b in both directions, and in this case, in a first direction from the first port 56 to the second port 58 or in a second direction from the second port 58 to the first port 56. Thus, under fluid pressure applied by the first pump P1, media may either flow in the first direction or the second direction (depending on the direction in which the first pump P1 operates), such that it recirculates along the second flow path 86b through the upper flow channel 48 of the bioreactor 36 via the first and second ports 56, 58. The second flow path 86b may be used to perform such functions as, e.g., clearing the fluid conduits 20 respectively fluidly connected to the first and second ports 56, 58. For example, while the media sources s1-s3 and media destinations d1-d2 are bypassed, saline may be recirculated along the second flow path 86b in either or both directions in an effort to corral any residual biological cells 2 and/or microbeads 4 that have been trapped in the fluid conduits 20 into the upper flow channel 48 of the bioreactor 36.

In still another arrangement illustrated in FIG. 19C, the valves V1-V4 may be operated to define a third flow path 86c entirely through the lower flow channel 50 via the third and fourth ports 60, 62 of the bioreactor 36. In particular, the third valve V3 is operated to fluidly couple a selected one of the media sources s4-s7 to the third port 60 of the bioreactor 36, and the fourth valve V4 is operated to fluidly couple the media destination d2 to the fourth port 60 of the bioreactor 36 (effectively opening the third and fourth ports 60, 62 between the selected one of the media sources s4-s7 and the media destination d2), while the first valve V1 is operated to fluidly decouple all of the media sources s1-s3, the media destination d1, and the second valve V2 from the first port 56 of the bioreactor 36, and the second valve V2 is operated to fluidly decouple the media destination d2 and the first valve V1 from the second port 60 of the bioreactor 36 (effectively closing the first and second ports 56, 60). The first pump P1 is turned off, while the second pump P2 is turned on.

The third flow path 86c may be uni-directional in that media can flow along the third flow path 86c in only one direction, and in this case, from the third port 60 to the fourth port 62. Under fluid pressure applied by the second pump P2, media flows in this direction from a selected one of the media sources s4-s7 along the third flow path 86c entirely through the lower flow channel 50 via the third and fourth ports 60, 62 to the media destination d2.

The third flow path 86c can be used to perform such functions as, e.g., priming of the bioreactor 36. For example, one of the media sources s4-s7 selected by the third valve V3 may be saline, and the media destination d2 may be waste, such that the saline (as priming media) may flow along the third flow path 86c entirely through the lower flow channel 50 to waste. Thus, any air within the lower flow channel 50 will be displaced (i.e., flushed out) by the saline. In this manner, when the lower flow channel 50 is pressurized, any media flowing through the lower flow channel 50 will be consistent and well-defined.

The third flow path 86c may also be used to perform such functions as, e.g., culturing the biological cells 2 residing in the upper flow channel 48 on the cell culture surface 54 of the porous membrane 46 with fresh media containing nutrient, reagent, or gas. For example, one of the media sources s4-s7 selected by the third valve V3 may be nutrient, reagent, or gas, and the media destination d2 may be waste, such that media containing nutrient, reagent, or gas may flow (continuously or intermittently) along the third flow path 86c entirely through the lower flow channel 50 to waste. Thus, nutrient, reagent, and gas will be delivered to the environment surrounding the population of biological cells 2, as well as extracting waste from the environment surrounding the population of biological cells 2 to facilitate expansion of the population of biological cells 2, and in particular, by diffusing the nutrient, reagent, and gas through the porous membrane 46 from the lower flow channel 50 into the upper flow channel 48, and diffusing the waste through the porous membrane 46 from the upper flow channel 48 into the lower flow channel 50.

In another arrangement illustrated in FIG. 19D, the valves V1-V4 may be operated to define a fourth flow path 86d entirely through the lower flow channel 50 via the third and fourth ports 60, 62 of the bioreactor 36 that effectively bypasses the media sources s4-s7 and media destination d2. In particular, the third and fourth valves V3, V4 are operated to fluidly couple to each other (effectively opening the first and second ports 56, 58 to each other), while the first valve V1 is operated to fluidly decouple all of the media sources s1-s3, the media destination d1, and the second valve V2 from the first port 56 of the bioreactor 36, and the second valve V2 is operated to fluidly decouple the media destination d2 and the first valve V1 from the second port 58 of the bioreactor 36 (effectively closing the first and second ports 56, 58). The first pump P1 is turned off, while the second pump P2 is turned on.

The fourth flow path 86d may be bi-directional in that media can flow along the fourth flow path 86d in both directions, and in this case, in a first direction from the third port 60 to the fourth port 62 or in a second direction from the fourth port 62 to the third port 60. Thus, under fluid pressure applied by the first pump P2, media may either flow in the first direction or the second direction (depending on the direction in which the first pump P2 operates), such that it recirculates along the fourth flow path 86d through the lower flow channel 50 of the bioreactor 36 via the third and fourth ports 60, 62. The fourth flow path 86d may be used to recirculate media containing nutrient, reagent, and gas through the lower flow channel 50. For example, while the media sources s4-s7 and media destination d2 are bypassed, the media containing the nutrient, reagent, or gas may be recirculated along the fourth flow path 86d in either or both directions until the nutrient, reagent, or gas in the media has been depleted, exhausted or spent, after which the fresh media with new nutrient, reagent, or gas may be flowed through the lower flow channel 50. In this manner, usage of the nutrient, reagent, or gas in the media may be maximized.

In yet another arrangement illustrated in FIG. 19E, the valves V1-V4 may be operated to define a fifth flow path 86e into the upper flow channel 48 via the first port 56, between the upper and lower flow channels 48, 50 via the porous membrane 46, and out of the lower flow channel 50 via the fourth port 62, of the bioreactor 36. In particular, the first valve V1 is operated to fluidly couple a selected one of the media sources s1-s3 to the first port 56 of the bioreactor 36, and the fourth valve V4 is operated to fluidly couple the media destination d2 to the fourth port 62 of the bioreactor 36 (effectively opening the first and fourth ports 56, 62 between the selected one of the media sources s1-s3 and the media destination d2), while the second valve V2 is operated to fluidly decouple the media destination d2 and the first valve V1 from the second port 58 of the bioreactor 36, and the third valve V3 is operated to fluidly decouple all of the media sources s4-s7 and the fourth valve V4 from the third port 60 of the bioreactor 36 (effectively closing the second and third ports 58, 60).

The fifth flow path 86e may be uni-directional in that media can flow along the fifth flow path 86e in only one direction, and in this case, from the first port 56 to the fourth port 62. As a result, under fluid pressure applied by the first pump P1, media flows from a selected one of the media sources s1-s3 along the fifth flow path 86e, into the upper flow channel 48 via the first port 56, perfuses through the porous membrane 46 from the upper flow channel 48 into the lower flow channel 50 (due to the pressure buildup in the upper flow channel 48 caused by the closed second port 58 that creates a pressure gradient between the flow channels 48, 50), and out of the lower flow channel 50 via the fourth port 62 to the media destination d2. The first pump P1 is turned on while the second pump P2 is turned off.

The fifth flow path 86e can be used to perform such functions as, e.g., seeding the bioreactor 36 with biological cells 2 and/or microbeads 4. For example, one of the media sources s1-s3 selected by the first valve V1 may be media containing biological cells 2 and/or microbeads 4, and the media destination d2 may be waste, such that the media containing the biological cells 2 and/or microbeads 4, may flow along the fifth flow path 86e into the upper flow channel 48, perfuse through the porous membrane 46 from the upper flow channel 48 to the lower flow channel 50 (without the biological cells 2 and/or microbeads 4), and then out of the lower flow channel 50 to waste. Thus, the porous membrane 46 captures (filters) the biological cells 2 and/or microbeads 4, thereby seeding the upper flow channel 48 with the population of biological cells 2, and in particular, urging the biological cells 2 and/or microbeads 4 against the cell culture surface 54 on the porous membrane 46 via the action of convection. Because the biological cells 2 and/or microbeads 4 are filtered and captured by the porous membrane 46 while the media perfuses through the porous membrane 46, loss of biological cells 2 and/or microbeads 4 out of the bioreactor 36 is minimized during seeding of the bioreactor 36 with the population of biological cells 2 and/or microbeads 4. In this manner, seeding of the bioreactor 36 with a population of biological cells 2 and/or microbeads 4 may be accomplished robustly and expeditiously without concern that some of the biological cells 2 and/or microbeads 4 will be lost with the outflow of the media out of the bioreactor 36.

The fifth flow path 86e can also be used to perform such functions as, e.g., washing the biological cells 2 and/or microbeads 4. For example, one of the media sources s1-s3 selected by the first valve V1 may wash buffer, and the media destination d2 may be waste, such that fresh wash buffer may flow along the fifth flow path 86e into the upper flow channel 48, perfuse through the porous membrane 46 from the upper flow channel 48 to the lower flow channel 50 as user wash buffer with undesirable material (e.g., unbound molecules, debris, dead cells, or other unwanted molecules), and then out of the lower flow channel 50 to waste. In the same manner that the porous membrane 46 filters the biological cells 2 and/or microbeads 4 during seeding of the bioreactor 36, the porous membrane 46 filters the biological cells 2 and/or microbeads 4 during washing.

The fifth flow path 86e can also be used to perform such functions as, e.g., clearing the fluid conduit 20 fluidly connected to the first port 56 after sampling of the biological cells 2 (described in further detail below) that might result in the trapping of some of the biological cells 2 in the fluid conduit 20 fluidly connected to the first port 56 or after an attempt to clear the fluid conduits 20 respectively fluidly connected to the first and second ports 56, 58 in the arrangement illustrated in FIG. 19B that results in the trapping of some of the biological cells 2 in the fluid conduit 20 fluidly connected to the first port 56. For example, one of the media sources s1-s3 selected by the first valve V1 may be saline, and the media destination d2 may be waste, such that the saline may flow along the fifth flow path 86e into the upper flow channel 48 (carrying any biological cells 2 and/or microbeads 4 trapped in the fluid conduit 20 fluidly connected to the first port 56), perfuse through the porous membrane 46 from the upper flow channel 48 to the lower flow channel 50, and then out of the lower flow channel 50 to waste. In the same manner that the porous membrane 46 filters the biological cells 2 and/or microbeads 4 during seeding of the bioreactor 36, the porous membrane 46 filters the biological cells 2 and/or microbeads 4 during clearing of the fluid conduit 20 fluidly connected to the first port 56.

In yet another arrangement illustrated in FIG. 19F, the valves V1-V4 may be operated to define a sixth flow path 86f into the lower flow channel 50 via the third port 60, between the lower and upper flow channels 50, 48 via the porous membrane 46, and out of the upper flow channel 48 via the second port 58, of the bioreactor 36. In particular, the second valve V2 is operated to fluidly couple the media destination d2 to the second port 58 of the bioreactor 36, and the third valve V3 is operated to fluidly couple a selected one of the media sources s4-s7 to the third port 60 of the bioreactor 36 (effectively opening the second and third ports 58, 60 between the selected one of the media sources s4-s7 and the media destination d2), while the first valve V1 is operated to fluidly decouple all of the media sources s1-s3, the media destination d1, and the second valve V2 from the first port 56 of the bioreactor 36, and the fourth valve V4 is operated to fluidly decouple the media destination d2 and the third valve V3 from the fourth port 62 of the bioreactor 36 (effectively closing the first and fourth ports 56, 62). The first pump P1 is turned off, while the second pump P2 is turned on.

The sixth flow path 86f may be uni-directional in that media can flow along the sixth flow path 86f in only one direction, and in this case, from the third port 60 to the second port 58. As a result, under fluid pressure applied by the second pump P2, media flows from a selected one of the media sources s4-s7 along the sixth flow path 86f, into the lower flow channel 50 via the third port 60, perfuses through the porous membrane 46 from the lower flow channel 50 into the upper flow channel 48 (due to the pressure buildup in the lower flow channel 50 caused by the closed fourth port 62 that creates a pressure gradient between the flow channels 48, 50), and out of the upper flow channel 48 via the second port 58 to the media destination d2.

The sixth flow path 86f may be used to perform such functions as, e.g., refreshing the media in the upper flow channel 48 or suspending the biological cells 2 and/or microbeads 4 in the upper flow channel 48. For example, one of the media sources s4-s7 selected by the third valve V3 may be saline, and the media destination d2 may be waste, such that the saline may flow along the sixth flow path 86f into the lower flow channel 50, perfuse through the porous membrane 46 from the lower flow channel 50 to the upper flow channel 48, and then out of the upper flow channel 48 and to waste. Depending upon the flow rate of the saline, the biological cells 2 and/or microbeads 4 may be displaced from the cell culture surface 54 of the porous membrane 46 and be suspended within the saline in the upper flow channel 48.

In yet another arrangement illustrated in FIG. 19G, the valves V1-V4 may be operated to define a seventh flow path 86g into the lower flow channel 50 via the third port 60, between the lower and upper flow channels 50, 48 via the porous membrane 46, and out of the upper flow channel 48 via the first port 56, of the bioreactor 36. In particular, the third valve V3 is operated to fluidly couple a selected one of the media sources s4-s7 to the third port 60 of the bioreactor 36, and the first valve V1 is operated to fluidly couple the media destination d1 to the first port 56 of the bioreactor 36 (effectively opening the third and first ports 60, 56 between the selected one of the media sources s4-s7 and the media destination d1), while the second valve V2 is operated to fluidly decouple the media destination d2 and the first valve V1 from the second port 58 of the bioreactor 36, and the fourth valve V4 is operated to fluidly decouple the media destination d2 and the third valve V3 from the fourth port 62 of the bioreactor 36 (effectively closing the second and fourth ports 58, 62). Both the first pump P1 and the second pump P2 are turned on.

The seventh flow path 86g may be uni-directional in that media can flow along the seventh flow path 86g in only one direction, and in this case, from the third port 60 to the first port 56. As a result, under fluid pressure applied by the second pump P2, media flows from a selected one of the media sources s4-s7 along the seventh flow path 86g, into the lower flow channel 50 via the third port 60, perfuses through the porous membrane 46 from the lower flow channel 50 into the upper flow channel 48 (due to the pressure buildup in the lower flow channel 50 caused by the closed fourth port 62 that creates a pressure gradient between the flow channels 48, 50), and out of the upper flow channel 48 via the first port 56 to the media destination d1.

The seventh flow path 86g may be used to perform such functions as, e.g., harvesting or sampling of the biological cells 2 from the bioreactor 36. For example, one of the media sources s4-s7 selected by the second valve V2 may be saline, and the media destination d1 may be a biological cell collection region or an analytic region, such that the saline may flow along the seventh flow path 86g into the lower flow channel 50, perfuse through the porous membrane 46 from the lower flow channel 50 to the upper flow channel 48, and then out of the upper flow channel 48 (carrying at least some of the biological cells 2 out of the upper flow channel 48) to the biological cell collection region or analytic region.

Although the valves V1-V4 have been described as being operated to define only one flow path at any given time, it should be appreciated that the valves V1-V4 may be operated to define multiple flow paths at any given time.

For example, in one arrangement illustrated in FIG. 19H, the valves V1-V4 may be operated to define the third flow path 86c entirely through the lower flow channel 50 via the third and fourth ports 60, 62 of the bioreactor 36, as well as to define the fifth flow path 86e into the upper flow channel 48 via the first port 56, between the upper and lower flow channels 48, 50 via the porous membrane 46, and out of the lower flow channel 50 via the fourth port 62, of the bioreactor 36. In particular, the third valve V3 is operated to fluidly couple a selected one of the media sources s4-s7 to the third port 60 of the bioreactor 36, the first valve V1 is operated to fluidly couple a selected one of the media sources s1-s3 to the first port 56 of the bioreactor 36, and the fourth valve V4 is operated to fluidly couple the media destination d2 to the fourth port 62 of the bioreactor 36 (effectively opening the first and fourth ports 56, 62 between the selected one of the media sources s1-s3, a selected one of the media sources s4-s7, and the media destination d2), while the second valve V2 is operated to fluidly decouple the media destination d2 and the first valve V1 from the second port 58 of the bioreactor 36 (effectively closing the second ports 58). Both the first pump P1 and the second pump P2 are turned on.

The third and fifth flow paths 86c, 86e may be used in combination to perform such functions as, e.g., suspending the biological cells 2 and/or microbeads 4 within the upper flow channel 48 to facilitate clogless perfusion of media from the upper flow channel 48 to the lower flow channel 50 via the porous membrane 46 during seeding of the bioreactor 36 with biological cells 2 and/or microbeads 4 or when washing the biological cells 2 and/or microbeads 4. For example, one of the media sources s1-s3 selected by the first valve V1 may be media containing biological cells 2 and/or microbeads 4, and the media destination d2 may be waste, such that the media containing the biological cells 2 and/or microbeads 4, may flow along the fifth flow path 86e into the upper flow channel 48, perfuse through the porous membrane 46 from the upper flow channel 48 to the lower flow channel 50 (without the biological cells 2 and/or microbeads 4), and then out of the lower flow channel 50 to waste. One of the media sources s4-s7 selected by the third valve V3 may be saline, and the media destination d2 may be waste, such that the saline may flow along the third flow path 86c entirely through the lower flow channel 50 to waste. The first pump P1 may pump the media containing biological cells 2 and/or microbeads 4 along the fifth flow path 86e in a continuous manner, while the second pump P2 may pump the saline along the third flow path 86c in a pulsatile or cyclical manner, such that pressure gradient between the flow channels 48, 50 periodically reverse. In this manner, the biological cells 2 and/or microbeads 4 may be periodically suspended in the upper flow channel 48, thereby allowing the media to more freely be perfused from the upper flow channel 48 to the lower flow channel 50 via the porous membrane 46.

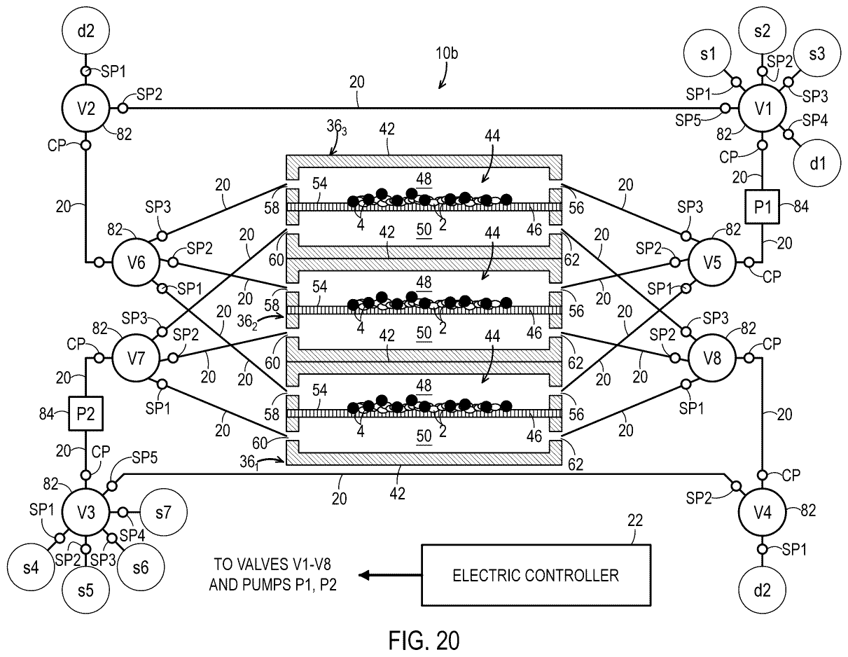

As described herein, multiple bioreactor units 36 (in this case, three bioreactor units 36₁-36₃) may be stacked in a bioreactor assembly 12 to provide a scalable bioreactor system 10b, as illustrated in FIG. 20. In this embodiment, the bioreactor units 36₁-36₃ are similar in structure and function as the bioreactor unit 36a illustrated in FIGS. 5-6, although it should be appreciated that the bioreactor units 36₁-36₃ can have any suitable structure or functions, e.g., similar in structure and function as the bioreactor unit 36b illustrated in FIG. 13.

Rather than selectively defining different flow paths between various ones of the media sources s1-s7 and media destinations d1-d2 as performed in the bioreactor system 10a illustrated in FIG. 18, the valves V1-V4 are merely configured for selecting the media sources s1-s7 and media destinations d1-d2 to a particular flow path, whereas a second set of four selector valves 82 (V5-V8) are fluidly coupled between the valves V1-V4 and the bioreactor units 36₁-36₃ for defining different flow paths between the media sources s1-s7 and media destinations d1-d2 selected by the valves V1-V4 and a selected one of the bioreactor units 36₁-36₃. To this end, each of the valves V5-V8 is associated with a particular one of the ports 56-62 associated with all three of the bioreactor units 36₁-36₃ (i.e., the fifth valve V5 is associated with the first ports 56 of all three bioreactor units 36₁-36₃; the sixth valve V6 is associated with the second ports 58 of all three bioreactor units 36₁-36₃; the seventh valve V7 is associated with the third ports 60 of all three bioreactor units 36₁-36₃; and the eighth valve V8 is associated with the fourth ports 62 of all three bioreactor units 36₁-36₃).

In particular, the fifth valve V5 comprises a common port CP fluidly connected to the common port CP of the first valve V1 via the first pump P1, and a plurality of selector ports SP1-SP3 respectively fluidly connected to the first ports 56 of the bioreactor units 36₁-36₃. The sixth valve V6 comprises a common port CP fluidly connected to the common port CP of the second valve V2, and a plurality of selector ports SP1-SP3 respectively fluidly connected to the second ports 58 of the bioreactor units 36₁-36₃. The seventh valve V7 comprises a common port CP fluidly connected to the common port CP of the third valve V3, and a plurality of selector ports SP1-SP3 respectively fluidly connected to the third ports 60 of the bioreactor units 36₁-36₃. The eighth valve V8 comprises a common port CP fluidly connected to the common port CP of the fourth valve V4, and a plurality of selector ports SP1-SP3 respectively fluidly connected to the fourth ports 62 of the bioreactor units 36₁-36₃.

Thus, the first valve V1 may be operated to fluidly couple a selected one of the media sources s1-s3, the media destination d1, and the second port 58 of the selected bioreactor unit 36₁-36₃ (via the second valve V2) to (or fluidly decouple all of the media sources s1-s3, media destination d1, and second port 58 of the selected bioreactor unit 36₁-36₃ from) the common port CP of the fifth valve V5. The second valve V2 may be operated to fluidly couple a selected one of the media destination d2 and the first port 56 of the selected bioreactor unit 36₁-36₃ (via the first valve V1) to (or fluidly decouple both of the media destination d2 and first port 56 of the selected bioreactor unit 36₁-36₃ from) the common port CP of the sixth valve V6. The third valve V3 may be operated to fluidly couple a selected one of the media sources s4-s7 and fourth port 62 of the selected bioreactor unit 36₁-36₃ (via the fourth valve V4) to (or fluidly decouple all of the media sources s4-s7 and fourth port 62 of the selected bioreactor unit 36₁-36₃ from) the common port CP of the seventh valve S7. The fourth valve V4 may be operated to fluidly couple a selected one of the media destination d2 and the third port 60 of the selected bioreactor unit 36₁-36₃ (via the third valve V3) to (or fluidly decouple both of the media destination d2 and third port 60 of the selected bioreactor unit 36₁-36₃ from) the common port CP of the eight valve V8.

The fifth valve V5 may be operated to select one of the bioreactor units 36₁-36₃, such that one of the media sources s1-s3, the media destination d1, and the second port 58 selected by the first valve V1 is fluidly coupled to the first port 56 of the selected one of the bioreactor units 36₁-36₃. The sixth valve V6 may be operated to select one of the bioreactor units 36₁-36₃, such that one of the media destination d2 and second port 58 selected by the second valve V2 is fluidly coupled to the first port 56 of the selected one of the bioreactor units 36₁-36₃. The seventh valve V7 may be operated to select one of the bioreactor units 36₁-36₃, such that one of the media sources s1-s3 and third second port 58 selected by the third valve V3 is fluidly coupled to the third port 60 of the selected one of the bioreactor units 36₁-36₃. The eighth valve V8 may be operated to select one of the bioreactor units 36₁-36₃, such that one of the media destination d2 and third port 60 selected by the fourth valve V4 is fluidly coupled to the fourth port 62 of the selected one of the bioreactor units 36₁-36₃.

Thus, it can be appreciated that the valves V1-V8 can be collectively controlled by the electric controller 22 select the media sources s1-s7 and media destinations d1, and to define a particular flow path between the selected ones of the media sources s1-s7 and media destinations d1-d2 and through the interior cavity 44 of a selected one of the bioreactor units 36₁-36₃. In the same manner described above with respect to the bioreactor system 10a illustrated in FIG. 18, the pumps P1, P2 are configured for providing the necessary fluid pressure for conveying various media along flow paths 86a-86h for the purposes of (1) priming the bioreactor assembly 12 (i.e., removing all air within the bioreactor assembly 12); (2) seeding the bioreactor assembly 12 with a population of biological cells (with or without microbeads); (3) culturing the biological cells, such that the population of biological cells are expanded; washing the biological cells; (4) harvesting or sampling the expanded population of biological cells from the bioreactor assembly 12; and (5) clearing the fluid conduits 20 (i.e., relocating stray biological cells from the fluid conduits 20 back into the bioreactor assembly 12).

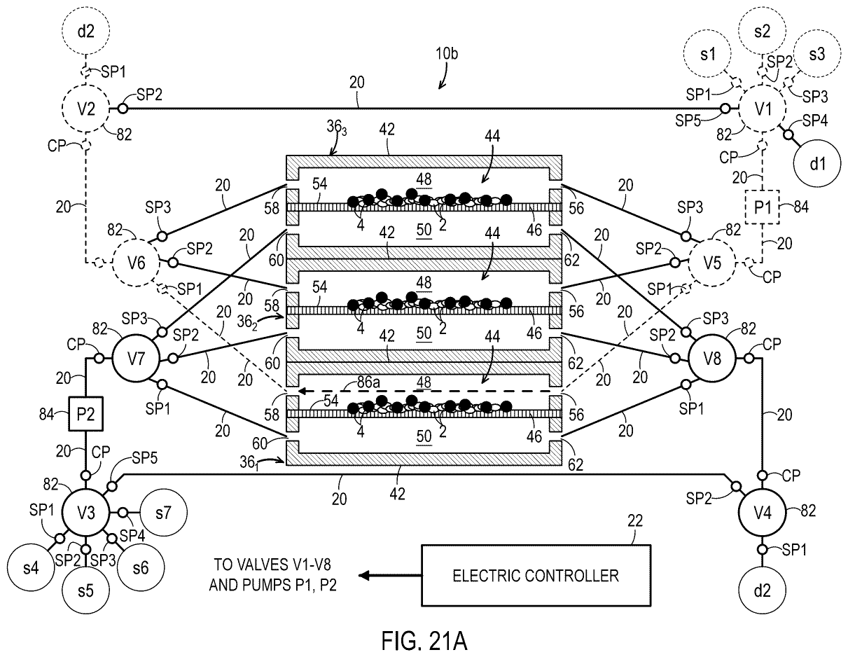

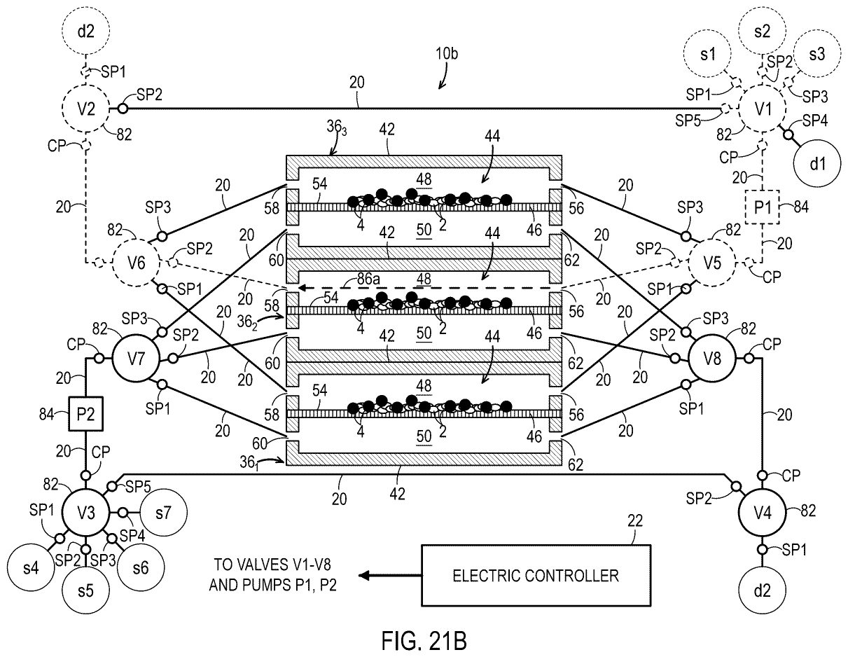

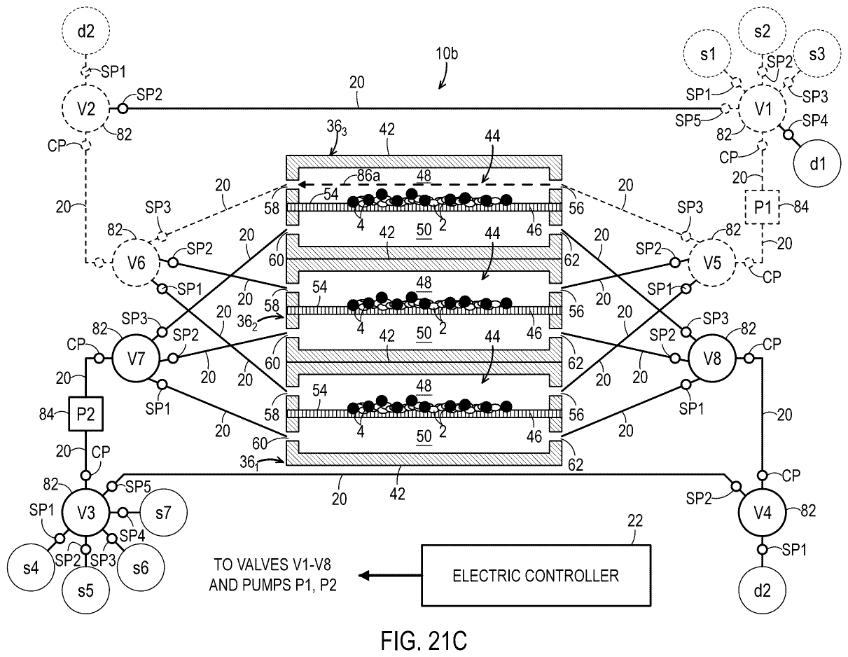

For example, referring to FIGS. 21A-21C, the valves V1-V8 are operated in conjunction with the media sources s1-s7 and media destinations d1-d2 in a variety of arrangements to define a particular flow path through a selected one of the bioreactor units 36. Notably, the particular media sources s1-s7, media destinations d1-d2, valves V1-V4, pumps P1, P2, and fluid conduits 20 from, through, and to which media associated with a particular flow is shown dashed for purposes of illustration in FIGS. 21A-21C.

In all of the arrangements illustrated in FIGS. 21A-21C, the valves V1-V8 may be operated to define the first flow path 86a entirely through the upper flow channels 48 of bioreactor units 36 one at a time via their first and second ports 56, 58 (e.g., to perform such functions as priming of the bioreactor units 36 in the same manner described above with respect to bioreactor 36 illustrated in FIG. 19A).

In particular, the third valve V3 is operated to fluidly decouple all of the media sources s4-s7 and fourth valve V4 from the common port CP of the seventh valve V7, which is operated to fluidly decouple the third valve V3 from the third ports 60 of all of the bioreactor units 36₁-36₃, while the fourth valve V4 is operated to fluidly decouple the media destination d2 and third valve V3 from the common port CP of the eighth valve V8, which is operated to fluidly decouple the fourth valve V4 from the fourth ports 62 of all of the bioreactor units 36₁-36₃ (effectively closing the third and fourth ports 60, 62 of all of the bioreactor units 36₁-36₃). The first pump P1 is turned on, while the second pump P2 is turned off.

The first valve V1 is operated to fluidly couple a selected one of the media sources s1-s3 to the common port CP of the fifth valve V5, while the second valve V2 is operated to fluidly couple the media destination d2 to the common port CP of the sixth valve V6. In the arrangement illustrated in FIG. 21A, the fifth valve V5 is operated to fluidly couple the media source s1-s3 selected by the first valve V1 to the first port 56 of first bioreactor unit 36₁, while the sixth valve V6 is operated to fluidly couple the media destination d2 selected by the second valve V2 to the second port 58 of the first bioreactor unit 36₁ (effectively opening the first and second ports 56, 58 of the first bioreactor unit 36₁ between the selected one of the media sources s4-s7 and the media destination d2). In the arrangement illustrated in FIG. 21B, the fifth valve V5 is operated to fluidly couple the media source s1-s3 selected by the first valve V1 to the first port 56 of the second bioreactor unit 36₂, while the sixth valve V6 is operated to fluidly couple the media destination d2 selected by the second valve V2 to the second port 58 of the second bioreactor unit 36₂ (effectively opening the first and second ports 56, 58 of the second bioreactor unit 36₂ between the selected one of the media sources s4-s7 and the media destination d2). In the arrangement illustrated in FIG. 21C, the fifth valve V5 is operated to fluidly couple the media source s1-s3 selected by the first valve V1 to the first port 56 of the third bioreactor unit 36₃, while the sixth valve V6 is operated to fluidly couple the media destination d2 selected by the second valve V2 to the second port 58 of the third bioreactor unit 36₃ (effectively opening the first and second ports 56, 58 of the third bioreactor unit 36₃ between the selected one of the media sources s4-s7 and the media destination d2).

The other flow paths 86b-86h may be defined through selected ones of the bioreactor units 36₁-36₃ by operated the valves V1-V4 in a similar manner as described above with respect to FIGS. 19B-19H, while operating the valves V5-V8 to select the bioreactor unit 36₁-36₃ through which each of the flow paths 86b-86h will extend. In alternative embodiments, a particular one of the flow paths 86b-86h may be defined through a series of two or more of the bioreactor units 36₁-36₃, in which case, additional or more complex selector valves (not shown) may be employed.

It should be appreciated that, as a general rule, it is desirable to minimize the number of valves and fluid conduits employed to selectively define the flow paths 86a-86h through a particular bioreactor (e.g., the single bioreactor 36 illustrated in FIG. 18) or through a selected one or combination of multiple bioreactor units (e.g., the bioreactor units 36₁-36₃ illustrated in FIG. 20), thereby decreasing the size and cost, while increasing the reliability, of the resulting bioreactor system.

Multi-function valves that minimize the number of valves and fluid conduits employed to selectively define the flow paths 86a-86h through a bioreactor will now be described. Each multi-function valve described herein advantageously combines the functions that would typically be performed by two valves with respect to controlling the source, destination, and flow path of media through the bioreactor.

Each multi-function valve comprises a first valve portion capable of fluidly coupling a common port to each of a plurality of selector ports (either input ports, output ports, or a combination thereof), and a second valve portion capable of either selectively fluidly coupling an output port to one or more return ports or selectively fluidly decoupling the output port to the return port(s) while the first valve portion fluidly couples the common port to each of the plurality of selector ports. In other words, the multi-function valve has a first plurality of positions in which the common port is respectively fluidly coupled to the plurality of selector ports while the output port and return port(s) are fluidly coupled to each other, and a second plurality of positions in which the common port is respectively fluidly coupled to the plurality of selector ports while the output port and return port(s) are fluidly decoupled from each other.

One embodiment of a multi-function valve described herein is operated to fluidly couple one of a plurality of different types of media sources and/or destination sources to a first port associated with a flow channel of a bioreactor, while fluidly coupling a media destination to a second port associated with the flow channel of the bioreactor or decoupling the media destination from the second port associated with the flow channel of the bioreactor. Such fluid coupling to (or fluidly decoupling from) a flow channel of the bioreactor may be made directly in the case of a stand-alone bioreactor or may be made indirectly through another valve arrangement in the case of multiple bioreactor units (e.g., in a stacked bioreactor assembly).

One of these multi-function valves can be associated with each flow channel of a bioreactor, such that the multi-function valves are operated together to perform a particular function with respect to the bioreactor (e.g., seeding, culturing, washing, or harvesting/sampling of biological cells) by controlling the flow of media in and out of the flow channels, thereby defining the particular flow path through the bioreactor, as well as the type of media that will flow along the particular flow path. In some embodiments, the multi-function valves associated with a bioreactor may even directly couple the ports of a particular flow channel to each other, thereby bypassing the media sources and media destination (e.g., to define flow paths 86b or 86d), such that media may be recycled along a particular flow path of the bioreactor. Each bioreactor described herein has two flows channels, and thus, the number of valves required to perform the media and flow path selection functions with respect to this bioreactor may be reduced from four conventional valves to two of the multi-function valves described herein.

Another embodiment of a multi-function valve described herein is operated to select a bioreactor unit, or different combinations of bioreactor units, through which media may be flowed, e.g., in a stacked bioreactor assembly. One of these multi-function valves can be associated with each set of corresponding flow channels of the bioreactor units within the stacked bioreactor assembly, such that, when combined with the multi-function valves briefly described above, may perform a particular function (e.g., seeding, culturing, washing, or harvesting/sampling of biological cells) in the selected bioreactor unit or selected combination of bioreactor units. In the case where each bioreactor units has two flow channels, the number of valves required to perform the bioreactor selection function may be reduced from eight conventional valves to four of the multi-function valves described herein regardless of the number of bioreactor units. The stacked bioreactor assembly may be scaled up or scaled down without changing the number of such multi-function valves.

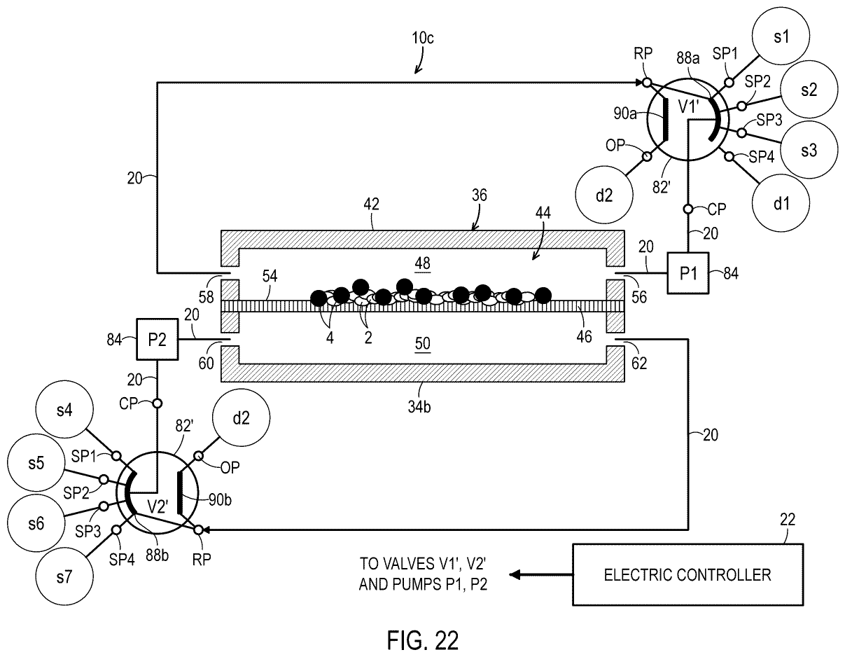

Referring to FIG. 22, one embodiment of a bioreactor system 10c constructed in accordance with the present inventions will be described. The bioreactor system 10c is similar to the bioreactor system 10a illustrated in FIG. 18 in that it generally comprises a single bioreactor 36, a plurality of media sources s1-s7, a plurality of media destinations d1-d2, two pumps 84 corresponding to the pump assembly 18 illustrated in FIG. 1 (in particular, a first pump P1 and a second pump P2), the arrangement of fluid conduits 20, and the electric controller 22. The bioreactor system 10c differs from the bioreactor system 10a illustrated in FIG. 18 in that, instead of comprising four single-function valves 82 (V1-V4), the bioreactor system 10c comprises two multi-function valves 82′ corresponding to the valve assembly 16 illustrated in FIG. 1 (in particular, a first multi-function valve V1′ and a second multi-function valve V2′).

In the illustrated embodiment, the first multi-function valve V1′ comprises a common port CP fluidly connected to the first port 56 of the bioreactor 36 via the first pump P1; a return port RP fluidly connected to the second port 58 of the bioreactor 36; a plurality of selector ports SP1-SP4 (three input ports SP1-SP3 and one output port SP4) respectively fluidly connected to the media sources s1-s3 and the media destination d1; and an output port OP fluidly connected to the media destination d2. Similarly, the second multi-function valve V2′ comprises a common port CP fluidly connected to the third port 60 of the bioreactor 36 via the second pump P2; a return port RP fluidly connected to the fourth port 62 of the bioreactor 36; a plurality of selector ports SP1-SP4 (all input ports) respectively fluidly connected to the media sources s4-s7; and an output port OP fluidly connected to the media destination d2.

The first valve V1′ combines the functions of the first and second valves V1, V2 illustrated in FIG. 18 in that it may be operated to both fluidly couple a selected one of the media sources s1-s3, the media destination d1, and the second port 58 of the bioreactor 36 to (or fluidly decouple all of the media sources s1-s3, media destination d1, and second port 58 of the bioreactor 36 from) the first port 56 of the bioreactor 36, and to fluidly couple a selected one of the media destination d2 and the first port 56 of the bioreactor 36 to (or fluidly decouple both of the media destination d2 and first port 56 of the bioreactor 36 from) the second port 58 of the bioreactor 36. Similarly, the second valve V2′ combines the functions of the second and third valves V3, V4 illustrated in FIG. 18 in that it may be operated to both fluidly couple a selected one of the media sources s4-s7 and fourth port 62 of the bioreactor 36 to (or fluidly decouple all of the media sources s4-s7 and fourth port 62 of the bioreactor 36 from) the third port 60 of the bioreactor 36, and to fluidly couple a selected one of the media destination d2 and the third port 60 of the bioreactor 36 to (or fluidly decouple both of the media destination d2 and third port 60 of the bioreactor 36 from) the fourth port 62 of the bioreactor 36.

To this end, the first multi-function valve V1′ comprises a first valve portion 88a that selectively fluidly couples its selector ports SP1-SP4 and return port RP to its common port CP, such that the media sources s1-s3, media destination d1, and second port 58 are fluidly coupled to the first port 56 of the bioreactor 36 (or fluidly decouples all of its selector ports SP1-SP4 and return port RP from its common port CP, such that all of the media sources s1-s3, media destination d1, and second port 58 of the bioreactor 36 are fluidly decoupled from the first port 56 of the bioreactor 36). The first multi-function valve V1′ further comprises a second valve portion 90a that either fluidly couples its output port OP to its return port RP, such that the media destination d2 is fluidly coupled to second port 58 of the bioreactor 36, or fluidly decouples its output port OP from its return port RP, such that the media destination d2 is fluidly decoupled from the second port 58 of the bioreactor 36.

Likewise, the second multi-function valve V2′ further comprises a first valve portion 88b that selectively fluidly couples its selector ports SP1-SP4 and return port RP to its common port CP, such that media sources s4-s7 and fourth port 62 are fluidly coupled to the third port 60 of the bioreactor 36 (or fluidly decouples all of its selector ports SP1-SP4 and return port RP from its common port CP, such that all of the media sources s4-s7 and fourth port 62 of the bioreactor 36 are fluidly decoupled from the third port 60 of the bioreactor 36). The second multi-function valve V2′ further comprises a second valve portion 90b that either fluidly couples its output port OP to its return port RP, such that the media destination d2 is fluidly coupled to fourth port 62 of the bioreactor 36, or fluidly decouples its output port OP from its return port RP, such that the media destination d2 is fluidly decoupled from the fourth port 62 of the bioreactor 36.

The first and second valve portions 88a, 90a of the first multi-function valve V1′ may be mechanically synchronized with each other, such that the first multi-function valve V1′ may be placed into one of several positions that facilitate coordinated fluid couplings (or fluid decouplings) between its selector ports SP1-SP4 and its common port CP, a fluid coupling (or fluid decoupling) between its output port OP and its return port RP, and a fluid coupling (or fluid decoupling) between its return port RP and its common port CP. Likewise, the first and second valve portions 88b, 90b of the second multi-function valve V2′ may be mechanically synchronized with each other, such that the second multi-function valve V2′ may be placed into one of several positions that facilitate coordinated fluid couplings (or fluid decouplings) between its selector ports SP1-SP4 and its common port CP, a fluid coupling (or fluid decoupling) between its output port OP and its return port RP, and a fluid coupling (or fluid decoupling) between its return port RP and its common port CP.

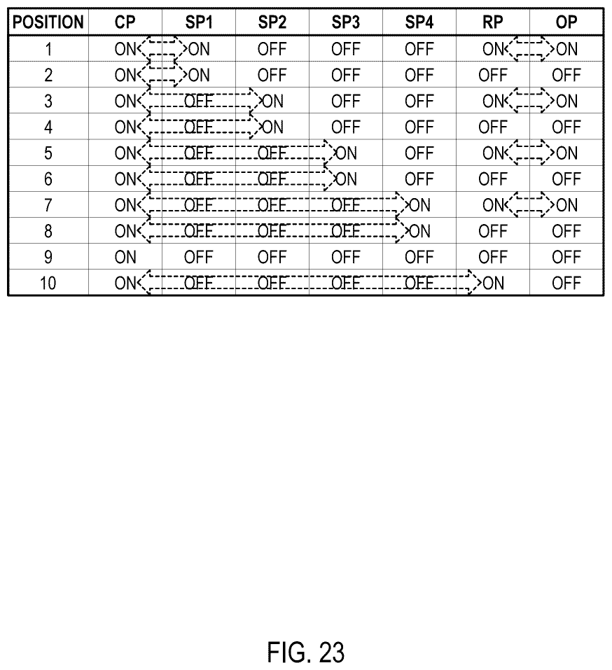

One exemplary embodiment of a multi-function valve that can be used as either of the first and second multi-function valves V1′, V2′ has a sequence of ten positions, as illustrated in FIG. 23. Notably, the arrows in FIG. 23 represent the fluid couplings between the respective ports. While the fluid couplings between the common port CP and the respective selector ports SP1-SP4 can be considered non-binary in nature, the fluid coupling between the return port RP and the output port OP may be considered binary in nature in that there is either a fluid coupling between the return port RP and the output port OP or no fluid coupling between the return port RP and the output port OP. It should be appreciated that, while the number of selector ports SP1-SP4 equals four, alternative embodiments of the multi-function valve may have more or less than four selector ports, including only one selector port, and may ultimately depend on the number of media sources/destinations that are to be fluidly coupled to the multi-function valve.

In Position 1, only the selector port SP1 is fluidly coupled to the common port CP, while the output port OP is fluidly coupled to the return port RP; in Position 2, only the selector port SP1 is fluidly coupled to the common port CP, while the output port OP is fluidly decoupled from the return port RP; in Position 3, only the selector port SP2 is fluidly coupled to the common port CP, while the output port OP is fluidly coupled to the return port RP; in Position 4, only the selector port SP2 is fluidly coupled to the common port CP, while the output port OP is fluidly decoupled from the return port RP; in Position 5, only the selector port SP3 is fluidly coupled to the common port CP, while the output port OP is fluidly coupled to the return port RP; in Position 6, only the selector port SP1 is fluidly coupled to the common port CP, while the output port OP is fluidly decoupled from the return port RP; in Position 7, only the selector port SP1 is fluidly coupled to the common port CP, while the output port OP is fluidly coupled to the return port RP; in Position 8, only the selector port SP4 is fluidly coupled to the common port CP, while the output port OP is fluidly decoupled from the return port RP; in Position 9, all of the selector ports SP1-SP4 are fluidly decoupled from the common port CP, while the outport port OP is fluidly decoupled from the return port RP; and in Position 10, all of the selector ports SP1-SP4 are fluidly decoupled from the common port CP, while the return port RP is fluidly coupled to the common port CP.

Thus, it can be appreciated from FIG. 23 that either of the multi-function valves V1′, V2′ may have a first plurality of positions (e.g., Positions 1, 3, 5 and 7) for respectively fluidly coupling its selector ports SP1-SP4 to its common port CP (thereby fluidly coupling the media sources s1-s3 and media destination d1 respectively to the first port 56 of the bioreactor 36 in the case of the first multi-function valve V1′ or fluidly coupling the media sources s4-s7 respectively to the third port 60 of the bioreactor 36 in the case of the second multi-function valve V2′), while fluidly coupling its output port OP to its return port RP (thereby fluidly coupling the media destination d2 to the second port 58 of the bioreactor 36 in the case of the first multi-function valve V1′ or fluidly coupling the media destination d1 to the fourth port 62 of the bioreactor 36 in the case of the second multi-function valve V2′), and while fluidly decoupling its return port RP from its common port CP (thereby directly fluidly decoupling the first and second ports 56, 58 of the bioreactor 36 from each other in the case of the first multi-function valve V1′, or directly fluidly decoupling the third and fourth ports 60, 62 of the bioreactor 36 from each other in the case of the second multi-function valve V2′).

Either of the multi-function valves V1′, V2′ may also have a second plurality of positions (e.g., Positions 2, 4, 6, and 8) for respectively fluidly coupling its selector ports SP1-SP4 to its common port CP (thereby fluidly coupling the media sources s1-s3 and media destination d1 respectively to the first port 56 of the bioreactor 36 in the case of the first multi-function valve V1′ or fluidly coupling the media sources s4-s7 respectively to the third port 60 of the bioreactor 36 in the case of the second multi-function valve V2′), while fluidly decoupling its output port OP from its return port RP (thereby fluidly decoupling the media destination d2 from the second port 58 of the bioreactor 36 in the case of the first multi-function valve V1′ or fluidly decoupling the media destination d2 from the fourth port 62 of the bioreactor 36 in the case of the second multi-function valve V2′), and while fluidly decoupling its return port RP from its common port CP (thereby directly fluidly decoupling the first and second ports 56, 58 of the bioreactor 36 from each other in the case of the first multi-function valve V1′, or directly fluidly decoupling the third and fourth ports 60, 62 of the bioreactor 36 from each other in the case of the second multi-function valve V2′).

Either of the multi-function valves V1′, V2′ may also have a third position (e.g., Position 9) for fluidly decoupling all of its selector ports SP1-SP4 from its common port CP (thereby fluidly decoupling all of the media sources s1-s3 and media destination d1 from the first port 56 of the bioreactor 36 in the case of the first multi-function valve V1′ or fluidly decoupling all of the media sources s4-s7 from the third port 60 of the bioreactor 36 in the case of the second multi-function valve V2′), while fluidly decoupling its output port OP from its return port RP (thereby fluidly decoupling the media destination d2 from the second port 58 of the bioreactor 36 in the case of the first multi-function valve V1′ or fluidly decoupling the media destination d2 from the fourth port 62 of the bioreactor 36 in the case of the second multi-function valve V2′), and while fluidly decoupling its return port RP from its common port CP (thereby directly fluidly decoupling the first and second ports 56, 58 of the bioreactor 36 from each other in the case of the first multi-function valve V1′, or directly fluidly decoupling the third and fourth ports 60, 62 of the bioreactor 36 from each other in the case of the second multi-function valve V2′).

Either of the multi-function valves V1′, V2′ may optionally have a fourth position (e.g., Position 10) for fluidly decoupling all of its selector ports SP1-SP4 from its common port CP (thereby fluidly decoupling all of the media sources s1-s3 and media destination d1 from the first port 56 of the bioreactor 36 in the case of the first multi-function valve V1′ or fluidly decoupling all of the media sources s4-s7 from the third port 60 of the bioreactor 36 in the case of the second multi-function valve V2′), while fluidly decoupling its output port OP from its return port RP (thereby fluidly decoupling the media destination d2 from the second port 58 of the bioreactor 36 in the case of the first multi-function valve V1′ or fluidly decoupling the media destination d2 from the fourth port 62 of the bioreactor 36 in the case of the second multi-function valve V2′), and while fluidly coupling its return port RP to its common port CP (thereby directly fluidly coupling the first and second ports 56, 58 of the bioreactor 36 to each other in the case of the first multi-function valve V1′, or directly fluidly coupling the third and fourth ports 60, 62 of the bioreactor 36 to each other in the case of the second multi-function valve V2′).

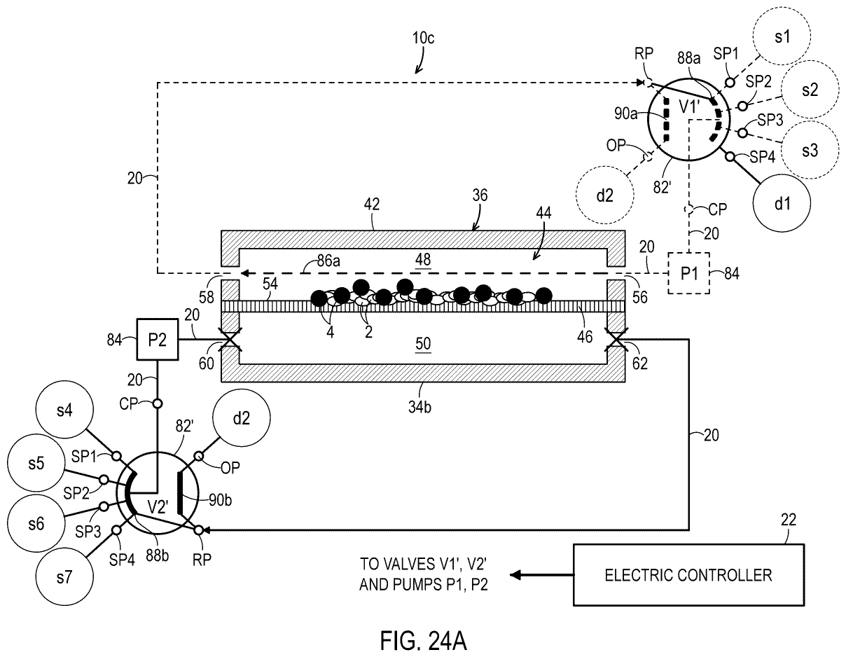

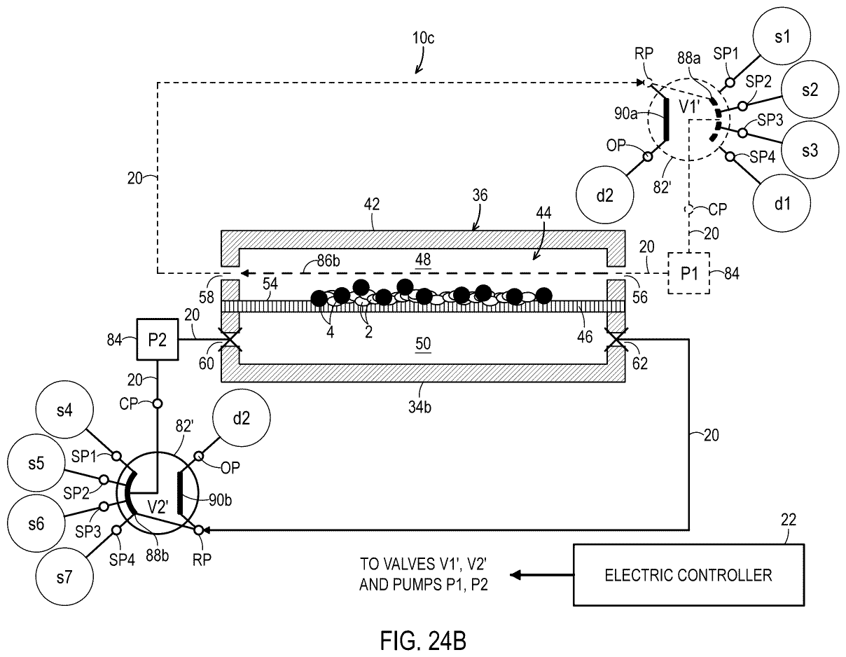

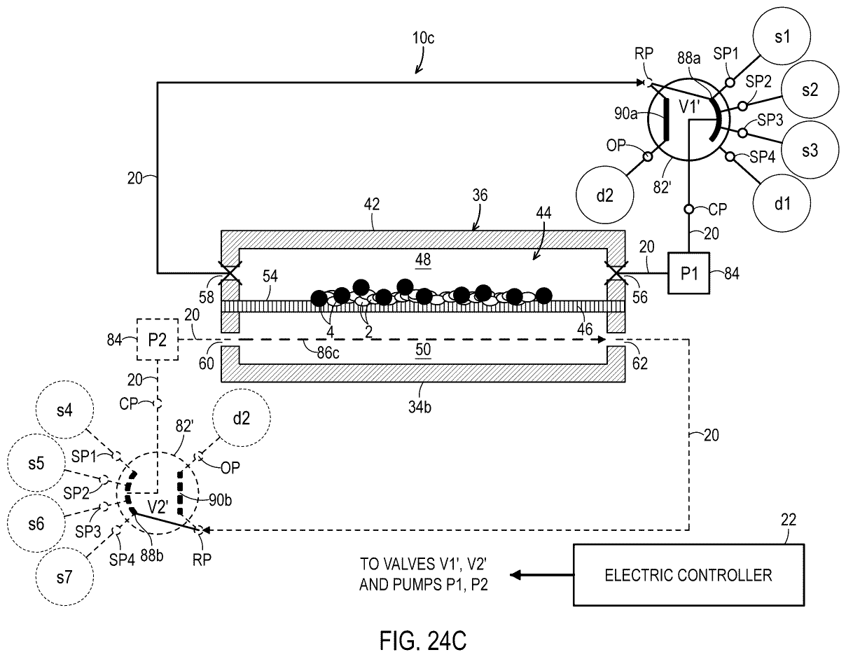

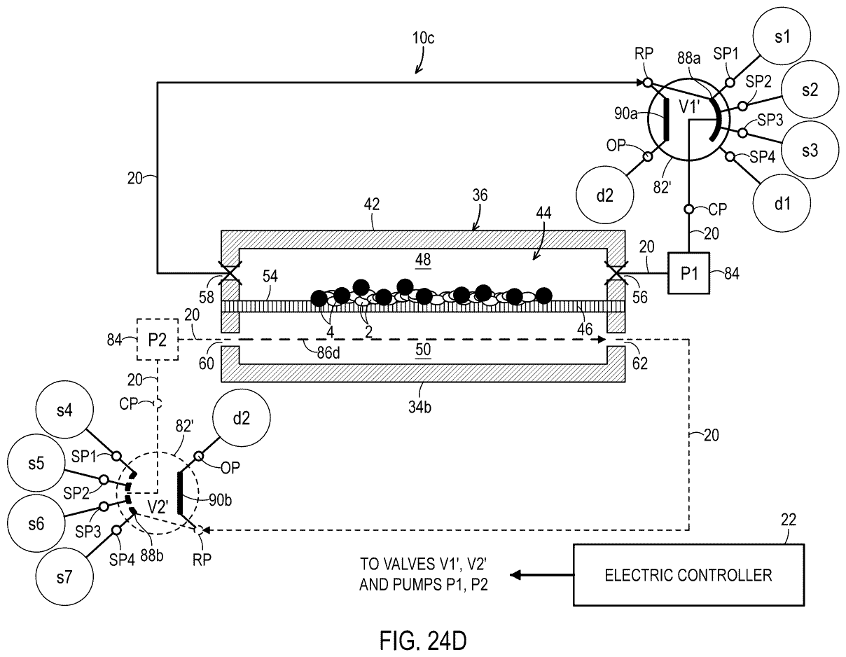

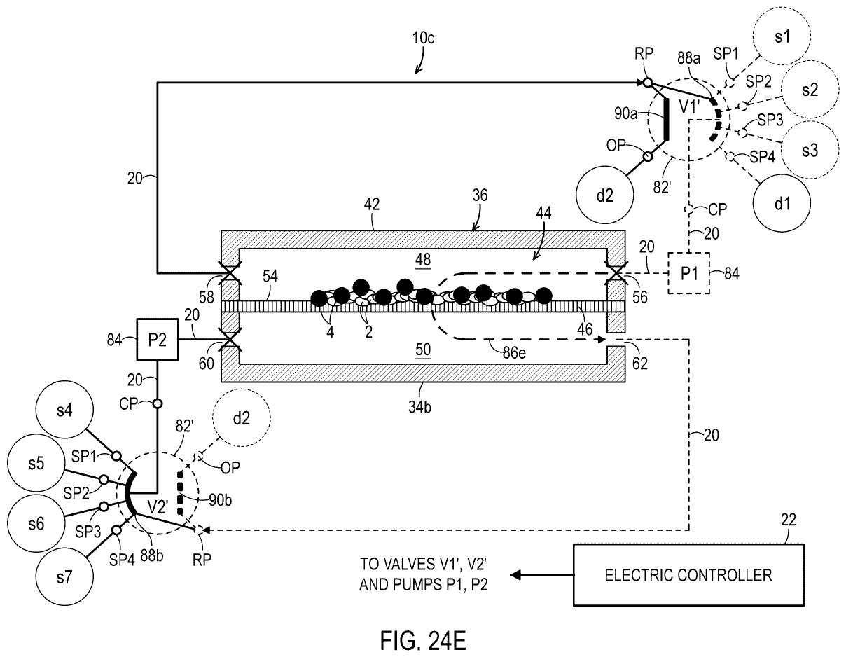

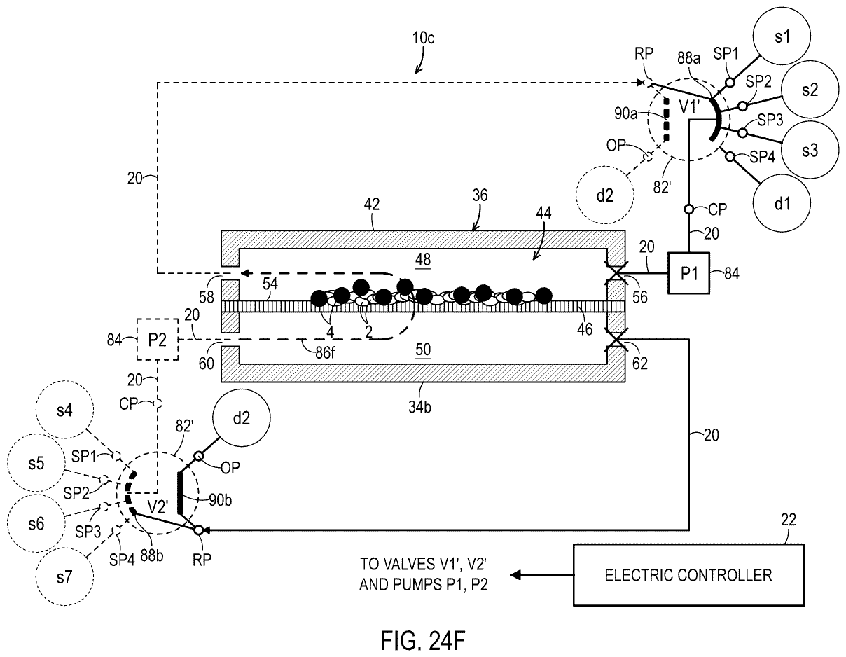

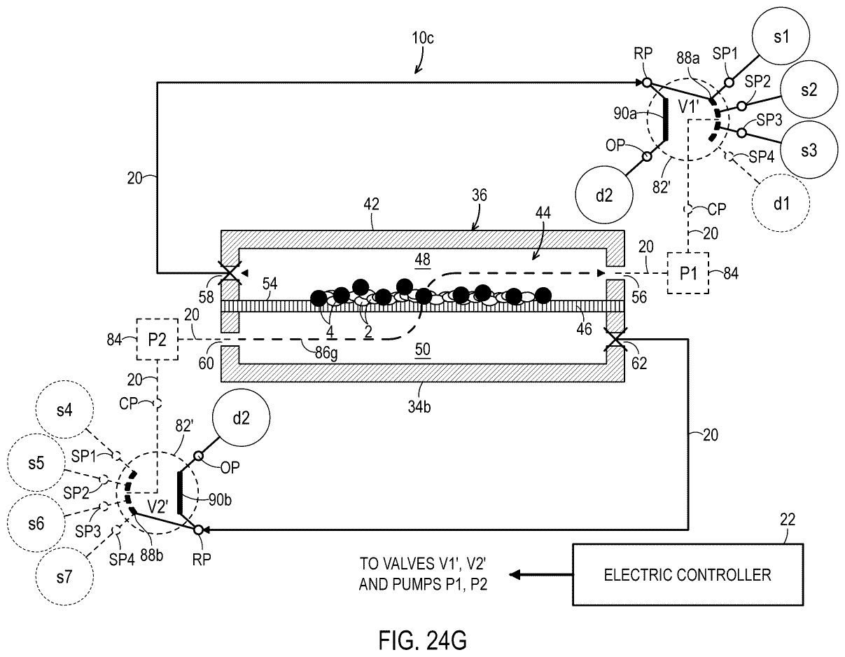

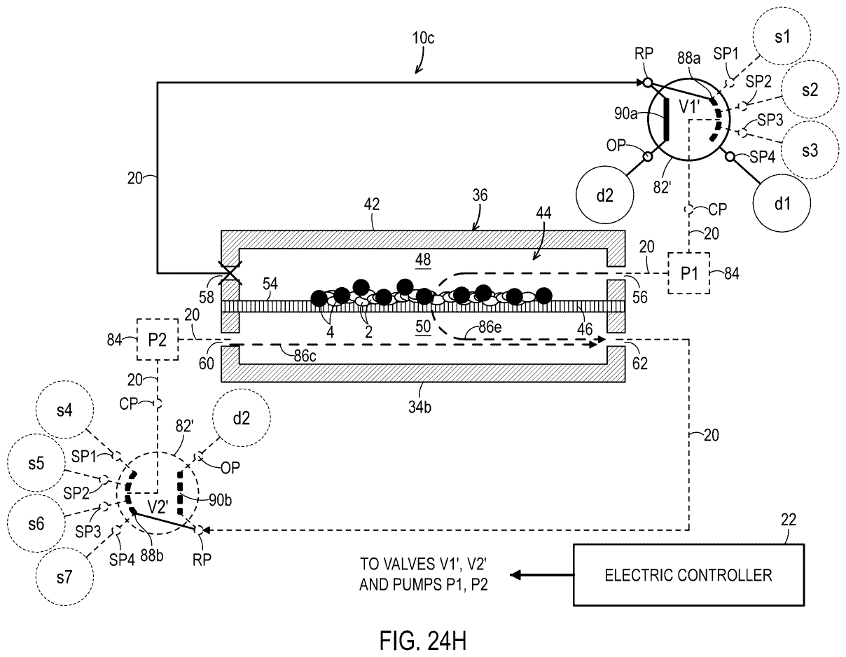

Referring to FIGS. 24A-24H, the first and second multi-function valves V1′, V2′ are operated in conjunction with the media sources s1-s7 and media destinations d1-d2 in a variety of arrangements to define various flow paths (and in particular the afore-described flow paths 86a-86h) through the bioreactor 36 to perform certain functions; e.g., (1) priming the bioreactor 36 (i.e., removing all air within the bioreactor 36); (2) seeding the bioreactor 36 with a population of biological cells (with or without microbeads); (3) culturing the biological cells, such that the population of biological cells are expanded; washing the biological cells; (4) harvesting or sampling the expanded population of biological cells from the bioreactor 36; and (5) clearing the fluid conduits 20 (i.e., relocating stray biological cells from the fluid conduits 20 back into the bioreactor 36). Notably, the particular media sources s1-s7, media destinations d1-d2, portions of the valves V1′, V2′, pumps P1, P2, and fluid conduits 20 from, through, and to which media associated with a particular flow is shown dashed for purposes of illustration in FIGS. 24A-24H.

In one arrangement illustrated in FIG. 24A, the multi-function valves V1′, V2′ may be operated to define the first flow path 86a entirely through the upper flow channel 48 via the first and second ports 56, 58 of the bioreactor 36 (e.g., to perform such functions as priming of the bioreactor 36 in the same manner described above with respect to bioreactor 36 illustrated in FIG. 19A).

In particular, the first multi-function valve V1′ is operated to fluidly couple a selected one of the media sources s1-s3 to the first port 56 of the bioreactor 36, and fluidly couple the media destination d2 to the second port 58 of the bioreactor 36, while the second multi-function valve V2′ is operated to fluidly decouple all of the media sources s4-s7 from the third port 60 of the bioreactor 36 and fluidly decouple the media destination d2 from the fourth port 62 of the bioreactor 36.

In this arrangement, the first multi-function valve V1′ may be placed in one of Positions 1, 3, or 5 (depending on the particular one of the media sources s1-s3 to be selected), and the second multi-function valve V2′ may be placed into Position 9 illustrated in FIG. 23, effectively opening the first and second ports 56, 58 between the selected one of the media sources s1-s3 and the media destination d2, closing the third port 60 to all of the media sources s4-s7, and closing the fourth port 62 to the media destination d2, as illustrated in FIG. 24A.

In another arrangement illustrated in FIG. 24B, the multi-function valves V1′, V2′ may be operated to define the second flow path 86b entirely through the upper flow channel 48 via the first and second ports 56, 58 of the bioreactor 36 that effectively bypasses the media sources s1-s3 and media destinations d1-d2 (e.g., to perform such functions as clearing the fluid conduits 20 respectively fluidly connected to the first and second ports 56, 58 of the bioreactor 36 in the same manner described above with respect to bioreactor 36 illustrated in FIG. 19B).

In particular, the first multi-function valve V1′ is operated to fluidly decouple all of the media sources s1-s3 and the media destination d1 from the first port 56 of the bioreactor 36, and fluidly decouple the media destination d2 from the second port 58 of the bioreactor 36, and the second multi-function valve V2′ may likewise be operated to fluidly decouple all of the media sources s4-s7 from the third port 60 of the bioreactor 36, and fluidly decouple the media destination d2 from the fourth port 62 of the bioreactor 36. The first multi-function valve V1′ may be further operated to fluidly couple the first and second ports 56, 58 of the bioreactor 36 to each other, and the second multi-function valve V2′ may be further operated to fluidly decouple the third and fourth ports 60, 62.

In this arrangement, the first multi-function valve V1′ may be placed into Position 10, and the second multi-function valve V2′ may be placed into Position 9 illustrated in FIG. 23, effectively closing the first and second ports 56, 58 to the media sources s1-s3 and media destination d1 and the media destination d2, but opening the first and second ports 56, 58 to each other, closing the third port 60 to all of the media sources s4-s7, and closing the fourth port 62 to the media destination d2, as illustrated in FIG. 24B.

In still another arrangement illustrated in FIG. 24C, the multi-function valves V1′, V2′ may be operated to define the third flow path 86c entirely through the lower flow channel 50 via the third and fourth ports 60, 62 of the bioreactor 36 (e.g., to perform such functions as priming of the base bioreactor unit 36₁ or culturing the biological cells 2 residing in the upper flow channel 48 on the cell culture surface 54 of the porous membrane 46 of the bioreactor 36 with fresh media containing nutrient, reagent, or gas in the same manner described above with respect to bioreactor 36 illustrated in FIG. 19C).

In particular, the first multi-function valve V1′ is operated to is operated to fluidly decouple all of the media sources s1-s3 and media destination d1 from the first port 56 of the bioreactor 36, and fluidly decouple the media destination d2 from the second port 58 of the bioreactor 36, and the second multi-function valve V2′ is operated to fluidly couple a selected one of the media sources s4-s7 to the third port 60 of the bioreactor 36 and fluidly couple the media destination d2 to the fourth port 62 of the bioreactor 36.

In this arrangement, the first multi-function valve V1′ may be placed into Position 9, and the second multi-function valve V2′ may be placed into any one of Positions 1, 3, 5, or 7 (depending on the particular one of the media sources s4-s7 to be selected) illustrated in FIG. 23, effectively opening the third and fourth ports 60, 62 between the selected one of the media sources s4-s7 and the media destination d2, closing the first port 56 to all of the media sources s1-s3 and media destination d1, and closing the second port 58 to the media destination d2, as illustrated in FIG. 24C.

In yet another arrangement illustrated in FIG. 24D, the multi-function valves V1′, V2′ may be operated to define the fourth flow path 86d entirely through the upper flow channel 48 via the first and second ports 56, 58 of the bioreactor 36 that effectively bypasses the media sources s4-s7 and media destination d2 (e.g., to perform such functions as recirculating media containing nutrient, reagent, and gas through the lower flow channel 50 of the bioreactor 36 in the same manner described above with respect to bioreactor 36 illustrated in FIG. 19D).

In particular, the first multi-function valve V1′ is operated to fluidly decouple all of the media sources s1-s3 and the media destination d1 from the first port 56 of the bioreactor 36, and fluidly decouple the media destination d2 from the second port 58 of the bioreactor 36, and the second multi-function valve V2′ may likewise be operated to fluidly decouple all of the media sources s4-s7 from the third port 60 of the bioreactor 36, and fluidly decouple the media destination d2 from the fourth port 62 of the bioreactor 36. However, the first multi-function valve V1′ may be further operated to fluidly decouple the first and second ports 56, 58 of the bioreactor 36 from each other, and the second multi-function valve V2′ may be further operated to fluidly couple the third and fourth ports 60, 62.

In this arrangement, the first multi-function valve V1′ may be placed into Position 9, and the second multi-function valve V2′ may be placed into Position 10 illustrated in FIG. 23, effectively closing the third and fourth ports 60, 62 to the media sources s1-s4 and the media destination d2, but opening the third and fourth ports 60, 62 to each other, closing the first port 56 to all of the media sources s1-s3 and media destination d1, and closing the second port 58 to the media destination d2, as illustrated in FIG. 24D.

In yet another arrangement illustrated in FIG. 24E, the multi-function valves V1′, V2′ may be operated to define the fifth flow path 86e into the upper flow channel 48 via the first port 56, between the upper and lower flow channels 48, 50 via the porous membrane 46, and out of the lower flow channel 50 via the fourth port 62, of the bioreactor 36 (e.g., to perform such functions as seeding the bioreactor 36 with biological cells 2 and/or microbeads 4, washing the biological cells 2 and/or microbeads 4, or clearing the fluid conduit 20 fluidly connected to the first port 56 of the bioreactor 36 after sampling of the biological cells 2 in the same manner described above with respect to bioreactor 36 illustrated in FIG. 19E).

In particular, the first multi-function valve V1′ is operated to fluidly couple a selected one of the media sources s1-s3 to the first port 56 of the bioreactor 36, and fluidly decouple the media destination d2 from the second port 58 of the bioreactor 36, the second pump P2 is deactivated to fluidly decouple all of the media sources s4-s7 from the third port 60 of the bioreactor 36, and the second multi-function valve V2′ is operated to fluidly couple the media destination d2 to the fourth port 62 of the bioreactor 36.

In this arrangement, the first multi-function valve V1′ may be placed into any one of Positions 2, 4, or 6 (depending on the particular one of the media sources s1-s3 to be selected), the second multi-function valve V2′ may be placed into any one of Positions 1, 3, 5, and 7 illustrated in FIG. 23, effectively opening the first port 56 to the selected one of the media sources s1-s3, closing the second port 58 to the media destination d2, closing the third port 60 to all of the media sources s4-s7, and opening the fourth port 62 to the media destination d2, as illustrated in FIG. 24E.

In yet another arrangement illustrated in FIG. 24F, the multi-function valves V1′, V2′ may be operated to define the sixth flow path 86f into the lower flow channel 50 via the third port 60, between the lower and upper flow channels 50, 48 via the porous membrane 46, and out of the upper flow channel 48 via the second port 58, of the bioreactor 36 (e.g., to perform such functions as refreshing the media in the upper flow channel 48 or suspending the biological cells 2 and/or microbeads 4 in the upper flow channel 48 of the bioreactor 36 in the same manner described above with respect to bioreactor 36 illustrated in FIG. 19F).

In particular, the first pump P1 is deactivated to fluidly decouple all of the media sources s1-s3 and the media destination d1 from the first port 56 of the bioreactor 36, the first multi-function valve V1′ is operated to fluidly couple the media destination d2 to the second port 58 of the bioreactor 36, and the second multi-function valve V2′ is operated to fluidly couple a selected one of the media sources s4-s7 to the third port 60 of the bioreactor 36 and fluidly decouple the media destination d2 from the fourth port 62 of the bioreactor 36.

In this arrangement, the first multi-function valve V1′ may be placed into any one of Positions 1, 3, 5, or 7, and the second multi-function valve V2′ may be placed into any one of Positions 2, 4, 6, or 8 (depending on the particular one of the media sources s4-s7 to be selected) illustrated in FIG. 23, effectively opening the third port 60 to the selected one of the media sources s4-s7, closing the fourth port 62 to the media destination d2, closing the first port 56 to all of the media sources s1-s3 and media destination d1, and opening the second port 58 to the media destination d2, as illustrated in FIG. 24F.

In yet another arrangement illustrated in FIG. 24G, the multi-function valves V1′, V2′ may be operated to define the seventh flow path 86g into the lower flow channel 50 via the third port 60, between the lower and upper flow channels 50, 48 via the porous membrane 46, and out of the upper flow channel 48 via the first port 56, of the bioreactor 36 (e.g., to perform such functions as harvesting or sampling of the biological cells 2 from the bioreactor 36 in the same manner described above with respect to bioreactor 36 illustrated in FIG. 19G).

In particular, the first multi-function valve V1′ is operated to fluidly couple the media destination d1 to the first port 56 of the bioreactor 36, and fluidly decouple the media destination d2 from the second port 58 of the bioreactor 36, and the second multi-function valve V2′ is operated to fluidly couple a selected one of the media sources s4-s7 to the third port 60 of the bioreactor 36 and fluidly decouple the media destination d2 from the fourth port 62 of the bioreactor 36. Notably, both pumps P1, P2 should be operated at the same speed to establish the seventh flow path 86g.

In this arrangement, the first multi-function valve V1′ may be placed into Position 8, and the second multi-function valve V2′ may be placed into any one of Positions 2, 4, 6, or 8 (depending on the particular one of the media sources s4-s7 to be selected) illustrated in FIG. 23, effectively opening the third port 60 to the selected one of the media sources s4-s7, closing the fourth port 62 to the media destination d2, and opening the first port 56 to the media destination d1, and closing the second port 58 to the media destination d2, as illustrated in FIG. 24G.

Although the first and second multi-function valves V1′, V2′ have been described as being operated to define only one flow path at any given time, it should be appreciated that the first and second multi-function valves V1′, V2′ is operated to define multiple flow paths at any given time.

For example, in one arrangement illustrated in FIG. 24H, the multi-function valves V1′, V2′ may be operated to define the fifth flow path 86e into the upper flow channel 48 via the first port 56, between the upper and lower flow channels 48, 50 via the porous membrane 46, and out of the lower flow channel 50 via the fourth port 62, of the bioreactor 36, as well as to define the third flow path 86c entirely through the lower flow channel 50 via the third and fourth ports 60, 62 of the bioreactor 36 (e.g., to perform such functions as suspending the biological cells 2 and/or microbeads 4 within the upper flow channel 48 to facilitate clogless perfusion of media from the upper flow channel 48 to the lower flow channel 50 via the porous membrane 46 during seeding of the bioreactor 36 with biological cells 2 and/or microbeads 4 or when washing the biological cells 2 and/or microbeads 4 within the bioreactor 36 in the same manner described above with respect to bioreactor 36 illustrated in FIG. 19H).

In particular, the first multi-function valve V1′ is operated to fluidly couple a selected one of the media sources s1-s3 to the first port 56 of the bioreactor 36, and fluidly decouple the media destination d2 from the second port 58 of the bioreactor 36, and the second multi-function valve V2′ is operated to fluidly couple a selected one of the media sources s4-s7 to the third port 60 of the bioreactor 36 and fluidly couple the media destination d2 to the fourth port 62 of the bioreactor 36.

In this arrangement, the first multi-function valve V1′ may be placed into any one of Positions 2, 4, or 6 (depending on the particular one of the media sources s1-s3 to be selected), and the second multi-function valve V2′ may be placed into any one of Positions 1, 3, 5, or 7 (depending on the particular one of the media sources s4-s7 to be selected) illustrated in FIG. 23, effectively opening the third and fourth ports 60, 62 between the selected one of the media sources s4-s7 and the media destination d2, opening the first port 56 to the selected one of the media sources s1-s3, and closing the second port 58 to the media destination d2, as illustrated in FIG. 24H.

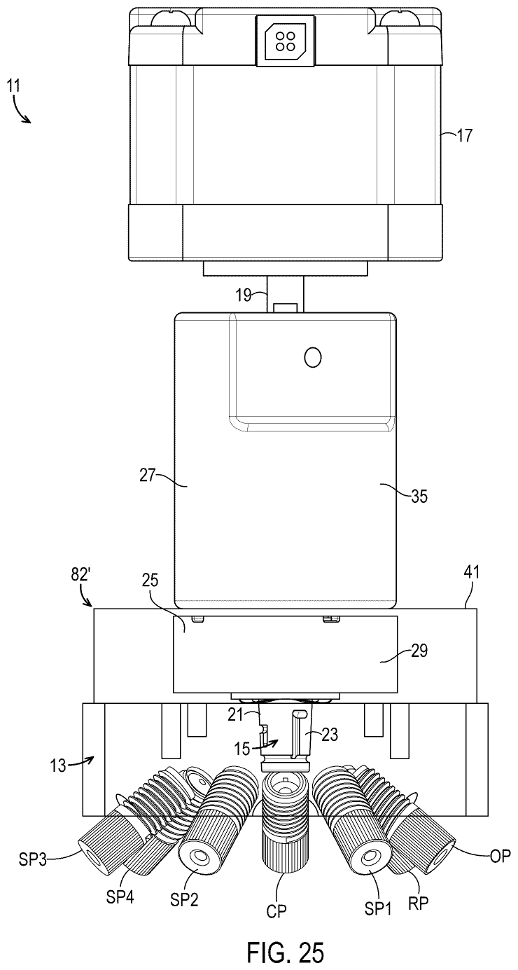

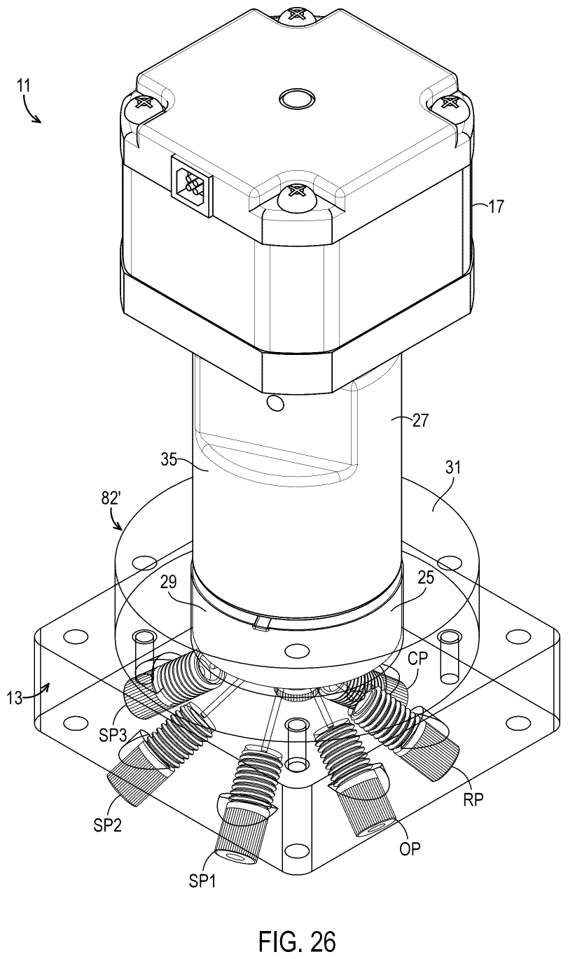

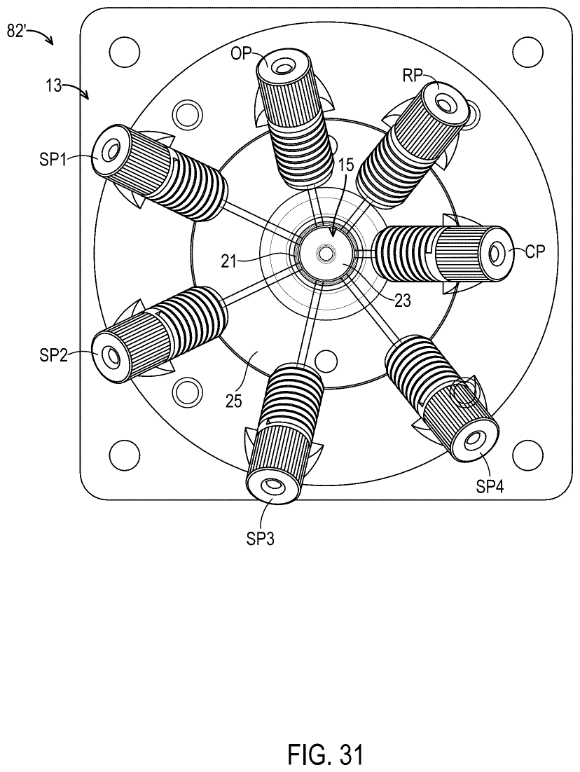

Referring now to FIGS. 25-31, one embodiment of a motorized valve assembly 11 for use in the bioreactor system 10c will now be described. The motorized valve assembly 11 comprises a rotary valve 82′ that can be employed as either of the multi-function valves V1′, V2′ of the bioreactor system 10c illustrated in FIG. 22. The rotary valve 82′ generally comprises a stator 13 and a rotor 15 configured for being rotated relative to the stator 13 in the afore-described positions. The rotary valve 82′ further comprises a common port CP, a return port RP, a plurality of selector ports SP1-SP4, and an output port OP affixed to the stator 13, such that selected ones of the ports can be fluidly coupled to each other via an open channel system (described in further detail below) formed on the outer surface of the rotor 15. In the illustrated embodiment, each of the common port CP, a return port RP, selector ports SP1 to SP4, and output port OP takes the form of a knurled fitting that can be threaded into a corresponding threaded bore formed into the stator 13. However, it should be appreciated that any of these ports can take the form of any port that is capable of fluidly communicating with the open channel system of the rotor 15 and to which fluid lines (e.g., the fluid conduits 20 described above) can be connected. The common port CP, return port RP, selector ports SP1 to SP4, and output port OP of the rotary valve 82′ can be fluidly connected, as either of the multi-function valves V1′, V2′, to the ports 56-62 of the bioreactor 36 in the same manner as that described above with respect to FIG. 22.

As best shown in FIGS. 25, 26, and 27, the motorized valve assembly 11 further comprises a motor 17 (e.g., a stepper motor) and a shaft 19 affixed between the motor 17 and the rotor 15 of the rotary valve 82′, such that operation of the motor 17 may rotate the rotor 15 relative to the stator 13. In the illustrated embodiment, one end of the shaft 19 is affixed to the motor 17, while the other end of the shaft 19 is magnetically coupled to the rotor 15 to mitigate the regulatory risks that may otherwise occur if the shaft 19 were in physical contact with the rotor 15 of the rotary valve 82′.

To this end, the stator 13 comprises a cylindrical cavity 21, while the rotor 15 comprises a cylindrical insert 23 configured for being rotatably disposed within the cylindrical cavity 21 of the stator 13, as best illustrated in FIGS. 25, 30, and 31. In the illustrated embodiment, a tapered cylindrical seal may be formed between the cylindrical insert 23 and the cylindrical cavity 21, such that the cylindrical insert 23 may be press fit into the cylindrical cavity 21. The rotor 15 may be composed of a low-durometer, low-friction, plastic, such as, e.g., polytetrafluoroethylene (PTFE), while the stator 13 may be composed of a rigid plastic, such as, e.g., polyetherimide (Ultem®).

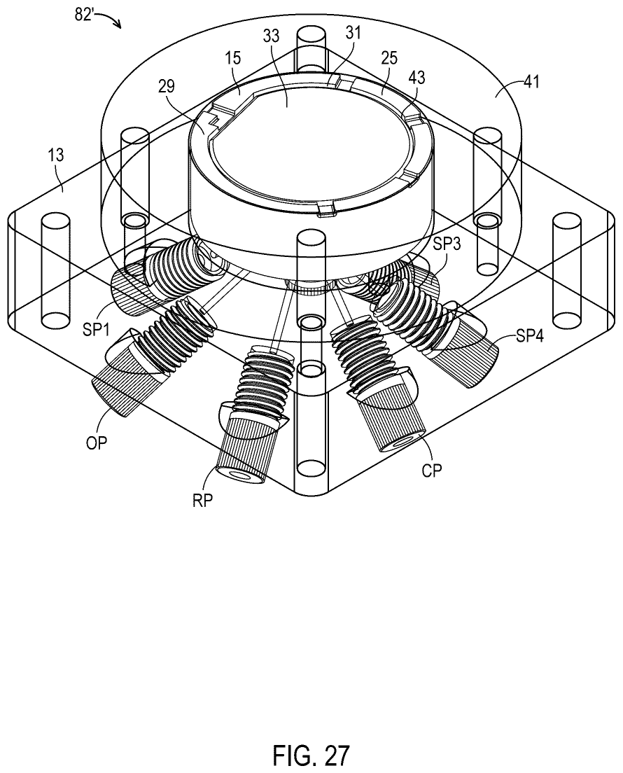

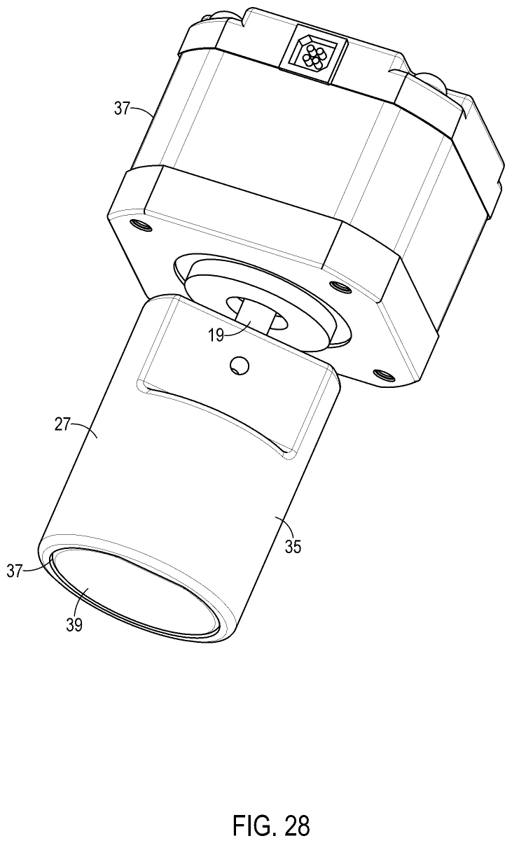

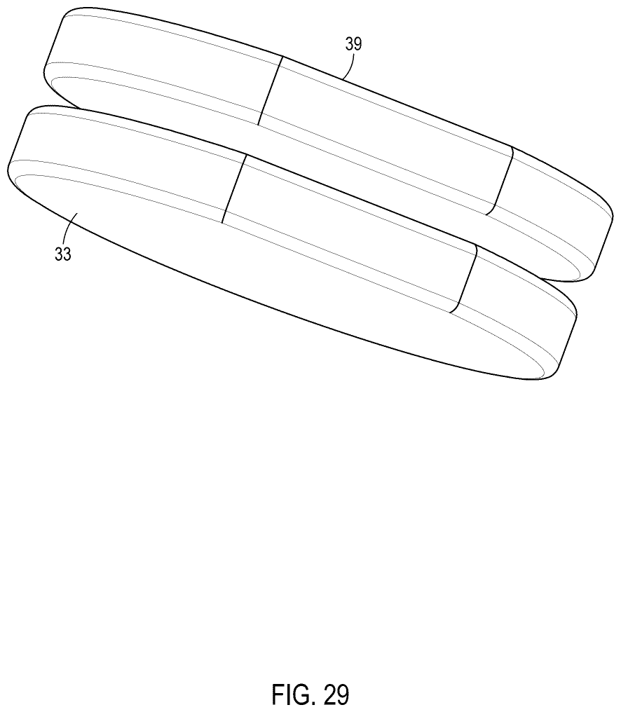

The motorized valve assembly 11 further comprises a pair of complementary magnetic couplings 25, 27 respectively affixed to the rotor 15 and to the end of the shaft 19 opposite the motor 17. In particular, the magnetic coupling 25 affixed to the rotor 15 resides outside of the cylindrical cavity 21 of the stator 13. As best illustrated in FIG. 27, the magnetic coupling 25 comprises an enlarged circular boss 29 having a low-profile cavity 31, and a magnet 33 affixed within the low-profile cavity 31 of the enlarged circular boss 29. Similarly, the magnetic coupling 27 affixed to the end of the shaft 19 comprises an enlarged circular boss 35 having a low-profile cavity 37, and a magnet 39 affixed within the low-profile cavity 37 of the enlarged circular boss 35, as best illustrated in FIG. 28. Each of the magnets 33, 39 may be, e.g., a polymagnet printed with poles spaced apart, e.g., by sixty degrees. As best illustrated in FIG. 29, the magnets 33, 39 are spaced from each other a small distance, such that they are not in physical contact with each other, but are still magnetic coupled to each other.

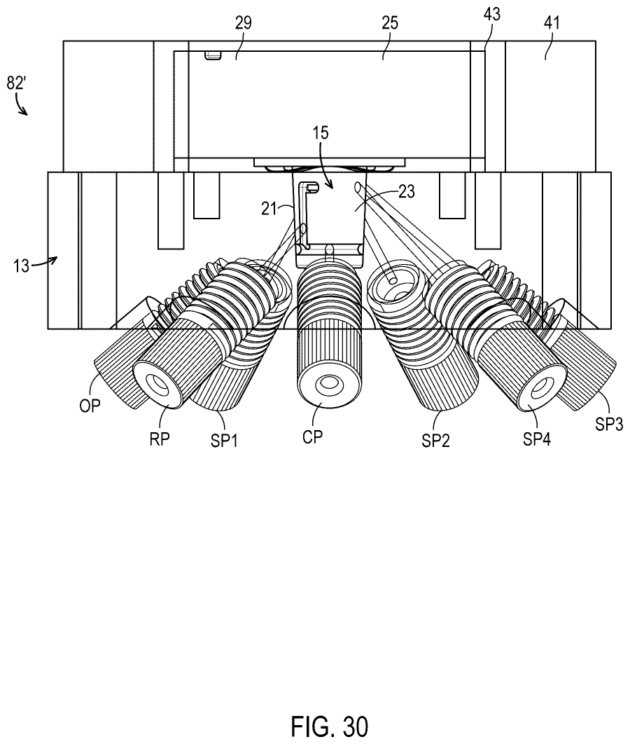

In this manner, in response to the rotation of the shaft 19 (in either rotational direction), the magnetic coupling 27 affixed to the end of the shaft 19 correspondingly rotates the magnetic coupling 25 affixed to the rotor 15, which correspondingly rotates the cylindrical insert 23 of the rotor 15 within the cylindrical cavity 21 of the stator 13. In an optional embodiment, the magnetic couplings 25, 27 may have a detent arrangement so as to mitigate any inherent backlash between the magnetic couplings 25, 27. Alternatively, a large accuracy tolerance may be built into the fluid couplings between the common port CP, return port RP, selector ports SP1 to SP4, and output port OP affixed within the stator 13 and the open channel system of the rotor 15 described in further detail below. The rotary valve 82′ further comprises a valve cap 41 affixed to the upper surface of the stator 13 for enclosing the rotor 15, and in particular, comprises a cylindrical cavity 43 in which the magnetic coupling 25 is rotatably disposed, as best illustrated in FIGS. 27 and 30.

The rotary valve 82′ is specifically configured, such that the rotor 15 may be rotated relative to the stator 13 in Positions 1-10 defined in FIG. 23 to effect the aforementioned combinations of fluid couplings and fluid decouplings between the common port CP, return ports RP, selector ports SP1-SP4, and output port OP.

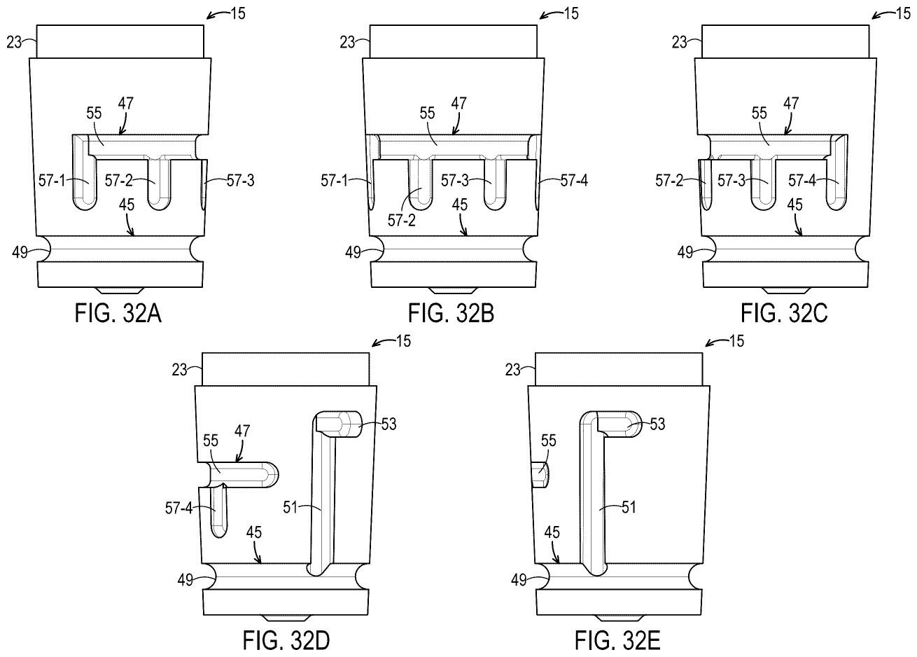

In particular, and with reference to FIGS. 32A-32E, the rotor 15 comprises a first open channel 45 (corresponding to the first valve portion 88a of the first multi-function valve V1′ or the first valve portion 88b of the second multi-function valve V2′ illustrated in FIG. 5) and a second open channel 47 (corresponding to the second valve portion 90a of the first multi-function valve V1′ or the second valve portion 90b of the second multi-function valve V2′ illustrated in FIG. 22) formed in the outer surface of cylindrical insert 23.

When the rotor 15 is rotated relative to the stator 13 in a first plurality of positions (in this case, Positions 1, 3, 5, and 7 defined in FIG. 23), the first open channel 45 fluidly couples the selector ports SP1-SP4 (i.e., selector ports SP1 to SP4) to the common port CP (i.e., the common port CP) (i.e., one of the selector ports SP1-SP4 and the common port CP concurrently coincide with the first open channel 45), while the second open channel 98 fluidly couples the output port OP (i.e., the output port OP) to the return port RP (i.e., the return port RP) (i.e., the output port OP and return port RP concurrently coincide with the second open channel 47). When the rotor 15 is rotated relative to the stator 13 in a second plurality of positions (in this case, Positions 2, 4, 6, and 8 defined in FIG. 23), the first open channel 45 fluidly couples the selector ports SP1-SP4 to the common port CP (i.e., one of the selector ports SP1-SP4 and the common port CP concurrently coincide with the first open channel 45), while the second open channel 47 does not fluidly couple the output port OP to the return port RP (i.e., the output OP and the return port RP do not concurrently coincide with the second open channel 47).

When the rotor 15 is rotated relative to the stator 13 in a third position (in this case, Position 9 defined in FIG. 23), the first open channel 45 does not fluidly couple any of the selector ports SP1-SP4 to the common port CP (i.e., none of the selector ports SP1-SP4 and the common port CP concurrently coincide with the first open channel 45), the second open channel 47 does not fluidly couple the output port OP to the return port RP (i.e., the output port OP and the return port RP do not concurrently coincide with the second open channel 47), and the first open channel 45 does not fluidly couple the return port RP to the common port CP (i.e., the return port RP and the common port CP do not concurrently coincide with the first open channel 45). When the rotor 15 is rotated relative to the stator 13 in a fourth position (in this case, Position 10 defined in FIG. 23), the first open channel 45 fluidly couples the return port RP to the common port CP (i.e., the return port RP and the common port CP concurrently coincide with the first open channel 45), while not fluidly coupling any of the selector ports SP1-SP4 to the common port CP (i.e., none of the selector ports SP1-SP4 and the common port CP concurrently coincide with the first open channel 45), and while the second open channel 47 does not fluidly couple the output port OP to the return port RP (i.e., the output port OP and the return port RP do not concurrently coincide with the second open channel 47).

In the illustrated embodiment, the first open channel 45 has a circumferential portion 49 with which the common port CP always coincides when the rotor 15 is rotated relative to the stator 13 in Positions 1-10. In the illustrated embodiment, the circumferential portion 49 extends entirely around the cylindrical insert 23. However, in alternative embodiments, the circumferential portion 49 only partially extends around the cylindrical insert 23. The first open channel 45 further has an axial portion 51 extending from the circumferential portion 49 and a circumferential spur portion 53 extending from the axial portion 51. In the illustrated embodiment, the circumferential spur portion 53 extends from the end (opposite to the circumferential portion 49) of the axial portion 51. The selector ports SP1-SP4 respectively coincide with the circumferential spur portion 53 when the rotor 15 is rotated relative to the stator 13 in Positions 1, 3, 5, and 7, and respectively coincide with the axial portion 51 when the rotor 15 is rotated relative to the stator 13 in Positions 2, 4, 6, and 8. In alternative embodiments, the selector ports SP1-SP4 may respectively coincide with the axial portion 51 when the rotor 15 is rotated relative to the stator 13 in Positions 1, 3, 5, and 7, and respectively coincide with the circumferential spur portion 53 when the rotor 15 is rotated relative to the stator 13 in Positions 2, 4, 6, and 8.

In the illustrated embodiment, the second open channel 47 has a circumferential portion 55 with which the output port OP always coincides when the rotor 15 is rotated relative to the stator 13 in Positions 1, 3, 5, or 7 or Positions 2, 4, 6, or 8. The second open channel 47 further a plurality of axial spur portions 57 (in this case, four axial spur portions 57-1 to 57-4 respectively corresponding to the four selector ports SP1-SP4) extending from the circumferential portion 55 and with which the return port RP respectively coincides when the rotor 15 is rotated relative to the stator 13 in Positions 1, 3, 5, and 7, and with which the return port RP does not coincide when the rotor 15 is rotated relative to the stator 13 in Positions 2, 4, 6, and 8.

When the rotor 15 is rotated relative to the stator 13 in Position 9, the common port CP coincides with the circumferential portion 49 of the first open channel 45, none of the selector ports SP1-SP4 coincides with the axial portion 51 or the circumferential spur portion 53 of the first open channel 45, the output port OP does not coincide with the circumferential portion 55 of the second open channel 47, and the return port RP does not coincide with any of the axial spur portions 57-1 to 57-4 of the second open channel 47 or with the axial portion 51 of the first open channel 45. When the rotor 15 is rotated relative to the stator 13 in Position 10, the common port CP coincides with the circumferential portion 49 of the first open channel 45, none of the selector ports SP1-SP4 coincide with the axial portion 51 or the circumferential spur portion 53 of the first open channel 45, and the return port RP coincides with axial portion 51 of the first open channel 45.

Notably, the end of the axial portion 51 and the circumferential spur portion 53 of the first open channel 45 (to which the different selector ports SP1-SP4 will be fluidly coupled) is at a different height than the axial spur portions 57-1 to 57-4 of the second open channel 47, thereby preventing coincidence of any of the selector ports SP1-SP4 with the axial spur portions 57-1 to 57-4, while also preventing coincidence of the return port RP with the circumferential spur portion 53.

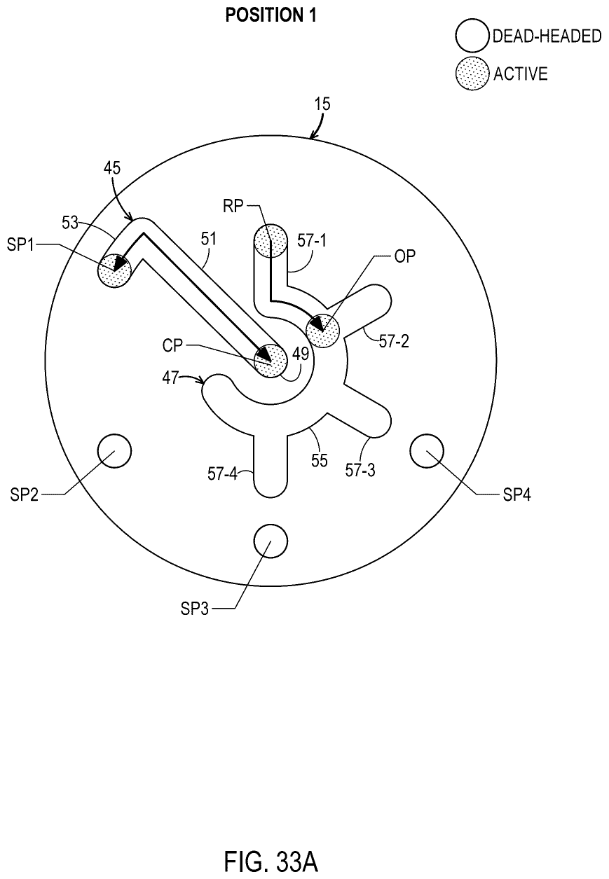

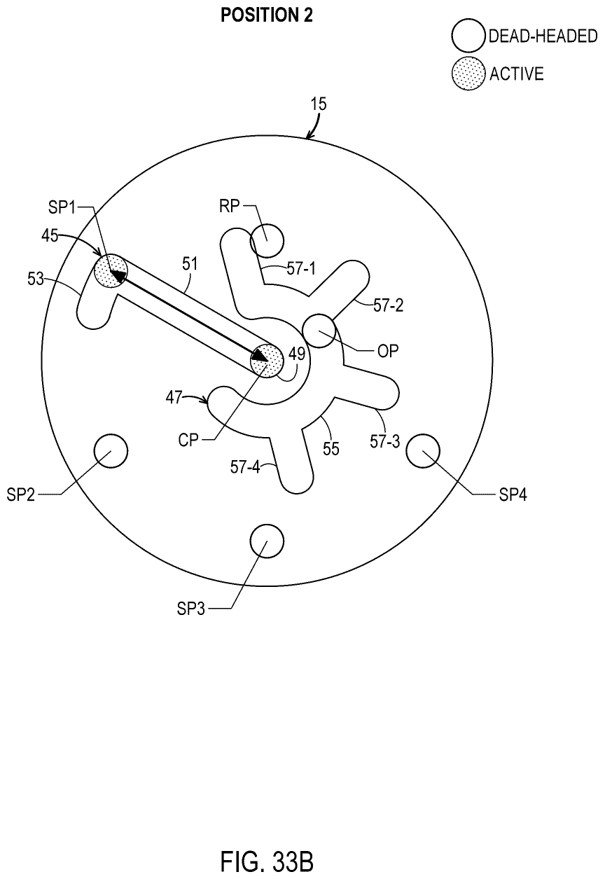

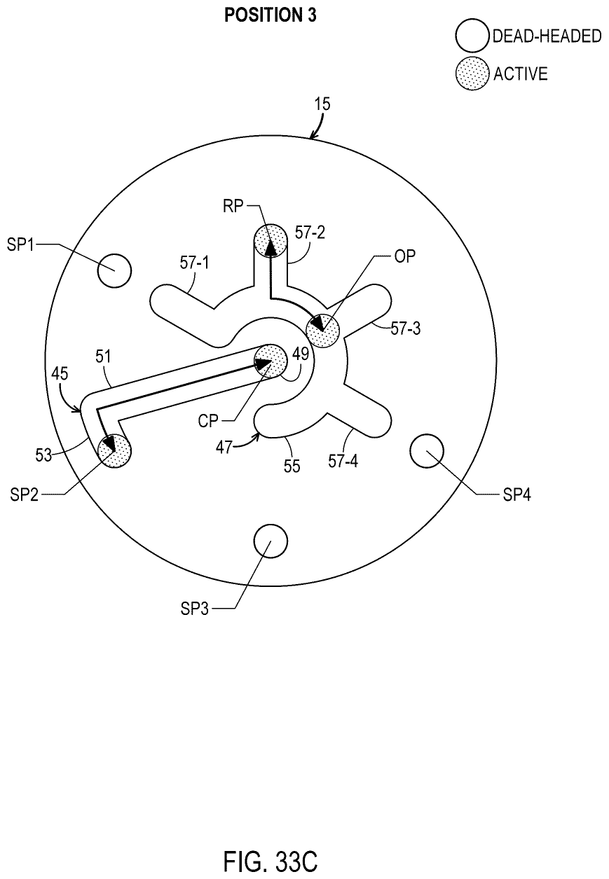

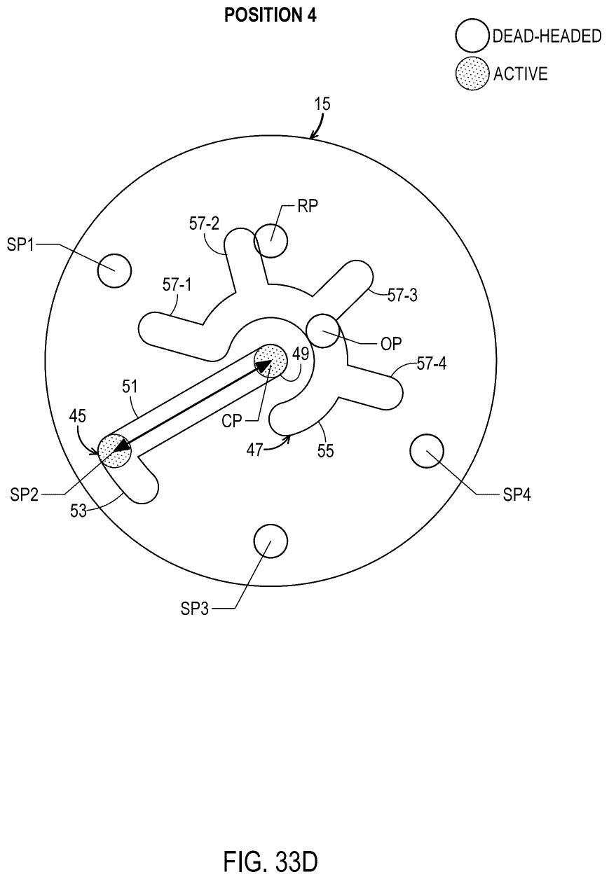

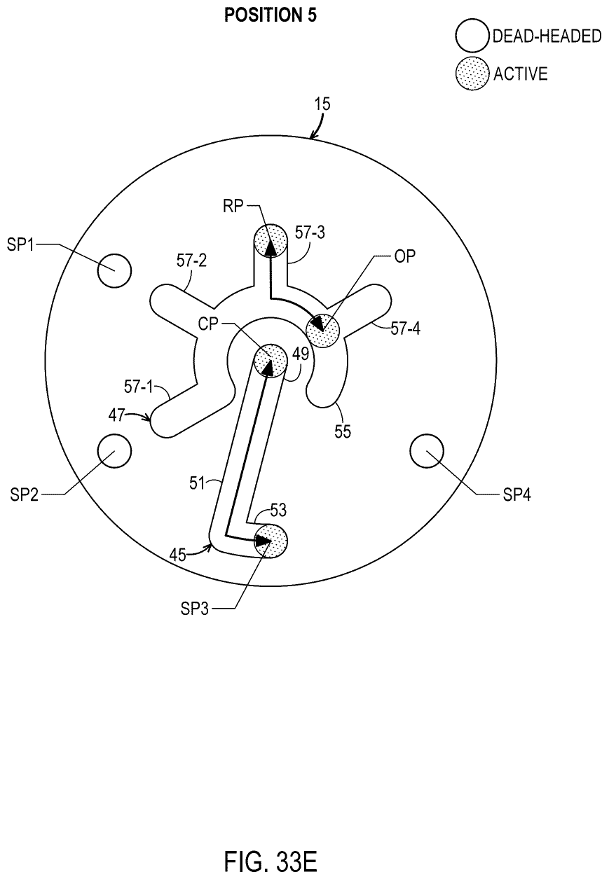

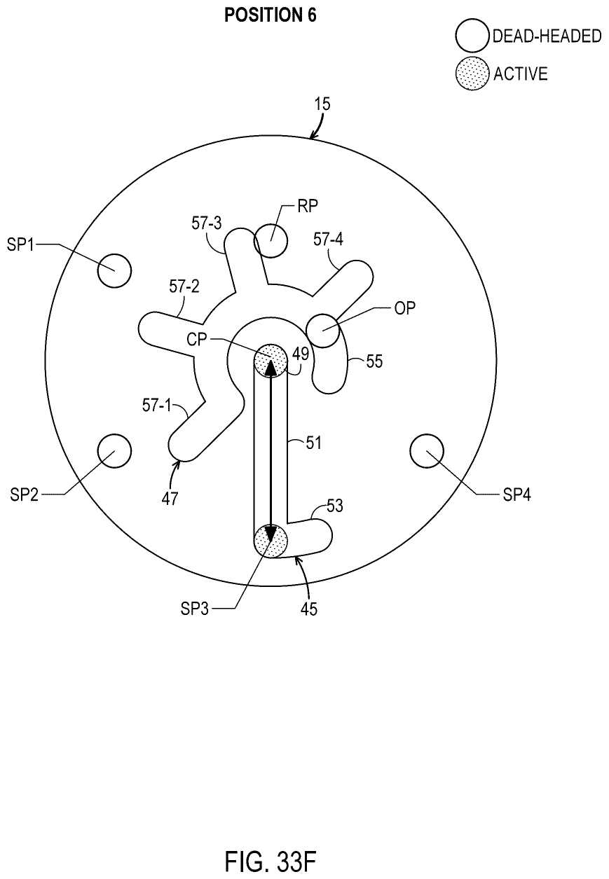

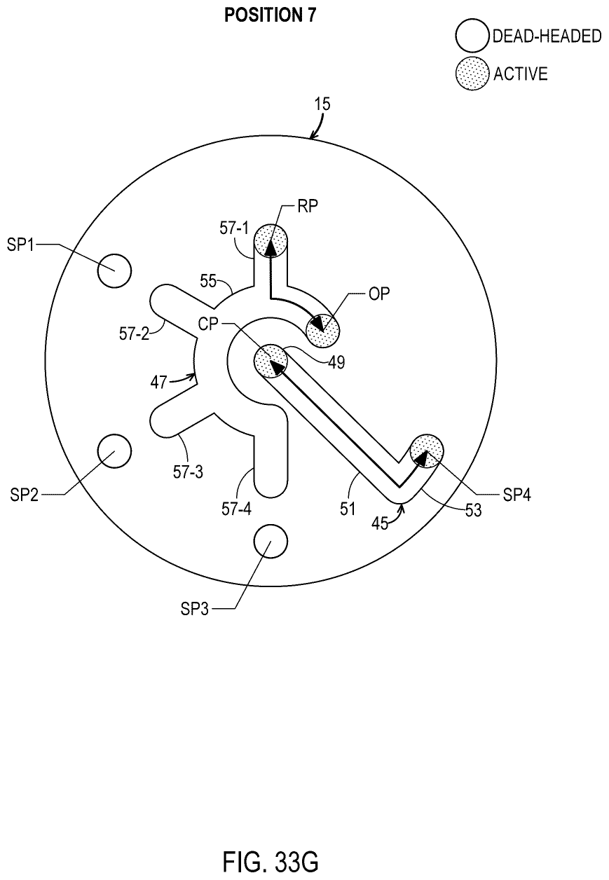

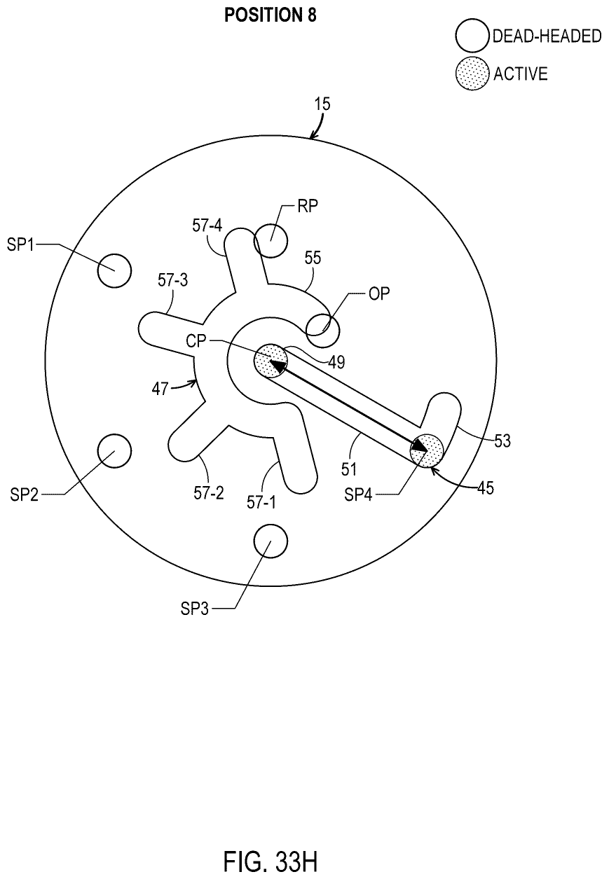

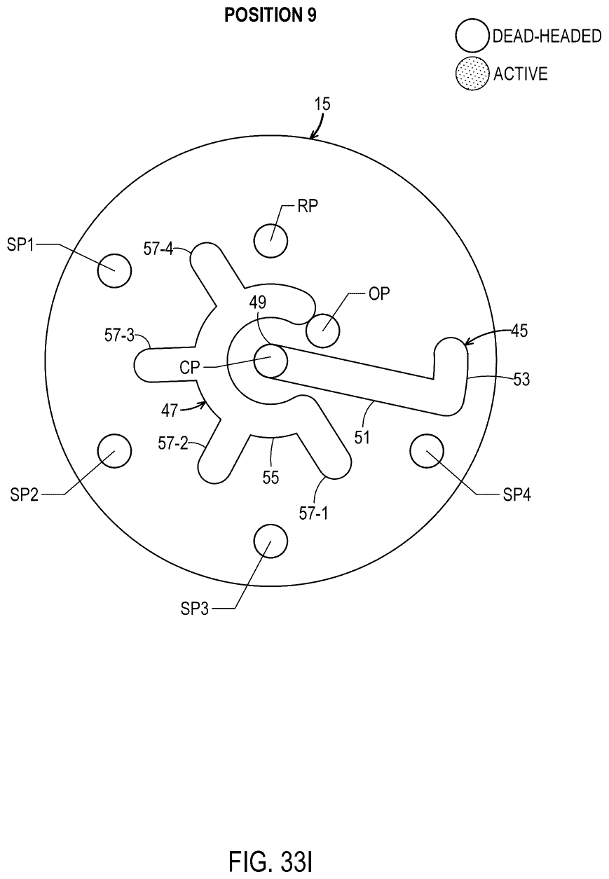

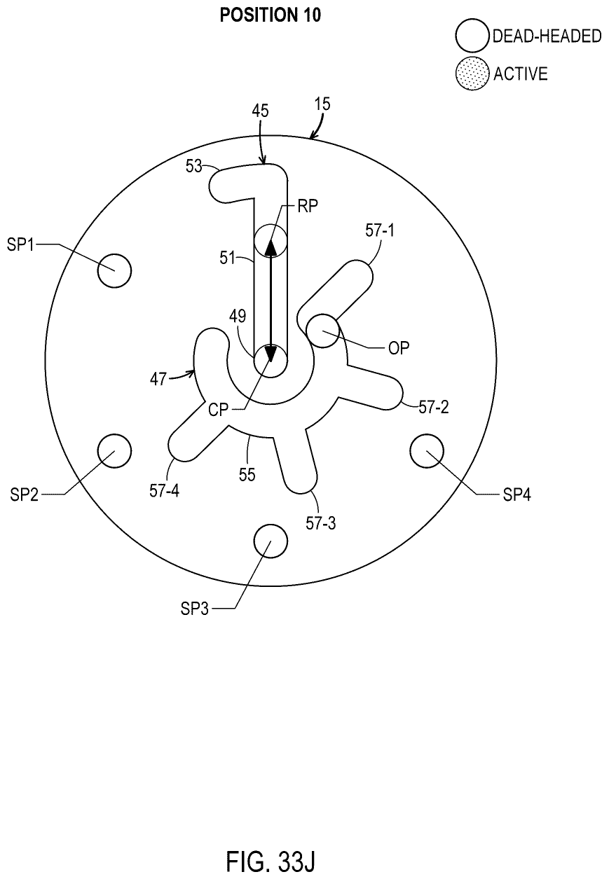

Referring now to FIGS. 33A-33J, the rotor 15 may be rotated relative to the stator 13 in accordance with a sequence of positions to effect the different fluid couplings and fluid decouplings between the different ones of the selector ports SP1-SP4 and the common port CP, between the output port OP and the return port RP, and between the return port RP and the common port CP, as illustrated in FIG. 23.

For example, when the rotor 15 is rotated relative to the stator 13 in Position 1 (FIG. 33A), the common port CP coincides with the circumferential portion 49 of the first open channel 45, the first selector port SP1 coincides with the circumferential spur portion 53 of the first open channel 45, the output port OP coincides with the circumferential portion 55 of the second open channel 47, and the return port RP coincides with the first axial spur portion 57-1 of the second open channel 47. As a result, the first selector port SP1 and the common port CP are activated, thereby fluidly coupling these two ports together (as shown by the arrow), while the remaining selector ports SP2-SP4 are deactivated (or deadheaded), and the return port RP and the output port OP are activated, thereby coupling these two ports together (as shown by the arrow).

When the rotor 15 is rotated relative to the stator 13 in Position 2 (FIG. 33B), the common port CP coincides with the circumferential portion 49 of the first open channel 45, the first selector port SP1 coincides with the axial portion 51 of the first open channel 45, the output port OP coincides with the circumferential portion 55 of the second open channel 47, and the return port RP does not coincide with any of the axial spur portions 57-1 to 57-4 of the second open channel 47. As a result, the first selector port SP1 and the common port CP are activated, thereby fluidly coupling these two ports together (as shown by the arrow), while the remaining selector ports SP2-SP4 are deactivated (or deadheaded), and the return port RP and the output port OP are deactivated (or deadheaded), thereby decoupling these two ports from each other.

When the rotor 15 is rotated relative to the stator 13 in Position 3 (FIG. 33C), the common port CP coincides with the circumferential portion 49 of the first open channel 45, the second selector port SP2 coincides with the circumferential spur portion 53 of the first open channel 45, the output port OP coincides with the circumferential portion 55 of the second open channel 47, and the return port RP coincides with the second axial spur portion 57-2 of the second open channel 47. As a result, the second selector port SP2 and the common port CP are activated, thereby fluidly coupling these two ports together (as shown by the arrow), while the remaining selector ports SP1 and SP3-SP4 are deactivated (or deadheaded), and the return port RP and the output port OP are activated, thereby coupling these two ports together (as shown by the arrow).

When the rotor 15 is rotated relative to the stator 13 in Position 4 (FIG. 33D), the common port CP coincides with the circumferential portion 49 of the first open channel 45, the second selector port SP2 coincides with the with the axial portion 51 of the first open channel 45, the output port OP coincides with the circumferential portion 55 of the second open channel 47, and the return port RP does not coincide with any of the axial spur portions 57-1 to 57-4 of the second open channel 47. As a result, the second selector port SP2 and the common port CP are activated, thereby fluidly coupling these two ports together (as shown by the arrow), while the remaining selector ports SP1 and SP3-SP4 are deactivated (or deadheaded), and the return port RP and the output port OP are deactivated (or deadheaded), thereby decoupling these two ports from each other.

When the rotor 15 is rotated relative to the stator 13 in Position 5 (FIG. 33E), the common port CP coincides with the circumferential portion 49 of the first open channel 45, the third selector port SP3 coincides with the circumferential spur portion 53 of the first open channel 45, the output port OP coincides with the circumferential portion 55 of the second open channel 47, and the return port RP coincides with the third axial spur portion 57-3 of the second open channel 47. As a result, the third selector port SP3 and the common port CP are activated, thereby fluidly coupling these two ports together (as shown by the arrow), while the remaining selector ports SP1-SP2 and SP4 are deactivated (or deadheaded), and the return port RP and the output port OP are activated, thereby coupling these two ports together (as shown by the arrow).

When the rotor 15 is rotated relative to the stator 13 in Position 6 (FIG. 33F), the common port CP coincides with the circumferential portion 49 of the first open channel 45, the third selector port SP3 coincides with the axial portion 51 of the first open channel 45, the output port OP coincides with the circumferential portion 55 of the second open channel 47, and the return port RP does not coincide with any of the axial spur portions 57-1 to 57-4 of the second open channel 47. As a result, the third selector port SP3 and the common port CP are activated, thereby fluidly coupling these two ports together (as shown by the arrow), while the remaining selector ports SP1-SP2 and SP4 are deactivated (or deadheaded), and the return port RP and the output port OP are deactivated (or deadheaded), thereby decoupling these two ports from each other.

When the rotor 15 is rotated relative to the stator 13 in Position 7 (FIG. 33G), the common port CP coincides with the circumferential portion 49 of the first open channel 45, the fourth selector port SP4 coincides with the circumferential spur portion 53 of the first open channel 45, the output port OP coincides with the circumferential portion 55 of the second open channel 47, and the return port RP coincides with the fourth axial spur portion 57-4 of the second open channel 47. As a result, the fourth selector port SP4 and the common port CP are activated, thereby fluidly coupling these two ports together (as shown by the arrow), while the remaining selector ports SP1-SP3 are deactivated (or deadheaded), and the return port RP and the output port OP are activated, thereby coupling these two ports together (as shown by the arrow).

When the rotor 15 is rotated relative to the stator 13 in Position 8 (FIG. 33H), the common port CP coincides with the circumferential portion 49 of the first open channel 45, the fourth selector port SP4 coincides with the axial portion 51 of the first open channel 45, the output port OP coincides with the circumferential portion 55 of the second open channel 47, and the return port RP does not coincide with any of the axial spur portions 57-1 to 57-4 of the second open channel 47. As a result, the fourth selector port SP4 and the common port CP are activated, thereby fluidly coupling these two ports together (as shown by the arrow), while the remaining selector ports SP1-SP3 are deactivated (or deadheaded), and the return port RP and the output port OP are deactivated (or deadheaded), thereby decoupling these two ports from each other.

When the rotor 15 is rotated relative to the stator 13 in Position 9 (FIG. 33I), the common port CP coincides with the circumferential portion 49 of the first open channel 45, none of the selector ports SP1-SP4 coincide with the axial portion 51 or the circumferential spur portion 53 of the first open channel 45, the output port OP does not coincide with the circumferential portion 55 of the second open channel 47, and the return port RP does not coincide with any of the axial spur portions 57 of the second open channel 47 or the axial portion 51 of the first open channel 45. As a result, all of the selector ports SP1-SP4 and the common port CP are deactivated (or deadheaded), thereby fluidly decoupling all of these ports from each other, the return port RP and the output port OP are deactivated (or deadheaded), thereby fluidly decoupling these two ports from each other, and the output port OP and the common port CP are deactivated (or deadheaded), thereby fluidly decoupling these ports from each other.

When the rotor 15 is rotated relative to the stator 13 in Position 10 (FIG. 33J), the common port CP coincides with the circumferential portion 49 of the first open channel 45, none of the selector ports SP1-SP4 coincide with the axial portion 51 or the circumferential spur portion 53 of the first open channel 45, the output port OP does not coincide with the circumferential portion 55 of the second open channel 47, and the return port RP coincides with the axial portion 51 of the first open channel 45. As a result, all of the selector ports SP1-SP4 are deactivated (or deadheaded), thereby fluidly decoupling these ports from the common port CP, the output port OP is deactivated (or deadheaded), thereby fluidly decoupling this port from the return port RP, and the return port RP and common port CP are activated, thereby fluidly coupling these two ports together.

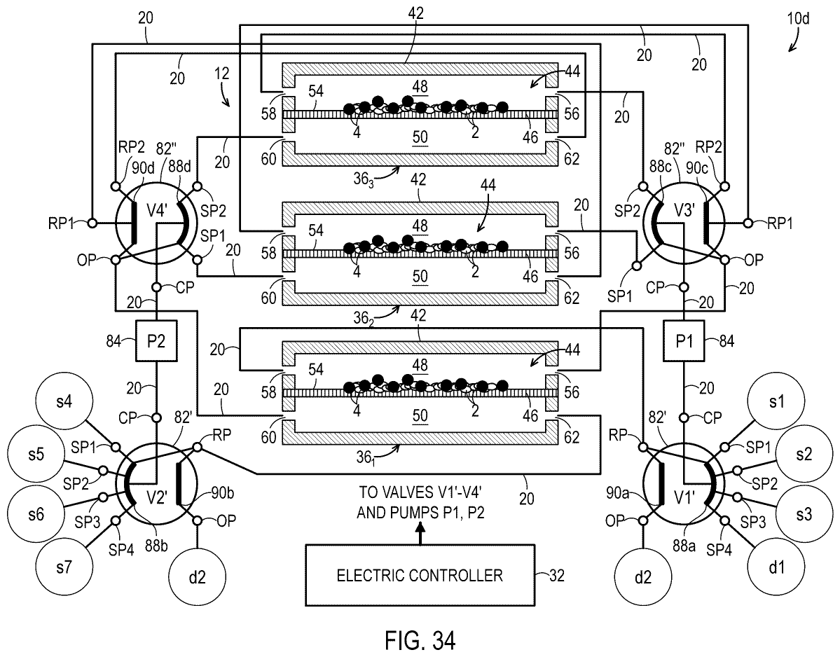

Referring now to FIG. 34, another embodiment of a scalable bioreactor system 10d constructed in accordance with the present inventions will be described. The bioreactor system 10d is similar to the bioreactor system 10b illustrated in FIG. 20 in that it generally comprises a bioreactor assembly 12 (comprising the stacked bioreactor unit 36₁-36₃), a plurality of media sources s1-s7, a plurality of media destinations d1-d2, two pumps 84 corresponding to the pump assembly 18 illustrated in FIG. 1 (in particular, a first pump P1 and a second pump P2), the arrangement of fluid conduits 20, and the electric controller 22. The bioreactor system 10d differs from the bioreactor system 10b illustrated in FIG. 18 in that, instead of comprising eight single-function valves 82 (V1-V8), the bioreactor system 10d comprises two multi-function valves 82′ (in particular, the previously described first multi-function valve V1′ and the second multi-function valve V2′), as well as two additional multi-function valves 82″ (in particular, a third multi-function valve V3′ and a fourth multi-function valve V4′). The multi-function valves V1′-V4′ correspond to the valve assembly 16 illustrated in FIG. 1. In the illustrated embodiment, the first bioreactor unit 36₁ may be referred to as a “base bioreactor unit,” whereas the second and third bioreactor units 36₂, 36₃ may be referred to as “supplemental bioreactor units.” Although two supplemental bioreactor units 36₂, 36₃ are illustrated, it should be appreciated that the bioreactor assembly 12 may comprise less supplemental bioreactor units (i.e., only one supplemental bioreactor unit) or more supplemental bioreactor units (i.e., more than two supplemental bioreactor units).

Rather than selectively defining different flow paths between various ones of the media sources s1-s7 and media destinations d1-d2 as performed in the bioreactor system 10c illustrated in FIG. 22, the first and second multi-function valves V1′, V2′ are merely configured for selecting the media sources s1-s7 and media destinations d1-d2 to a particular flow path, whereas the third and fourth multi-function valves 82″ (V3′, V4′) are fluidly coupled between the multi-function valves V1′, V2′ and the bioreactor units 36₁-36₃ for defining different flow paths between the media sources s1-s7 and media destinations d1-d2 selected by the multi-function valves V1′, V2′ and a selected one of the bioreactor units 36₁-36₃. To this end, the third multi-function valve V3′ is associated with the upper flow channels 48 of all three of the bioreactor units 36₁-36₃, and the fourth multi-function valve V4′ is associated with the upper flow channels 50 of all three of the bioreactor units 36₁-36₃.

As will be described in further detail below, the third and fourth multi-function valves V3′, V4′ are operated in a manner, such that the defined flow paths (e.g., the flow path 86a-86g or the combined flow paths 86c, 86e illustrated in FIGS. 19A-19H) bypass both of the supplemental bioreactor units 36₂, 36₃ and only pass through the base bioreactor unit 36₁; the defined flow paths that pass entirely through a respective one of the flow channels 48, 50 (e.g., the flow paths 86a-86d illustrated in FIGS. 19A-19D) pass only through a selected one of the bioreactors 36₁-36₃; the defined flow paths that pass between the flow path channels 48, 50 (e.g., the flow paths 86e-86g illustrated in FIGS. 19E-19G) pass through the base bioreactor unit 36₁ and a selected one of the bioreactor units 36₂, 36₃).

The first and second multi-function valves V1′, V2′ in the bioreactor system 10d illustrated in FIG. 34 are fluidly connected to the base bioreactor unit 36 in the same manner as the first and second multi-function valves V1′, V2′ are fluidly connected to the bioreactor 36 in the bioreactor system 10c illustrated in FIG. 22, with the exception that the common port CP of the first multi-function valve V1′ is indirectly fluidly connected to the first port 56 of the base bioreactor unit 36₁ via the third multi-function valve V3′ (instead of being directly fluidly connected to the first port 56 of the base bioreactor unit 36), while the common port CP of the second multi-function valve V2′ is indirectly fluidly connected to the third port 60 of the base bioreactor unit 36₁ via the fourth multi-function valve V4′ (instead of being directly fluidly connected to the third port 60 of the base bioreactor unit 36₁).

In particular, the common port CP of the first multi-function valve V1′ is fluidly connected to a corresponding common port (described in further detail below) of the third multi-function valve V3′ via the first pump P1, and the return port RP is fluidly connected to the second port 58 of the base bioreactor unit 36₁, while the common port CP of the second multi-function valve V2′ is fluidly connected to a corresponding common port (described in further detail below) of the fourth multi-function valve V4′ via the second pump P2, and the return port RP of the second multi-function valve V2′ to the fourth port 62 of the base bioreactor unit 36₁.

The first and second multi-function valves V1′, V2′ in the bioreactor system 10d illustrated in FIG. 34 are also fluidly connected to the media sources s1-s7 and media destinations d1-d2 in the same manner as the first and second multi-function valves V1′, V2′ in the bioreactor system 10c illustrated in FIG. 22 are connected to the media sources s1-s7 and media destinations d1-d2. In particular, the selector ports SP1-SP4 (there input ports SP1-SP3 and one output port SP4) of the first multi-function valve V1′ are respectively fluidly connected to the media sources s1-s3 and the media destination d1, and the output port OP of the first multi-function valve V1′ is fluidly connected to the media destination d2; while the selector ports SP1-SP4 (all input ports) of the second multi-function valve V2′ are respectively fluidly connected to the media sources s4-s7, and the output port OP of the second multi-function valve V2′ is fluidly connected to the media destination d2.

In the illustrated embodiment, the third multi-function valve V3′ comprises a common port CP fluidly connected to the common port CP of the first multi-function valve V1′ via the first pump P1; a plurality of return ports (in this case, two return ports RP1, RP2) respectively fluidly connected to the second ports 58 of both of the supplemental bioreactor units 36₂, 36₃; a plurality of selector ports (in this case, two selector ports SP1, SP2) respectively fluidly connected to the first ports 56 of both of the supplemental bioreactor units 36₂, 36₃, and an output port OP fluidly connected to the first port 56 of the base bioreactor unit 36₁. Similarly, the fourth multi-function valve V4′ comprises a common port CP fluidly connected to the common port CP of the second multi-function valve V2′ via the second pump P2; a plurality of return ports (in this case, two return ports RP1, RP2) respectively fluidly connected to the fourth ports 62 of both of the supplemental bioreactor units 36₂, 36₃; a plurality of selector ports (in this case, two selector ports SP1, SP2) respectively fluidly connected to the third ports 60 of both of the supplemental bioreactor units 36₂, 36₃, and an output port 56d fluidly connected to the third port 60 of the base bioreactor unit 36₁.

Thus, the first multi-function valve V1′, along with the first pump P1, are fluidly connected (via the third multi-function valve V3′) between the first and second ports 56, 58 of the bioreactor units 36₁-36₃, thereby associating it with the upper flow channels 48 of the bioreactor units 36₁-36₃. Furthermore, the first multi-function valve V1′ is fluidly connected to the media sources s1-s3 and the media destination d1, such that they can be selectively fluidly coupled to the first ports 56 of the bioreactor units 36₁-36₃ (via the third multi-function valve V3′ for all of the bioreactor units 36), and is fluidly connected to the media destination d2, such that it can be fluidly coupled to the second ports 58 of the bioreactor units 36₁-36₃ (via the third multi-function valve V3′ for both of the supplemental bioreactor units 36₂, 36₃).

As will be described in further detail below, the third multi-function valve V3′ is operated to select the upper channels 48 of one or more of the bioreactor units 36₁-36₃ between which the selected one of the media sources s1-s3 and media destination d1 and the media destination d2 will be fluidly coupled. Thus, in conjunction with the third multi-function valve V3′, the first multi-function valve V1′ may only fluidly couple a selected one of the media sources s1-s3 and the media destination d1, only fluidly couple the media destination d2, or both fluidly couple a selected one of the media sources s1-s3 and media destination d1 and fluidly couple the media destination d2, to the upper flow channels 48 of one or more of the bioreactor units 36₁-36₃.

Similarly, the second multi-function valve V2′, along with the second pump P2, are fluidly connected (via the fourth multi-function valve V4′) between the third and fourth ports 60, 62 of the bioreactor units 36₁-36₃, thereby associating it with the lower flow channels 50 of the bioreactor units 36₁-36₃. Furthermore, the second multi-function valve V2′ is fluidly connected to the media sources s1-s3 and the media destination d1, such that they can be selectively fluidly coupled to the third ports 60 of the bioreactor units 36₁-36₃ (via the third multi-function valve V3′ for all of the bioreactor units 36), and is fluidly connected to the media destination d2, such that it can be fluidly coupled to the fourth ports 58 of the bioreactor units 36₁-36₃ (via the fourth multi-function valve V4′ for both of the supplemental bioreactor units 36₂, 36₃).

As will be described in further detail below, the fourth multi-function valve V4′ is operated to select the lower channels 50 of one or more of the bioreactor units 36₁-36₃ between which the selected one of the media sources s4-s7 and the media destination d2 will be fluidly coupled. Thus, in conjunction with the fourth multi-function valve V4′, the second multi-function valve V2′ may only fluidly couple a selected one of the media sources s4-s7, only fluidly couple the media destination d2, or both fluidly couple a selected one of the media sources s4-s7 and fluidly couple the media destination d2, to the lower flow channels 50 of one or more of the bioreactor units all of the bioreactor units 36.

The first valve portion 88a of the first multi-function valve V1′ respectively fluidly couples its selector ports SP1-SP4 and its return port RP to its common port CP, such that media sources s1-s3, media destination d1, and second port 58 of the base bioreactor unit 36₁ are selectively fluidly coupled to the common port CP of the third multi-function valve V3′, and thus, indirectly fluidly coupled to the first port 56 of one of the bioreactor units 36₁-36₃ selected by the third multi-function valve V3′ (or fluidly decouples all of its selector ports SP1-SP4 and its return port RP from its common port CP, such that all of the media sources s1-s3, media destination d1, and second port 58 of the base bioreactor unit 36₁ are fluidly decoupled from the common port CP of the third multi-function valve V3′, and thus, indirectly fluidly decoupled from the first port 56 of one of the bioreactor units 36₁-36₃ selected by the third multi-function valve V3′. The second valve portion 90a of the first multi-function valve V1′ either fluidly couples its output port OP to its return port RP, such that the media destination d2 is fluidly coupled to the second port 58 of the base bioreactor unit 36₁, or its output port OP is fluidly decoupled from its return port RP, such that the media destination d2 is fluidly decoupled from the second port 58 of the base bioreactor unit 36₁.

Likewise, the first valve portion 88b of the second multi-function valve V2′ respectively fluidly couples its selector ports SP1-SP4 and its return port RP to its common port CP, such that media sources s4-s7 and fourth port 62 of the base bioreactor unit 36₁ are selectively fluidly coupled to the common port CP of the fourth multi-function valve V4′, and thus, indirectly fluidly coupled to the third port 60 of one of the bioreactor units 36₁-36₃ selected by the fourth multi-function valve V4′ (or fluidly decouples all of its selector ports SP1-SP4 and its return port RP from its common port CP, such that all of the media sources s4-s7 and fourth port 62 of the base bioreactor unit 36₁ are fluidly decoupled from the common port CP of the fourth multi-function valve V4′, and thus, indirectly fluidly decoupled from the third port 60 of one of the bioreactor units 36₁-36₃ selected by the fourth multi-function valve V4′. The second valve portion 90b of the second multi-function valve V2′ either fluidly couples its output port OP to its return port RP, such that the media destination d2 is fluidly coupled to the fourth port 62 of the base bioreactor unit 36₁, or its output port OP is fluidly decoupled from its return port RP, such that the media destination d2 is fluidly decoupled from the fourth port 62 of the base bioreactor unit 36₁.

In the same manner described above, the multi-function valves V1′, V2′ may be placed into any of the ten different positions defined in FIG. 23 to provide different fluid couplings or fluid decouplings between selected ones of the media sources s1-s7 and media destinations d1-d2 and the common ports 48c, 48d of the third and fourth multi-function valves V3′, V4′, as well as the second and fourth ports 58, 62 of the base bioreactor unit 36₁.

In particular, each of the multi-function valves V1′, V2′ may be placed into a first plurality of positions (e.g., Positions 1, 3, 5 and 7) for respectively fluidly coupling its selector ports SP1-SP4 to its common port CP (thereby fluidly coupling the media sources s1-s3 and media destination d1 respectively to the common port CP of the third multi-function valve V3′ in the case of the first multi-function valve V1′ or fluidly coupling the media sources s4-s7 respectively to the common port CP of the fourth multi-function valve V4′ in the case of the second multi-function valve V2′), while fluidly coupling its output port OP to its return port RP (thereby fluidly coupling the media destination d2 to the second port 58 of the base bioreactor unit 36₁ in the case of the first multi-function valve V1′ or fluidly coupling the media destination d1 to the fourth port 62 of the base bioreactor unit 36₁ in the case of the second multi-function valve V2′), and while fluidly decoupling its return port RP from its common port CP (thereby fluidly decoupling the first and second ports 56, 58 of the base bioreactor unit 36₁ from each other in the case of the first multi-function valve V1′, or fluidly decoupling the third and fourth ports 60, 62 of the base bioreactor unit 36₁ from each other in the case of the second multi-function valve V2′).

Each of the multi-function valves V1′, V2′ may also be placed into a second plurality of positions (e.g., Positions 2, 4, 6, and 8) for respectively fluidly coupling its selector ports SP1-SP4 to its common port CP (thereby fluidly coupling the media sources s1-s3 and media destination d1 respectively to the common port CP of the third multi-function valve V3′ in the case of the first multi-function valve V1′ or fluidly coupling the media sources s4-s7 respectively to the common port CP of the fourth multi-function valve V4′ in the case of the second multi-function valve V2′), while fluidly decoupling its output port OP from its return port RP (thereby fluidly decoupling the media destination d2 from the second port 58 of the base bioreactor unit 36₁ in the case of the first multi-function valve V1′ or fluidly decoupling the media destination d2 from the fourth port 62 of the base bioreactor unit 36₁ in the case of the second multi-function valve V2′), and while fluidly decoupling its return port RP from its common port CP (thereby fluidly decoupling the first and second ports 56, 58 of the base bioreactor unit 36₁ from each other in the case of the first multi-function valve V1′, or fluidly decoupling the third and fourth ports 60, 62 of the base bioreactor unit 36₁ from each other in the case of the second multi-function valve V2′).

Each of the multi-function valves V1′, V2′ may also be placed into a third position (e.g., Position 9) for fluidly decoupling all of its selector ports SP1-SP4 from its common port CP (thereby fluidly decoupling all of the media sources s1-s3 and media destination d1 from the common port CP of the third multi-function valve V3′ in the case of the first multi-function valve V1′ or fluidly decoupling all of the media sources s4-s7 from the common port CP of the fourth multi-function valve V4′ in the case of the second multi-function valve V2′), while fluidly decoupling its output port OP from its return port RP (thereby fluidly decoupling the media destination d2 from the second port 58 of the base bioreactor unit 36₁ in the case of the first multi-function valve V1′ or fluidly decoupling the media destination d2 from the fourth port 62 of the base bioreactor unit 36₁ in the case of the second multi-function valve V2′), and while fluidly decoupling its return port RP from its common port CP (thereby fluidly decoupling the first and second ports 56, 58 of the base bioreactor unit 36 from each other in the case of the first multi-function valve V1′, or fluidly decoupling the third and fourth ports 60, 62 of the base bioreactor unit 36₁ from each other in the case of the second multi-function valve V2′).

Each of the multi-function valves V1′, V2′ may optionally be placed into a fourth position (e.g., Position 10) for fluidly decoupling all of its selector ports SP1-SP4 from its common port CP (thereby fluidly decoupling all of the media sources s1-s3 and media destination d1 from the common port CP of the third multi-function valve V3′ in the case of the first multi-function valve V1′ or fluidly decoupling all of the media sources s4-s7 from the common port CP of the fourth multi-function valve V4′ in the case of the second multi-function valve V2′), while fluidly decoupling its output port OP from its return port RP (thereby fluidly decoupling the media destination d2 from the second port 58 of the base bioreactor unit 36₁ in the case of the first multi-function valve V1′ or fluidly decoupling the media destination d2 from the fourth port 62 of the base bioreactor unit 36₁ in the case of the second multi-function valve V2′), and while fluidly coupling its return port RP to the common port CP (thereby fluidly coupling the first and second ports 56, 58 of the base bioreactor unit 36₁ to each other via the third multi-function valve V3′ in the case of the first multi-function valve V1′, or fluidly coupling the third and fourth ports 60, 62 of the base bioreactor unit 36₁ to each other via the fourth multi-function valve V4′ in the case of the second multi-function valve V2′).

Like the first and second multi-function valves V1′, V2′, the third and fourth multi-function valves V3′, V4′ respectively comprise first and second valve portions 88c, 90c and first and second valve portions 88d, 90d.

The first valve portion 88c of the third multi-function valve V3′ respectively fluidly couples its selector ports SP1, SP2 and output port OP to its common port CP, such that the first ports 56 of the supplemental bioreactor units 36₂, 36₃ and the base bioreactor unit 36₁ are selectively fluidly coupled to the common port CP of the first multi-function valve V1′, and thus, indirectly fluidly coupled to one of the media sources s1-s3 and media destination d1 selected by the first multi-function valve V1′ (or fluidly decouples both of its selector ports SP1, SP2 and its output port OP from its common port CP, such that the first ports 56 of both of the supplemental bioreactor units 36₂, 36₃ and the base bioreactor unit 36₁ are fluidly decoupled from the common port CP of the first multi-function valve V1′, and thus, fluidly decoupled from one of the media sources s1-s3 and media destination d1 selected by the first multi-function valve V1′. The second valve portion 90c of the third multi-function valve V3′ selectively fluidly couples its return ports RP1, RP2 to its output port OP, such that the second ports 58 of the supplemental bioreactor units 36₂, 36₃ are selectively fluidly coupled to the first port 56 of the base bioreactor unit 36₁ (or fluidly decouples both its return ports RP1, RP2 from its output port OP, such that the second ports 58 of the supplemental bioreactor units 36₂, 36₃ are fluidly decoupled from the first port 56 of the base bioreactor unit 36₁).

Similarly, the first valve portion 88d of the fourth multi-function valve V4′ respectively fluidly couples its selector ports SP1, SP2 and output port OP to its common port CP, such that the third ports 60 of the supplemental bioreactor units 36₂, 36₃ and the base bioreactor unit 36₁ are selectively fluidly coupled to the common port CP of the second multi-function valve V2′, and thus, indirectly fluidly coupled to one of the media sources s4-s7 selected by the second multi-function valve V2′ (or fluidly decouples both of its selector ports SP1, SP2 and its output port OP from its common port CP, such that the third ports 60 of both of the supplemental bioreactor units 36₂, 36₃ and the base bioreactor unit 36₁ are fluidly decoupled from the common port CP of the second multi-function valve V2′, and thus, fluidly decoupled from one of the media sources s4-s7 selected by the second multi-function valve V2′. The second valve portion 90d of the fourth multi-function valve V4′ selectively fluidly couples its return ports RP1, RP2 to its output port OP, such that the fourth ports 62 of the supplemental bioreactor units 36₂, 36₃ are selectively fluidly coupled to the third port 60 of the base bioreactor unit 36₁ (or fluidly decouples both its return ports RP1, RP2 from its output port OP, such that the fourth ports 62 of the supplemental bioreactor units 36₂, 36₃ are fluidly decoupled from the third port 60 of the base bioreactor unit 36₁).

The first and second valve portions 88c, 90c of the third multi-function valve V3′ may be mechanically synchronized with each other, such that the third multi-function valve V3′ may be placed into one of several positions that facilitate coordinated fluid couplings (or fluid decouplings) between its selector ports SP1, SP2 and output port OP and its common port CP, and fluid couplings (or fluid decouplings) between its return ports RP1, RP2 and its output port OP. Likewise, the first and second valve portions 88d, 90d of the fourth multi-function valve V4′ may be mechanically synchronized with each other, such that the third multi-function valve V4′ may be placed into one of several positions that facilitate coordinated fluid couplings (or fluid decouplings) between its selector ports SP1, SP2 and output port OP and its common port CP, and fluid couplings (or fluid decouplings) between its return ports RP1, RP2 and its output port OP.

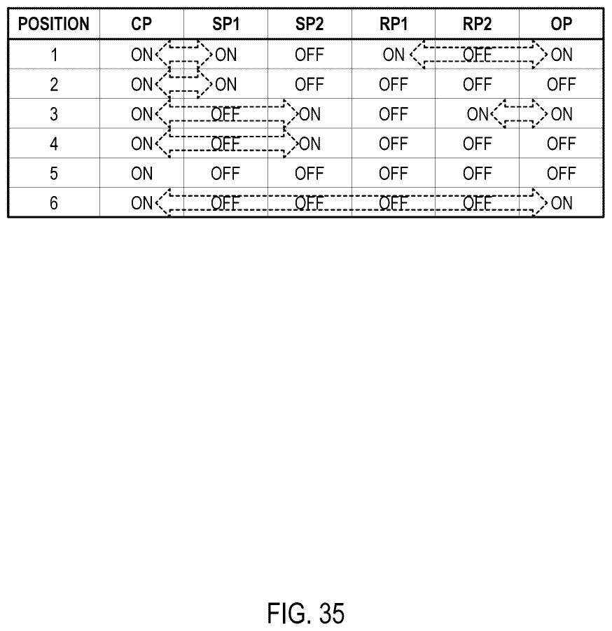

One exemplary embodiment of a multi-function valve that can be used as either of the third and fourth multi-function valves V3′, V4′ has a sequence of six positions, as illustrated in FIG. 35. Notably, the arrows in FIG. 35 represent the fluid couplings between the respective ports. It should be appreciated that, while the number of selector ports SP1, SP2 and the number of return ports RP1, RP2 equals four, alternative embodiments of the multi-function valve may have more or less than four selector ports, including only one selector port and only one return port, and may ultimately depend on the number of supplemental bioreactor units in the bioreactor assembly 12.

In Position 1, only the selector port SP1 is fluidly coupled to the common port CP, while only the return port RP1 is fluidly coupled to the output port OP; in Position 2, only the selector port SP1 is fluidly coupled to the common port CP, while both of the return ports RP1, RP2 are fluidly decoupled from the output port OP; in Position 3, only the selector port SP2 is fluidly coupled to the common port CP, while only the return port RP2 is fluidly coupled to the output port OP; in Position 4, only the selector port SP2 is fluidly coupled to the common port CP, while both of the return ports RP1, RP2 are fluidly decoupled from the output port OP; in Position 5, both of the selector ports SP1, SP2 are fluidly decoupled from the common port CP, while both of the return ports RP1, RP2, are fluidly decoupled from the output port OP; and in Position 6, both of the selector ports SP1, SP2 are fluidly decoupled from the common port CP, while both of the return ports RP1, RP2, are fluidly decoupled from the output port OP, and the output port OP is fluidly coupled to the common port CP.

Thus, it can be appreciated from FIG. 35 that either of the multi-function valves V3′, V4′ has a first plurality of positions (e.g., Positions 1 and 3) for respectively fluidly coupling its selector ports SP1, SP2 to its common port CP (thereby fluidly coupling the first ports 56 of both of the supplemental bioreactor units 36₂, 36₃ respectively to the common port CP of the first multi-function valve V1′, and thus, to one of the media sources s1-s3 and media destination d1 selected by the first multi-function valve V1′ in the case of the third multi-function valve V3′, or fluidly coupling the third ports 60 of both of the supplemental bioreactor units 36₂, 36₃ respectively to the common port CP of the second multi-function valve V2′, and thus, to one of the media sources s4-s7 selected by the second multi-function valve V2′ in the case of the fourth multi-function valve V4′), while respectively fluidly coupling its return ports RP1, RP2 to the output port OP (thereby fluidly coupling the second ports 58 of both of the supplemental bioreactor units 36₂, 36₃ respectively to the first port 56 of the base bioreactor unit 36₁ in the case of the third multi-function valve V3′, or fluidly coupling the fourth ports 58 of both of the supplemental bioreactor units 36₂, 36₃ respectively to the third port 60 of the base bioreactor unit 36₁ in the case of the fourth multi-function valve V4′), and while fluidly decoupling its output port OP from its common port CP (thereby fluidly decoupling the first port 56 of the base bioreactor unit 36₁ from the common port CP of the first multi-function valve V1′, and thus, from all of the media sources s1-s3 and destination source d1 in the case of the third multi-function valve V3′, or fluidly decoupling the third port 60 of the base bioreactor unit 36₁ from the common port CP of the second multi-function valve V2′, and thus, from all of the media sources s4-s7 in the case of the fourth multi-function valve V4′).

Each of the multi-function valves V3′, V4′ may also have a second plurality of positions (e.g., Positions 2 and 4) for respectively fluidly coupling its selector ports SP1, SP2 to its common port CP (thereby fluidly coupling the first ports 56 of both of the supplemental bioreactor units 36₂, 36₃ respectively to the common port CP of the first multi-function valve V1′, and thus, to one of the media sources s1-s3 and media destination d1 selected by the first multi-function valve V1′ in the case of the third multi-function valve V3′, or fluidly coupling the third ports 60 of both of the supplemental bioreactor units 36₂, 36₃ respectively to the common port CP of the second multi-function valve V2′, and thus, to one of the media sources s4-s7 selected by the second multi-function valve V2′ in the case of the fourth multi-function valve V4′), while fluidly decoupling both of its return ports RP1, RP2 from its output port OP (thereby fluidly decoupling the second ports 58 of both of the supplemental bioreactor units 36₂, 36₃ from the first port 56 of the base bioreactor unit 36₁ in the case of the third multi-function valve V3′, or fluidly decoupling the fourth ports 58 of both of the supplemental bioreactor units 36₂, 36₃ from the third port 60 of the base bioreactor unit 36₁ in the case of the fourth multi-function valve V4′), and while fluidly decoupling its output port OP from its common port CP (thereby fluidly decoupling the first port 56 of the base bioreactor unit 36₁ from the common port CP of the first multi-function valve V1′, and thus, from all of the media sources s1-s3 and destination source d1 in the case of the third multi-function valve V3′, or fluidly decoupling the third port 60 of the base bioreactor unit 36₁ from the common port CP of the second multi-function valve V2′, and thus, from all of the media sources s4-s7 in the case of the fourth multi-function valve V4′).

Each of the multi-function valves V3′, V4′ may also have a third position (e.g., Position 5) for fluidly decoupling both of its selector ports SP1, SP2 from its common port CP (thereby fluidly decoupling the first ports 56 of both of the supplemental bioreactor units 36₂, 36₃ respectively from the common port CP of the first multi-function valve V1′, and thus, from all of the media sources s1-s3 and media destination d1 in the case of the third multi-function valve V3′, or fluidly decoupling the third ports 60 of both of the supplemental bioreactor units 36₂, 36₃ respectively from the common port CP of the second multi-function valve V2′, and thus, from all of the media sources s4-s7 in the case of the fourth multi-function valve V4′), while fluidly decoupling its return ports RP1, RP2 from its output port OP (thereby fluidly decoupling the second ports 58 of both of the supplemental bioreactor units 36₂, 36₃ from the first port 56 of the base bioreactor unit 36₁ in the case of the third multi-function valve V3′, or fluidly decoupling the fourth ports 58 of both of the supplemental bioreactor units 36₂, 36₃ from the third port 60 of the base bioreactor unit 36₁ in the case of the fourth multi-function valve V4′), and while fluidly decoupling its output port OP from its common port CP (thereby fluidly decoupling the first port 56 of the base bioreactor unit 36₁ from the common port CP of the first multi-function valve V1′, and thus, from all of the media sources s1-s3 and destination source d1 in the case of the third multi-function valve V3′, or fluidly decoupling the third port 60 of the base bioreactor unit 36₁ from the common port CP of the second multi-function valve V2′, and thus, from all of the media sources s4-s7 in the case of the fourth multi-function valve V4′).

Each of the multi-function valves V3′, V4′ may optionally have a fourth position (e.g., Position 6) for fluidly decoupling both of its selector ports SP1, SP2 from its common port CP (thereby fluidly decoupling the first ports 56 of both of the supplemental bioreactor units 36₂, 36₃ respectively from the common port CP of the first multi-function valve V1′, and thus, from all of the media sources s1-s3 and media destination d1 in the case of the third multi-function valve V3′, or fluidly decoupling the third ports 60 of the both of supplemental bioreactor units 36₂, 36₃ respectively from the common port CP of the second multi-function valve V2′, and thus, from all of the media sources s4-s7 in the case of the fourth multi-function valve V4′), while fluidly decoupling its return ports RP1, RP2 from its output port OP (thereby fluidly decoupling the second ports 58 of both of the supplemental bioreactor units 36₂, 36₃ from the first port 56 of the base bioreactor unit 36₁ in the case of the third multi-function valve V3′, or fluidly decoupling the fourth ports 58 of both of the supplemental bioreactor units 36₂, 36₃ from the third port 60 of the base bioreactor unit 36₁ in the case of the fourth multi-function valve V4′), and while fluidly coupling its output port OP to its common port CP (thereby fluidly coupling the first port 56 of the base bioreactor unit 36₁ to the common port CP of the first multi-function valve V1′, and thus, to one of the media sources s1-s3 and destination source d1 selected by the first multi-function valve V1′ in the case of the third multi-function valve V3′, or fluidly coupling the third port 60 of the base bioreactor unit 36₁ to the common port CP of the second multi-function valve V2′, and thus, to one of the media sources s4-s7 selected by the second multi-function valve V2′ in the case of the fourth multi-function valve V4′).

Thus, it can be appreciated that the multi-function valves V1′-V4′ can be collectively controlled by the electric controller 22 select the media sources s1-s7 and media destinations d1, and to define a particular flow path between the selected ones of the media sources s1-s7 and media destinations d1-2 and through the interior cavity 44 of a selected one of the bioreactor units 36₁-36₃. In the same manner described above with respect to the bioreactor system 10b illustrated in FIG. 18, the pumps P1, P2 are configured for providing the necessary fluid pressure for conveying various media along flow paths 86a-86h for the purposes of (1) priming the bioreactor assembly 12 (i.e., removing all air within the bioreactor assembly 12); (2) seeding the bioreactor assembly 12 with a population of biological cells (with or without microbeads); (3) culturing the biological cells, such that the population of biological cells are expanded; washing the biological cells; (4) harvesting or sampling the expanded population of biological cells from the bioreactor assembly 12; and (5) clearing the fluid conduits 20 (i.e., relocating stray biological cells from the fluid conduits 20 back into the bioreactor assembly 12).

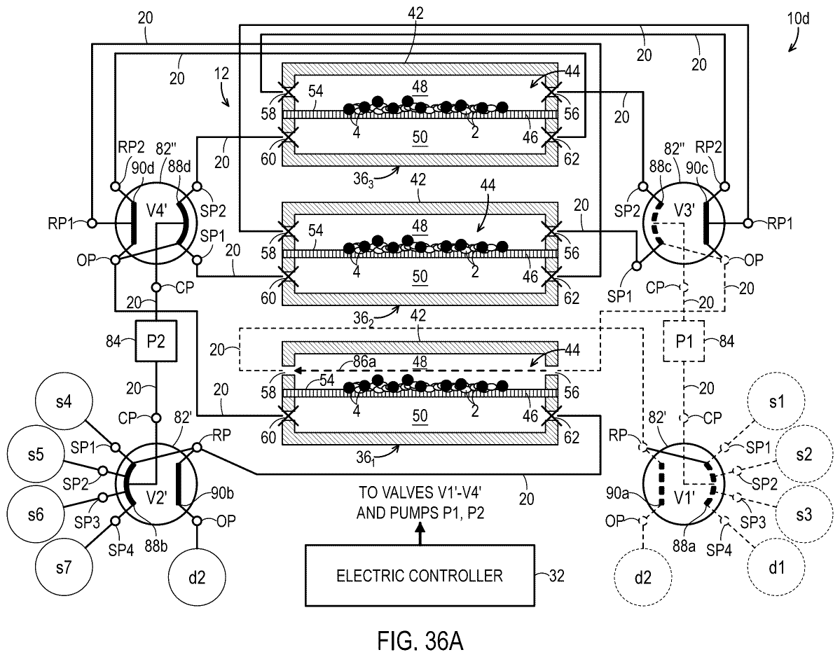

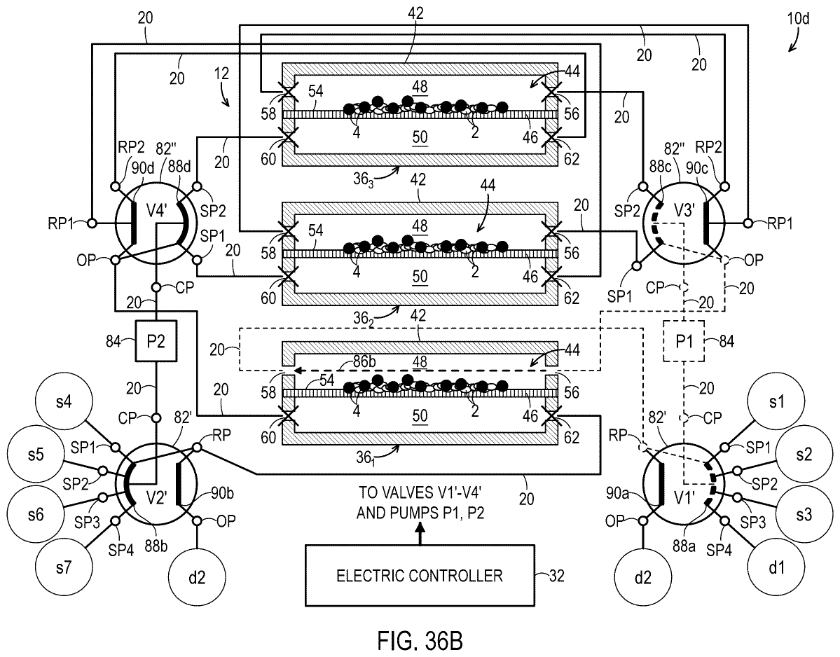

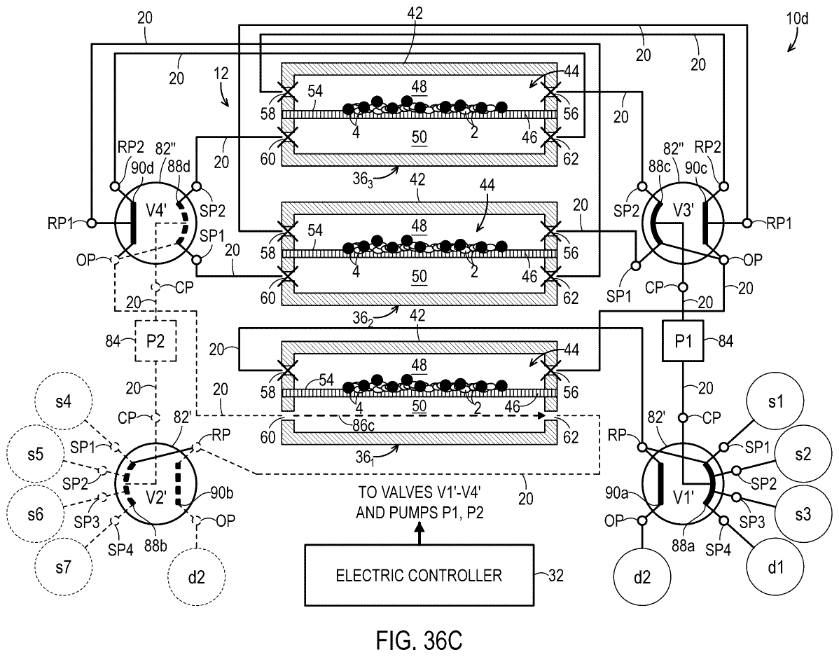

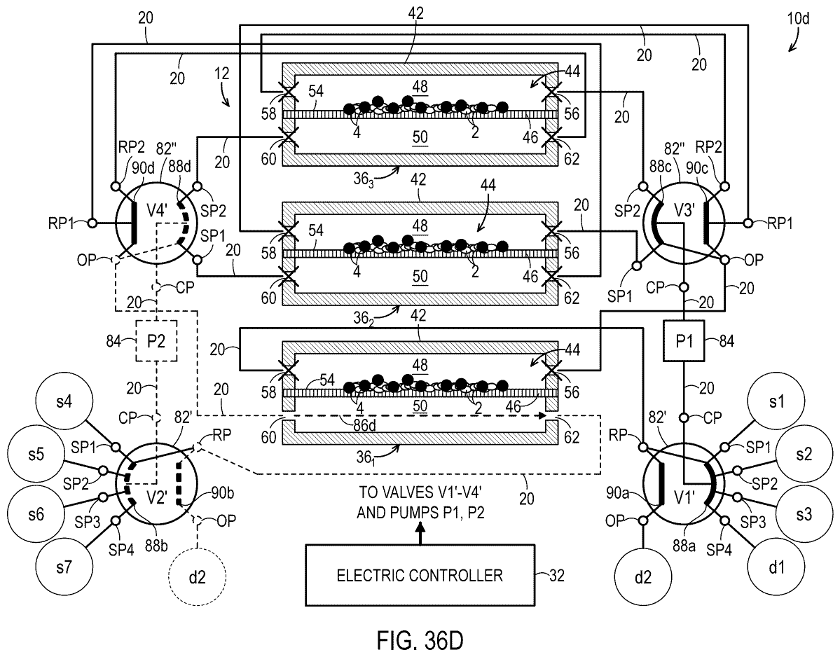

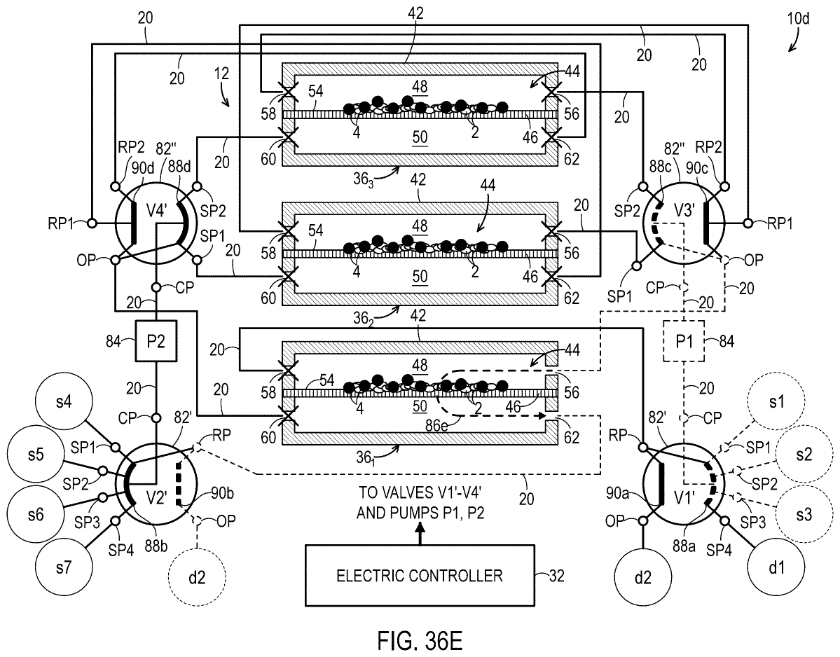

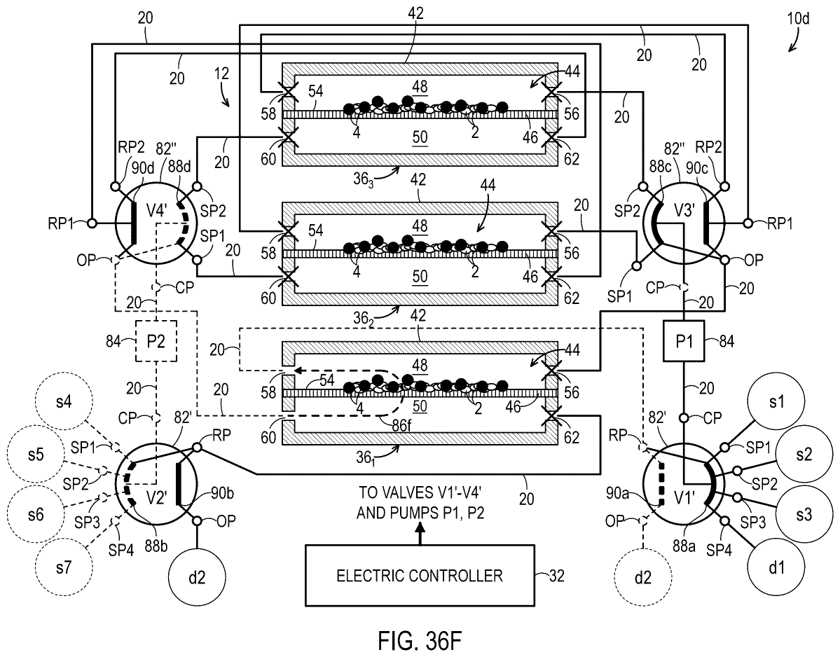

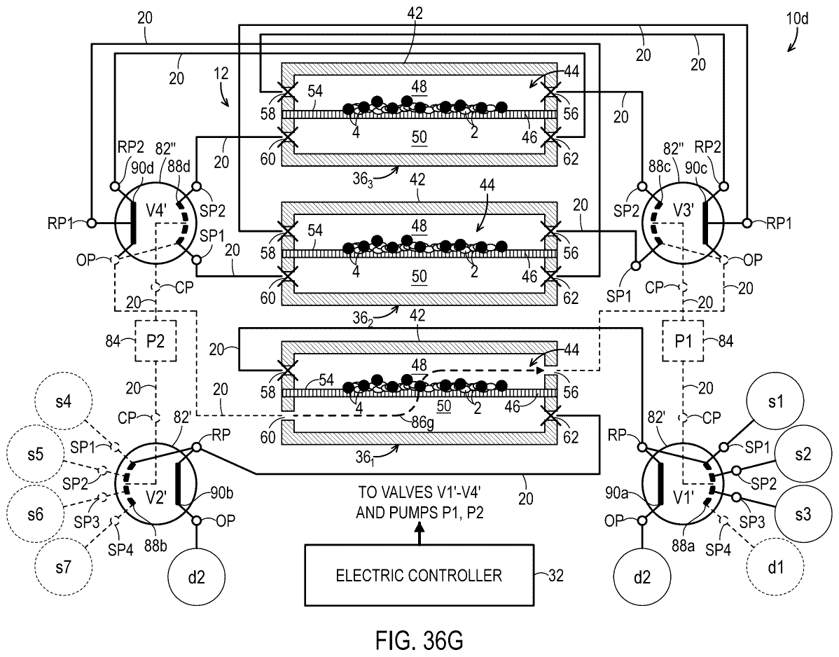

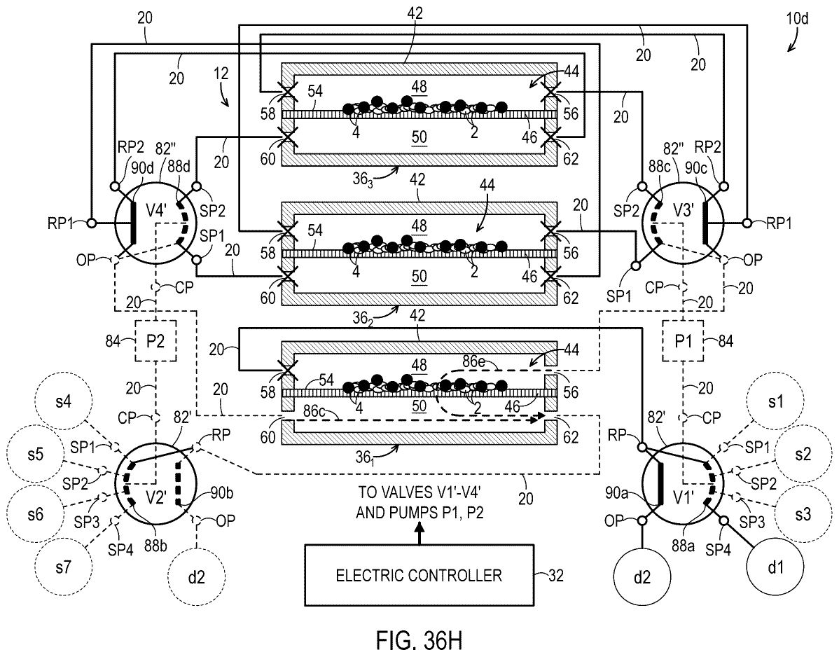

For example, in certain arrangements illustrated in FIGS. 36A-36H, the third and fourth multi-function valves V3′, V4′ may be operated in a manner (essentially by being placed into Position 6 defined in FIG. 35) that define flow paths only through the base bioreactor unit 36₁ (e.g., any of the flow paths illustrated in FIGS. 19A-19H), effectively bypassing both of the supplemental bioreactor units 36₂, 36₃. Notably, the particular media sources s1-s7, media destinations d1-d2, portions of the valves V1′-V4′, pumps P1, P2, and fluid conduits 20 from, through, and to which media associated with a particular flow is shown dashed for purposes of illustration in FIGS. 36A-36H.

In one arrangement illustrated in FIG. 36A, the multi-function valves V1′-V4′ may be operated to define a first flow path 86a entirely through the upper flow channel 48 of only the base bioreactor unit 36₁ via its first and second ports 56, 58 (e.g., to perform such functions as priming of the base bioreactor 36₁ in the same manner described above with respect to bioreactor 36 illustrated in FIG. 19A).

In particular, the first multi-function valve V1′ is operated to fluidly couple a selected one of the media sources s1-s3 to the third multi-function valve V3′, and to fluidly couple the media destination d2 to the second port 58 of the base bioreactor unit 36₁; the third multi-function valve V3′ is operated to fluidly couple the selected one of the media sources s1-s3 to the first port 56 of the base bioreactor unit 36₁, while fluidly decoupling the selected one of the media sources s1-s3 from the first ports 56 of both of the supplemental bioreactor units 36₂, 36₃, and fluidly decoupling the second ports 58 of both of the supplemental bioreactor units 36₂, 36₃ from the first port 56 of the base bioreactor unit 36₁; the second multi-function valve V2′ is operated to fluidly decouple all of the media sources s4-s7 from the fourth multi-function valve V4′, and thus from the third ports 60 of all of the bioreactor units 36, and to fluidly decouple the media destination d2 from the fourth port 62 of the base bioreactor unit 36₁; and the fourth multi-function valve V4′ is operated to fluidly decouple the fourth ports 62 of both of the supplemental bioreactor units 36₂, 36₃ from the third port 60 of the base bioreactor unit 36₁.

In this arrangement, the first multi-function valve V1′ may be placed in any one of Positions 1, 3, or 5 defined in FIG. 23 (depending on the particular one of the media sources s1-s3 to be selected), the second multi-function valve V2′ may be placed into Position 9 defined in FIG. 23, the third multi-function valve V3′ may be placed into Position 6 defined in FIG. 35, and the fourth multi-function valve V4′ may be placed into Position 5 defined in FIG. 35, effectively opening the first and second ports 56, 58 of the base bioreactor unit 36₁ between the selected one of the media sources s1-s3 and the media destination d2, while closing the first ports 56 of both of the supplemental bioreactor units 36₂, 36₃ to the selected one of the media sources s1-s3, closing the second ports 58 of both of the supplemental bioreactor units 36₂, 36₃ to the media destination d2, closing the third ports 60 of all of the bioreactor units 36 to all of the media sources s4-s7, and closing the fourth ports 62 of all of the bioreactor units 36 to the media destination d2, as illustrated in FIG. 36A.

In another arrangement illustrated in FIG. 36B, the multi-function valves V1′-V4′ may be operated to define a second flow path 86b entirely through the upper flow channel 48 of only the base bioreactor unit 36₁ via its first and second ports 56, 58 that bypasses the media sources s1-s7 and media destinations d1-d2 (e.g., to perform such functions as clearing the fluid conduits 20 respectively fluidly connected to the first and second ports 56, 58 of the base bioreactor unit 36₁ in the same manner described above with respect to bioreactor 36 illustrated in FIG. 19B).

In particular, the first multi-function valve V1′ is operated to fluidly decouple all of the media sources s1-s3 and media destination d1 from the third multi-function valve V3′, and thus from the first ports 56 of all of the bioreactor units 36, and fluidly decouple the media destination d2 from the second port 58 of the base bioreactor unit 36₁; the first multi-function valve V1′ and the third multi-function valve V3′ are operated to fluidly couple the first and second ports 56, 58 of the base bioreactor unit 36₁ to each other, while fluidly decoupling the second ports 58 of both of the supplemental bioreactor units 36₂, 36₃ from the first port 56 of the base bioreactor unit 36₁; the second multi-function valve V2′ is operated to fluidly decouple all of the media sources s4-s7 from the fourth multi-function valve V4′, and thus from the third ports 60 of all of the bioreactor units 36, and to fluidly decouple the media destination d2 from the fourth port 62 of the base bioreactor unit 36₁; and the fourth multi-function valve V4′ is operated to fluidly decouple the fourth ports 62 of both of the supplemental bioreactor units 36₂, 36₃ from the third port 60 of the base bioreactor unit 36₁.

In this arrangement, the first multi-function valve V1′ may be placed into Position 10 defined in FIG. 23, the second multi-function valve V2′ may be placed into Position 9 defined in FIG. 23, the third multi-function valve V3′ may be placed into Position 6 defined in FIG. 35, and the fourth multi-function valve V4′ may be placed into Position 5 defined in FIG. 35, effectively opening the first and second ports 56, 58 of the base bioreactor unit 36₁ to each other, while closing the first port 56 of the base bioreactor unit 36₁ to all the media sources s1-s3 and media destination d1, closing the second port 58 of the base bioreactor unit 36₁ to the media destination d2, closing the first ports 56 of both of the supplemental bioreactor units 36₂, 36₃ to all the media sources s1-s3, closing the second ports 58 of both of the supplemental bioreactor units 36₂, 36₃ to the media destination d2, closing the third ports 60 of all of the bioreactor units 36 to all of the media sources s4-s7, and closing the fourth ports 62 of all of the bioreactor units 36 to the media destination d2, as illustrated in FIG. 36B.

In still another arrangement illustrated in FIG. 36C, the multi-function valves V1′-V4′ may be operated to define a third flow path 86c entirely through the lower flow channel 50 of only the base bioreactor unit 36₁ via its third and fourth ports 60, 62 (e.g., to perform such functions as priming of the base bioreactor unit 36₁ or culturing the biological cells 2 residing in the upper flow channel 48 on the cell culture surface 54 of the porous membrane 46 of the base bioreactor unit 36₁ with fresh media containing nutrient, reagent, or gas in the same manner described above with respect to bioreactor 36 illustrated in FIG. 19C).

In particular, the second multi-function valve V2′ is operated to fluidly couple a selected one of the media sources s4-s7 to the fourth multi-function valve V4′, and to fluidly couple the media destination d2 to the fourth port 62 of the base bioreactor unit 36₁; the fourth multi-function valve V4′ is operated to fluidly couple the selected one of the media sources s4-s7 to the third port 60 of the base bioreactor unit 36₁, while fluidly decoupling the selected one of the media sources s4-s7 from the third ports 60 of both of the supplemental bioreactor units 36₂, 36₃, and while fluidly decoupling the fourth ports 62 of both of the supplemental bioreactor units 36₂, 36₃ from the third port 60 of the base bioreactor unit 36₁; the first multi-function valve V1′ is operated to fluidly decouple all of the media sources s1-s3 and media destination d1 from the second multi-function valve V2′, and thus from the first ports 56 of all of the bioreactor units 36, and to fluidly decouple the media destination d2 from the second port 58 of the base bioreactor unit 36₁; and the third multi-function valve V3′ is operated to fluidly decouple the second ports 58 of both of the supplemental bioreactor units 36₂, 36₃ from the first port 56 of the base bioreactor unit 36₁.

In this arrangement, the first multi-function valve V1′ may be placed into Position 9 defined in FIG. 23, the second multi-function valve V2′ may be placed in any one of Positions 1, 3, 5, or 7 (depending on the particular one of the media sources s4-s7 to be selected) defined in FIG. 23, the third multi-function valve may be placed in Position 5 defined in FIG. 35, and the fourth multi-function valve V4′ may be placed into Position 6 defined in FIG. 35, effectively opening the third and fourth ports 60, 62 of the base bioreactor unit 36₁ between the selected one of the media sources s4-s7 and the media destination d2, while closing the first ports 56 of all of the bioreactor units 36 to all of the media sources s1-s3 and media destination d1, closing the second ports 58 of all of the bioreactor units 36 to the media destination d2, closing the third ports 60 of both of the supplemental bioreactor units 36₂, 36₃ to the selected one of the media sources s4-s7, and closing the fourth ports 62 of both of the supplemental bioreactor units 36₂, 36₃ to the media destination d2, as illustrated in FIG. 36C.

In yet another arrangement illustrated in FIG. 36D, the multi-function valves V1′-V4′ may be operated to define a fourth flow path 86d entirely through the lower flow channel 50 of only the base bioreactor unit 36₁ via its third and fourth ports 60, 62 that bypasses the media sources s1-s7 and media destinations d1-d2 (e.g., to perform such functions as recirculating media containing nutrient, reagent, and gas through the lower flow channel 50 of the base bioreactor unit 36₁ in the same manner described above with respect to bioreactor 36 illustrated in FIG. 19D).

In particular, the second multi-function valve V2′ is operated to fluidly decouple all of the media sources s4-s7 from the fourth multi-function valve V4′, and thus from the third ports 60 of all of the bioreactor units 36, and fluidly decouple the media destination d2 from the fourth port 62 of the base bioreactor unit 36₁; the second multi-function valve V2′ and the fourth multi-function valve V4′ are operated to fluidly couple the third and fourth ports 60, 62 of the base bioreactor unit 36₁ to each other, while fluidly decoupling the fourth ports 62 of both of the supplemental bioreactor units 36₂, 36₃ from the third port 60 of the base bioreactor unit 36₁; the first multi-function valve V1′ is operated to fluidly decouple all of the media sources s1-s3 and media destination d1 from the third multi-function valve V3′, and thus from the first ports 56 of all of the bioreactor units 36, and to fluidly decouple the media destination d2 from the second port 58 of the base bioreactor unit 36₁; and the third multi-function valve V3′ is operated to fluidly decouple the second ports 58 of both of the supplemental bioreactor units 36₂, 36₃ from the first port 56 of the base bioreactor unit 36₁.

In this arrangement, the first multi-function valve V1′ may be placed into Position 9 defined in FIG. 23, the second multi-function valve V2′ may be placed into Position 10 defined in FIG. 23, the third multi-function valve V3′ may be placed into Position 5 defined in FIG. 35, and the fourth multi-function valve V4′ may be placed into Position 6 defined in FIG. 35, effectively opening the third and fourth 60, 62 of the base bioreactor unit 36₁ to each other, while closing the third port 60 of the base bioreactor unit 36₁ to all the media sources s4-s7, closing the fourth port 62 of the base bioreactor unit 36₁ to the media destination d2, closing the first ports 56 of all of the bioreactor units 36 to all of the media sources s1-s3 and media destination d1, closing the second ports 58 of all of the bioreactor units 36 to the media destination d2, closing the third ports 60 of both of the supplemental bioreactor units 36₂, 36₃ to all the media sources s4-s7, and closing the fourth ports 62 of both of the supplemental bioreactor units 36₂, 36₃ to the media destination d2, as illustrated in FIG. 36D.

In yet another arrangement illustrated in FIG. 36E, the multi-function valves V1′-V4′ may be operated to define a fifth flow path 86e into the upper flow channel 48 via the first port 56, between the upper and lower flow channels 48, 50 via the porous membrane 46, and out of the lower flow channel 50 via the fourth port 62, of only the base bioreactor unit 36₁ (e.g., to perform such functions as seeding the bioreactor 36 with biological cells 2 and/or microbeads 4, washing the biological cells 2 and/or microbeads 4, or clearing the fluid conduit 20 fluidly connected to the first port 56 of the base bioreactor unit 36₁ after sampling of the biological cells 2 in the same manner described above with respect to bioreactor 36 illustrated in FIG. 19E).

In particular, the first multi-function valve V1′ is operated to fluidly couple a selected one of the media sources s1-s3 to the third multi-function valve V3′, and to fluidly decouple the media destination d2 from the second port 58 of the base bioreactor unit 36₁; the third multi-function valve V3′ is operated to fluidly couple the selected one of the media sources s1-s3 to the first port 56 of the base bioreactor unit 36₁, while fluidly decoupling the selected one of the media sources s1-s3 from the first ports 56 of both of the supplemental bioreactor units 36₂, 36₃, and while fluidly decoupling the second ports 58 of both of the supplemental bioreactor units 36₂, 36₃ from the first port 56 of the base bioreactor unit 36₁; the second pump P2 is deactivated to fluidly decouple all of the media sources s4-s7 from the fourth multi-function valve V4′, and thus from the third ports 60 of all of the bioreactor units 36; the second multi-function valve V2′ is operated to fluidly couple the media destination d2 to the fourth port 62 of the base bioreactor unit 36₁; and the fourth multi-function valve V4′ is operated to fluidly decouple the fourth ports 62 of both of the supplemental bioreactor units 36₂, 36₃ from the third port 60 of the base bioreactor unit 36₁.

In this arrangement, the first multi-function valve V1′ may be placed in any one of Positions 2, 4, or 6 defined in FIG. 23 (depending on the particular one of the media sources s1-s3 to be selected), the second multi-function valve V2′ may be placed in any one of Positions 1, 3, 5, or 7 defined in FIG. 23), the third multi-function valve V3′ may be placed into Position 6 defined in FIG. 35, and the fourth multi-function valve V4′ may be placed into Position 5 defined in FIG. 35, effectively opening the first port 56 of the base bioreactor unit 36₁ to the selected one of the media sources s1-s3, and opening the fourth port 62 of the base bioreactor unit 36₁ to the media destination d2, while closing the first ports 56 of both of the supplemental bioreactor units 36₂, 36₃ to the selected one of the media sources s1-s3, closing the second ports 58 of all of the bioreactor units 36 to the media destination d2, closing the third ports 60 of all of the bioreactor units 36 to all of the media sources s4-s7, and closing the fourth ports 62 of both of the supplemental bioreactor units 36₂, 36₃ to the media destination d2, as illustrated in FIG. 36E.

In yet another arrangement illustrated in FIG. 36F, the multi-function valves V1′-V4′ may be operated to define a sixth flow path 86f into the lower flow channel 50 via the third port 60, between the upper and lower flow channels 48, 50 via the porous membrane 46, and out of the upper flow channel 48 via the second port 58, of only the base bioreactor unit 36₁ (e.g., to perform such functions as refreshing the media in the upper flow channel 48 or suspending the biological cells 2 and/or microbeads 4 in the upper flow channel 48 of the base bioreactor unit 36₁ in the same manner described above with respect to bioreactor 36 illustrated in FIG. 19F).

In particular, the second multi-function valve V2′ is operated to fluidly couple a selected one of the media sources s4-s7 to the fourth multi-function valve V4′, and to fluidly decouple the media destination d2 from the fourth port 62 of the base bioreactor unit 36₁; the fourth multi-function valve V4′ is operated to fluidly couple the selected one of the media sources s4-s7 to the third port 60 of the base bioreactor unit 36₁, while fluidly decoupling the selected one of the media sources s4-s7 from the third ports 60 of both of the supplemental bioreactor units 36₂, 36₃, and fluidly decoupling the fourth ports 62 of both of the supplemental bioreactor units 36₂, 36₃ from the third port 60 of the base bioreactor unit 36₁; the first pump P1 is deactivated to fluidly decouple all of the media sources s1-s3 and media destination d1 from the third multi-function valve V3′, and thus from the first ports 56 of all of the bioreactor units 36; the first multi-function valve V1′ is operated to fluidly couple the media destination d1 to the second port 58 of the base bioreactor unit 36₁; and the third multi-function valve V3′ is operated to fluidly decouple the second ports 58 of both of the supplemental bioreactor units 36₂, 36₃ from the first port 56 of the base bioreactor unit 36₁.

In this arrangement, the first multi-function valve V1′ may be placed in any one of Positions 1, 3, 5, or 7 defined in FIG. 23, the second multi-function valve V2′ may be placed in any one of Positions 2, 4, 6, or 8 defined in FIG. 23 (depending on the particular one of the media sources s4-s7 to be selected), the third multi-function valve V3′ may be placed into Position 5 defined in FIG. 35, and the fourth multi-function valve V4′ may be placed into Position 6 defined in FIG. 35, effectively opening the third port 60 of the base bioreactor unit 36₁ to the selected one of the media sources s4-s7, and opening the second port 58 of the base bioreactor unit 36₁ to the media destination d2, while closing the first ports 56 of all of the bioreactor units 36 to all of the media sources s1-s3 and media destination d1, closing the second ports 58 of both of the supplemental bioreactor units 36₂, 36₃ to the media destination d2, closing the third ports 60 of both of the supplemental bioreactor units 36₂, 36₃ to the selected one of the media sources s4-s7, and closing the fourth ports 62 of all of the bioreactor units 36 to the media destination d2, as illustrated in FIG. 36F.

In yet another arrangement illustrated in FIG. 36G, the multi-function valves V1′-V4′ may be operated to define a seventh flow path 86g into the lower flow channel 50 via the third port 60, between the lower and upper flow channels 50, 48 via the porous membrane 46, and out of the upper flow channel 48 via the first port 56, of only the base bioreactor unit 36₁ (e.g., to perform such functions as harvesting or sampling of the biological cells 2 from the base bioreactor unit 36₁ in the same manner described above with respect to bioreactor 36 illustrated in FIG. 19G).

In particular, the second multi-function valve V2′ is operated to fluidly couple a selected one of the media sources s4-s7 to the fourth multi-function valve V4′, and to fluidly decouple the media destination d2 from the fourth port 62 of the base bioreactor unit 36₁; the fourth multi-function valve V4′ is operated to fluidly couple the selected one of the media sources s4-s7 to the third port 60 of the base bioreactor unit 36₁, while fluidly decoupling the selected one of the media sources s4-s7 from the third ports 60 of both of the supplemental bioreactor units 36₂, 36₃, and while fluidly decoupling the fourth ports 62 of both of the supplemental bioreactor units 36₂, 36₃ from the third port 60 of the base bioreactor unit 36₁; the first multi-function valve V1′ is operated to fluidly couple the media destination d1 to the third multi-function valve V3′, and to fluidly decouple the media destination d2 from the second port 58 of the base bioreactor unit 36₁; the third multi-function valve V3′ is operated to fluidly couple the media destination d1 to the first port 56 of the base bioreactor unit 36₁, while fluidly decoupling the second ports 58 of both of the supplemental bioreactor units 36₂, 36₃ from the first port 56 of the base bioreactor unit 36₁, and to fluidly decouple the media destination d1 from the first ports 56 of both of the supplemental bioreactor units 36₂, 36₃,

In this arrangement, the first multi-function valve V1′ may be placed into Position 8 defined in FIG. 23, the second multi-function valve V2′ may be placed in any of one of Positions 2, 4, 6, or 8 defined in FIG. 23) (depending on the particular one of the media sources s4-s7 to be selected), the third multi-function valve V3′ may be placed into Position 6 defined in FIG. 35 (with the caveat that media flows into the output port OP of the third multi-function valve V3′ in a retrograde manner), and the fourth multi-function valve V4′ may be placed into Position 6 defined in FIG. 35, effectively opening the third port 60 of the base bioreactor unit 36₁ to the selected one of the media sources s4-s7, and opening the first port 56 of the base bioreactor unit 36₁ to the media destination d1, while closing the first ports 56 of both of the supplemental bioreactor units 36₂, 36₃ to the media destination d1, closing the second ports 58 of all of the bioreactor units 36 to the media destination d2, closing the third ports 60 of both of the supplemental bioreactor units 36₂, 36₃ to the selected one of the media sources s4-s7, and closing the fourth ports 62 of all of the bioreactor units 36 to the media destination d2, as illustrated in FIG. 36G.

In yet another arrangement illustrated in FIG. 36H, the multi-function valves V1′-V4′ may be operated to define the fifth flow path 86e into the upper flow channel 48 via the first port 56, between the upper and lower flow channels 48, 50 via the porous membrane 46, and out of the lower flow channel 50 via the fourth port 62, of only the base bioreactor unit 36₁, as well as defining the third flow path 86c entirely through the lower flow channel 50 via the third and fourth ports 60, 62 of only the base bioreactor unit 36₁ (e.g., to perform such functions as suspending the biological cells 2 and/or microbeads 4 within the upper flow channel 48 to facilitate clogless perfusion of media from the upper flow channel 48 to the lower flow channel 50 via the porous membrane 46 during seeding of the base bioreactor unit 36₁ with biological cells 2 and/or microbeads 4 or when washing the biological cells 2 and/or microbeads 4 within the base bioreactor unit 36₁ in the same manner described above with respect to bioreactor 36 illustrated in FIG. 19H).

The combination of the third and fifth flow paths 86c and 86e may be used to perform such functions, as, suspending the biological cells 2 and/or microbeads 4 within the upper flow channel 48 to facilitate clogless perfusion of media from the upper flow channel 48 to the lower flow channel 50 via the porous membrane 46 during seeding of the base bioreactor unit 36₁ with biological cells 2 and/or microbeads 4 or when washing the biological cells 2 and/or microbeads 4 within the base bioreactor unit 36₁ in the same manner described above with respect to FIG. 24H.

In particular, the first multi-function valve V1′ is operated to fluidly couple a selected one of the media sources s1-s3 to the third multi-function valve V3′, and to fluidly decouple the media destination d2 from the second port 58 of the base bioreactor unit 36₁; the third multi-function valve V3′ is operated to fluidly couple the selected one of the media sources s1-s3 to the first port 56 of the base bioreactor unit 36₁, while fluidly decoupling the selected one of the media sources s1-s3 from the first ports 56 of both of the supplemental bioreactor units 36₂, 36₃, and while fluidly decoupling the second ports 58 of both of the supplemental bioreactor units 36₂, 36₃ from the first port 56 of the base bioreactor unit 36₁; the second multi-function valve V2′ is operated to fluidly couple a selected one of the media sources s4-s7 to the fourth multi-function valve V4′, and to fluidly couple the media destination d2 to the fourth port 62 of the base bioreactor unit 36₁; the fourth multi-function valve V4′ is operated to fluidly couple the selected one of the media sources s4-s7 to the third port 60 of the base bioreactor unit 36₁, while fluidly decoupling the selected one of the media sources s4-s7 from the third ports 60 of both of the supplemental bioreactor units 36₂, 36₃, and while fluidly decoupling the fourth ports 62 of both of the supplemental bioreactor units 36₂, 36₃ from the third port 60 of the base bioreactor unit 36₁.

In this arrangement, the first multi-function valve V1′ may be placed into any one of Positions 2, 4, or 6 illustrated in FIG. 23 (depending on the particular one of the media sources s1-s3 to be selected), the second multi-function valve V2′ may be placed into any one of Positions 1, 3, 5, or 7 (depending on the particular one of the media sources s4-s7 to be selected) illustrated in FIG. 23, the third multi-function valve V3′ may be placed into Position 6 defined in FIG. 35, and the fourth multi-function valve V4′ may be placed into Position 6 defined in FIG. 35, effectively opening the third and fourth ports 60, 62 of the base bioreactor unit 36₁ between the selected one of the media sources s4-s7 and the media destination d2, and opening the first port 56 of the base bioreactor unit 36₁ to the selected one of the media sources s1-s3, while closing the first ports 56 of both of the supplemental bioreactor units 36₂, 36₃ to the selected one of the media sources media s1-s3, closing the second ports 58 of all of the bioreactor units 36 to the media destination d2, closing the third ports 60 of both of the supplemental bioreactor units 36₂, 36₃ to the selected one of the media sources s4-s7, and closing the fourth ports 62 of both of the supplemental bioreactor units 36₂, 36₃ to the media destination d2, as illustrated in FIG. 36H.

Significantly, the third and fourth multi-function valves V3′, V4′ may be operated to replicate any particular flow path that can be defined for the base bioreactor unit 36₁ (e.g., any of the flow paths 86a-86g and 86c/86e illustrated in FIGS. 36A-36H) within a selected one of the supplemental bioreactor units 36₂, 36₃.

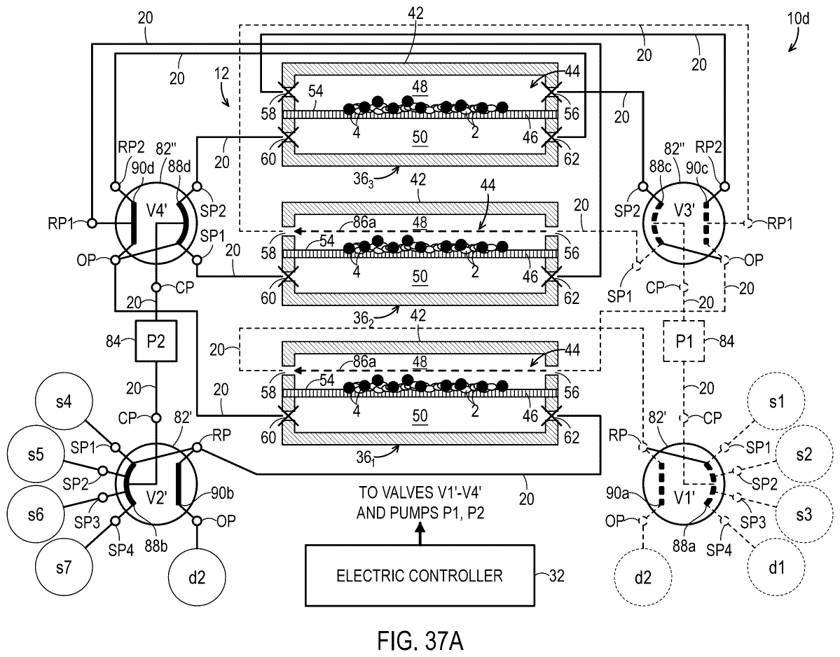

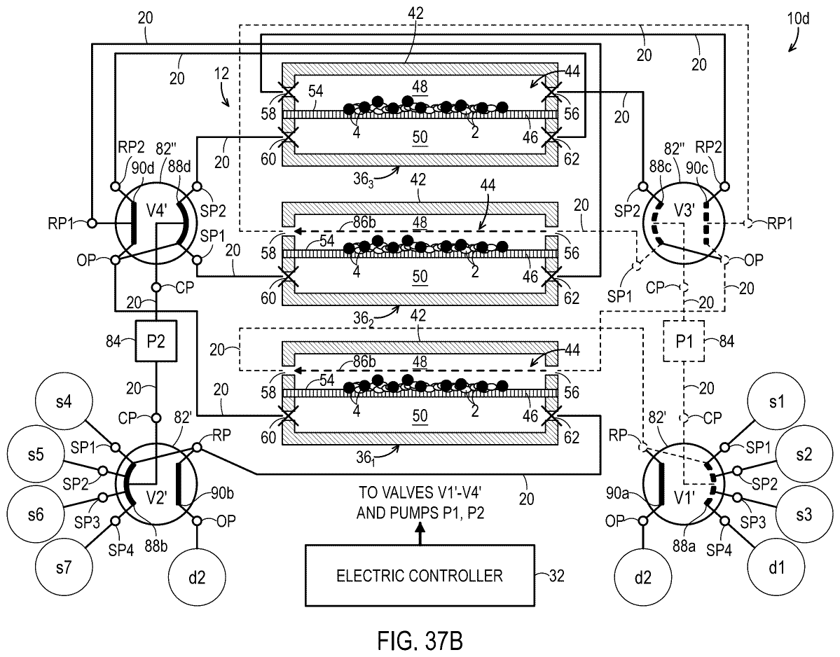

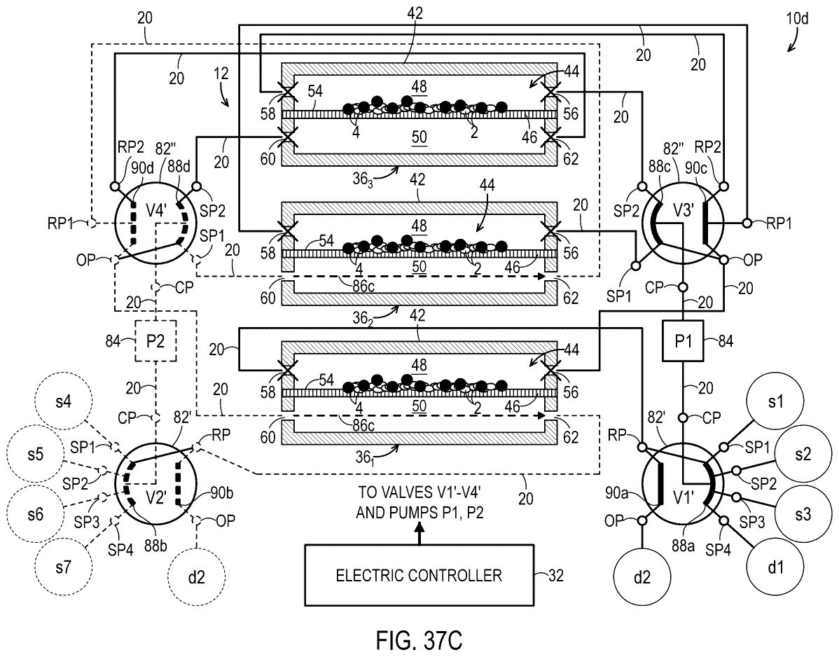

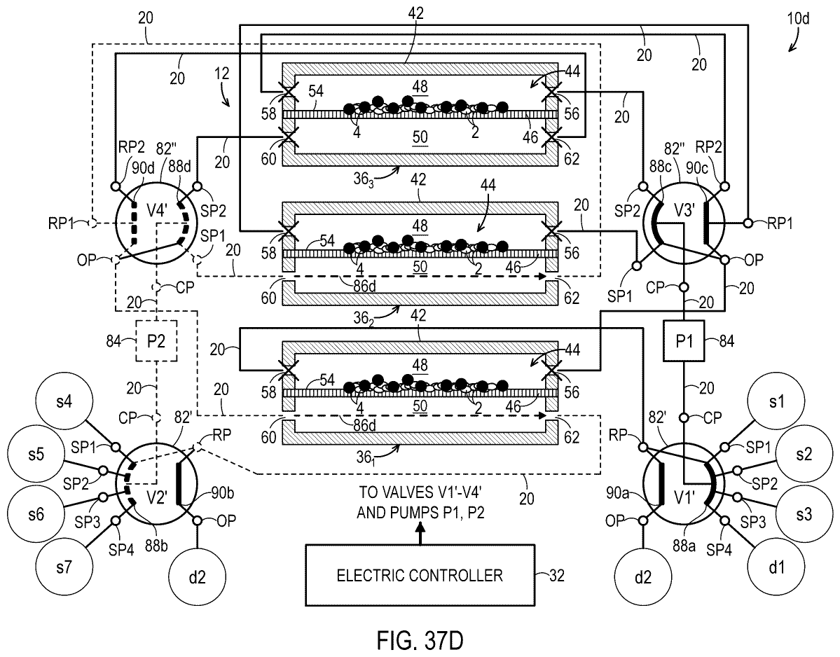

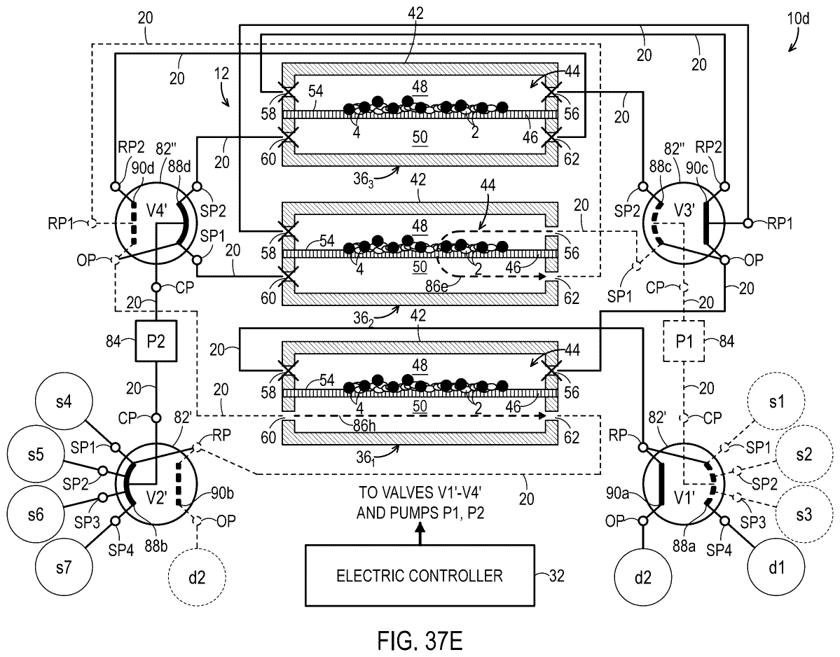

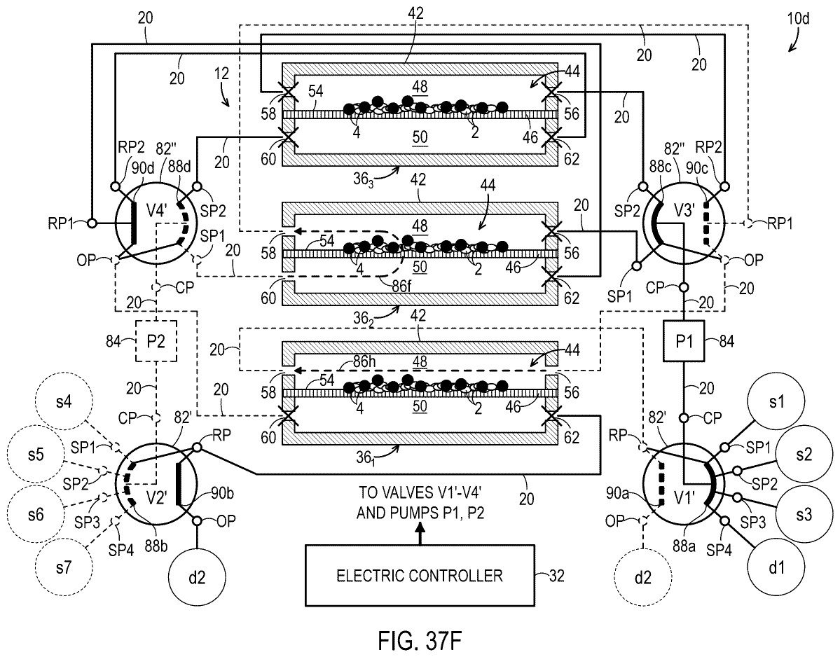

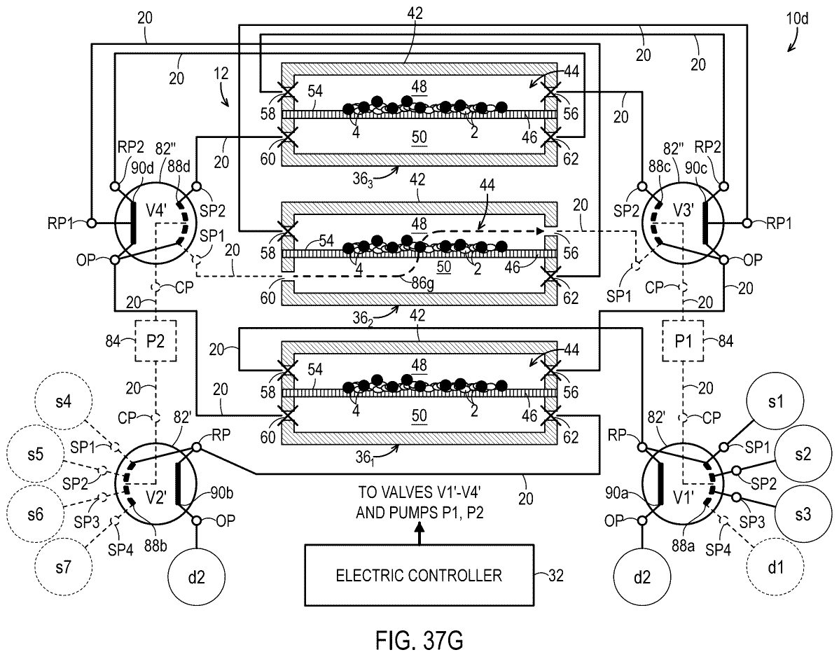

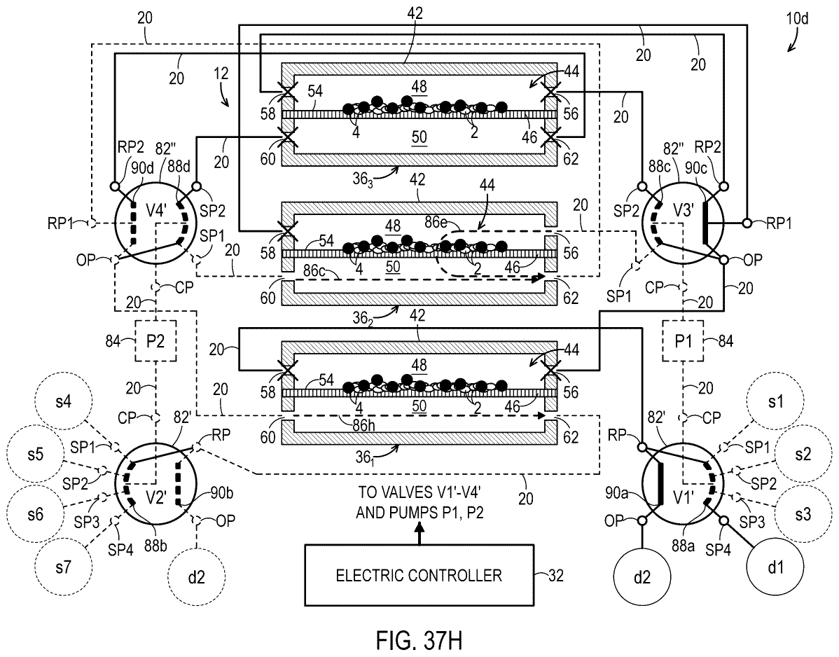

For example, in certain arrangements illustrated in FIGS. 37A-37H, the third and fourth multi-function valves V3′, V4′ may be operated in a manner (essentially by being placed into Positions 1 or 2 defined in FIG. 35) that define the flow paths 86a-86g and 86c/86e through the first supplemental bioreactor unit 36₂. Notably, in certain arrangements with flow paths that flow entirely through a respective one of the flow channels 48, 50 of the first supplemental bioreactor 36₂ (e.g., the flow paths 86a-86d), the respective one of the flow channels 48, 50 of the first supplemental bioreactor 36₂ will be coupled in series with the corresponding one of the flow channels 48, 50 of the base bioreactor unit 36₁, effectively defining the same flow path through the corresponding one of the flow channels 48, 50 of the base bioreactor unit 36₁ (i.e., the same flow path extends through the corresponding ones of the flow channels 48, 50 of the both the base bioreactor unit 36₁ and the first supplemental bioreactor 36₂). In other arrangements with flow paths that extend between the flow channels 48, 50 via the porous membrane 46 of the first supplemental bioreactor 36₂ (e.g., the flow paths 86e-86g), if necessary one of the flow channels 48, 50 of the base bioreactor unit 36₁ will be used to return media from the first supplemental bioreactor 36₂ to the media destination d2 via the first multi-function valve V1′ or the second multi-function valve V2′. Notably, the particular media sources s1-s7, media destinations d1-d2, portions of the valves V1′-V4′, pumps P1, P2, and fluid conduits 20 from, through, and to which media associated with a particular flow is shown dashed for purposes of illustration in FIGS. 37A-37H.

In one arrangement illustrated in FIG. 37A, the multi-function valves V1′-V4′ may be operated to define a first flow path 86a entirely through the upper flow channels 48 of the base bioreactor unit 36₁ and first supplemental bioreactor 36₂ via their first and second ports 56, 58 (e.g., to perform such functions as priming of the base bioreactor 36₁ and first supplemental bioreactor 36₂ in the same manner described above with respect to bioreactor 36 illustrated in FIG. 19A).

In particular, the first multi-function valve V1′ is operated to fluidly couple a selected one of the media sources s1-s3 to the third multi-function valve V3′, and to fluidly couple the media destination d2 to the second port 58 of the base bioreactor unit 36₁; the third multi-function valve V3′ is operated to fluidly couple the selected one of the media sources s1-s3 to the first port 56 of the first supplemental bioreactor 36₂, while fluidly decoupling the selected one of the media sources s1-s3 from the first ports 56 of the base bioreactor unit 36₁ and the second supplemental bioreactor 36₃, and to fluidly couple the second port 58 of the first supplemental bioreactor 36₂ to the first port 56 of the base bioreactor unit 36₁, while fluidly decoupling the second port 58 of the second supplemental bioreactor 36₃ from the first port 56 of the base bioreactor unit 36₁; the second multi-function valve V2′ is operated to fluidly decouple all of the media sources s4-s7 from the fourth multi-function valve V4′, and thus from the third ports 60 of all of the bioreactor units 36, and to fluidly decouple the media destination d2 from the fourth port 62 of the base bioreactor unit 36₁; and the fourth multi-function valve V4′ is operated to fluidly decouple the fourth ports 62 of both of the supplemental bioreactor units 36₂, 36₃ from the third port 60 of the base bioreactor unit 36₁.

In this arrangement, the first multi-function valve V1′ may be placed in one of Positions 1, 3, or 5 defined in FIG. 23 (depending on the particular one of the media sources s1-s3 to be selected), the second multi-function valve V2′ may be placed into Position 9 defined in FIG. 23, the third multi-function valve V3′ may be placed into Position 1 defined in FIG. 35, and the fourth-multi-function valve V4′ may be placed into Position 5 defined in FIG. 35, effectively opening the first port 56 of the first supplemental bioreactor 36₂ to the selected one of the media sources s1-s3, opening the second port 58 of the first supplemental bioreactor 36₂ and the first port 56 of the base bioreactor unit 36₁ to each other, and opening the second port 58 of the base bioreactor unit 36₁ to the media destination d2, while closing the first port 56 of the second supplemental bioreactor 36₃ to the selected one of the media sources s1-s3, closing the second port 58 of the second supplemental bioreactor 36₃ to the media destination d2, closing the third ports 60 of all of the bioreactor units 36 to all of the media sources s4-s7, and closing the fourth ports 62 of all of the bioreactor units 36 to the media destination d2, as illustrated in FIG. 37A.

In another arrangement illustrated in FIG. 37B, the multi-function valves V1′-V4′ may be operated to define a second flow path 86b entirely through the upper flow channels 48 of the base bioreactor unit 36₁ and first supplemental bioreactor 36₂ via their first and second ports 56, 58 that effectively bypasses the media sources s1-s7 and media destinations d1-d2 (e.g., to perform such functions as clearing the fluid conduits 20 respectively fluidly connected to the first and second ports 56, 58 of the base bioreactor unit 36₁ and first supplemental bioreactor unit 36₂ in the same manner described above with respect to bioreactor 36 illustrated in FIG. 19B).

In particular, the first multi-function valve V1′ is operated to fluidly decouple all of the media sources s1-s3 and media destination d1 from the third multi-function valve V3′, and thus from the first ports 56 of all of the bioreactor units 36, and fluidly decouple the media destination d2 from the second port 58 of the base bioreactor unit 36₁; the first multi-function valve V1′ and the third multi-function valve V3′ are further operated to fluidly couple the second port 58 of the first supplemental bioreactor 36₂ to the first port 56 of the base bioreactor unit 36₁, while fluidly decoupling the second port 58 of the second supplemental bioreactor 36₃ from the first port 56 of the base bioreactor unit 36₁, and to fluidly couple the second port 58 of the base bioreactor unit 36₁ to the first port 56 of the first supplemental bioreactor 36₂; the second multi-function valve V2′ is operated to fluidly decouple all of the media sources s4-s7 from the fourth multi-function valve V4′, and thus from the third ports 60 of all of the bioreactor units 36, and to fluidly decouple the media destination d2 from the fourth port 62 of the base bioreactor unit 36₁; and the fourth multi-function valve V4′ is operated to fluidly decouple the fourth ports 62 of both of the supplemental bioreactor units 36₂, 36₃ from the third port 60 of the base bioreactor unit 36₁.

In this arrangement, the first multi-function valve V1′ may be placed into Position 10 defined in FIG. 23, second multi-function valve V2′ may be placed into Position 9 defined in FIG. 23, the third multi-function valve V3′ may be placed into Position 1 defined in FIG. 35, and the fourth multi-function valve V4′ may be placed into Position 5 defined in FIG. 35, effectively opening the second port 58 of the first supplemental bioreactor 36₂ and the first port 56 of the base bioreactor unit 36₁ to each other, and opening the second port 58 of the base bioreactor unit 36₁ and the first port 56 of the first supplemental bioreactor 36₂ to each other, while closing the first ports 56 of all of the bioreactor units 36 to all the media sources s1-s3 and media destination d1, closing the second port 58 of the second supplemental bioreactor 36₃ to the media destination d2, closing the third ports 60 of all of the bioreactor units 36 to all of the media sources s4-s7, and closing the fourth ports 62 of all of the bioreactor units 36 to the media destination d2, as illustrated in FIG. 37B.

In still another arrangement illustrated in FIG. 37C, the multi-function valves V1′-V4′ may be operated to define a third flow path 86c entirely through the lower flow channels 50 of the base bioreactor unit 36₁ and first supplemental bioreactor 36₂ via their first and second ports 56, 58 (e.g., to perform such functions as priming of the base bioreactor unit 36₁ and first supplemental bioreactor unit 36₂ or culturing the biological cells 2 residing in the upper flow channel 48 on the cell culture surface 54 of the porous membrane 46 of the base bioreactor unit 36₁ and first supplemental bioreactor unit 36₂ with fresh media containing nutrient, reagent, or gas in the same manner described above with respect to bioreactor 36 illustrated in FIG. 19C).

In particular, the second multi-function valve V2′ is operated to fluidly couple a selected one of the media sources s4-s7 to the fourth multi-function valve V4′, and to fluidly couple the media destination d2 to the fourth port 62 of the base bioreactor unit 36₁; the fourth multi-function valve V4′ is operated to fluidly couple the selected one of the media sources s4-s7 to the third port 60 of the first supplemental bioreactor 36₂, while fluidly decoupling the selected one of the media sources s4-s7 from the third ports 60 of the base bioreactor unit 36₁ and the second supplemental bioreactor 36₃, and to fluidly couple the fourth port 62 of the first supplemental bioreactor 36₂ to the third port 60 of the base bioreactor unit 36₁, while fluidly decoupling the fourth port 62 of the second supplemental bioreactor 36₃ from the third port 60 of the base bioreactor unit 36₁; the first multi-function valve V1′ is operated to fluidly decouple all of the media sources s1-s3 and media destination d1 from the third multi-function valve V3′, and thus from the first ports 56 of all of the bioreactor units 36, and to fluidly decouple the media destination d2 from the second port 58 of the base bioreactor unit 36₁; and the second multi-function valve V2′ is operated to fluidly decouple the second ports 58 of both of the supplemental bioreactor units 36₂, 36₃ from the first port 56 of the base bioreactor unit 36₁.

In this arrangement, the first multi-function valve V1′ may be placed into Position 9 defined in FIG. 23, the second multi-function valve V2′ may be placed into any one of Positions 1, 3, 5, or 7 defined in FIG. 23 (depending on the particular one of the media sources s4-s7 to be selected), the third multi-function valve V3′ may be placed into Position 5 defined in FIG. 35, and the fourth multi-function valve V4′ may be placed into Position 1, effectively opening the third port 60 of the first supplemental bioreactor 36₂ to the selected one of the media sources s4-s7, opening the fourth port 62 of the first supplemental bioreactor 36₂ and the third port 60 of the base bioreactor unit 36₁ to each other, and opening the fourth port 62 of the base bioreactor unit 36₁ to the media destination d2, while closing the third port 60 of the second supplemental bioreactor 36₃ to the selected one of the media sources s4-s7, closing the fourth port 62 of the second supplemental bioreactor 36₃ to the media destination d2, closing the first ports 56 of all of the bioreactor units 36 to all of the media sources s1-s3 and media destination d1, and closing the second ports 58 of all of the bioreactor units 36 to the media destination d2, as illustrated in FIG. 37C.

In yet another arrangement illustrated in FIG. 37D, the multi-function valves V1′-V4′ may be operated to define a fourth flow path 86d entirely through the lower flow channels 50 of the base bioreactor unit 36₁ and first supplemental bioreactor 36₂ via their third and fourth ports 60, 62 that effectively bypasses the media sources s1-s7 and media destinations d1-d2 (e.g., to perform such functions as recirculating media containing nutrient, reagent, and gas through the lower flow channel 50 of the base bioreactor unit 36₁ and first supplemental bioreactor unit 36₂ in the same manner described above with respect to bioreactor 36 illustrated in FIG. 19D).

In particular, the second multi-function valve V2′ is operated to fluidly decouple all of the media sources s4-s7 from the fourth multi-function valve V4′, and thus from the third ports 60 of all of the bioreactor units 36, and fluidly decouple the media destination d2 from the fourth port 62 of the base bioreactor unit 36₁; the second multi-function valve V2′ and the fourth multi-function valve V3′ are further operated to fluidly couple the fourth port 62 of the first supplemental bioreactor 36₂ to the third port 60 of the base bioreactor unit 36₁, while fluidly decoupling the fourth port 62 of the second supplemental bioreactor 36₃ from the third port 60 of the base bioreactor unit 36₁, and to fluidly couple the fourth port 62 of the base bioreactor unit 36₁ to the third port 60 of the first supplemental bioreactor 36₂; the first multi-function valve V1′ is operated to fluidly decouple all of the media sources s1-s3 and media destination d1 from the third multi-function valve V3′, and thus from the first ports 56 of all of the bioreactor units 36, and to fluidly decouple the media destination d2 from the second port 58 of the base bioreactor unit 36₁; and the third multi-function valve V3′ is operated to fluidly decouple the second ports 58 of both of the supplemental bioreactor units 36₂, 36₃ from the first port 56 of the base bioreactor unit 36₁.

In this arrangement, the first multi-function valve V1′ may be placed into Position 9 defined in FIG. 23, second multi-function valve V2′ may be placed into Position 10 defined in FIG. 23, the third multi-function valve V3′ may be placed into Position 5 defined in FIG. 35, and the fourth multi-function valve V4′ may be placed into Position 1 defined in FIG. 35, effectively opening the fourth port 62 of the first supplemental bioreactor 36₂ and the third port 60 of the base bioreactor unit 36₁ to each other, and opening the fourth port 62 of the base bioreactor unit 36₁ and the third port 60 of the first supplemental bioreactor 36₂ to each other, while closing the third ports 60 of all of the bioreactor units 36 to all the media sources s4-s7, closing the fourth port 62 of the second supplemental bioreactor 36₃ to the media destination d2, and closing the second ports 58 of all of the bioreactor units 36 to the media destination d2, as illustrated in FIG. 37D.

In yet another arrangement illustrated in FIG. 37E, the multi-function valves V1′-V4′ may be operated to define a fifth flow path 86e into the upper flow channel 48 via the first port 56, between the upper and lower flow channels 48, 50 via the porous membrane 46, and out of the lower flow channel 50 via the fourth port 62, of only the first supplemental bioreactor 36₂, while providing a return flow path 86h through the lower flow channel 50 of the base bioreactor unit 36₁ (e.g., to perform such functions as seeding the bioreactor 36 with biological cells 2 and/or microbeads 4, washing the biological cells 2 and/or microbeads 4, or clearing the fluid conduit 20 fluidly connected to the first port 56 of the first supplemental bioreactor unit 36₂ after sampling of the biological cells 2 in the same manner described above with respect to bioreactor 36 illustrated in FIG. 19E).

In particular, the first multi-function valve V1′ is operated to fluidly couple a selected one of the media sources s1-s3 to the third multi-function valve V3′, and to fluidly decouple the media destination d2 from the second port 58 of the base bioreactor unit 36₁; the third multi-function valve V3′ is operated to fluidly couple the selected one of the media sources s1-s3 to the first port 56 of the first supplemental bioreactor 36₂, while fluidly decoupling the selected one of the media sources s1-s3 from the first ports 56 of the base bioreactor unit 36₁ and the second supplemental bioreactor 36₃, and to fluidly decouple the second port 58 of the first supplemental bioreactor 36₂ from the first port 56 of the base bioreactor unit 36₁; the second pump P2 is deactivated to fluidly decouple all of the media sources s4-s7 from the fourth multi-function valve V4′, and thus from the third ports 60 of all of the bioreactor units 36; the second multi-function valve V2′ is operated to fluidly couple the media destination d2 to the fourth port 62 of the base bioreactor unit 36₁; and the fourth multi-function valve V4′ is operated to fluidly couple the fourth port 62 of the first supplemental bioreactor 36₂ to the third port 60 of the base bioreactor unit 36₁, while fluidly decoupling the fourth port 62 of the second supplemental bioreactor 36₃ from the third port 60 of the base bioreactor unit 36₁.

In this arrangement, the first multi-function valve V1′ may be placed in any one of Positions 2, 4, or 6 defined in FIG. 23 (depending on the particular one of the media sources s1-s3 to be selected), the second multi-function valve V2′ may be placed in any one of Positions 1, 3, 5, or 7 defined in FIG. 23), the third multi-function valve V3′ may be placed into Position 2 defined in FIG. 35, and the fourth multi-function valve V4′ may be placed into Position 1 defined in FIG. 35, effectively opening the first port 56 of the first supplemental bioreactor 36₂ to the selected one of the media sources s1-s3, opening the fourth port 62 of the first supplemental bioreactor 36₂ to the third port 60 of the base bioreactor unit 36₁, and opening the fourth port 62 of the base bioreactor unit 36₁ to the media destination d2, while closing the first ports 56 of the base bioreactor unit 36₁ and the second supplemental bioreactor 36₃ to the selected one of the media sources s1-s3, closing the second ports 58 of all of the bioreactor units 36 to the media destination d2, closing the third ports 60 of both of the supplemental bioreactor units 36₂, 36₃ to all of the media sources s4-s7, and closing the fourth port 62 of the second supplemental bioreactor 36₃ to the media destination d2, as illustrated in FIG. 37E.

In yet another arrangement illustrated in FIG. 37F, the multi-function valves V1′-V4′ may be operated to define a sixth flow path 86f into the lower flow channel 50 via the third port 60, between the upper and lower flow channels 48, 50 via the porous membrane 46, and out of the upper flow channel 48 via the second port 58, of only the first supplemental bioreactor 36₂, while providing a return flow path 86h through the upper flow channel 48 of the base bioreactor unit 36₁ (e.g., to perform such functions as refreshing the media in the upper flow channel 48 or suspending the biological cells 2 and/or microbeads 4 in the upper flow channel 48 of the first supplemental bioreactor unit 36₂ in the same manner described above with respect to bioreactor 36 illustrated in FIG. 19F).

In particular, the second multi-function valve V2′ is operated to fluidly couple a selected one of the media sources s4-s7 to the fourth multi-function valve V4′, and to fluidly decouple the media destination d2 from the fourth port 62 of the base bioreactor unit 36₁; the fourth multi-function valve V4′ is operated to fluidly couple the selected one of the media sources s4-s7 to the third port 60 of the first supplemental bioreactor 36₂, while fluidly decoupling the selected one of the media sources s4-s7 from the third ports 60 of the base bioreactor unit 36₁ and the second supplemental bioreactor 36₃, and to fluidly decouple the fourth port 62 of the first supplemental bioreactor 36₂ from the third port 60 of the base bioreactor unit 36₁; the first pump P1 is deactivated to fluidly decouple all of the media sources s1-s3 and media destination d1 from the third multi-function valve V3′, and thus from the first ports 56 of all of the bioreactor units 36; the first multi-function valve V1′ is operated to fluidly couple the media destination d2 to the second port 58 of the base bioreactor unit 36₁; and the third multi-function valve V3′ is operated to fluidly couple the second port 58 of the first supplemental bioreactor 36₂ to the first port 56 of the base bioreactor unit 36₁, while fluidly decoupling the second port 58 of the second supplemental bioreactor 36₃ from the first port 56 of the base bioreactor unit 36₁.

In this arrangement, the first multi-function valve V1′ may be placed in any one of Positions 1, 3, or 5 defined in FIG. 23 (depending on the particular one of the media sources s1-s3 to be selected), the second multi-function valve V2′ may be placed in any one of Positions 2, 4, 6, or 8 defined in FIG. 23), the third multi-function valve V3′ may be placed into Position 1 defined in FIG. 35, and the fourth multi-function valve V4′ may be placed into Position 2 defined in FIG. 35, effectively opening the third port 60 of the first supplemental bioreactor 36₂ to the selected one of the media sources s4-s7, opening the second port 58 of the first supplemental bioreactor 36₂ to the first port 56 of the base bioreactor unit 36₁, and opening the second port 58 of the base bioreactor unit 36₁ to the media destination d2, while closing the third ports 60 of the base bioreactor unit 36₁ and the second supplemental bioreactor 36₃ to the selected one of the media sources s4-s7, closing the fourth ports 62 of all of the bioreactor units 36 to the media destination d2, closing the first ports 56 of both of the supplemental bioreactor units 36₂, 36₃ to all of the media sources s1-s3 and media destination d1, and closing the second port 58 of the second supplemental bioreactor 36₃ to the media destination d2, as illustrated in FIG. 37F.

In yet another arrangement illustrated in FIG. 37G, the multi-function valves V1′-V4′ may be operated to define a seventh flow path 86g into the lower flow channel 50 via the third port 60, between the upper and lower flow channels 48, 50 via the porous membrane 46, and out of the upper flow channel 48 via the second port 58, of only the first supplemental bioreactor 36₂ (e.g., to perform such functions as harvesting or sampling of the biological cells 2 from the first supplemental bioreactor unit 36₂ in the same manner described above with respect to bioreactor 36 illustrated in FIG. 19G).

In particular, the second multi-function valve V2′ is operated to fluidly couple a selected one of the media sources s4-s7 to the fourth multi-function valve V4′, and to fluidly decouple the media destination d2 from the fourth port 62 of the base bioreactor unit 36₁; the fourth multi-function valve V4′ is operated to fluidly couple the selected one of the media sources s4-s7 to the third port 60 of the first supplemental bioreactor 36₂, while fluidly decoupling the selected one of the media sources s4-s7 from the third ports 60 of the base bioreactor unit 36₁ and the second supplemental bioreactor 36₃, and to fluidly decouple the fourth port 62 of the first supplemental bioreactor 36₂ from the third port 60 of the base bioreactor unit 36₁; the first multi-function valve V1′ is operated to fluidly couple the media destination d1 to the third multi-function valve V3′, and to fluidly decouple the media destination d2 from the second port 58 of the base bioreactor unit 36₁; the third multi-function valve V3′ is operated to fluidly couple the media destination d1 to the first port 56 of the first supplemental bioreactor 36₂, while fluidly decoupling the media destination d1 from the first ports 56 of the base bioreactor unit 36₁ and the second supplemental bioreactor 36₃, and to fluidly decouple the second port 58 of the first supplemental bioreactor 36₂ from the first port 56 of the base bioreactor unit 36₁.

In this arrangement, the first multi-function valve V1′ may be placed into Position 8 defined in FIG. 23, the second multi-function valve V2′ may be placed in any of one of Positions 2, 4, 6, or 8 defined in FIG. 23) (depending on the particular one of the media sources s4-s7 to be selected), the third multi-function valve V3′ may be placed into Position 2 defined in FIG. 35 (with the caveat that media flows into the output port OP of the third multi-function valve V3′ in a retrograde manner), and the fourth multi-function valve V4′ may be placed into Position 2 defined in FIG. 35, effectively opening the third port 60 of the first supplemental bioreactor 36₂ to the selected one of the media sources s4-s7, and opening the first port 56 of the first supplemental bioreactor 36₂ to the media destination d1, while closing the first ports 56 of the base bioreactor unit 36₁ and second supplemental bioreactor 36₃ to the media destination d1, closing the second ports 58 of all of the bioreactor units 36 to the media destination d2, closing the third ports 60 of the base bioreactor unit 36₁ and the second supplemental bioreactor unit 36₃ to the selected one of the media sources s4-s7, and closing the fourth ports 62 of all of the bioreactor units 36 to the media destination d2, as illustrated in FIG. 37G.

In yet another arrangement illustrated in FIG. 37H, the multi-function valves V1′-V4′ may be operated to define the fifth flow path 86e into the upper flow channel 48 via the first port 56, between the upper and lower flow channels 48, 50 via the porous membrane 46, and out of the lower flow channel 50 via the fourth port 62, of only the first supplemental bioreactor 36₂, as well as defining the third flow path 86c entirely through the lower flow channel 50 via the third and fourth ports 60, 62 of only the first supplemental bioreactor 36₂, while providing a return flow path 86h through the lower flow channel 50 of the base bioreactor unit 36₁ (e.g., to perform such functions as suspending the biological cells 2 and/or microbeads 4 within the upper flow channel 48 to facilitate clogless perfusion of media from the upper flow channel 48 to the lower flow channel 50 via the porous membrane 46 during seeding of the base bioreactor unit 36₁ with biological cells 2 and/or microbeads 4 or when washing the biological cells 2 and/or microbeads 4 within the first supplemental bioreactor unit 36₂ in the same manner described above with respect to bioreactor 36 illustrated in FIG. 19H).

In particular, the second multi-function valve V2′ is operated to fluidly couple a selected one of the media sources s4-s7 to the fourth multi-function valve V4′, and to fluidly couple the media destination d2 to the fourth port 62 of the base bioreactor unit 36₁; the fourth multi-function valve V4′ is operated to fluidly couple the selected one of the media sources s4-s7 to the third port 60 of the first supplemental bioreactor 36₂, while fluidly decoupling the selected one of the media sources s4-s7 from the third ports 60 of the base bioreactor unit 36₁ and the second supplemental bioreactor 36₃, and to fluidly couple the fourth port 62 of the first supplemental bioreactor 36₂ to the third port 60 of the base bioreactor unit 36₁, while fluidly decoupling the fourth port 62 of the second supplemental bioreactor 36₃ from the third port 60 of the base bioreactor unit 36₁; the first multi-function valve V1′ is operated to fluidly couple a selected one of the media sources s1-s3 to the third multi-function valve V3′, and to fluidly decouple the media destination d2 from the second port 58 of the base bioreactor unit 36₁; the third multi-function valve V3′ is operated to fluidly couple the selected one of the media sources s1-s3 to the first port 56 of the first supplemental bioreactor 36₂, while fluidly decoupling the selected one of the media sources s1-s3 from the first ports 56 of the base bioreactor unit 36₁ and the second supplemental bioreactor 36₃, and to fluidly decouple the second port 58 of the first supplemental bioreactor 36₂ from the first port 56 of the base bioreactor unit 36₁.

In this arrangement, the first multi-function valve V1′ may be placed into any of positions 2, 4, or 6 defined in FIG. 23 (depending on the particular one of the media sources s1-s3 to be selected), the second multi-function valve V2′ may be placed into any one of Positions 1, 3, 5, or 7 defined in FIG. 23 (depending on the particular one of the media sources s4-s7 to be selected), the third multi-function valve V3′ may be placed into Position 2 defined in FIG. 35, and the fourth multi-function valve V4′ may be placed into Position 1, effectively opening the first port 56 of the first supplemental bioreactor 36₂ to the selected one of the media sources s1-s3, opening the third port 60 of the first supplemental bioreactor 36₂ to the selected one of the media sources s4-s7, opening the fourth port 62 of the first supplemental bioreactor 36₂ to the third port 60 of the base bioreactor unit 36₁, and opening the fourth port 62 of the base bioreactor unit 36₁ to the media destination d2, while closing the third port 60 of the second supplemental bioreactor 36₃ to the selected one of the media sources s4-s7, closing the fourth port 62 of the second supplemental bioreactor 36₃ to the media destination d2, closing the first ports 56 of the base bioreactor unit 36₁ and the second supplemental bioreactor 36₃ to all of the media sources s1-s3 and media destination d1, and closing the second ports 58 of all of the bioreactor units 36 to the media destination d2, as illustrated in FIG. 37H.

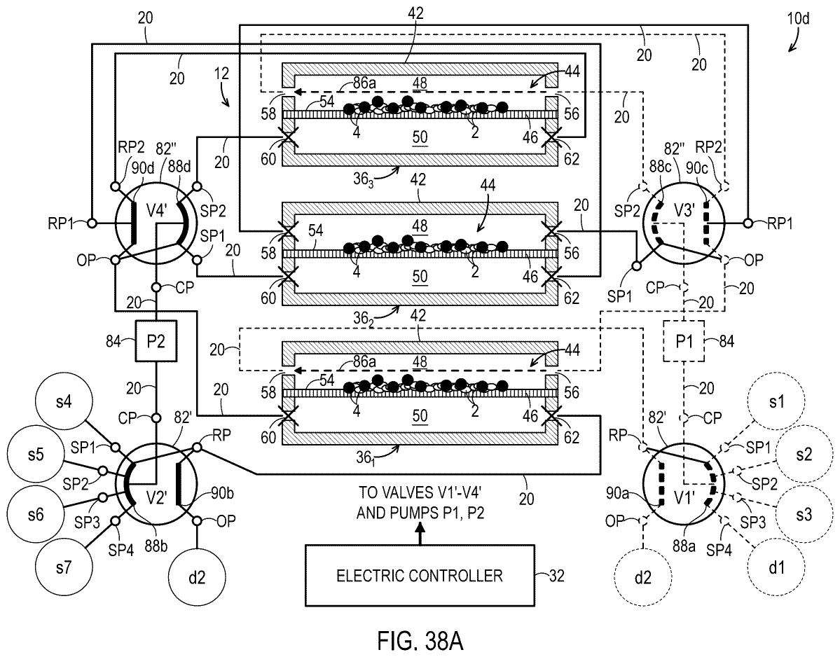

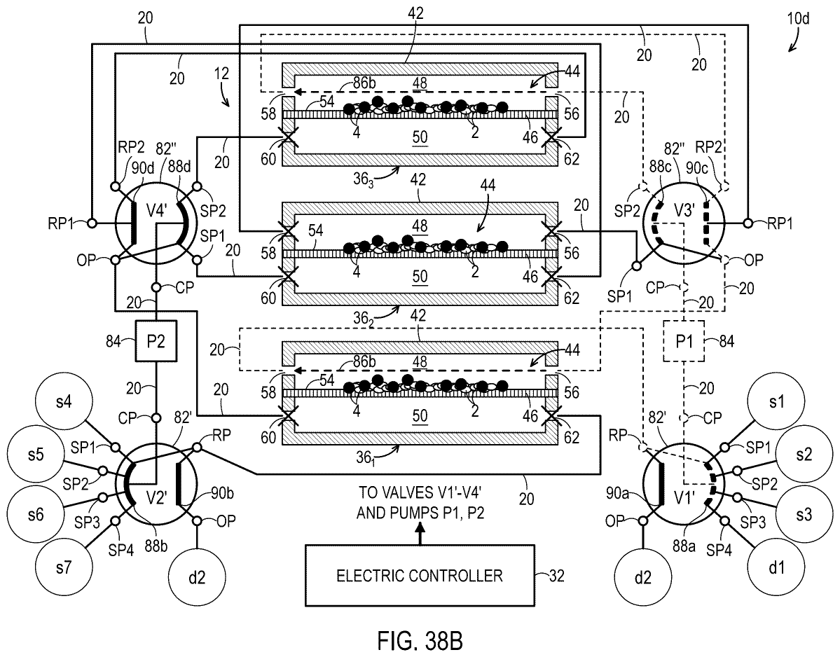

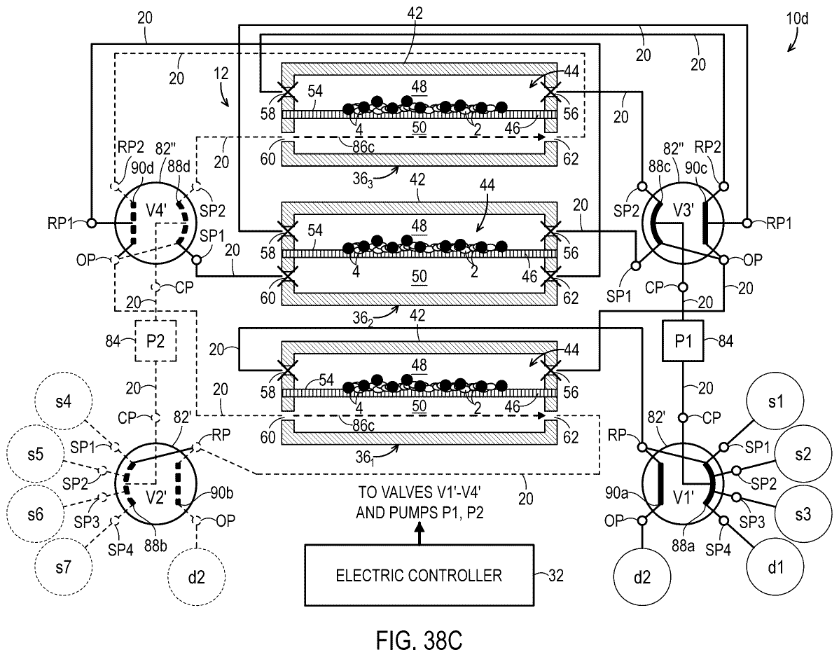

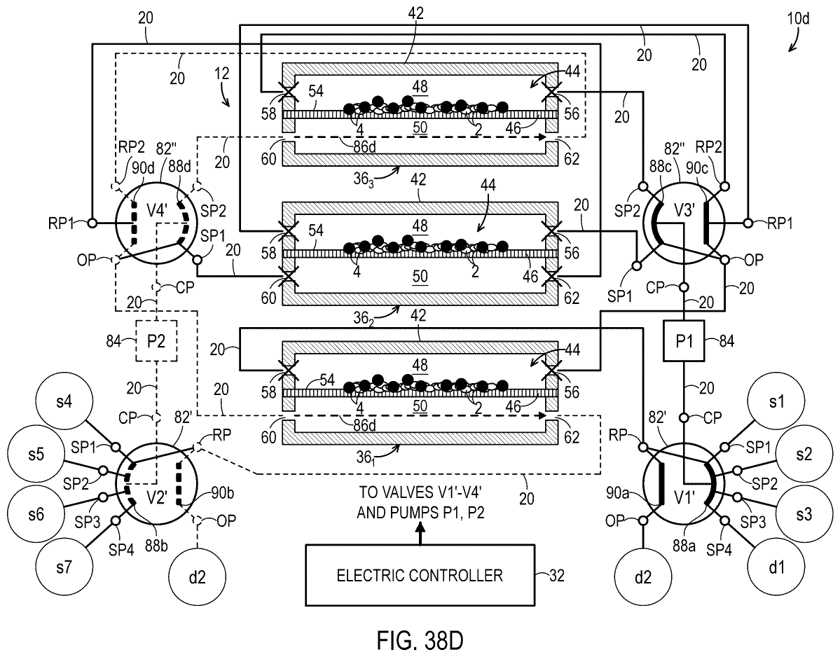

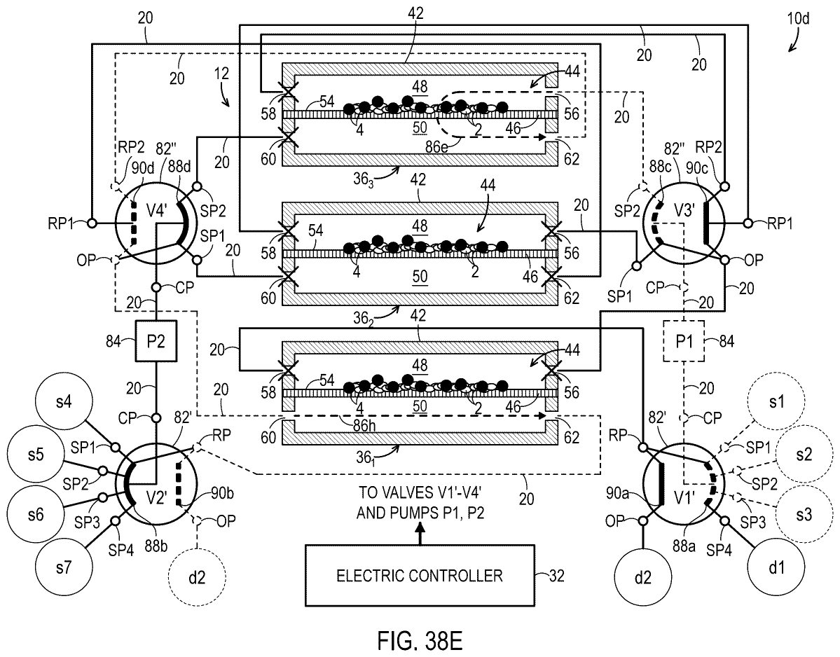

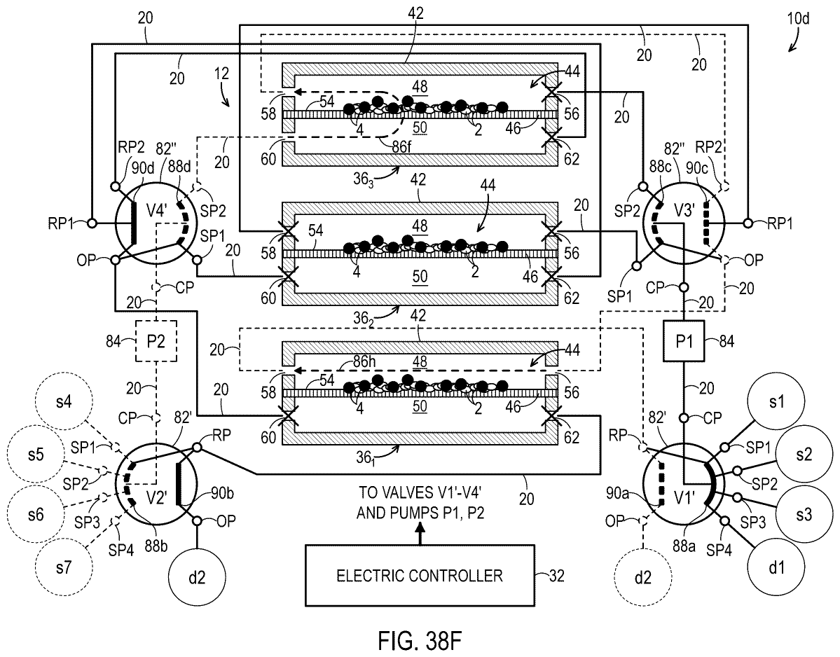

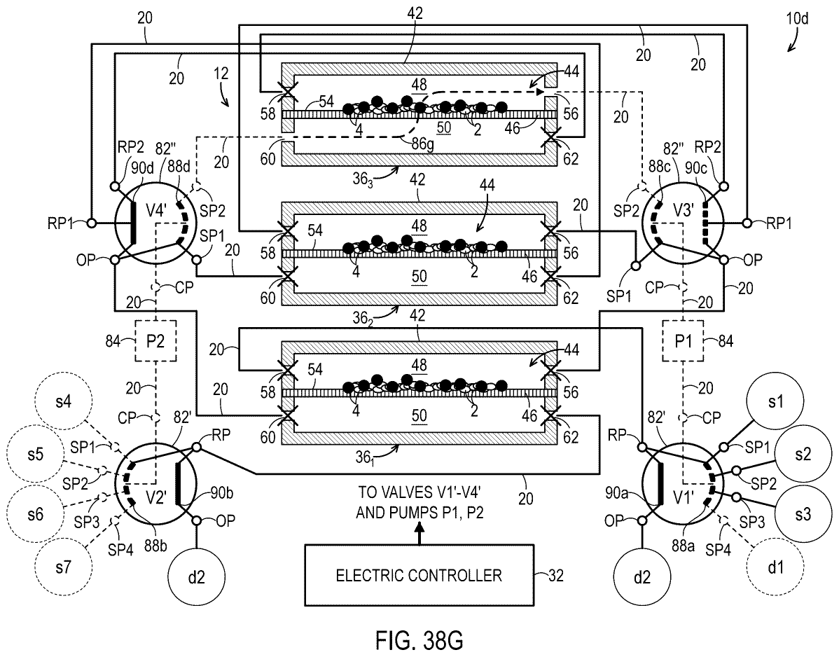

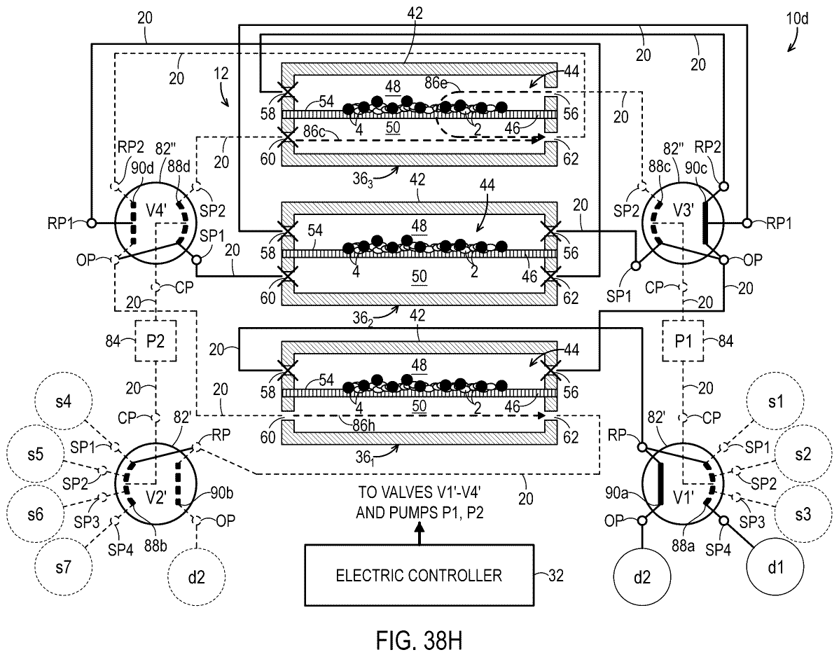

In certain arrangements illustrated in FIGS. 38A-38H, the third and fourth multi-function valves V3′, V4′ may be operated in a manner (essentially by being placed into Positions 3 or 4 defined in FIG. 35) that define flow paths through the second supplemental bioreactor 36₂ (e.g., any of the flow paths 86a-86g and 86c/86e illustrated in FIGS. 19A-19H. Notably, in certain arrangements with flow paths that flow entirely through a respective one of the flow channels 48, 50 of the second supplemental bioreactor 36₃ (e.g., the flow paths 86a-86d), the respective one of the flow channels 48, 50 of the second supplemental bioreactor 36₂ will be coupled in series with the corresponding one of the flow channels 48, 50 of the base bioreactor unit 36₁, effectively defining the same flow path through the corresponding one of the flow channels 48, 50 of the base bioreactor unit 36₁ (i.e., the same flow path extends through the corresponding ones of the flow channels 48, 50 of the both the base bioreactor unit 36₁ and the second supplemental bioreactor 36₂). In other arrangements with flow paths that extend between the flow channels 48, 50 via the porous membrane 46 of the first supplemental bioreactor 36₂ (e.g., the flow paths 86e-86g), if necessary, one of the flow channels 48, 50 of the base bioreactor unit 36₁ will be used to return media from the second supplemental bioreactor 36₂ to the media destination d2 via the first multi-function valve V1′ or the second multi-function valve V2′. Notably, the particular media sources s1-s7, media destinations d1-d2, portions of the valves V1′-V4′, pumps P1, P2, and fluid conduits 20 from, through, and to which media associated with a particular flow is shown dashed for purposes of illustration in FIGS. 38A-38H.

In one arrangement illustrated in FIG. 38A, the multi-function valves V1′-V4′ may be operated to define a first flow path 86a entirely through the upper flow channels 48 of the base bioreactor unit 36₁ and second supplemental bioreactor 36₃ via their first and second ports 56, 58 (e.g., to perform such functions as priming of the base bioreactor 36₁ and second supplemental bioreactor 36₃ in the same manner described above with respect to bioreactor 36 illustrated in FIG. 19A).

In particular, the first multi-function valve V1′ is operated to fluidly couple a selected one of the media sources s1-s3 to the third multi-function valve V3′, and to fluidly couple the media destination d2 to the second port 58 of the base bioreactor unit 36₁; the third multi-function valve V3′ is operated to fluidly couple the selected one of the media sources s1-s3 to the first port 56 of the second supplemental bioreactor 36₃, while fluidly decoupling the selected one of the media sources s1-s3 from the first ports 56 of the base bioreactor unit 36₁ and the first supplemental bioreactor 36₂, and to fluidly couple the second port 58 of the second supplemental bioreactor 36₃ to the first port 56 of the base bioreactor unit 36₁, while fluidly decoupling the second port 58 of the first supplemental bioreactor 36₂ from the first port 56 of the base bioreactor unit 36₁; the second multi-function valve V2′ is operated to fluidly decouple all of the media sources s4-s7 from the fourth multi-function valve V4′, and thus from the third ports 60 of all of the bioreactor units 36, and to fluidly decouple the media destination d2 from the fourth port 62 of the base bioreactor unit 36₁; and the fourth multi-function valve V4′ is operated to fluidly decouple the fourth ports 62 of both of the supplemental bioreactor units 36₂, 36₃ from the third port 60 of the base bioreactor unit 36₁.

In this arrangement, the first multi-function valve V1′ may be placed in one of Positions 1, 3, or 5 defined in FIG. 23 (depending on the particular one of the media sources s1-s3 to be selected), the second multi-function valve V2′ may be placed into Position 9 defined in FIG. 23, the third multi-function valve V3′ may be placed into Position 3 defined in FIG. 35, and the fourth-multi-function valve V4′ may be placed into Position 5 defined in FIG. 35, effectively opening the first port 56 of the second supplemental bioreactor 36₃ to the selected one of the media sources s1-s3, opening the second port 58 of the second supplemental bioreactor 36₃ and the first port 56 of the base bioreactor unit 36₁ to each other, and opening the second port 58 of the base bioreactor unit 36₁ to the media destination d2, while closing the first port 56 of the first supplemental bioreactor 36₂ to the selected one of the media sources s1-s3, closing the second port 58 of the first supplemental bioreactor 36₂ to the media destination d2, closing the third ports 60 of all of the bioreactor units 36 to all of the media sources s4-s7, and closing the fourth ports 62 of all of the bioreactor units 36 to the media destination d2, as illustrated in FIG. 38A.

In another arrangement illustrated in FIG. 38B, the multi-function valves V1′-V4′ may be operated to define a second flow path 86b entirely through the upper flow channels 48 of the base bioreactor unit 36₁ and second supplemental bioreactor 36₃ via their first and second ports 56, 58 that effectively bypasses the media sources s1-s7 and media destinations d1-d2 (e.g., to perform such functions as clearing the fluid conduits 20 respectively fluidly connected to the first and second ports 56, 58 of the base bioreactor unit 36₁ and second supplemental bioreactor unit 36₃ in the same manner described above with respect to bioreactor 36 illustrated in FIG. 19B).

In particular, the first multi-function valve V1′ is operated to fluidly decouple all of the media sources s1-s3 and media destination d1 from the third multi-function valve V3′, and thus from the first ports 56 of all of the bioreactor units 36, and fluidly decouple the media destination d2 from the second port 58 of the base bioreactor unit 36₁; the first multi-function valve V1′ and the third multi-function valve V3′ are further operated to fluidly couple the second port 58 of the second supplemental bioreactor 36₃ to the first port 56 of the base bioreactor unit 36₁, while fluidly decoupling the second port 58 of the first supplemental bioreactor 36₂ from the first port 56 of the base bioreactor unit 36₁, and to fluidly couple the second port 58 of the base bioreactor unit 36₁ to the first port 56 of the second supplemental bioreactor 36₃; the second multi-function valve V2′ is operated to fluidly decouple all of the media sources s4-s7 from the fourth multi-function valve V4′, and thus from the third ports 60 of all of the bioreactor units 36, and to fluidly decouple the media destination d2 from the fourth port 62 of the base bioreactor unit 36₁; and the fourth multi-function valve V4′ is operated to fluidly decouple the fourth ports 62 of both of the supplemental bioreactor units 36₂, 36₃ from the third port 60 of the base bioreactor unit 36₁.

In this arrangement, the first multi-function valve V1′ may be placed into Position 10 defined in FIG. 23, second multi-function valve V2′ may be placed into Position 9 defined in FIG. 23, the third multi-function valve V3′ may be placed into Position 3 defined in FIG. 35, and the fourth multi-function valve V4′ may be placed into Position 5 defined in FIG. 35, effectively opening the second port 58 of the second supplemental bioreactor 36₃ and the first port 56 of the base bioreactor unit 36₁ to each other, and opening the second port 58 of the base bioreactor unit 36₁ and the first port 56 of the second supplemental bioreactor 36₃ to each other, while closing the first ports 56 of all of the bioreactor units 36 to all the media sources s1-s3 and media destination d1, closing the second port 58 of the first supplemental bioreactor 36₂ to the media destination d2, closing the third ports 60 of all of the bioreactor units 36 to all of the media sources s4-s7, and closing the fourth ports 62 of all of the bioreactor units 36 to the media destination d2, as illustrated in FIG. 38B.

In still another arrangement illustrated in FIG. 38C, the multi-function valves V1′-V4′ may be operated to define a third flow path 86c entirely through the lower flow channels 50 of the base bioreactor unit 36₁ and second supplemental bioreactor 36₃ via their first and second ports 56, 58 (e.g., to perform such functions as priming of the base bioreactor unit 36₁ and second supplemental bioreactor unit 36₃ or culturing the biological cells 2 residing in the upper flow channel 48 on the cell culture surface 54 of the porous membrane 46 of the base bioreactor unit 36₁ and second supplemental bioreactor unit 36₃ with fresh media containing nutrient, reagent, or gas in the same manner described above with respect to bioreactor 36 illustrated in FIG. 19C).

In particular, the second multi-function valve V2′ is operated to fluidly couple a selected one of the media sources s4-s7 to the fourth multi-function valve V4′, and to fluidly couple the media destination d2 to the fourth port 62 of the base bioreactor unit 36₁; the fourth multi-function valve V4′ is operated to fluidly couple the selected one of the media sources s4-s7 to the third port 60 of the second supplemental bioreactor 36₃, while fluidly decoupling the selected one of the media sources s4-s7 from the third ports 60 of the base bioreactor unit 36₁ and the first supplemental bioreactor 36₂, and to fluidly couple the fourth port 62 of the second supplemental bioreactor 36₃ to the third port 60 of the base bioreactor unit 36₁, while fluidly decoupling the fourth port 62 of the first supplemental bioreactor 36₂ from the third port 60 of the base bioreactor unit 36₁; the first multi-function valve V1′ is operated to fluidly decouple all of the media sources s1-s3 and media destination d1 from the third multi-function valve V3′, and thus from the first ports 56 of all of the bioreactor units 36, and to fluidly decouple the media destination d2 from the second port 58 of the base bioreactor unit 36₁; and the second multi-function valve V2′ is operated to fluidly decouple the second ports 58 of both of the supplemental bioreactor units 36₂, 36₃ from the first port 56 of the base bioreactor unit 36₁.

In this arrangement, the first multi-function valve V1′ may be placed into Position 9 defined in FIG. 23, the second multi-function valve V2′ may be placed into any one of Positions 1, 3, 5, or 7 defined in FIG. 23 (depending on the particular one of the media sources s4-s7 to be selected), the third multi-function valve V3′ may be placed into Position 5 defined in FIG. 35, and the fourth multi-function valve V4′ may be placed into Position 3, effectively opening the third port 60 of the second supplemental bioreactor 36₃ to the selected one of the media sources s4-s7, opening the fourth port 62 of the second supplemental bioreactor 36₃ and the third port 60 of the base bioreactor unit 36₁ to each other, and opening the fourth port 62 of the base bioreactor unit 36₁ to the media destination d2, while closing the third port 60 of the first supplemental bioreactor 36₂ to the selected one of the media sources s4-s7, closing the fourth port 62 of the first supplemental bioreactor 36₂ to the media destination d2, closing the first ports 56 of all of the bioreactor units 36 to all of the media sources s1-s3 and media destination d1, and closing the second ports 58 of all of the bioreactor units 36 to the media destination d2, as illustrated in FIG. 38C.

In yet another arrangement illustrated in FIG. 38D, the multi-function valves V1′-V4′ may be operated to define a fourth flow path 86d entirely through the lower flow channels 50 of the base bioreactor unit 36₁ and second supplemental bioreactor 36₃ via their third and fourth ports 60, 62 that effectively bypasses the media sources s1-s7 and media destinations d1-d2 (e.g., to perform such functions as recirculating media containing nutrient, reagent, and gas through the lower flow channel 50 of the base bioreactor unit 36₁ and second supplemental bioreactor unit 36₃ in the same manner described above with respect to bioreactor 36 illustrated in FIG. 19D).

In particular, the second multi-function valve V2′ is operated to fluidly decouple all of the media sources s4-s7 from the fourth multi-function valve V4′, and thus from the third ports 60 of all of the bioreactor units 36, and fluidly decouple the media destination d2 from the fourth port 62 of the base bioreactor unit 36₁; the second multi-function valve V2′ and the fourth multi-function valve V3′ are further operated to fluidly couple the fourth port 62 of the second supplemental bioreactor 36₃ to the third port 60 of the base bioreactor unit 36₁, while fluidly decoupling the fourth port 62 of the first supplemental bioreactor 36₂ from the third port 60 of the base bioreactor unit 36₁, and to fluidly couple the fourth port 62 of the base bioreactor unit 36₁ to the third port 60 of the second supplemental bioreactor 36₃; the first multi-function valve V1′ is operated to fluidly decouple all of the media sources s1-s3 and media destination d1 from the third multi-function valve V3′, and thus from the first ports 56 of all of the bioreactor units 36, and to fluidly decouple the media destination d2 from the second port 58 of the base bioreactor unit 36₁; and the third multi-function valve V3′ is operated to fluidly decouple the second ports 58 of both of the supplemental bioreactor units 36₂, 36₃ from the first port 56 of the base bioreactor unit 36₁.

In this arrangement, the first multi-function valve V1′ may be placed into Position 9 defined in FIG. 23, second multi-function valve V2′ may be placed into Position 10 defined in FIG. 23, the third multi-function valve V3′ may be placed into Position 5 defined in FIG. 35, and the fourth multi-function valve V4′ may be placed into Position 3 defined in FIG. 35, effectively opening the fourth port 62 of the second supplemental bioreactor 36₃ and the third port 60 of the base bioreactor unit 36₁ to each other, and opening the fourth port 62 of the base bioreactor unit 36₁ and the third port 60 of the second supplemental bioreactor 36₃ to each other, while closing the third ports 60 of all of the bioreactor units 36 to all the media sources s4-s7, closing the fourth port 62 of the first supplemental bioreactor 36₂ to the media destination d2, and closing the second ports 58 of all of the bioreactor units 36 to the media destination d2, as illustrated in FIG. 38D.

In yet another arrangement illustrated in FIG. 38E, the multi-function valves V1′-V4′ may be operated to define a fifth flow path 86e into the upper flow channel 48 via the first port 56, between the upper and lower flow channels 48, 50 via the porous membrane 46, and out of the lower flow channel 50 via the fourth port 62, of only the second supplemental bioreactor 36₃, while providing a return flow path 86h through the lower flow channel 50 of the base bioreactor unit 36₁ (e.g., to perform such functions as seeding the bioreactor 36 with biological cells 2 and/or microbeads 4, washing the biological cells 2 and/or microbeads 4, or clearing the fluid conduit 20 fluidly connected to the first port 56 of the second supplemental bioreactor unit 36₃ after sampling of the biological cells 2 in the same manner described above with respect to bioreactor 36 illustrated in FIG. 19E).

In particular, the first multi-function valve V1′ is operated to fluidly couple a selected one of the media sources s1-s3 to the third multi-function valve V3′, and to fluidly decouple the media destination d2 from the second port 58 of the base bioreactor unit 36₁; the third multi-function valve V3′ is operated to fluidly couple the selected one of the media sources s1-s3 to the first port 56 of the second supplemental bioreactor 36₃, while fluidly decoupling the selected one of the media sources s1-s3 from the first ports 56 of the base bioreactor unit 36₁ and the first supplemental bioreactor 36₂, and to fluidly decouple the second port 58 of the second supplemental bioreactor 36₃ from the first port 56 of the base bioreactor unit 36₁; the second pump P2 is deactivated to fluidly decouple all of the media sources s4-s7 from the fourth multi-function valve V4′, and thus from the third ports 60 of all of the bioreactor units 36; the second multi-function valve V2′ is operated to fluidly couple the media destination d2 to the fourth port 62 of the base bioreactor unit 36₁; and the fourth multi-function valve V4′ is operated to fluidly couple the fourth port 62 of the second supplemental bioreactor 36₃ to the third port 60 of the base bioreactor unit 36₁, while fluidly decoupling the fourth port 62 of the first supplemental bioreactor 36₂ from the third port 60 of the base bioreactor unit 36₁.

In this arrangement, the first multi-function valve V1′ may be placed in any one of Positions 2, 4, or 6 defined in FIG. 23 (depending on the particular one of the media sources s1-s3 to be selected), the second multi-function valve V2′ may be placed in any one of Positions 1, 3, 5, or 7 defined in FIG. 23), the third multi-function valve V3′ may be placed into Position 4 defined in FIG. 35, and the fourth multi-function valve V4′ may be placed into Position 3 defined in FIG. 35, effectively opening the first port 56 of the second supplemental bioreactor 36₃ to the selected one of the media sources s1-s3, opening the fourth port 62 of the second supplemental bioreactor 36₃ to the third port 60 of the base bioreactor unit 36₁, and opening the fourth port 62 of the base bioreactor unit 36₁ to the media destination d2, while closing the first ports 56 of the base bioreactor unit 36₁ and the second supplemental bioreactor 36₃ to the selected one of the media sources s1-s3, closing the second ports 58 of all of the bioreactor units 36 to the media destination d2, closing the third ports 60 of both of the supplemental bioreactor units 36₂, 36₃ to all of the media sources s4-s7, and closing the fourth port 62 of the first supplemental bioreactor 36₂ to the media destination d2, as illustrated in FIG. 38E.

In yet another arrangement illustrated in FIG. 38F, the multi-function valves V1′-V4′ may be operated to define a sixth flow path 86f into the lower flow channel 50 via the third port 60, between the upper and lower flow channels 48, 50 via the porous membrane 46, and out of the upper flow channel 48 via the second port 58, of only the second supplemental bioreactor 36₃, while providing a return flow path 86h through the upper flow channel 48 of the base bioreactor unit 36₁ (e.g., to perform such functions as refreshing the media in the upper flow channel 48 or suspending the biological cells 2 and/or microbeads 4 in the upper flow channel 48 of the second supplemental bioreactor unit 36₃ in the same manner described above with respect to bioreactor 36 illustrated in FIG. 19F).

In particular, the second multi-function valve V2′ is operated to fluidly couple a selected one of the media sources s4-s7 to the fourth multi-function valve V4′, and to fluidly decouple the media destination d2 from the fourth port 62 of the base bioreactor unit 36₁; the fourth multi-function valve V4′ is operated to fluidly couple the selected one of the media sources s4-s7 to the third port 60 of the second supplemental bioreactor 36₃, while fluidly decoupling the selected one of the media sources s4-s7 from the third ports 60 of the base bioreactor unit 36₁ and the first supplemental bioreactor 36₂, and to fluidly decouple the fourth port 62 of the second supplemental bioreactor 36₃ from the third port 60 of the base bioreactor unit 36₁; the first pump P1 is deactivated to fluidly decouple all of the media sources s1-s3 and media destination d1 from the third multi-function valve V3′, and thus from the first ports 56 of all of the bioreactor units 36; the first multi-function valve V1′ is operated to fluidly couple the media destination d2 to the second port 58 of the base bioreactor unit 36₁; and the third multi-function valve V3′ is operated to fluidly couple the second port 58 of the second supplemental bioreactor 36₃ to the first port 56 of the base bioreactor unit 36₁, while fluidly decoupling the second port 58 of the first supplemental bioreactor 36₂ from the first port 56 of the base bioreactor unit 36₁.

In this arrangement, the first multi-function valve V1′ may be placed in any one of Positions 1, 3, or 5 defined in FIG. 23 (depending on the particular one of the media sources s1-s3 to be selected), the second multi-function valve V2′ may be placed in any one of Positions 2, 4, 6, or 8 defined in FIG. 23), the third multi-function valve V3′ may be placed into Position 3 defined in FIG. 35, and the fourth multi-function valve V4′ may be placed into Position 4 defined in FIG. 35, effectively opening the third port 60 of the second supplemental bioreactor 36₃ to the selected one of the media sources s4-s7, opening the second port 58 of the second supplemental bioreactor 36₃ to the first port 56 of the base bioreactor unit 36₁, and opening the second port 58 of the base bioreactor unit 36₁ to the media destination d2, while closing the third ports 60 of the base bioreactor unit 36₁ and the second supplemental bioreactor 36₃ to the selected one of the media sources s4-s7, closing the fourth ports 62 of all of the bioreactor units 36 to the media destination d2, closing the first ports 56 of both of the supplemental bioreactor units 36₂, 36₃ to all of the media sources s1-s3 and media destination d1, and closing the second port 58 of the first supplemental bioreactor 36₂ to the media destination d2, as illustrated in FIG. 38F.

In yet another arrangement illustrated in FIG. 38G, the multi-function valves V1′-V4′ may be operated to define a seventh flow path 86g into the lower flow channel 50 via the third port 60, between the upper and lower flow channels 48, 50 via the porous membrane 46, and out of the upper flow channel 48 via the second port 58, of only the second supplemental bioreactor 36₃ (e.g., to perform such functions as harvesting or sampling of the biological cells 2 from the second supplemental bioreactor unit 36₃ in the same manner described above with respect to bioreactor 36 illustrated in FIG. 19G).

In particular, the second multi-function valve V2′ is operated to fluidly couple a selected one of the media sources s4-s7 to the fourth multi-function valve V4′, and to fluidly decouple the media destination d2 from the fourth port 62 of the base bioreactor unit 36₁; the fourth multi-function valve V4′ is operated to fluidly couple the selected one of the media sources s4-s7 to the third port 60 of the second supplemental bioreactor 36₃, while fluidly decoupling the selected one of the media sources s4-s7 from the third ports 60 of the base bioreactor unit 36₁ and the first supplemental bioreactor 36₂, and to fluidly decouple the fourth port 62 of the second supplemental bioreactor 36₃ from the third port 60 of the base bioreactor unit 36₁; the first multi-function valve V1′ is operated to fluidly couple the media destination d1 to the third multi-function valve V3′, and to fluidly decouple the media destination d2 from the second port 58 of the base bioreactor unit 36₁; the third multi-function valve V3′ is operated to fluidly couple the media destination d1 to the first port 56 of the second supplemental bioreactor 36₃, while fluidly decoupling the media destination d1 from the first ports 56 of the base bioreactor unit 36₁ and the first supplemental bioreactor 36₂, and to fluidly decouple the second port 58 of the second supplemental bioreactor 36₃ from the first port 56 of the base bioreactor unit 36₁.

In this arrangement, the first multi-function valve V1′ may be placed into Position 8 defined in FIG. 23, the second multi-function valve V2′ may be placed in any of one of Positions 2, 4, 6, or 8 defined in FIG. 23) (depending on the particular one of the media sources s4-s7 to be selected), the third multi-function valve V3′ may be placed into Position 4 defined in FIG. 35 (with the caveat that media flows into the output port OP of the third multi-function valve V3′ in a retrograde manner), and the fourth multi-function valve V4′ may be placed into Position 4 defined in FIG. 35, effectively opening the third port 60 of the second supplemental bioreactor 36₃ to the selected one of the media sources s4-s7, and opening the first port 56 of the second supplemental bioreactor 36₃ to the media destination d1, while closing the first ports 56 of the base bioreactor unit 36₁ and second supplemental bioreactor 36₃ to the media destination d1, closing the second ports 58 of all of the bioreactor units 36 to the media destination d2, closing the third ports 60 of the base bioreactor unit 36₁ and the second supplemental bioreactor units 36₃ to the selected one of the media sources s4-s7, and closing the fourth ports 62 of all of the bioreactor units 36 to the media destination d2, as illustrated in FIG. 38G.

In yet another arrangement illustrated in FIG. 38H, the multi-function valves V1′-V4′ may be operated to define the fifth flow path 86e into the upper flow channel 48 via the first port 56, between the upper and lower flow channels 48, 50 via the porous membrane 46, and out of the lower flow channel 50 via the fourth port 62, of only the second supplemental bioreactor 36₃, as well as defining the third flow path 86c entirely through the lower flow channel 50 via the third and fourth ports 60, 62 of only the second supplemental bioreactor 36₃, while providing a return flow path 86h through the lower flow channel 50 of the base bioreactor unit 36₁ (e.g., to perform such functions as suspending the biological cells 2 and/or microbeads 4 within the upper flow channel 48 to facilitate clogless perfusion of media from the upper flow channel 48 to the lower flow channel 50 via the porous membrane 46 during seeding of the base bioreactor unit 36₁ with biological cells 2 and/or microbeads 4 or when washing the biological cells 2 and/or microbeads 4 within the second supplemental bioreactor unit 36₃ in the same manner described above with respect to bioreactor 36 illustrated in FIG. 19H).

In particular, the second multi-function valve V2′ is operated to fluidly couple a selected one of the media sources s4-s7 to the fourth multi-function valve V4′, and to fluidly couple the media destination d2 to the fourth port 62 of the base bioreactor unit 36₁; the fourth multi-function valve V4′ is operated to fluidly couple the selected one of the media sources s4-s7 to the third port 60 of the second supplemental bioreactor 36₃, while fluidly decoupling the selected one of the media sources s4-s7 from the third ports 60 of the base bioreactor unit 36₁ and the first supplemental bioreactor 36₂, and to fluidly couple the fourth port 62 of the second supplemental bioreactor 36₃ to the third port 60 of the base bioreactor unit 36₁, while fluidly decoupling the fourth port 62 of the first supplemental bioreactor 36₂ from the third port 60 of the base bioreactor unit 36₁; the first multi-function valve V1′ is operated to fluidly couple a selected one of the media sources s1-s3 to the third multi-function valve V3′, and to fluidly decouple the media destination d2 from the second port 58 of the base bioreactor unit 36₁; the third multi-function valve V3′ is operated to fluidly couple the selected one of the media sources s1-s3 to the first port 56 of the second supplemental bioreactor 36₃, while fluidly decoupling the selected one of the media sources s1-s3 from the first ports 56 of the base bioreactor unit 36₁ and the first supplemental bioreactor 36₂, and to fluidly decouple the second port 58 of the second supplemental bioreactor 36₃ from the first port 56 of the base bioreactor unit 36₁.

In this arrangement, the first multi-function valve V1′ may be placed into any of positions 2, 4, or 6 defined in FIG. 23 (depending on the particular one of the media sources s1-s3 to be selected), the second multi-function valve V2′ may be placed into any one of Positions 1, 3, 5, or 7 defined in FIG. 23 (depending on the particular one of the media sources s4-s7 to be selected), the third multi-function valve V3′ may be placed into Position 4 defined in FIG. 35, and the fourth multi-function valve V4′ may be placed into Position 3, effectively opening the first port 56 of the second supplemental bioreactor 36₃ to the selected one of the media sources s1-s3, opening the third port 60 of the second supplemental bioreactor 36₃ to the selected one of the media sources s4-s7, opening the fourth port 62 of the second supplemental bioreactor 36₃ to the third port 60 of the base bioreactor unit 36₁, and opening the fourth port 62 of the base bioreactor unit 36₁ to the media destination d2, while closing the third port 60 of the first supplemental bioreactor 36₂ to the selected one of the media sources s4-s7, closing the fourth port 62 of the first supplemental bioreactor 36₂ to the media destination d2, closing the first ports 56 of the base bioreactor unit 36₁ and the first supplemental bioreactor 36₂ to all of the media sources s1-s3 and media destination d1, and closing the second ports 58 of all of the bioreactor units 36 to the media destination d2, as illustrated in FIG. 38H.

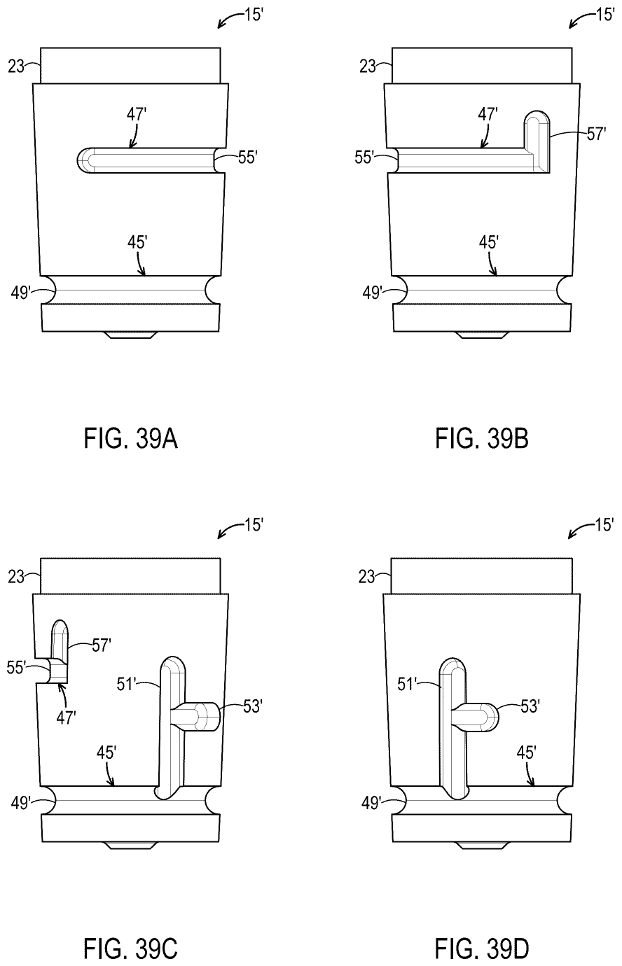

In one embodiment, motorized valve assemblies, such as the motorized valve assembly 11 illustrated in FIGS. 25-31 may be employed in the bioreactor system 10d illustrated in FIG. 34. The rotary valve 82′ of the motorized valve assembly 11 may be employed as either of the first and second multi-function valves V1′, V2′, whereas different rotary valves may be employed as either of the third and fourth multi-function valves V3′, V4′. Such rotary valves are similar to the rotary valve 82′ illustrated in FIGS. 25-31, with the exception that each comprises a common port CP, two return ports RP1, RP2 (rather than a single return port RP), two selector ports SP1, SP2 (as opposed to four selector ports SP1-SP4), and an output port OP affixed to a stator, and furthermore, each comprises a rotor 15′ (illustrated in FIGS. 39A-39D) on which a different open channel system is formed for fluidly coupling the ports to each other. The common port CP, return ports RP1, RP2, selector ports SP1, SP2, and output port OP respectively correspond to the common port CP of the third multi-function valve V3′ or the common port CP of the fourth multi-function valve V4′, the return ports RP1, RP2 of the third multi-function valve V3′ or the return ports RP1, RP2 of the fourth multi-function valve V4′, the selector ports SP1, SP2 of the third multi-function valve V3′ or the selector ports SP1, SP2 of the fourth multi-function valve V4′, and the output port OP of the third multi-function valve V3′ or the output port 56d of the fourth multi-function valve V4′ illustrated in FIG. 34.

Such rotary valve is specifically configured, such that the rotor 15′ may be rotated relative to the stator to Positions 1-6 defined in FIG. 35 to effect the aforementioned combinations of fluid couplings and fluid decouplings between the common port CP, return ports RP1, RP2, selector ports SP1, SP2, and output port OP.

In particular, and with reference to FIGS. 39A-39D, the rotor 15′ comprises a cylindrical insert 23′, and a first open channel 45′ (corresponding to the first valve portion 88c of the third multi-function valve V3′ or the first valve portion 88d of the fourth multi-function valve V4′ illustrated in FIG. 34) and a second open channel 47′ (corresponding to the second valve portion 90c of the third multi-function valve V3′ or the second valve portion 90d of the fourth multi-function valve V4′ illustrated in FIG. 34) formed in the outer surface of cylindrical insert 23′.

When the rotor 15′ is rotated relative to the stator 13 in a first plurality of positions (in this case, Positions 1 and 3 defined in FIG. 35), the first open channel 45′ fluidly couples the selector ports SP1, SP2 to the common port CP (i.e., one of the selectors ports SP1, SP2 and the common port CP concurrently coincide with the first open channel 45′), while the second open channel 47′ respectively fluidly couples the return ports RP1, RP2 to the output port OP (i.e., one of the return ports RP1, RP2 and the output port OP concurrently coincide with the second open channel 47′). When the rotor 15′ is rotated relative to the stator 13 in a second plurality of positions (in this case, Positions 2 and 4 defined in FIG. 35), the first open channel 45′ fluidly couples the selector ports SP1, SP2 to the common port CP (i.e., one of the selectors ports SP1, SP2 and the common port CP concurrently coincide with the first open channel 45′), while the second open channel 47′ does not fluidly couple either of the return ports RP1, RP2 to the output port OP (neither of the return ports RP1, RP2 and the output port OP concurrently coincide with the second open channel 47′).

When the rotor 15′ is rotated relative to the stator 13 in a third position (in this case, Position 5 defined in FIG. 35), the first open channel 45′ does not fluidly couple either of the selector ports SP1, SP2 to the common port CP (neither of the selector ports SP1, SP2 and the common port CP concurrently coincide with the first open channel 45′), the second open channel 47′ does not fluidly couple either of the return ports RP1, RP2 to the output port OP (i.e., neither of the return ports RP1, RP2 and the output port OP concurrently coincide with the second open channel 47′), and the first open channel 45′ does not fluidly couple the output port OP to the common port CP (i.e., the output port and the common port CP do not concurrently coincide with the first open channel 45′). When the rotor 15′ is rotated relative to the stator 13 in a fourth position (in this case, Position 6 defined in FIG. 35), the first open channel 45′ fluidly couples the output port OP to the common port CP (i.e., the output port OP and the common port CP concurrently coincide with the first open channel 45′), while not fluidly coupling either of the selector ports SP1, SP2 to the common port CP (neither of the selector ports SP1, SP2 and the common port CP concurrently coincide with the first open channel 45′), and while the second open channel 47′ does not fluidly couple either of the return ports RP1, RP2 to the output port OP (i.e., neither of the return ports RP1, RP2 and the output port OP concurrently coincide with the second open channel 47′).

In the illustrated embodiment, the first open channel 45′ has a circumferential portion 49′ with which the common port CP always coincides when the rotor 15′ is rotated relative to the stator 13 in Positions 1-6. In the illustrated embodiment, the circumferential portion 49′ extends entirely around the cylindrical insert 23′. However, in alternative embodiments, the circumferential portion 49′ only partially extends around the cylindrical insert 23′. The first open channel 45′ further has an axial portion 51′ extending from the circumferential portion 49′ and a circumferential spur portion 53′ extending from the axial portion 51′. In the illustrated embodiment, the circumferential spur portion 53′ extends from the axial portion 51′ from a location between the circumferential portion 49′ and the free end of the axial portion 51′. The selector ports SP1, SP2 respectively coincide with the axial portion 51′ when the rotor 15′ is rotated relative to the stator 13 in Positions 1 and 3, and respectively coincide with the circumferential spur portion 53′ when the rotor 15′ is rotated relative to the stator 13 in Positions 2 and 4. In alternative embodiments, the selector ports SP1, SP2 may respectively coincide with the circumferential spur portion 53′ when the rotor 15′ is rotated relative to the stator 13 in Positions 1 and 3, and respectively coincide with the axial portion 51′ when the rotor 15′ is rotated relative to the stator 13 in Positions 2 and 4.

In the illustrated embodiment, the second open channel 47′ has a circumferential portion 55′ with which the output port OP coincides when the rotor 15′ is rotated relative to the stator 13 in Positions 1 or 3, and an axial spur portion 57′ extending from the circumferential portion 55′ and with which the return ports RP1, RP2 respectively coincide when the rotor 15′ is rotated relative to the stator 13 in Positions 1 and 3, and with which the return ports RP1, RP2 do not respectively coincide when the rotor 15′ is rotated relative to the stator 13 in Positions 2 and 4.

When the rotor 15′ is rotated relative to the stator 13 in Position 5, the common port CP coincides with the circumferential portion 49′ of the first open channel 45′, neither of the selector ports SP1, SP2 coincides with the axial portion 51′ or the circumferential spur portion 53′ of the first open channel 45′, the output port OP does not coincide with the circumferential portion 55′ of the second open channel 47′, and neither of the return ports RP1, RP2 coincides with the axial spur portion 57′ of the second open channel 47′. When the rotor 15′ is rotated relative to the stator 13 in Position 6, the common port CP coincides with the circumferential portion 49′ of the first open channel 45′, neither of the selector ports SP1, SP2 coincides with the axial portion 51′ or the circumferential spur portion 53′ of the first open channel 45′, neither of the return ports RP1, RP2 coincides with axial spur portion 57′ of the second open channel 47, and the output port OP coincide with the axial portion 51′ of the first open channel 45′.

Notably, the circumferential spur portion 53′ of the first open channel 45′ (to which the different selector ports SP1, SP2 will be fluidly coupled) is at a different height than the axial spur portion 57′ of the second open channel 47′, thereby preventing coincidence of either of the selector ports SP1, SP2 with the axial spur portion 57′ of the second open channel 47′, while also preventing coincidence of either of the return ports RP1, RP2 with the circumferential spur portion 53′ of the first open channel 45′

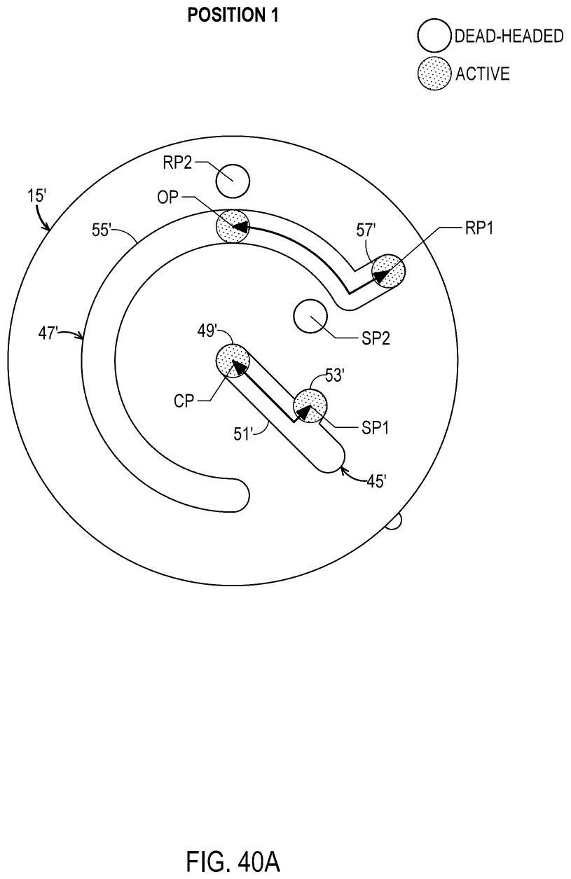

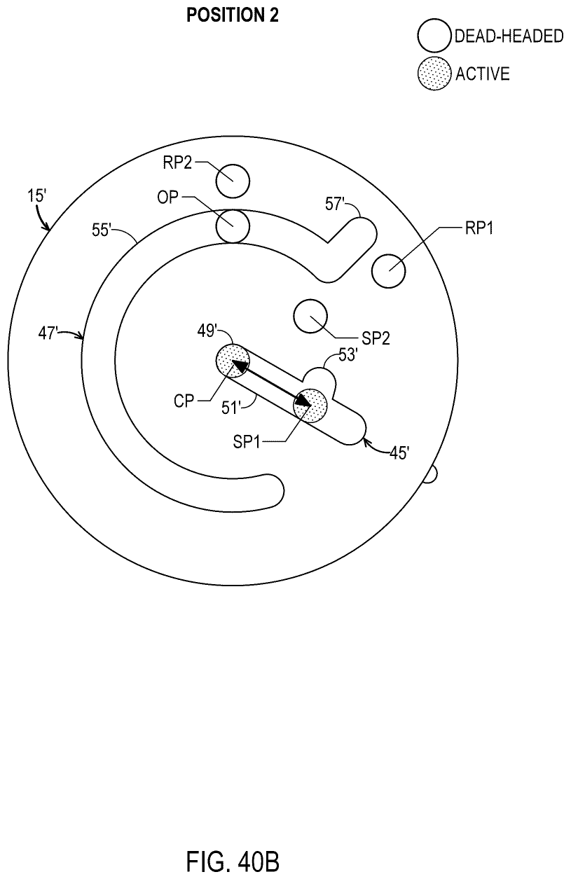

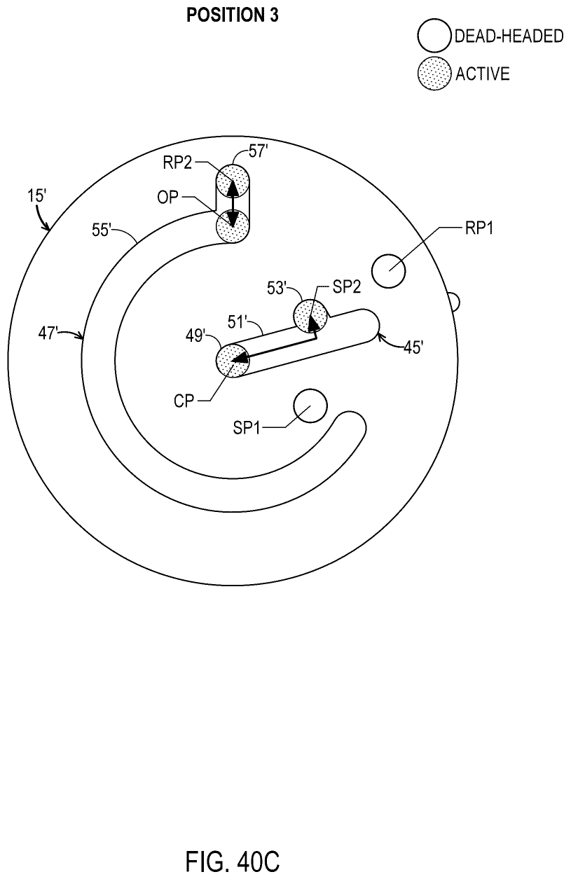

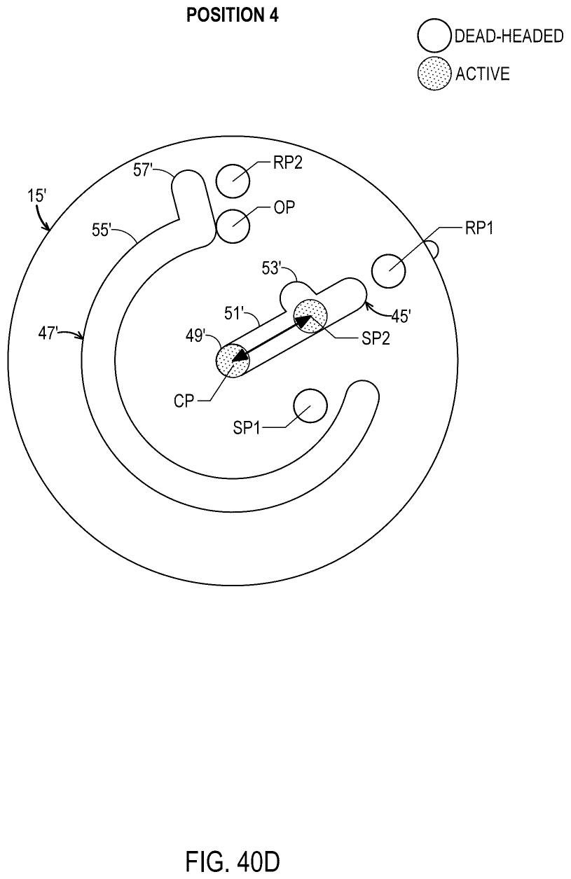

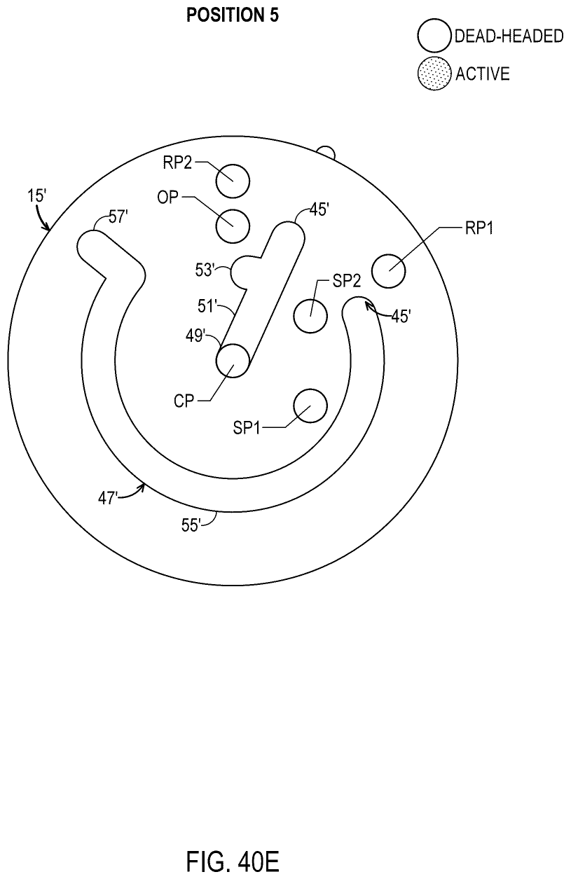

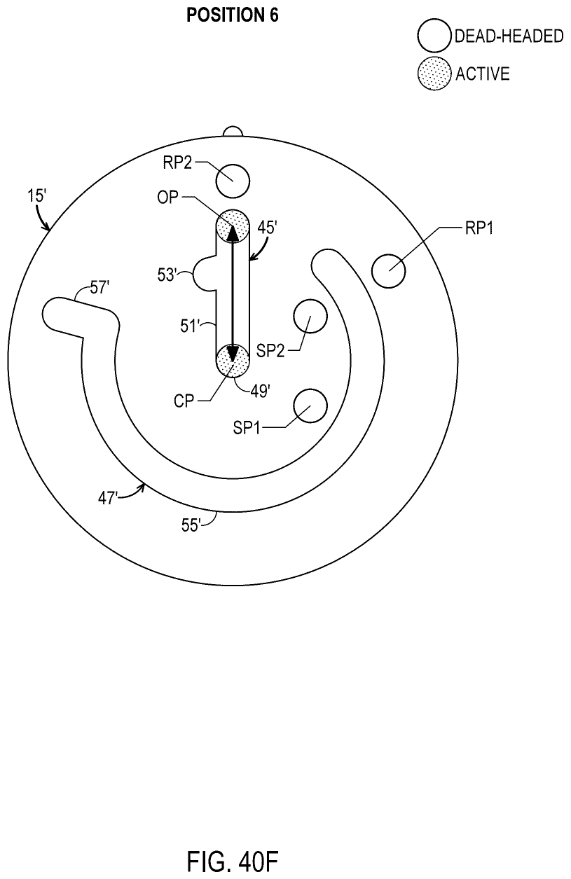

Referring now to FIGS. 40A-40F, the rotor 15′ may be rotated relative to the stator 13 in accordance with a sequence of positions to effect the different fluid couplings and fluid decouplings between the different ones of the selector ports SP1, SP2 and the common port CP, between different ones of the return ports RP1, RP2 and the output port OP, and between the output port OP and the common port CP, as illustrated in FIG. 35.

For example, when the rotor 15′ is rotated relative to the stator 13 in Position 1 (FIG. 40A), the common port CP coincides with the circumferential portion 49′ of the first open channel 45′, the first selector port SP1 coincides with the circumferential spur portion 53′ of the first open channel 45′, the output port OP coincides with the circumferential portion 55′ of the second open channel 47′, and the first return port RP1 coincides with the axial spur portion 57′ of the second open channel 47′. As a result, the first selector port SP1 and the common port CP are activated, thereby fluidly coupling these two ports together (as shown by the arrow), while the second selector port SP2 is deactivated (or deadheaded), and the first return port RP1 and the output port OP are activated, thereby coupling these two ports together (as shown by the arrow), while the second return port RP2 is deactivated (or deadheaded).

When the rotor 15′ is rotated relative to the stator 13 in Position 2 (FIG. 40B), the common port CP coincides with the circumferential portion 49′ of the first open channel 45′, the first selector port SP1 coincides with the axial portion 51′ of the first open channel 45′, the output port OP coincides with the circumferential portion 55′ of the second open channel 47′, and neither of the return ports RP1, RP2 coincides with the axial portion 57′ of the second open channel 47′. As a result, the first selector port SP1 and the common port CP are activated, thereby fluidly coupling these two ports together (as shown by the arrow), while the second selector port SP2 is deactivated (or deadheaded), and the return ports RP1, RP2 and the output port OP are deactivated (or deadheaded), thereby decoupling these ports from each other.

When the rotor 15′ is rotated relative to the stator 13 in Position 3 (FIG. 40C), the common port CP coincides with the circumferential portion 49′ of the first open channel 45′, the second selector port SP2 coincides with the circumferential spur portion 53′ of the first open channel 45′, the output port OP coincides with the circumferential portion 55′ of the second open channel 47′, and the second return port RP2 coincides with the axial spur portion 57′ of the second open channel 47′. As a result, the first selector port SP2 and the common port CP are activated, thereby fluidly coupling these two ports together (as shown by the arrow), while the first selector port SP1 is deactivated (or deadheaded), and the second return port RP2 and the output port OP are activated, thereby coupling these two ports together (as shown by the arrow), while the first return port RP1 is deactivated (or deadheaded).

When the rotor 15′ is rotated relative to the stator 13 in Position 4 (FIG. 40D), the common port CP coincides with the circumferential portion 49′ of the first open channel 45′, the second selector port SP2 coincides with the axial portion 51′ of the first open channel 45′, the output port OP coincides with the circumferential portion 55′ of the second open channel 47′, and neither of the return ports RP1, RP2 coincides with the axial portion 57′ of the second open channel 47′. As a result, the second selector port SP2 and the common port CP are activated, thereby fluidly coupling these two ports together (as shown by the arrow), while the first selector port SP1 is deactivated (or deadheaded), and the return ports RP1, RP2 and the output port OP are deactivated (or deadheaded), thereby decoupling these ports from each other.

When the rotor 15′ is rotated relative to the stator 13 in Position 5 (FIG. 40E), the common port CP coincides with the circumferential portion 49′ of the first open channel 45′, neither of the selector ports SP1, SP2 coincides with the axial portion 51′ or the circumferential spur portion 53′ of the first open channel 45′, the output port OP does not coincide with the circumferential portion 55′ of the second open channel 47′ or the axial portion 51′ of the first open channel 45′, and neither of the return ports RP1, RP2 coincides with the axial spur portion 57′ of the second open channel 47′. As a result, both of the selector ports SP1, SP2 and the common port CP are deactivated (or deadheaded), thereby fluidly decoupling all of these ports from each other, both of the return ports RP1, RP2 and the output port OP are deactivated (or deadheaded), thereby fluidly decoupling these ports from each other, and the output port OP and the common port CP are deactivated (or deadheaded), thereby fluidly decoupling these ports from each other.

When the rotor 15′ is rotated relative to the stator 13 in Position 6 (FIG. 40F), the common port CP coincides with the circumferential portion 49′ of the first open channel 45′, neither of the selector ports SP1, SP2 coincides with the axial portion 51′ or the circumferential spur portion 53′ of the first open channel 45, the output port OP does not coincide with the circumferential portion 55′ of the second open channel 47′, neither of the return ports RP1, RP2 coincides with the axial spur portion 57′ of the second open channel 47′, and the output port OP coincides with the axial portion 51′ of the first open channel 45′. As a result, both of the selector ports SP1, SP2 and the common port CP are deactivated (or deadheaded), thereby fluidly decoupling all of these ports from each other, both of the return ports RP1, RP2 and the output port OP are deactivated (or deadheaded), thereby fluidly decoupling these ports from each other, and the output port OP and the common port CP are activated, thereby fluidly coupling these ports to each other.

### NUMBERED EMBODIMENTS OF THE INVENTION

1. A bioreactor assembly, comprising: a plurality of bioreactor units arranged in a flow sequence and at least one flow path extending through the bioreactor units.
2. The bioreactor assembly of embodiment 1, wherein the plurality of bioreactor units is stacked on top of each other.
3. The bioreactor assembly of embodiment 1 or 2, wherein each of the plurality of bioreactor units is configured for expanding a population of biological cells.
4. The bioreactor assembly of any one of embodiments 1-3, wherein the at least one flow path comprises at least two different flow paths.
5. The bioreactor assembly of embodiment 4, wherein the at least one flow path comprises three different flow paths.
6. The bioreactor assembly of any one of embodiments 1-5, wherein the at least one flow path selectively extends through the plurality of bioreactor units, such that selected ones of the plurality of bioreactor units may be added to the at least one flow path, and selected ones of the plurality of bioreactor units may be removed from the at least one flow path.
7. The bioreactor assembly of any one of embodiments 1-6, wherein each of the plurality of bioreactor units comprises at least one flow channel into which media can be flowed; at least one inlet port respectively fluidly coupled to the at least one flow channel, such that media can be respectively flowed into the at least one flow channel; and at least one outlet port respectively fluidly coupled to the at least one flow channel, such that media can be respectively flowed out of the at least one flow channel; wherein each of the at least one outlet port of each of the plurality of bioreactor units preceding the last bioreactor unit in the flow sequence is respectively fluidly coupled to an immediately succeeding bioreactor unit in the flow sequence, such that the at least one flow path respectively extends entirely through the at least one flow channel of each of the plurality of bioreactor units.
8. The bioreactor assembly of embodiment 7, further comprising a single housing that respectively contains the plurality of bioreactor units.
9. The bioreactor assembly of embodiment 8, wherein the at least inlet port and the at least one outlet port of each of the plurality of bioreactor units is internal to the single housing.
10. The bioreactor assembly of embodiment 8, wherein the at least inlet port and the at least one outlet port of each of the plurality of bioreactor units is external to the single housing.
11. The bioreactor assembly of embodiment 7, wherein each of the plurality of bioreactor units comprises a separate housing.
12. The bioreactor assembly of embodiment 7, wherein the at least one flow channel of each of the plurality of bioreactor units comprises two flow channels; wherein the at least one inlet port of each of the plurality of bioreactor units comprises two inlet ports respectively fluidly coupled to the two flow channels; wherein the at least one outlet port of each of the plurality of bioreactor units comprises two outlet ports respectively fluidly coupled to the two flow channels; and wherein the at least one flow path comprises a first flow path extending in through the inlet port and out through the outlet port of a first one of the two flow channels of each of the plurality of bioreactor units, and a second flow path different from the first flow path extending in through the inlet port and out through the outlet port of a second one of the two flow channels of each of the plurality of bioreactor units.
13. The bioreactor assembly of embodiment 12, wherein the first flow channel is an upper flow channel, and the second flow channel is a lower flow channel.
14. The bioreactor assembly of embodiment 12, wherein the at least one flow path comprises a third flow path different from the first flow path and the second flow path extending in through the inlet port of the first flow channel, between the first flow channel and the second flow channel, and out through the outlet port of the second flow channel, of each of the plurality of bioreactor units.
15. The bioreactor assembly of embodiment 12, wherein each of the plurality of bioreactor units comprises a cell culture surface contained within the first flow channel of the respective bioreactor unit; wherein first media containing biological cells can be flowed into the first flow channel, thereby disposing a population of biological cells on the cell culture surface of each of the plurality of bioreactor units; and wherein second media containing one or more of nutrient, reagent, and gas can be flowed along the second flow path through the second flow channel of each of the plurality of bioreactor units, thereby facilitating the expansion of the populations of biological cells respectively within the plurality of bioreactor units.
16. The bioreactor assembly of embodiment 15, wherein third media can be flowed along the first flow path through the first flow channel of each of the plurality of bioreactor units, thereby harvesting the expanded populations of biological cells from the respective plurality of bioreactor units.
17. The bioreactor assembly of embodiment 15, wherein each of the plurality of bioreactor units comprises a first membrane between the first flow channel and the second flow channel of the respective bioreactor unit, the first membrane being substantially permeable to the one or more of nutrient, reagent, and gas contained within the second media, and waste produced by the populations of biological cells, thereby facilitating expansion of the populations of biological cells within the first flow channels of the respective plurality of bioreactor units, while being substantially impermeable to the populations of biological cells.
18. The bioreactor assembly of embodiment 17, wherein the at least one flow path comprises a third flow path extending in through the inlet port of the first flow channel, between the first flow channel and the second flow channel, and out through the outlet port of the second flow channel, of each of the plurality of bioreactor units; and wherein the first membrane of each of the plurality of bioreactor units is a porous membrane configured for allowing perfusion of the first media along the third flow path from the first flow channels into the second flow channels of the respective plurality of bioreactor units, while filtering the populations of biological cells from the first media, such that the populations of biological cells are retained within the first flow channels of the respective plurality of bioreactor units.
19. The bioreactor assembly of embodiment 18, wherein each of the plurality of bioreactor units comprises further comprises a second membrane, wherein the first flow channels are disposed between the first membranes and the second membranes of the respective plurality of bioreactor units, and wherein the second membrane of each of the plurality of bioreactor units is substantially permeable to gas from a space exterior to the first flow channels of the respective plurality of bioreactor units, thereby facilitating expansion of the populations of biological cells within the first flow channels of the respective plurality of bioreactor units, while being substantially impermeable to the populations of biological cells and the first media.
20. The bioreactor assembly of embodiment 19, wherein the cell culture surface is formed on the second membrane.
21. A bioreactor system, comprising: the bioreactor assembly of any one of embodiments 1-20; and a pump assembly configured for conveying media along the at least one flow path.
22. The bioreactor system of embodiment 21, wherein the pump assembly comprises a peristaltic pump.
23. The bioreactor system of embodiment 21, further comprising a valve assembly configured for selectively fluidly coupling ones of the plurality of bioreactor units to the at least one flow path, and selectively fluidly decoupling ones of the plurality of bioreactor units from the at least one flow path.
24. A bioreactor system, comprising: the bioreactor assembly of embodiment 7; and a valve assembly configured for selectively fluidly coupling each of the at least one outlet port of each of the plurality of bioreactor units preceding the last bioreactor unit of the flow sequence respectively to the immediately succeeding bioreactor unit in the flow sequence.
25. A method of operating the bioreactor assembly of embodiment 1, comprising: seeding the plurality of bioreactor units respectively with populations of biological cells; flowing first media containing one or more of nutrient, reagent, and gas along a first one of the at least one flow path, thereby facilitating the expansion of the populations of biological cells respectively within the plurality of bioreactor units; and harvesting the expanded populations of biological cells respectively from the plurality of bioreactor units.
26. The method of embodiment 25, wherein seeding the plurality of bioreactor units respectively with the populations of biological cells comprises flowing second media containing biological cells along a second one of the at least one flow path different from the first flow path.
27. The method of embodiment 26, wherein harvesting the expanded populations of biological cells respectively from the plurality of bioreactor units comprises flowing third media along a third one of the at least one flow path different from the first flow path and the second flow path.
28. The method of any one of embodiments 25-27, wherein flowing the first media through the first flow channel comprises pumping the first media.
29. The method of any one of embodiments 25-28, wherein the biological cells are T lymphocytes (T-cells).
30. A method of operating the bioreactor assembly of embodiment 15, comprising: flowing the first media containing the biological cells into the first flow channels of the respective plurality of bioreactor units, thereby respectively depositing the populations of biological cells on the cell culture surfaces of the respective plurality of bioreactor units; and flowing the second media containing the one or more of nutrient, reagent, and gas along the second flow path through the second flow channel of each of the plurality of bioreactor units, thereby facilitating the expansion of the populations of biological cells within the second flow channels of the respective plurality of bioreactor units.
31. The method of embodiment 30, further comprising: flowing third media along the first flow path into the first flow channels of the respective plurality of bioreactor units, such that the expanded populations of biological cells are suspended in the third media; and flowing the third media with the suspended expanded populations of biological cells along the first flow path out of the second flow channels of the respective plurality of bioreactor units.
32. The method of embodiment 30 or 31, wherein flowing the first media into the first flow channel and flowing the second media through the second flow channel comprises pumping the first media and the second media.
33. The method of any one of embodiments 30-32, wherein the biological cells are T lymphocytes (T-cells).
34. A method of operating the bioreactor assembly of embodiment 18, comprising: flowing the first media containing the biological cells along the third flow path into the first flow channels of the respective plurality of bioreactor units; perfusing the first media along the third flow path from the first flow channels, through the first porous membranes, and into the second flow channels of the respective plurality of bioreactor units, while substantially retaining the biological cells within the first flow channels of the respective plurality of bioreactor units, such that the populations of biological cells are disposed on the cell culture surfaces of the respective plurality of bioreactor units; flowing the perfused first media along the third flow path out of the second flow channels of the respective plurality of bioreactor units; flowing the second media containing the one or more of nutrient, reagent, and gas along the second flow path through the second flow channels of the respective plurality of bioreactor units; and diffusing the one or more of nutrient, reagent, and gas from the second flow channels, through the first membranes, and into the second flow channels, and diffusing the waste from the first flow channels, through the first membranes, and into the second flow channels, of the respective plurality of bioreactor units, thereby facilitating the expansion of the populations of biological cells within the first flow channels of the respective plurality of biological cells.
35. The method of embodiment 34, further comprising: flowing third media along the first flow path into the first flow channels of the respective plurality of bioreactor units, such that the expanded populations of biological cells are suspended in the third media; and flowing the third media with the suspended expanded populations of biological cells along the first flow path out of the second flow channels of the respective plurality of bioreactor units.
36. The method of embodiment 34 or 35, wherein flowing the first media into the first flow channel and flowing the second media through the second flow channel comprises pumping the first media and the second media.
37. The method of any one of embodiments 34-36, wherein the biological cells are T lymphocytes (T-cells).
38. A method of expanding a population of biological cells using a plurality of bioreactor units, comprising: (a) adding a flow sequence of a plurality of bioreactor units to at least one flow path; (b) seeding the plurality of bioreactor units respectively with populations of biological cells; (c) flowing first media containing one or more of nutrient, reagent, and gas along a first one of the at least one flow path, thereby facilitating the expansion of the populations of biological cells respectively within the plurality of bioreactor units; and (d) harvesting the expanded populations of biological cells respectively from the plurality of bioreactor units.
39. The method of embodiment 38, wherein (b) seeding the plurality of bioreactor units respectively with the populations of biological cells comprises flowing second media containing biological cells along a second one of the at least one flow path different from the first flow path.
40. The method of embodiment 39, wherein (d) harvesting the expanded populations of biological cells respectively from the plurality of bioreactor units comprises flowing third media along a third one of the at least one flow path different from the first flow path and the second flow path.
41. The method of any one of embodiments 38-40, wherein the flow sequence of the plurality of bioreactor units is increased over time by incrementally adding at least one succeeding bioreactor unit to the at least one flow path, wherein steps (b) and (c) are performed for at least one preceding bioreactor unit in the flow sequence prior to each addition of the at least one succeeding bioreactor unit in the flow sequence to the at least one flow path.
42. The method of embodiment 41, wherein seeding each at least one succeeding bioreactor unit with a respective population of biological cells comprises isolating at least one aliquot of biological cells from at least one expanded population of biological cells respectively within the at least one preceding bioreactor unit, and seeding the respective each at least one succeeding bioreactor unit with the at least one aliquot of biological cells respectively isolated from the preceding bioreactor unit.
43. The method of embodiment 42, wherein isolating the at least one aliquot of the biological cells from the at least one expanded population of biological cells within the at least one preceding bioreactor unit comprises flowing third media through the at least one preceding bioreactor unit, such that the at least one aliquot of biological cells is suspended in the third media; and wherein seeding the respective each at least one succeeding bioreactor unit with the at least one aliquot of biological cells respectively isolated in the at least one preceding bioreactor unit comprises flowing the third media containing the suspended at least one aliquot of biological cells from the at least one preceding bioreactor unit into the each at least one succeeding bioreactor unit.
44. The method of embodiment 42, wherein at least one multilayer expanded population of biological cells is respectively within the at least one preceding bioreactor unit, and wherein isolating the at least one aliquot of biological cells from the at least one expanded population of biological cells respectively within the at least one preceding bioreactor unit comprises isolating at least one upper layer of the at least one multilayer expanded population of biological cells, such that a monolayer or one or more lower layers of the at least one expanded population of biological cells remain respectively within the at least one preceding bioreactor unit.
45. The method of any one of embodiments 38-44, wherein step (b) is performed for each preceding bioreactor unit subsequent to adding the flow sequence of the plurality of bioreactor units to the at least one flow path.
46. The method of any one of embodiments 38-45, further comprising (e) sequentially stacking the plurality of bioreactor units atop each other.
47. The method of any one of embodiments 38-46, wherein steps (a)-(c) are performed for each immediately preceding bioreactor unit in the flow sequence prior to stacking each of at least one succeeding bioreactor unit in the flow sequence atop the immediately preceding bioreactor unit.
48. The method of any one of embodiments 38-47, wherein the plurality of bioreactor units is stacked together prior to performing step (a) on a first one of the flow sequence of the plurality of bioreactor units.
49. The method of any one of embodiments 38-48, wherein each of the plurality of bioreactor units comprises two flow channels, a first membrane disposed between the two flow channels, and a cell culture surface contained within the first flow channel; wherein (b) seeding the plurality of bioreactor units respectively with the populations of biological cells comprises flowing second media containing biological cells into the first flow channels of the respective plurality of bioreactor units, thereby respectively disposing the populations of biological cells on the cell culture surfaces of the respective plurality of bioreactor units; and wherein (c) flowing the first media containing one or more of nutrient, reagent, and gas along the first flow path comprises flowing the first media through the second flow channels of the respective plurality of bioreactor units, such that the one or more of nutrient, reagent, and gas diffuses from the second flow channels, through the first membranes, and into the second flow channels, and waste produced by the populations of biological cells diffuses from the first flow channels, through the first membranes, and into the second flow channels, of the respective plurality of bioreactor units, thereby facilitating the expansion of the populations of biological cells within the first flow channels of the respective plurality of biological cells.
50. The method of embodiment 49, wherein (d) harvesting the expanded populations of biological cells respectively from the plurality of bioreactor units comprises: (d1) flowing third media along the first flow path into the first flow channels of the respective plurality of bioreactor units, such that the expanded populations of biological cells are suspended in the third media; and (d2) flowing the third media with the suspended expanded populations of biological cells along the first flow path out of the second flow channels of the respective plurality of bioreactor units.
51. The method of embodiment 49 or 50, wherein first membrane is a porous membrane, and wherein (b) seeding the plurality of bioreactor units respectively with the populations of biological cells comprises: (b1) flowing the second media containing the biological cells along forward portion of the second flow path into the first flow channels of the respective plurality of bioreactor units; (b2) perfusing the second media along bypass portions of the second flow path from the first flow channels, through the porous membranes, and into the second flow channels, of the respective plurality of bioreactor units, while substantially retaining the biological cells within the first flow channels of the respective plurality of bioreactor units, such that the populations of biological cells are disposed on the cell culture surfaces of the respective plurality of bioreactor units; and (b3) flowing the perfused second media along return portion of the second flow path out of the second flow channels of the respective plurality of bioreactor units.
52. The method of embodiment 51, wherein steps (b1)-(b3) are incrementally performed in each of the plurality of bioreactor units in the order of the flow sequence.
53. The method of embodiment 51 or 52, wherein the cell culture surfaces are disposed on the respective porous membranes of the respective plurality of bioreactor units; and wherein the incremental perfusion of the second media with the biological cells along the bypass portion of the second flow path from the first flow channel, through the porous membrane, and into the second flow channel urges the biological cells towards the cell culture surface on the porous membrane via the action of convection of a current bioreactor unit preceding the last bioreactor unit in the flow sequence until at least one layer of the biological cells are disposed on the cell culture surface, thereby substantially blocking perfusion of the second media through the porous membrane of the current bioreactor unit, such that the second media with substantially no biological cells initially flows along the forward portion of the second flow path out of the current bioreactor unit into a next unseeded bioreactor unit in the flow sequence, and such that the second media with substantially all of the biological cells subsequently flows further along the forward portion of the second flow path out of the current bioreactor unit into the next unseeded bioreactor unit.
54. The method of any one of embodiments 51-53, wherein each of the plurality of bioreactor units further comprises a second membrane, wherein the first flow channels are disposed between the first membranes and the second membranes of the respective plurality of bioreactor units, the method further comprising (e) diffusing gas from a space exterior to the first flow channels, through the second membranes, and into the first flow channels, of the respective plurality of bioreactor units, thereby facilitating the expansion of the populations of biological cells within the first flow channels of the respective plurality of biological cells.
55. The method of any one of embodiments 38-54, wherein flowing the first media into the first flow channel.
56. A bioreactor system, comprising: at least one bioreactor, each configured for containing a population of biological cells and comprising a first flow channel, a second flow channel, a porous membrane disposed between the first and second flow channels; a first multi-function valve having a first at least one position for defining a first flow path through the first flow channel of each of the at least one bioreactor, and a second at least one position different from the first at least one position for defining a second flow path into the first flow channel, between the first and second flow channels via the porous membrane, and out of the second flow channel of each of the at least one bioreactor; and a first pump configured for conveying media along the first flow paths of the at least one bioreactor when the first multi-function valve is in the first at least one position, and for conveying media along the second flow paths of the at least one bioreactor when the multi-function valve is in the second at least one position.
57. The bioreactor system of embodiment 56, wherein the first at least one position comprises a first plurality of positions.
58. The bioreactor system of embodiment 56 or 57, wherein the second at least one position comprises a second plurality of positions.
59. The bioreactor system of any one of embodiments 56-58, further comprising: a second multi-function valve having a first at least one position for defining a third flow path through the second flow channel of each of the at least one bioreactor, and a second at least one position different from the first at least one position for defining a fourth flow path into the second flow channel, between the second and first flow channels via the porous membrane, and out of the first flow channel of each of the at least one bioreactor; and a second pump fluidly configured for conveying media along the third flow path of each of the at least one bioreactor when the second multi-function valve is in the first at least one position, and configured for conveying media along the fourth flow path of each of the at least one bioreactor when the multi-function valve is in the second at least one position.
60. The bioreactor system of any one of embodiments 56-59, wherein the at least one bioreactor is a single bioreactor.
61. The bioreactor system of embodiment 60, wherein the first multi-function valve is configured for respectively fluidly coupling at least one media source to the first flow channel of the single bioreactor while fluidly coupling a media destination to the first flow channel of the single bioreactor when the first multi-function valve is respectively in the first at least one position, thereby defining the first flow path for the single bioreactor; and wherein the first multi-function valve is configured for respectively fluidly coupling the at least one media source to the first flow channel of the single bioreactor while fluidly coupling no media destination to the first flow channel of the single bioreactor when the first multi-function valve is respectively in the second at least one position, thereby defining the second flow path for the single bioreactor.
62. The bioreactor system of embodiment 61, wherein the at least one media source comprises a plurality of media sources, and the first at least one position comprises a first plurality of positions, and the second at least one position comprises a second plurality of positions.
63. The bioreactor system of embodiment 61, wherein the first multi-function valve has a third at least one position configured for fluidly coupling no media source and no media destination to the first flow channel of the single bioreactor.
64. The bioreactor system of any one of embodiments 56-63, wherein the at least one bioreactor comprises a plurality of bioreactor units.
65. The bioreactor system of embodiment 64, wherein the first at least one position comprises a first plurality of positions, and the second at least one position comprises a second plurality of positions; wherein the first multi-function valve is configured for respectively fluidly coupling a media source to the first flow channels of the plurality of bioreactor units while fluidly coupling a media destination to the first flow channels of the plurality of bioreactor units when the first multi-function valve is respectively in the first plurality of positions, thereby respectively defining the first flow paths for the plurality of bioreactor units; and wherein the first multi-function valve is configured for respectively fluidly coupling the media source to the first flow channels of the plurality of bioreactor units while fluidly coupling no media destination to the first flow channels of the plurality of bioreactor units when the first multi-function valve is respectively in the second plurality of positions, thereby respectively defining the second flow paths for the plurality of bioreactor units.
66. The bioreactor system of embodiment 65, wherein the first multi-function valve has a third position configured for fluidly coupling no media source and no media destination to the first flow channels of the plurality of bioreactor units.
67. The bioreactor system of any one of embodiments 56-66, wherein the first multi-function valve is a rotary valve.
68. The bioreactor system of any one of embodiments 56-67, further comprising an electric controller configured for operating the first multi-function valve and the first pump.
69. The bioreactor system of any one of embodiments 56-68, wherein the porous membrane of each of the at least one bioreactor is configured for supporting the respective population of biological cells thereon.
70. A bioreactor system, comprising: at least one bioreactor, each configured for containing a population of biological cells and comprising a first flow channel, a second flow channel, a porous membrane disposed between the first and second flow channels, and a first port and a second port associated with the first flow channel; a first multi-function valve comprising a common port fluidly coupled to the first port of one of the at least one bioreactor, a return port fluidly coupled to the second port of the one bioreactor, a plurality of selector ports configured for respectively being fluidly coupled to a first plurality of media sources and/or media destinations, and an output port configured for being fluidly coupled to a first media destination, the first multi-function valve having: a first plurality of positions for respectively fluidly coupling the plurality of selector ports to the common port, while fluidly coupling the output port to the return port; and a second plurality of positions different from the first plurality of positions for respectively fluidly coupling the plurality of selector ports to the common port, while fluidly decoupling the output port from the return port; and a first pump fluidly coupled to one of the common port and the return port of the first multi-function valve.
71. The bioreactor system of embodiment 70, wherein the first multi-function valve has a third position different from the first plurality of positions and the second plurality of positions for fluidly decoupling all of the plurality of selector ports from the common port, for fluidly decoupling the output port from the return port, and for fluidly decoupling the return port from the common port.
72. The bioreactor system of embodiment 70 or 71, wherein the first multi-function valve has a third position different from the first plurality of positions and the second plurality of positions for fluidly decoupling all of the plurality of selector ports from the common port, while fluidly decoupling the output port from the return port, and while fluidly coupling the return port to the common port.
73. The bioreactor system of any one of embodiments 70-72, wherein the first plurality of media sources and/or media destinations comprises one or more of a biological cell source, microbead source, nutrient source, reagent source, gas source, wash buffer source, saline source, and biological cell collection destination.
74. The bioreactor system of embodiment 73, wherein the first media destination comprises a waste destination.
75. The bioreactor system of any one of embodiments 70-74, wherein each of the at least one bioreactor further comprises a third port and a fourth port associated with the second flow channel, and the bioreactor system further comprising a second multi-function valve comprising a common port fluidly coupled to the third port of the one bioreactor, a return port fluidly coupled to the fourth port of the one bioreactor, a plurality of selector ports configured for respectively being fluidly coupled to a second plurality of media sources and/or media destinations, and an output port configured for being fluidly coupled to a second media destination, the second multi-function valve having: a first plurality of positions for respectively fluidly coupling the plurality of selector ports to the common port, while fluidly coupling the output port to the return port; and a second plurality of positions different from the first plurality of positions for respectively fluidly coupling the plurality of selector ports to the common port, while fluidly decoupling the output port from the return port; and a second pump fluidly coupled to one of the common port and the return port of the second multi-function valve.
76. The bioreactor system of embodiment 75, wherein at least some of the first plurality of media sources and/or media destinations are respectively the same as at least some of the second plurality of media sources and/or media destinations.
77. The bioreactor system of embodiment 75, wherein the first media destination and the second media destination are the same.
78. The bioreactor system of any one of embodiments 70-77, wherein the output port is the only output port of the first multi-function valve.
79. The bioreactor system of any one of embodiments 70-78, wherein the first multi-function valve comprises a first valve portion that respectively fluidly couples the plurality of selector ports to the common port when the first multi-function valve is respectively in the first plurality of positions or the second plurality of positions, and a second valve portion that fluidly couples the output port to the return port when the multi-function valve is in any of the first plurality of positions, and fluidly decouples the output port from the return port when the first multi-function valve is in any of the second plurality of positions.
80. The bioreactor system of any one of embodiments 70-79, wherein the at least one bioreactor comprises a plurality of bioreactor units, and wherein the one bioreactor is a base bioreactor unit within the plurality of bioreactor units, the bioreactor system further comprising: a second multi-function valve comprising a common port fluidly coupled to the common port of the first multi-function valve, one or more selector ports respectively fluidly coupled to the first ports of one or more of the plurality of bioreactor units different from the base bioreactor unit, one or more return ports respectively fluidly coupled to the second ports of the one or more bioreactor units, and an output port fluidly coupled to the first port of the base bioreactor unit, the second multi-function valve having: first one or more positions for respectively fluidly coupling the one or more selector ports to the common port, while fluidly coupling the one or more return ports to the output port; second one or more positions different from the first one or more positions for respectively fluidly coupling the one or more selector ports to the common port, while fluidly decoupling all of the one or more return ports from the output port; and a third position for respectively fluidly coupling the output port to the common port, while fluidly decoupling the one or more selector ports from the common port, and while fluidly decoupling the one or more return ports from the output port.
81. The bioreactor system of embodiment 80, wherein the one or more bioreactor units comprises two or more bioreactor units, the one or more selector ports comprises two or more selector ports, the one or more return ports comprises two or more return ports, the first one or more positions comprises two or more positions, and the second one or more positions comprises two or more positions.
82. The bioreactor system of any one of embodiments 70-82, wherein the first multi-function valve is a rotary valve.
83. The bioreactor system of embodiment 82, wherein the rotary valve comprises a stator in which the common port, the return port, the plurality of selector ports, and the output port are affixed, and a rotor configured for being rotated relative to the stator to the first plurality of positions and the second plurality of positions, the rotor comprising a first open channel and a second open channel, wherein, when the rotor is rotated relative to the stator to the first plurality of positions, the first open channel respectively fluidly couples the plurality of selector ports to the common port, while the second open channel fluidly couples the output port to the return port, and wherein, when the rotor is rotated relative to the stator to the second plurality of positions, the first open channel respectively fluidly couples the plurality of selector ports to the common port, while the second open channel does not fluidly couple the output port to the return port.
84. The bioreactor system of embodiment 83, wherein, when the rotor is rotated relative to the stator to each of the first plurality of positions, a respective one of the plurality of selector ports and the common port concurrently coincide with the first open channel, while the return port and the output port concurrently coincide with the second open channel; and wherein, when the rotor is rotated relative to the stator to each of the second plurality of positions, a respective one of the plurality of selector ports and the common port concurrently coincide with the first open channel, while the return port and the output port do not concurrently coincide with the second open channel.
85. The bioreactor system of embodiment 83, wherein, when the rotor is rotated relative to the stator to a third position different from the first plurality of positions and the second plurality of positions, the first open channel does not fluidly couple any of the plurality of selector ports to the common port, the second open channel does not fluidly couple the output port to the return port, and the first open channel does not fluidly couple the return port to the common port.
86. The bioreactor system of embodiment 85, wherein, when the rotor is rotated relative to the stator in the third position, the plurality of selector ports and the common port do not concurrently coincide with the first open channel, the return port and the output port do not concurrently coincide with the second open channel, and the return port and the common port do not concurrently coincide with the first open channel.
87. The bioreactor system of embodiment 85, wherein, when the rotor is rotated relative to the stator to a fourth position different from the first plurality of positions, the second plurality of positions, and the third position, the first open channel does not fluidly couple any of the plurality of selector ports to the common port, the second open channel does not fluidly couple the output port to the return port, and the first open channel fluidly couples the return port to the common port.
88. The bioreactor system of embodiment 87, wherein, when the rotor is rotated relative to the stator to the fourth position, the plurality of selector ports and the common port do not concurrently coincide with the first open channel, the return port and the output port do not concurrently coincide with the second open channel, and the return port and the common port concurrently coincide with the first open channel.
89. The bioreactor system of embodiment 83, wherein the first open channel has a circumferential portion with which the common port always coincides when the rotor is rotated relative to the stator to the first plurality of positions and the second plurality of positions, an axial portion extending from the circumferential portion, and a circumferential spur portion extending from the axial portion, wherein the plurality of selector ports respectively coincide with one of the axial portion and the circumferential spur portion of the first open channel when the rotor is rotated relative to the stator to the first plurality of positions, and respectively coincide with the other of the axial portion and the circumferential spur portion of the first open channel when the rotor is rotated relative to the stator to the second plurality of positions; and wherein the second open channel has a circumferential portion with which the output port always coincides when the rotor is rotated relative to the stator to the first plurality of positions and the second plurality of positions, and a plurality of axial spur portions extending from the circumferential portion and with which the return port respectively coincides when the rotor is rotated relative to the stator to the first plurality of positions, and with which the return port does not coincide when the rotor is rotated relative to the stator to the second plurality of positions.
90. The bioreactor system of embodiment 89, wherein the one of the axial portion and the circumferential spur portion of the first open channel is the circumferential spur portion, and the other of the axial portion and the circumferential spur portion is the axial portion.
91. The bioreactor system of embodiment 89, wherein, when the rotor is rotated relative to the stator to a third position different from the first plurality of positions and the second plurality of positions, none of the plurality of selector ports coincide with the axial spur portion or the circumferential spur portion of the first open channel, the return port does not coincide with any of the plurality of axial spur portions of the second open channel, and the return port does not coincide with the axial portion of the first open channel.
92. The bioreactor system of embodiment 91, wherein, when the rotor is rotated relative to the stator to a fourth position different from the first plurality of positions, the second plurality of positions, and the third position, none of the plurality of selector ports coincide with the axial spur portion or the circumferential spur portion of the first open channel, the return port does not coincide with any of the plurality of axial spur portions of the second open channel, and the return port coincides with the axial portion of the first open channel.
93. The bioreactor system of any one of embodiments 83-92, further comprising a motor configured for rotating the rotor relative to the stator.
94. The bioreactor system of embodiment 93, further comprising a shaft having one end affixed to the motor, and another end magnetically coupled to the rotor.
95. The bioreactor system of any one of embodiments 70-94, further comprising an electric controller configured for operating the first multi-function valve and the first pump.
96. The bioreactor system of embodiment 70, wherein the first pump is fluidly coupled between the first multi-function valve and the first port of the one bioreactor.
97. The bioreactor system of embodiment 70, wherein the porous membrane of each of the at least one bioreactor is configured for supporting the respective population of biological cells thereon.
98. A bioreactor system, comprising: a plurality of bioreactor units, each configured for containing a population of biological cells and comprising a first flow channel, a second flow channel, a porous membrane disposed between the first and second flow channels, and a first port and a second port associated with the first flow channel; a first multi-function valve comprising a common port configured for being fluidly coupled to one of a first media source and a first media destination, a plurality of selector ports respectively fluidly coupled to the first ports of the plurality of bioreactor units, a plurality of return ports respectively fluidly coupled to the second ports of the plurality of bioreactor units, and an output port configured for being fluidly coupled to a second media destination, the first multi-function valve having: a first plurality of positions for respectively fluidly coupling the plurality of selector ports to the common port, while respectively fluidly coupling the plurality of return ports to the output port; and a second plurality of positions for respectively fluidly coupling the plurality of selector ports to the common port, while respectively fluidly decoupling all of the plurality of return ports from the output port; and a first pump fluidly coupled to one of the common port and the output port of the first multi-function valve.
99. The bioreactor system of embodiment 98, wherein the first multi-function valve has a third position different from the first plurality of positions and the second plurality of positions for fluidly decoupling all of the plurality of selector ports from the common port, fluidly decoupling all of the plurality of return ports from the output port, and for fluidly decoupling the output port from the common port.
100. The bioreactor system of embodiment 98, wherein the first multi-function valve has a third position different from the first plurality of positions and the second plurality of positions for fluidly coupling the output port to the common port, while fluidly decoupling all of the plurality of selector ports from the common port, and fluidly decoupling all of the plurality of return ports from the output port.
101. The bioreactor system of any one of embodiments 98-100, wherein the one of the first media source and the first media destination comprises one of a biological cell source, a microbead source, a nutrient source, a reagent source, a gas source, a wash buffer source, a saline source, and a biological cell collection destination.
102. The bioreactor system of embodiment 101, wherein the second media destination comprises a waste destination.
103. The bioreactor system of any one of embodiments 98-102, wherein each of the plurality of bioreactor units further comprises a third port and a fourth port associated with the second flow channel, and the bioreactor system further comprising a second multi-function valve comprising a common port configured for being fluidly coupled to the one of the first media source and the first media destination, a plurality of selector ports respectively fluidly coupled to the third ports of the plurality of bioreactor units, a plurality of return ports respectively fluidly coupled to the fourth ports of the plurality of bioreactor units, and an output port configured for being fluidly coupled to the second media destination, the first multi-function valve having: a first plurality of positions for respectively fluidly coupling the plurality of selector ports to the common port, while respectively fluidly coupling the plurality of return ports to the output port; and a second plurality of positions for respectively fluidly coupling the plurality of selector ports to the common port, while respectively fluidly decoupling all of the plurality of return ports from the output port; and a second pump fluidly coupled to one of the common port and the output port of the second multi-function valve.
104. The bioreactor system of any one of embodiments 98-103, wherein the first multi-function valve comprises a first valve portion that respectively fluidly couples the plurality of selector ports to the common port when the first multi-function valve is respectively in the first plurality of positions or the second plurality of positions, and a second first valve portion that respectively fluidly couples the plurality of return ports to the output port when the multi-function valve is in the first plurality of positions, and fluidly decouples all of the plurality of return ports from the output port when the first multi-function valve is in the second plurality of positions.
105. The bioreactor system of any one of embodiments 98-104, further comprising a base bioreactor unit different from the plurality of bioreactor units, the bioreactor system further comprising: a second multi-function valve comprising a common port fluidly coupled to the common port of the first multi-function valve, a return port fluidly coupled to the second port of the base bioreactor unit, a plurality of selector ports configured for respectively being fluidly coupled to a plurality of media sources and/or media destinations, and an output port configured for being fluidly coupled to the second media destination, the first multi-function valve having: a first plurality of positions for respectively fluidly coupling the plurality of selector ports to the common port, such that one of the plurality of media sources and/or media destinations is selected as the one of the first media source and the first media destination that is fluidly coupled to the common port of the first multi-function valve, while fluidly coupling the output port to the return port; and a second plurality of positions different from the first plurality of positions for respectively fluidly coupling the plurality of selector ports to the common port, such that one of the plurality of media sources is selected as the one of the first media source and the first media destination that is fluidly coupled to the common port of the first multi-function valve, while fluidly decoupling the output port from the return port.
106. The bioreactor system of any one of embodiments 98-105, wherein the first multi-function valve is a rotary valve.
107. The bioreactor system of embodiment 106, wherein the rotary valve comprises a stator in which the common port, the plurality of selector ports, the plurality of return ports, and the output port are affixed, and a rotor configured for being rotated relative to the stator to the first plurality of positions and the second plurality of positions, the rotor comprising a first open channel and a second open channel, wherein, when the rotor is rotated relative to the stator to the first plurality of positions, the first open channel respectively fluidly couples the plurality of selector ports to the common port, while the second open channel respectively fluidly couples the plurality of return ports to the output port, and wherein, when the rotor is rotated relative to the stator to the second plurality of positions, the first open channel respectively fluidly couples the plurality of selector ports to the common port, while the second open channel does not fluidly couple any of the plurality of return ports to the output port.
108. The bioreactor system of embodiment 107, wherein, when the rotor is rotated relative to the stator to each of the first plurality of positions, a respective one of the plurality of selector ports and the common port concurrently coincide with the first open channel, while a respective one of the plurality of return ports and the output port concurrently coincide with the second open channel; and wherein, when the rotor is rotated relative to the stator to each of the second plurality of positions, a respective one of the plurality of selector ports and the common port concurrently coincide with the first open channel, while none of the plurality of return ports and the output port concurrently coincide with the second open channel.
109. The bioreactor system of embodiment 107 or 108, wherein, when the rotor is rotated relative to the stator to a third position different from the first plurality of positions and the second plurality of positions, the first open channel does not fluidly couple any of the plurality of selector ports to the common port, the second open channel does not fluidly couple any of the plurality of return ports to the output port, and the first open channel does not fluidly couple the output port to the common port.
110. The bioreactor system of embodiment 109, wherein, when the rotor is rotated relative to the stator in the third position, the plurality of selector ports and the common port do not concurrently coincide with the first open channel, the plurality of return ports and the output port do not concurrently coincide with the second open channel, and the output port and the common port do not concurrently coincide with the first open channel.
111. The bioreactor system of embodiment 109 or 110, wherein, when the rotor is rotated relative to the stator to a fourth position different from the first plurality of positions, the second plurality of positions, and the third position, the first open channel does not fluidly couple any of the plurality of selector ports to the common port, the second open channel does not fluidly couple any of the plurality of return ports to the output port, and the first open channel fluidly couples the output port to the common port.
112. The bioreactor system of embodiment 111, wherein, when the rotor is rotated relative to the stator to the fourth position, the plurality of selector ports and the common port do not concurrently coincide with the first open channel, the plurality of return ports and the output port do not concurrently coincide with the second open channel, and the output port and the common port concurrently coincide with the first open channel.
113. The bioreactor system of any one of embodiments 107-112, wherein the first open channel has a circumferential portion with which the common port always coincides when the rotor is rotated relative to the stator to the first plurality of positions and the second plurality of positions, an axial portion extending from the circumferential portion, and a circumferential spur portion extending from the axial portion, wherein the plurality of selector ports respectively coincide with one of the axial portion and the circumferential spur portion of the first open channel when the rotor is rotated relative to the stator to the first plurality of positions, and respectively coincide with the other of the axial portion and the circumferential spur portion of the first open channel when the rotor is rotated relative to the stator to the second plurality of positions; and wherein the second open channel has a circumferential portion with which the output port coincides when the rotor is rotated relative to the stator to the first plurality of positions and the second plurality of positions, and an axial spur portion extending from the circumferential portion, wherein the plurality of return ports respectively coincide with the axial spur portion when the rotor is rotated relative to the stator to the first plurality of positions, and none of the plurality of return ports coincide with axial spur portion when the rotor is rotated relative to the stator to the second plurality of positions.
114. The bioreactor system of embodiment 113, wherein the one of the axial portion and the circumferential spur portion of the first open channel is the circumferential spur portion, and the other of the axial portion and the circumferential spur portion is the axial portion.
115. The bioreactor system of embodiment 114, wherein, when the rotor is rotated relative to the stator to a third position different from the first plurality of positions and the second plurality of positions, none of the plurality of selector ports coincide with the axial spur portion or the circumferential spur portion of the first open channel, none of the plurality of the return ports coincide with the axial spur portion of the second open channel, and the output port does not coincide with the axial portion of the first open channel.
116. The bioreactor system of embodiment 115, wherein, when the rotor is rotated relative to the stator to a fourth position different from the first plurality of positions, the second plurality of positions, and the third position, none of the plurality of selector ports coincide with the axial spur portion or the circumferential spur portion of the first open channel, none of the plurality of the return ports coincide with the axial spur portion of the second open channel, and the output port coincides with the axial portion of the first open channel.
117. The bioreactor system of any one of embodiments 107-116, further comprising a motor configured for rotating the rotor relative to the stator.
118. The bioreactor system of embodiment 117, further comprising a shaft having one end affixed to the motor, and another end magnetically coupled to the rotor.
119. The bioreactor system of any one of embodiments 98-118, further comprising an electric controller configured for operating the first multi-function valve and the first pump.
120. The bioreactor system of any one of embodiments 98-119, wherein the first pump is fluidly coupled between the first multi-function valve and the first port of the one bioreactor.
121. The bioreactor system of any one of embodiments 98-120, wherein the porous membrane of each of the plurality of bioreactor units is configured for supporting the respective population of biological cells thereon.
122. A bioreactor system, comprising: a plurality of bioreactor units, each configured for containing a population of biological cells and comprising a first flow channel, a second flow channel, a porous membrane disposed between the first and second flow channels, and a first port and a second port associated with the first flow channel, the plurality of bioreactor units comprising a base bioreactor unit and at least one supplemental bioreactor unit; a first multi-function valve comprising a common port, a return port fluidly coupled to the second port of the base bioreactor unit, at least one selector port configured for respectively being fluidly coupled to a first at least one media source and/or media destination, and an output port configured for being fluidly coupled to a first media destination, the first multi-function valve having: a first at least one position for respectively fluidly coupling the at least one selector port to the common port, while fluidly coupling the output port to the return port; and a second at least one position different from the first at least one position for respectively fluidly coupling the at least one selector port to the common port, while fluidly decoupling the output port from the return port; a second multi-function valve comprising a common port fluidly coupled to the common port of the first multi-function valve, and at least one selector port respectively fluidly coupled to the first ports of the at least one supplemental bioreactor unit, at least one return port respectively fluidly coupled to the second ports of the at least one supplemental bioreactor unit, and an output port configured for being fluidly coupled to the first port of the base bioreactor unit, the second multi-function valve having: a first at least one position for respectively fluidly coupling the at least one selector port to the common port, while respectively fluidly coupling the at least one return port to the output port; and a second at least one position for respectively fluidly coupling the at least one selector port to the common port, while respectively fluidly decoupling all of the at least one return port from the output port; and a first pump fluidly coupled between the common port of the first multi-function valve and the common port of the second multi-function valve, or between the output port of the second multi-function valve and the first port of the base bioreactor unit, or between the second port of the base bioreactor unit and the return port of the first multi-function valve.
123. The bioreactor system of embodiment 122, wherein the first at least one media source and/or media destination comprises a first plurality of media sources and/or media destinations, and the at least one selector port of the first multi-function valve comprises a plurality of selector ports; and wherein the at least one supplemental bioreactor unit comprises a plurality of supplemental bioreactor units, the at least one selector port of the second multi-function valve comprises a plurality of selector ports, and the at least one return port of the second multi-function valve comprises a plurality of return ports.
124. The bioreactor system of embodiment 122 or 123, wherein the first multi-function valve has a third position different from the first at least one position and the second at least one position for fluidly decoupling all of the at least one selector port from the common port, for fluidly decoupling the output port from the return port, and for fluidly decoupling the return port from the common port; and wherein the second multi-function valve has a third position different from the first at least one position and the second at least one position for fluidly decoupling all of the at least one selector port from the common port, fluidly decoupling all of the at least one return port from the output port, and for fluidly decoupling the output port from the common port.
125. The bioreactor system of embodiment 122 or 123, wherein the first multi-function valve has a third position different from the first at least one position and the second at least one position for fluidly coupling the return port to the common port, while fluidly decoupling all of the at least one selector port from the common port, and while fluidly decoupling the output port from the return port.
126. The bioreactor system of embodiment 122 or 123, wherein the first multi-function valve has a third position different from the first at least one position and the second at least one position for fluidly coupling the output port to the common port, while fluidly decoupling all of the plurality of selector ports from the common port, and fluidly decoupling all of the plurality of return ports from the output port.
127. The bioreactor system of any one of embodiments 122-126, wherein the first at least one media source and/or media destination comprises one of a biological cell source, a microbead source, a nutrient source, a reagent source, a gas source, a wash buffer source, a saline source, and a biological cell collection destination.
128. The bioreactor system of embodiment 127, wherein the first media destination comprises a waste destination.
129. The bioreactor system of any one of embodiments 122-128, wherein each of the plurality of bioreactor units further comprises a third port and a fourth port associated with the second flow channel, and the bioreactor system further comprising: a third multi-function valve comprising a common port, a return port fluidly coupled to the fourth port of the one bioreactor, at least one selector port configured for respectively being fluidly coupled to a second at least one media source and/or media destination, and an output port configured for being fluidly coupled to a second media destination, the third multi-function valve having: a first at least one position for respectively fluidly coupling the at least one selector port to the common port, while fluidly coupling the output port to the return port; and a second at least one position different from the third position for respectively fluidly coupling the at least one selector port to the common port, while fluidly decoupling the output port from the return port; and a fourth multi-function valve comprising a common port fluidly coupled to the common port of the third multi-function valve, and at least one selector port respectively fluidly coupled to the third ports of the at least one supplemental bioreactor unit, at least one return port respectively fluidly coupled to the fourth ports of the at least one supplemental bioreactor unit, and an output port configured for being fluidly coupled to the third port of the base bioreactor unit, the fourth multi-function valve having: a first at least one position for respectively fluidly coupling the at least one selector port to the common port, while respectively fluidly coupling the at least one return port to the output port; and a second at least one position for respectively fluidly coupling the at least one selector port to the common port, while respectively fluidly decoupling all of the at least one return port from the output port; and a second pump fluidly coupled between the common port of the third multi-function valve and the common port of the fourth multi-function valve, or between the output port of the fourth multi-function valve and the third port of the base bioreactor unit, or between the fourth port of the base bioreactor unit and the return port of the third multi-function valve.
130. The bioreactor system of embodiment 129, wherein at least some of the first at least one media source and/or media destination are respectively the same as at least some of the second at least one media source and/or media destination.
131. The bioreactor system of embodiment 129, wherein the first media destination and the second media destination are the same.
132. The bioreactor system of any one of embodiments 122-131, wherein the first multi-function valve comprises a first valve portion that respectively fluidly couples the at least one selector port to the common port when the first multi-function valve is respectively in the first at least one position or the second at least one position, and a second valve portion that fluidly couples the output port to the return port when the multi-function valve is in any of the first at least one position, and fluidly decouples the output port from the return port when the first multi-function valve is in any of the second at least one position; and wherein the second multi-function valve comprises a first valve portion that respectively fluidly couples the at least one selector port to the common port when the second multi-function valve is respectively in the first at least one position or the second at least one position, and a second valve portion that respectively fluidly couples the at least one return port to the output port when the second multi-function valve is in the first at least one position, and fluidly decouples all of the at least one return port from the output port when the second multi-function valve is in the second at least one position.
133. The bioreactor system of any one of embodiments 122-132, wherein the first multi-function valve is a first rotary valve, and the second multi-function valve is a second rotary valve.
134. The bioreactor system of embodiment 133, wherein the first rotary valve comprises a stator in which the common port, the return port, the at least one selector port, and the output port are affixed, and a rotor configured for being rotated relative to the stator to the first at least one position and the second at least one position; and wherein the second rotary valve comprises a stator in which the common port, the at least one selector port, the output port, and the return port are affixed, and a rotor configured for being rotated relative to the stator to the first at least one position and the second at least one position.
135. The bioreactor system of embodiment 134, further comprising: a first motor configured for rotating the rotor relative to the stator of the first rotary valve; and a second motor configured for rotating the rotor relative to the stator of the second rotary valve.
136. The bioreactor system of embodiment 135, further comprising: a first shaft having one end affixed to the first motor, and another end magnetically coupled to the rotor of the first rotary valve; and a second shaft having one end affixed to the second motor, and another end magnetically coupled to the rotor of the second rotary valve.
137. The bioreactor system of any one of embodiments 122-136, further comprising an electric controller configured for operating the first multi-function valve, the second multi-function valve, and the first pump.
138. The bioreactor system of any one of embodiments 122-137, wherein the porous membrane of each of the plurality of bioreactor units is configured for supporting the respective population of biological cells thereon.
139. A rotary valve, comprising: a stator comprising a common port, a plurality of selector ports, an output port, and at least one return port; and a rotor configured for being rotated relative to the stator to a first plurality of positions that respectively fluidly couples the plurality of selector ports to the common port, while respectively fluidly coupling the at least one return port to the output port, and for being rotated relative to the stator to a second plurality of positions different from the first plurality of positions that fluidly couples the plurality of selector ports to the common port, while fluidly decoupling all of the at least one return port from the output port.
140. The rotary valve of embodiment 139, wherein the at least one return port is a single return port.
141. The rotary valve of embodiment 140, wherein the rotor is configured for being rotated relative to the stator to a third position different from the first plurality of positions and the second plurality of positions that fluidly decouples all of the plurality of selector ports from the common port, fluidly decouples the single return port from the output port, and fluidly decouples the single return port from the common port.
142. The rotary valve of embodiment 140, wherein the rotor is configured for being rotated relative to the stator to a third position different from the first plurality of positions and the second plurality of positions that fluidly couples the single return port to the common port, while fluidly decoupling all of the plurality of selector ports from the common port, and fluidly decoupling the single return port from the output port.
143. The rotary valve of embodiment 139, wherein the at least one return port is a plurality of return ports.
144. The rotary valve of embodiment 143, wherein the rotor is configured for being rotated relative to the stator to a third position different from the first plurality of positions and the second plurality of positions that fluidly decouples all of the plurality of selector ports from the common port, fluidly decouples all of the plurality of return ports from the output port, and fluidly decouples the output port from the common port.
145. The rotary valve of embodiment 144, wherein the rotor is configured for being rotated relative to the stator to a fourth position different from the first plurality of positions, the second plurality of positions, and the third position, that fluidly couples the output port to the common port, while fluidly decoupling all of the plurality of selector ports from the common port, and fluidly decoupling all of the plurality of return ports from the output port.
146. The rotary valve of any one of embodiments 139-145, wherein the rotor has a first open channel that respectively fluidly couples the plurality of selector ports to the common port when the rotor is rotated relative to the stator respectively in the first plurality of positions or the second plurality of positions, and a second open channel that respectively fluidly couples the at least one return port to the output port when the rotor is rotated relative to the stator to the first plurality of positions, and fluidly decouples all of the at least one return port from the output port when the rotor is rotated relative to the stator to the second plurality of positions.
147. The rotary valve of embodiment 146, wherein, when the rotor is rotated relative to the stator to a third position different from the first plurality of positions and the second plurality of positions, the first open channel does not fluidly couple any of the plurality of selector ports to the common port, the second open channel does not fluidly any of couple the at least one return port to the output port, and the first open channel does not fluidly couple any of the at least one return port or the output port to the common port.
148. The rotary valve of embodiment 147, wherein, when the rotor is rotated relative to the stator to a fourth position different from the first plurality of positions, the second plurality of positions, and the third position, the first open channel does not fluidly couple any of the plurality of selector ports to the common port, the second open channel does not fluidly couple any of the at least one return port to the output port, and the first open channel fluidly couples either one of the at least one return port or the output port to the common port.
149. The rotary valve of embodiment 148, wherein the at least one return port is a single return port, the first open channel does not fluidly couple the single return port to the common port when the rotor is rotated relative to the stator to the third position, and fluidly couples the single return port to the common port when the rotor is rotated relative to the stator to the fourth position.
150. The rotary valve of embodiment 148, wherein the at least one return port is a plurality of return ports, the first open channel does not fluidly couple any of the plurality of return ports to the common port when the rotor is rotated relative to the stator to the third position, and fluidly couples the output to the common port when the rotor is rotated relative to the stator to the fourth position.
151. The rotary valve of embodiment 146, wherein, when the rotor is rotated relative to the stator to each of the first plurality of positions, a respective one of the plurality of selector ports and the common port concurrently coincide with the first open channel, while a respective one of the at least one return port and the output port concurrently coincide with the second open channel; and wherein, when the rotor is rotated relative to the stator to each of the second plurality of positions, a respective one of the plurality of selector ports and the common port concurrently coincide with the first open channel, while none of the at least one return port and the output port concurrently coincide with the second open channel.
152. The rotary valve of embodiment 151, wherein, when the rotor is rotated relative to the stator in a third position different from the first plurality of positions and the second plurality of positions, the plurality of selector ports and the common port do not concurrently coincide with the first open channel, the at least one return port and the output port do not concurrently coincide with the second open channel, and the return port, the output port, and the common port do not concurrently coincide with the first open channel.
153. The rotary valve of embodiment 152, wherein, when the rotor is rotated relative to the stator to a fourth position different from the first plurality of positions, the second plurality of positions, and the third position, the plurality of selector ports and the common port do not concurrently coincide with the first open channel, the at least one return port and the output port do not concurrently coincide with the second open channel, and either the at least one return port or the output port and the common port concurrently coincide with the first open channel.
154. The rotary valve of embodiment 153, wherein the at least one return port is a single return port, and the first open channel fluidly couples the single return port to the common port when the rotor is rotated relative to the stator to the fourth position.
155. The rotary valve of embodiment 153, wherein the at least one return port is a plurality of return ports, and the first open channel fluidly couples the output to the common port when the rotor is rotated relative to the stator to the fourth position.
156. The rotary valve of embodiment 146, wherein the first open channel has a circumferential portion with which the common port always coincides when the rotor is rotated relative to the stator to the first plurality of positions and the second plurality of positions, an axial portion extending from the circumferential portion, and a circumferential spur portion extending from the axial portion, wherein the plurality of selector ports respectively coincide with one of the axial portion and the circumferential spur portion of the first open channel when the rotor is rotated relative to the stator to the first plurality of positions, and respectively coincide with the other of the axial portion and the circumferential spur portion of the first open channel when the rotor is rotated relative to the stator to the second plurality of positions; and wherein the second open channel has a circumferential portion with which the output port always coincides when the rotor is rotated relative to the stator to the first plurality of positions and the second plurality of positions, and at least one axial spur portion extending from the circumferential portion and with which one of the at least one return port coincides when the rotor is rotated relative to the stator to the first plurality of positions, and with which none of the at least one return port coincides when the rotor is rotated relative to the stator to the second plurality of positions.
157. The bioreactor system of embodiment 156, wherein the one of the axial portion and the circumferential spur portion of the first open channel is the circumferential spur portion, and the other of the axial portion and the circumferential spur portion is the axial portion.
158. The rotary valve of embodiment 157, wherein the at least one return port is a single return port, and the at least one axial spur portion comprises a plurality of axial spur portions with which the single return port respectively coincides when the rotor is rotated relative to the stator to the first plurality of positions, and with which the single return port does not coincide when the rotor is rotated relative to the stator to the second plurality of positions.
159. The rotary valve of embodiment 157, wherein the at least one return port is a plurality of return ports, the at least one axial spur portion is a single axial spur portion with which the plurality of return ports respectively coincide when the rotor is rotated relative to the stator to the first plurality of positions, and with which the plurality of return ports respectively do not coincide when the rotor is rotated relative to the stator to the second plurality of positions.
160. The rotary valve of embodiment 157, wherein, when the rotor is rotated relative to the stator to a third position different from the first plurality of positions and the second plurality of positions, none of the plurality of selector ports coincide with the axial spur portion or the circumferential spur portion of the first open channel, none of the at least one return port coincides with the axial spur portion of the second open channel, and none of the at least one return output port and the output port coincides with the axial portion of the first open channel.
161. The rotary valve of embodiment 160, wherein, when the rotor is rotated relative to the stator to a fourth position different from the first plurality of positions, the second plurality of positions, and the third position, none of the plurality of selector ports coincide with the axial spur portion or the circumferential spur portion of the first open channel, none of the plurality of the return ports coincides with the axial spur portion of the second open channel, and either one of the at least one return port or the output port coincides with the axial portion of the first open channel.
162. The rotary valve of embodiment 161, wherein the at least one return port is a single return port that coincides with the axial portion of the first open channel when the rotor is rotated relative to the stator to the fourth position.
163. The rotary valve of embodiment 161, wherein the at least one return port is a plurality of return ports, and the output port coincides with the axial portion of the first open channel when the rotor is rotated relative to the stator to the fourth position

Although particular embodiments have been shown and described herein, it will be understood by those skilled in the art that they are not intended to limit the disclosed inventions, and it will be obvious to those skilled in the art that various changes, permutations, and modifications may be made (e.g., the dimensions of various parts, combinations of parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

1. A bioreactor assembly, comprising:
a plurality of bioreactor units arranged in a flow sequence; and
at least one flow path extending through the bioreactor units.

2. The bioreactor assembly of claim 1, wherein the plurality of bioreactor units is stacked on top of each other.

3. The bioreactor assembly of claim 1, wherein each of the plurality of bioreactor units is configured for expanding a population of biological cells.

4. The bioreactor assembly of claim 1, wherein the at least one flow path comprises at least two different flow paths.

5. The bioreactor assembly of claim 1, wherein the at least one flow path comprises three different flow paths.

6. The bioreactor assembly of claim 1, wherein the at least one flow path selectively extends through the plurality of bioreactor units, such that selected ones of the plurality of bioreactor units may be added to the at least one flow path, and selected ones of the plurality of bioreactor units may be removed from the at least one flow path.

7. The bioreactor assembly of claim 1, wherein each of the plurality of bioreactor units comprises:
at least one flow channel into which media can be flowed;
at least one inlet port respectively fluidly coupled to the at least one flow channel, such that media can be respectively flowed into the at least one flow channel; and
at least one outlet port respectively fluidly coupled to the at least one flow channel, such that media can be respectively flowed out of the at least one flow channel;
wherein each of the at least one outlet port of each of the plurality of bioreactor units preceding the last bioreactor unit in the flow sequence is respectively fluidly coupled to an immediately succeeding bioreactor unit in the flow sequence, such that the at least one flow path respectively extends entirely through the at least one flow channel of each of the plurality of bioreactor units.

8-11. (canceled)

12. The bioreactor assembly of claim 7,
wherein the at least one flow channel of each of the plurality of bioreactor units comprises two flow channels;
wherein the at least one inlet port of each of the plurality of bioreactor units comprises two inlet ports respectively fluidly coupled to the two flow channels;
wherein the at least one outlet port of each of the plurality of bioreactor units comprises two outlet ports respectively fluidly coupled to the two flow channels; and
wherein the at least one flow path comprises a first flow path extending in through the inlet port and out through the outlet port of a first one of the two flow channels of each of the plurality of bioreactor units, and a second flow path different from the first flow path extending in through the inlet port and out through the outlet port of a second one of the two flow channels of each of the plurality of bioreactor units.

13. (canceled)

14. The bioreactor assembly of claim 12, wherein the at least one flow path comprises a third flow path different from the first flow path and the second flow path extending in through the inlet port of the first flow channel, between the first flow channel and the second flow channel, and out through the outlet port of the second flow channel, of each of the plurality of bioreactor units.

15. The bioreactor assembly of claim 12,
wherein each of the plurality of bioreactor units comprises a cell culture surface contained within the first flow channel of the respective bioreactor unit;
wherein first media containing biological cells can be flowed into the first flow channel, thereby disposing a population of biological cells on the cell culture surface of each of the plurality of bioreactor units; and
wherein second media containing one or more of nutrient, reagent, and gas can be flowed along the second flow path through the second flow channel of each of the plurality of bioreactor units, thereby facilitating the expansion of the populations of biological cells respectively within the plurality of bioreactor units.

16. The bioreactor assembly of claim 15, wherein third media can be flowed along the first flow path through the first flow channel of each of the plurality of bioreactor units, thereby harvesting the expanded populations of biological cells from the respective plurality of bioreactor units.

17. The bioreactor assembly of claim 15, wherein each of the plurality of bioreactor units comprises a first membrane between the first flow channel and the second flow channel of the respective bioreactor unit, the first membrane being substantially permeable to the one or more of nutrient, reagent, and gas contained within the second media, and waste produced by the populations of biological cells, thereby facilitating expansion of the populations of biological cells within the first flow channels of the respective plurality of bioreactor units, while being substantially impermeable to the populations of biological cells.

18. The bioreactor assembly of claim 17,
wherein the at least one flow path comprises a third flow path extending in through the inlet port of the first flow channel, between the first flow channel and the second flow channel, and out through the outlet port of the second flow channel, of each of the plurality of bioreactor units; and
wherein the first membrane of each of the plurality of bioreactor units is a porous membrane configured for allowing perfusion of the first media along the third flow path from the first flow channels into the second flow channels of the respective plurality of bioreactor units, while filtering the populations of biological cells from the first media, such that the populations of biological cells are retained within the first flow channels of the respective plurality of bioreactor units.

19. The bioreactor assembly of claim 18, wherein each of the plurality of bioreactor units comprises further comprises a second membrane, wherein the first flow channels are disposed between the first membranes and the second membranes of the respective plurality of bioreactor units, and wherein the second membrane of each of the plurality of bioreactor units is substantially permeable to gas from a space exterior to the first flow channels of the respective plurality of bioreactor units, thereby facilitating expansion of the populations of biological cells within the first flow channels of the respective plurality of bioreactor units, while being substantially impermeable to the populations of biological cells and the first media.

20. The bioreactor assembly of claim 19, wherein the cell culture surface is formed on the second membrane.

21. A bioreactor system, comprising:
the bioreactor assembly of claim 1; and
a a valve assembly configured for selectively fluidly coupling ones of the plurality of bioreactor units to the at least one flow path, and selectively fluidly decoupling ones of the plurality of bioreactor units from the at least one flow path.

22-23. (canceled)

24. A bioreactor system, comprising:
the bioreactor assembly of claim 7; and
a valve assembly configured for selectively fluidly coupling each of the at least one outlet port of each of the plurality of bioreactor units preceding the last bioreactor unit of the flow sequence respectively to the immediately succeeding bioreactor unit in the flow sequence.

25. A method of operating the bioreactor assembly of claim 1, comprising:
seeding the plurality of bioreactor units respectively with populations of biological cells;
flowing first media containing one or more of nutrient, reagent, and gas along a first one of the at least one flow path, thereby facilitating the expansion of the populations of biological cells respectively within the plurality of bioreactor units; and
harvesting the expanded populations of biological cells respectively from the plurality of bioreactor units.

26-29. (canceled)

30. A method of operating the bioreactor assembly of claim 15, comprising:
flowing the first media containing the biological cells into the first flow channels of the respective plurality of bioreactor units, thereby respectively depositing the populations of biological cells on the cell culture surfaces of the respective plurality of bioreactor units; and
flowing the second media containing the one or more of nutrient, reagent, and gas along the second flow path through the second flow channel of each of the plurality of bioreactor units, thereby facilitating the expansion of the populations of biological cells within the second flow channels of the respective plurality of bioreactor units.

31-33. (canceled)

34. A method of operating the bioreactor assembly of claim 18, comprising:
flowing the first media containing the biological cells along the third flow path into the first flow channels of the respective plurality of bioreactor units;
perfusing the first media along the third flow path from the first flow channels, through the first porous membranes, and into the second flow channels of the respective plurality of bioreactor units, while substantially retaining the biological cells within the first flow channels of the respective plurality of bioreactor units, such that the populations of biological cells are disposed on the cell culture surfaces of the respective plurality of bioreactor units;
flowing the perfused first media along the third flow path out of the second flow channels of the respective plurality of bioreactor units;
flowing the second media containing the one or more of nutrient, reagent, and gas along the second flow path through the second flow channels of the respective plurality of bioreactor units; and
diffusing the one or more of nutrient, reagent, and gas from the second flow channels, through the first membranes, and into the second flow channels, and diffusing the waste from the first flow channels, through the first membranes, and into the second flow channels, of the respective plurality of bioreactor units, thereby facilitating the expansion of the populations of biological cells within the first flow channels of the respective plurality of biological cells.

35-51. (canceled)
